SEQUENCE LISTING (136 sequences)

SEQ ID NO: 1 - DNA, 7154 bp, synthetic construct
  source 1..7154: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatcgattacaaggatgacgatgacaagggtggatcgccaaagaaaaagaggaaagtcggcagtggacctaaaaagaaacgaaaggttgggtcaggtccgaagaaaaagcgcaaggtcgaagcgggatccgcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaaagccccaagaagaagagaaaggtggaggccagcttagcatcgggaggtggttcgggtggaggaagtggcgggtcagggtcgggtggcactagtaagatgaaggagggtgaaaataataaacccagggagaagtcagaaagtaacaagaggaaatccaatttctcaaacagtgccgatgacatcaaatctaaaaaaaagagagagcagagcaatgatatcgctcggggctttgagagaggactggaaccagaaaagatcattggggcaacagattcctgtggtgatttaatgttcctaatgaaatggaaagacacagatgaagctgacctggttcttgcaaaagaagctaatgtgaaatgtccacaaattgtgatagcattttatgaagagagactgacatggcatgcatatgctagcggaggtggttcgccaaagaagaaacggaaggtgtgagcatgcaataaaacatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtccctgaaaagggactaaaataaagagtttgcgggactctgcggggttacaatcccctaaaaccgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 2949 bp, synthetic construct
  source 1..2949: mol_type = other DNA
gcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaa

SEQ ID NO: 3 - DNA, 7028 bp, synthetic construct
  source 1..7028: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatcgattacaaggatgacgatgacaagggtggatcgccaaagaaaaagaggaaagtcggcagtggacctaaaaagaaacgaaaggttgggtcaggtccgaagaaaaagcgcaaggtcgaagcgggatccgcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaaagccccaagaagaagagaaaggtggaggccagcttagcatcgggaggtggttcgggtggaggaagtggcgggtcagggtcgggtggcactagtgcagtagacccaggcctgccttctgtgaagcaagagccacctgacccagaggaggacaaggaggagaacaaggatgattctgcctccaaattggccccagaggaagaggcaggaggggctggcacacccgtgatcacggagattttcagcctgggtggaacccgcttccgagatacagcagtctggttgccagctagcggaggtggttcgccaaagaagaaacggaaggtgtgagcatgcaataaaacatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtccctgaaaagggactaaaataaagagtttgcgggactctgcggggttacaatcccctaaaaccgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 4 - DNA, 7004 bp, synthetic construct
  source 1..7004: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatcgattacaaggatgacgatgacaagggtggatcgccaaagaaaaagaggaaagtcggcagtggacctaaaaagaaacgaaaggttgggtcaggtccgaagaaaaagcgcaaggtcgaagcgggatccgcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaaagccccaagaagaagagaaaggtggaggccagcttagcatcgggaggtggttcgggtggaggaagtggcgggtcagggtcgggtggcactagtgatgtctactacttcagtccaagtggtaagaagttcagaagcaagcctcagttggcaaggtacctgggaaatactgttgatctcagcagttttgacttcagaactggaaagatgatgcctagtaaattacagaagaacaaacagagactgcgaaacgatcctctcaatgctagcggaggtggttcgccaaagaagaaacggaaggtgtgagcatgcaataaaacatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtccctgaaaagggactaaaataaagagtttgcgggactctgcggggttacaatcccctaaaaccgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 5 - DNA, 7082 bp, synthetic construct
  source 1..7082: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatcgattacaaggatgacgatgacaagggtggatcgccaaagaaaaagaggaaagtcggcagtggacctaaaaagaaacgaaaggttgggtcaggtccgaagaaaaagcgcaaggtcgaagcgggatccgcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaaagccccaagaagaagagaaaggtggaggccagcttagcatcgggaggtggttcgggtggaggaagtggcgggtcagggtcgggtggcactagtatgggtggagaggatgatgaactcaagggcttactggggcttccagaggaggaaactgagcttgataacctgacagagttcaacactgcccacaacaagcggatttctacccttaccattgaggagggaaatctggacattcaaagaccaaagaggaagaggaagaactcacgggtgacattcagtgaggatgatgagatcatcaacccagaggatgtggatccctcagttggtcgattcaggaacgctagcggaggtggttcgccaaagaagaaacggaaggtgtgagcatgcaataaaacatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtccctgaaaagggactaaaataaagagtttgcgggactctgcggggttacaatcccctaaaaccgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 6 - DNA, 7022 bp, synthetic construct
  source 1..7022: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatcgattacaaggatgacgatgacaagggtggatcgccaaagaaaaagaggaaagtcggcagtggacctaaaaagaaacgaaaggttgggtcaggtccgaagaaaaagcgcaaggtcgaagcgggatccgcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaaagccccaagaagaagagaaaggtggaggccagcttagcatcgggaggtggttcgggtggaggaagtggcgggtcagggtcgggtggcactagtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccgctagcggaggtggttcgccaaagaagaaacggaaggtgtgagcatgcaataaaacatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtccctgaaaagggactaaaataaagagtttgcgggactctgcggggttacaatcccctaaaaccgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 7 - DNA, 7157 bp, synthetic construct
  source 1..7157: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatcgattacaaggatgacgatgacaagggtggatcgccaaagaaaaagaggaaagtcggcagtggacctaaaaagaaacgaaaggttgggtcaggtccgaagaaaaagcgcaaggtcgaagcgggatccgcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaaagccccaagaagaagagaaaggtggaggccagcttagcatcgggaggtggttcgggtggaggaagtggcgggtcagggtcgggtggcactagttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggaggctagcggaggtggttcgccaaagaagaaacggaaggtgtgagcatgcaataaaacatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtccctgaaaagggactaaaataaagagtttgcgggactctgcggggttacaatcccctaaaaccgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 7367 bp, synthetic construct
  source 1..7367: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatcgattacaaggatgacgatgacaagggtggatcgccaaagaaaaagaggaaagtcggcagtggacctaaaaagaaacgaaaggttgggtcaggtccgaagaaaaagcgcaaggtcgaagcgggatccgcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaaagccccaagaagaagagaaaggtggaggccagcttagcatcgggaggtggttcgggtggaggaagtggcgggtcagggtcgggtggcactagtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggaggctagcggaggtggttcgccaaagaagaaacggaaggtgtgagcatgcaataaaacatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtccctgaaaagggactaaaataaagagtttgcgggactctgcggggttacaatcccctaaaaccgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 9 - DNA, 7772 bp, synthetic construct
  source 1..7772: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatcgattacaaggatgacgatgacaagggtggatcgccaaagaaaaagaggaaagtcggcagtggacctaaaaagaaacgaaaggttgggtcaggtccgaagaaaaagcgcaaggtcgaagcgggatccgcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaaagccccaagaagaagagaaaggtggaggccagcttagcatcgggaggtggttcgggtggaggaagtggcgggtcagggtcgggtggcactagtccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtggctagcggaggtggttcgccaaagaagaaacggaaggtgtgagcatgcaataaaacatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcgctgggccaggtctctgggaggttttagtccctgaaaagggactaaaataaagagtttgcgggactctgcggggttacaatcccctaaaaccgcttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 10 - DNA, 7339 bp, synthetic construct
  source 1..7339: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatagattacaaggatgacgatgacaagggtggatcgccaaagaaaaagaggaaagtcgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccggtggaggaagtggcgggtcagggtcgggtggcactagtaagatgaaggagggtgaaaataataaacccagggagaagtcagaaagtaacaagaggaaatccaatttctcaaacagtgccgatgacatcaaatctaaaaaaaagagagagcagagcaatgatatcgctcggggctttgagagaggactggaaccagaaaagatcattggggcaacagattcctgtggtgatttaatgttcctaatgaaatggaaagacacagatgaagctgacctggttcttgcaaaagaagctaatgtgaaatgtccacaaattgtgatagcattttatgaagagagactgacatggcatgcatatgctagcggaggtggttcgccaaagaagaaacggaaggtgggatcctgaaataaaagatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - DNA, 7306 bp, synthetic construct
  source 1..7306: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatagattacaaggatgacgatgacaagggtggatcgccaaagaaaaagaggaaagtcgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccggtggaggaagtggcgggtcagggtcgggtggcactagtaaatcagagggaggcaagcgcaaagctgattctgattctgaagataagggagaggagagcaaaccaaagaagaagaaagaagagtcagaaaagccacgaggctttgctcgaggtttggagccggagcggattattggagctacagactccagtggagagctcatgttcctgatgaaatggaaaaactctgatgaggctgacctggtccctgccaaggaagccaatgtcaagtgcccacaggttgtcatatccttctatgaggaaaggctgacgtggcattcctacgctagcggaggtggttcgccaaagaagaaacggaaggtgggatcctgaaataaaagatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 12 - DNA, 7213 bp, synthetic construct
  source 1..7213: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatagattacaaggatgacgatgacaagggtggatcgccaaagaaaaagaggaaagtcgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccggtggaggaagtggcgggtcagggtcgggtggcactagtgcagtagacccaggcctgccttctgtgaagcaagagccacctgacccagaggaggacaaggaggagaacaaggatgattctgcctccaaattggccccagaggaagaggcaggaggggctggcacacccgtgatcacggagattttcagcctgggtggaacccgcttccgagatacagcagtctggttgccagctagcggaggtggttcgccaaagaagaaacggaaggtgggatcctgaaataaaagatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 13 - DNA, 7189 bp, synthetic construct
  source 1..7189: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatagattacaaggatgacgatgacaagggtggatcgccaaagaaaaagaggaaagtcgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccggtggaggaagtggcgggtcagggtcgggtggcactagtgatgtctactacttcagtccaagtggtaagaagttcagaagcaagcctcagttggcaaggtacctgggaaatactgttgatctcagcagttttgacttcagaactggaaagatgatgcctagtaaattacagaagaacaaacagagactgcgaaacgatcctctcaatgctagcggaggtggttcgccaaagaagaaacggaaggtgggatcctgaaataaaagatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 14 - DNA, 7267 bp, synthetic construct
  source 1..7267: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatagattacaaggatgacgatgacaagggtggatcgccaaagaaaaagaggaaagtcgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccggtggaggaagtggcgggtcagggtcgggtggcactagtatgggtggagaggatgatgaactcaagggcttactggggcttccagaggaggaaactgagcttgataacctgacagagttcaacactgcccacaacaagcggatttctacccttaccattgaggagggaaatctggacattcaaagaccaaagaggaagaggaagaactcacgggtgacattcagtgaggatgatgagatcatcaacccagaggatgtggatccctcagttggtcgattcaggaacgctagcggaggtggttcgccaaagaagaaacggaaggtgggatcctgaaataaaagatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 15 - DNA, 7207 bp, synthetic construct
  source 1..7207: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatagattacaaggatgacgatgacaagggtggatcgccaaagaaaaagaggaaagtcgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccggtggaggaagtggcgggtcagggtcgggtggcactagtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagcccgctagcggaggtggttcgccaaagaagaaacggaaggtgggatcctgaaataaaagatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 16 - DNA, 7342 bp, synthetic construct
  source 1..7342: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatagattacaaggatgacgatgacaagggtggatcgccaaagaaaaagaggaaagtcgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccggtggaggaagtggcgggtcagggtcgggtggcactagttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggaggctagcggaggtggttcgccaaagaagaaacggaaggtgggatcctgaaataaaagatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 17 - DNA, 7552 bp, synthetic construct
  source 1..7552: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatagattacaaggatgacgatgacaagggtggatcgccaaagaaaaagaggaaagtcgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccggtggaggaagtggcgggtcagggtcgggtggcactagtcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggaggctagcggaggtggttcgccaaagaagaaacggaaggtgggatcctgaaataaaagatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 7956 bp, synthetic construct
  source 1..7956: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactacaaagaccatgacggtgattataaagatcatgacatagattacaaggatgacgatgacaagggtggatcgccaaagaaaaagaggaaagtcgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccggtggaggaagtggcgggtcagggtcgggtggcactagtccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtggctagcggaggtggttcgccaaagaagaaacggaaggtgggatcctgaaataaaagatctttgttttcattagatctgtgtgttggttttttgtgtgcggccggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacctcccagagacctggcccagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 19 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 20 - protein, 1118 aa, Staphylococcus aureus
MDYKDHDGDYKDHDIDYKDDDDKGGSPKKKRKVAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGS

SEQ ID NO: 21 - protein, 1145 aa, Campylobacter jejuni
MDYKDHDGDYKDHDIDYKDDDDKGGSPKKKRKVGSGPKKKRKVGSGPKKKRKVEAGSARILAFAIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDAIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKKSPKKKRKVEALASGGGSGGGSGGSGSGGTSRGFARGLEPERIIGATDSSGELMFLMKWKNSDEADLVPAKEANVKCPQVVISFYEERLTWHSYASGGGSPKKKRKV

SEQ ID NO: 22 - DNA, 4272 bp, Streptococcus pyogenes
  source 1..4272: mol_type = other DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagtga

SEQ ID NO: 23 - DNA, 3306 bp, Staphylococcus aureus
  source 1..3306: mol_type = other DNA
atggactacaaagaccatgacggtgattataaagatcatgacatagattacaaggatgacgatgacaagggtggatcgccaaagaaaaagaggaaagtcgccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggctaa

SEQ ID NO: 24 - DNA, 3156 bp, Staphylococcus aureus
  source 1..3156: mol_type = other DNA
aagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 25 - DNA, 3147 bp, Campylobacter jejuni
  source 1..3147: mol_type = other DNA
atggactacaaagaccatgacggtgattataaagatcatgacatcgattacaaggatgacgatgacaagggtggatcgccaaagaaaaagaggaaagtcggcagtggacctaaaaagaaacgaaaggttgggtcaggtccgaagaaaaagcgcaaggtcgaagcgggatccgcccgcatcctcgctttcgccatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgacgccatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaaagccccaagaagaagagaaaggtgtaa

SEQ ID NO: 26 - DNA, 2949 bp, Campylobacter jejuni; identical to SEQ ID NO: 2
  source 1..2949: mol_type = other DNA

SEQ ID NO: 27 - DNA, 4107 bp, synthetic construct
  source 1..4107: mol_type = other DNA
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgactga

SEQ ID NO: 28 - protein, 108 aa, Homo sapiens
MSKMKEGENNKPREKSESNKRKSNFSNSADDIKSKKKREQSNDIARGFERGLEPEKIIGATDSCGDLMFLMKWKDTDEADLVLAKEANVKCPQIVIAFYEERLTWHAY

SEQ ID NO: 29 - protein, 191 aa, Homo sapiens
MGKKTKRTADSSSSEDEEEYVVEKVLDRRVVKGQVEYLLKWKGFSEEHNTWEPEKNLDCPELISEFMKKYKKMKEGENNKPREKSESNKRKSNFSNSADDIKSKKKREQSNDIARGFERGLEPEKIIGATDSCGDLMFLMKWKDTDEADLVLAKEANVKCPQIVIAFYEERLTWHAYPEDAENKEKETAKS

SEQ ID NO: 30 - protein, 96 aa, Homo sapiens
MKSEGGKRKADSDSEDKGEESKPKKKKEESEKPRGFARGLEPERIIGATDSSGELMFLMKWKNSDEADLVPAKEANVKCPQVVISFYEERLTWHSY

SEQ ID NO: 31 - protein, 185 aa, Homo sapiens
MGKKQNKKKVEEVLEEEEEEYVVEKVLDRRVVKGKVEYLLKWKGFSDEDNTWEPEENLDCPDLIAEFLQSQKTAHETDKSEGGKRKADSDSEDKGEESKPKKKKEESEKPRGFARGLEPERIIGATDSSGELMFLMKWKNSDEADLVPAKEANVKCPQVVISFYEERLTWHSYPSEDDDKKDDKN

SEQ ID NO: 32 - protein, 65 aa, synthetic construct
MAVDPGLPSVKQEPPDPEEDKEENKDDSASKLAPEEEAGGAGTPVITEIFSLGGTRFRDTAVWLP

SEQ ID NO: 33 - protein, 605 aa, Homo sapiens
MAEDWLDCPALGPGWKRREVFRKSGATCGRSDTYYQSPTGDRIRSKVELTRYLGPACDLTLFDFKQGILCYPAPKAHPVAVASKKRKKPSRPAKTRKRQVGPQSGEVRKEAPRDETKADTDTAPASFPAPGCCENCGISFSGDGTQRQRLKTLCKDCRAQRIAFNREQRMFKRVGCGECAACQVTEDCGACSTCLLQLPHDVASGLFCKCERRRCLRIVERSRGCGVCRGCQTQEDCGHCPICLRPPRPGLRRQWKCVQRRCLRGKHARRKGGCDSKMAARRRPGAQPLPPPPPSQSPEPTEPHPRALAPSPPAEFIYYCVDEDELQPYTNRRQNRKCGACAACLRRMDCGRCDFCCDKPKFGGSNQKRQKCRWRQCLQFAMKRLLPSVWSESEDGAGSPPPYRRRKRPSSARRHHLGPTLKPTLATRTAQPDHTQAPTKQEAGGGFVLPPPGTDLVFLREGASSPVQVPGPVAASTEALLQEAQCSGLSWVVALPQVKQEKADTQDEWTPGTAVLTSPVLVPGCPSKAVDPGLPSVKQEPPDPEEDKEENKDDSASKLAPEEEAGGAGTPVITEIFSLGGTRFRDTAVWLPRSKDLKKPGARKQ

SEQ ID NO: 34 - protein, 655 aa, Homo sapiens
MAEDWLDCPALGPGWKRREVFRKSGATCGRSDTYYQSPTGDRIRSKVELTRYLGPACDLTLFDFKQGILCYPAPKAHPVAVASKKRKKPSRPAKTRKRQVGPQSGEVRKEAPRDETKADTDTAPASFPAPGCCENCGISFSGDGTQRQRLKTLCKDCRAQRIAFNREQRMFKRVGCGECAACQVTEDCGACSTCLLQLPHDVASGLFCKCERRRCLRIVERSRGCGVCRGCQTQEDCGHCPICLRPPRPGLRRQWKCVQRRCLRGKHARRKGGCDSKMAARRRPGAQPLPPPPPSQSPEPTEPHPRALAPSPPAEFIYYCVDEDELQPYTNRRQNRKCGACAACLRRMDCGRCDFCCDKPKFGGSNQKRQKCRWRQCLQFAMKRLLPSVWSESEDGAGSPPPYRRRKRPSSARRHHLGPTLKPTLATRTAQPDHTQAPTKQEAGGGFVLPPPGTDLVFLREGASSPVQVPGPVAASTEALLQEAQCSGLSWVVALPQVKQEKADTQDEWTPGTAVLTSPVLVPGCPSKAVDPGLPSVKQEPPDPEEDKEENKDDSASKLAPEEEAGGAGTPVITEIFSLGGTRFRDTAVWLPSLQGRHSGREDGCKVWETEDTVEPTSTSWNPRGWPGTHVSLSPPPASMMWVSCRRSWCPSSQS

SEQ ID NO: 35 - protein, 57 aa, Homo sapiens
MDVYYFSPSGKKFRSKPQLARYLGNTVDLSSFDFRTGKMMPSKLQKNKQRLRNDPLN

SEQ ID NO: 36 - protein, 410 aa, Homo sapiens
MRAHPGGGRCCPEQEEGESAAGGSGAGGDSAIEQGGQGSALAPSPVSGVRREGARGGGRGRGRWKQAGRGGGVCGRGRGRGRGRGRGRGRGRGRGRPPSGGSGLGGDGGGCGGGGSGGGGAPRREPVPFPSGSAGPGPRGPRATESGKRMDCPALPPGWKKEEVIRKSGLSAGKSDVYYFSPSGKKFRSKPQLARYLGNTVDLSSFDFRTGKMMPSKLQKNKQRLRNDPLNQNKGKPDLNTTLPIRQTASIFKQPVTKVTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKKLEEALMADILSRAADTEEMDIEMDSGDE

SEQ ID NO: 37 - protein, 82 aa, Homo sapiens
MGGEDDELKGLLGLPEEETELDNLTEFNTAHNKRISTLTIEEGNLDIQRPKRKRKNSRVTFSEDDEIINPEDVDPSVGRFRN

SEQ ID NO: 38 - protein, 351 aa, Homo sapiens
MAAAANSGSSLPLFDCPTWAGKPPPGLHLDVVKGDKLIEKLIIDEKKYYLFGRNPDLCDFTIDHQSCSRVHAALVYHKHLKRVFLIDLNSTHGTFLGHIRLEPHKPQQIPIDSTVSFGASTRAYTLREKPQTLPSAVKGDEKMGGEDDELKGLLGLPEEETELDNLTEFNTAHNKRISTLTIEEGNLDIQRPKRKRKNSRVTFSEDDEIINPEDVDPSVGRFRNMVQTAVVPVKKKRVEGPGSLGLEESGSRRMQNFAFSGGLYGGLPPTHSEAGSQPHGIHGTALIGGLPMPYPNLAPDVDLTPVVPSAVNMNPAPNPAVYNPEAVNEPKKKKYAKEAWPGKKPTPSLLI

SEQ ID NO: 39 - protein, 63 aa, synthetic construct
MRTLVTFKDVFVDFTREEWKLLDTAQQIVYRNVMLENYKNLVSLGYQLTKPDVILRLEKGEEP

SEQ ID NO: 40 - protein, 108 aa, Homo sapiens
MSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRE

SEQ ID NO: 41 - protein, 178 aa, synthetic construct
MRTLVTFKDVFVDFTREEWKLLDTAQQIVYRNVMLENYKNLVSLGYQLTKPDVILRLEKGEEPSGGGSGGGSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRD

SEQ ID NO: 42 - protein, 311 aa, Homo sapiens
PSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 43 - DNA, 327 bp, Homo sapiens
  source 1..327: mol_type = other DNA
atgagtaagatgaaggagggtgaaaataataaacccagggagaagtcagaaagtaacaagaggaaatccaatttctcaaacagtgccgatgacatcaaatctaaaaaaaagagagagcagagcaatgatatcgctcggggctttgagagaggactggaaccagaaaagatcattggggcaacagattcctgtggtgatttaatgttcctaatgaaatggaaagacacagatgaagctgacctggttcttgcaaaagaagctaatgtgaaatgtccacaaattgtgatagcattttatgaagagagactgacatggcatgcatattga

SEQ ID NO: 44 - DNA, 318 bp, synthetic construct
  source 1..318: mol_type = other DNA
aagatgaaggagggtgaaaataataaacccagggagaagtcagaaagtaacaagaggaaatccaatttctcaaacagtgccgatgacatcaaatctaaaaaaaagagagagcagagcaatgatatcgctcggggctttgagagaggactggaaccagaaaagatcattggggcaacagattcctgtggtgatttaatgttcctaatgaaatggaaagacacagatgaagctgacctggttcttgcaaaagaagctaatgtgaaatgtccacaaattgtgatagcattttatgaagagagactgacatggcatgcatat

SEQ ID NO: 45 - DNA, 291 bp, Homo sapiens
  source 1..291: mol_type = other DNA
atgaaatcagagggaggcaagcgcaaagctgattctgattctgaagataagggagaggagagcaaaccaaagaagaagaaagaagagtcagaaaagccacgaggctttgctcgaggtttggagccggagcggattattggagctacagactccagtggagagctcatgttcctgatgaaatggaaaaactctgatgaggctgacctggtccctgccaaggaagccaatgtcaagtgcccacaggttgtcatatccttctatgaggaaaggctgacgtggcattcctactaa

SEQ ID NO: 46 - DNA, 285 bp, synthetic construct
  source 1..285: mol_type = other DNA
aaatcagagggaggcaagcgcaaagctgattctgattctgaagataagggagaggagagcaaaccaaagaagaagaaagaagagtcagaaaagccacgaggctttgctcgaggtttggagccggagcggattattggagctacagactccagtggagagctcatgttcctgatgaaatggaaaaactctgatgaggctgacctggtccctgccaaggaagccaatgtcaagtgcccacaggttgtcatatccttctatgaggaaaggctgacgtggcattcctac

SEQ ID NO: 47 - DNA, 198 bp, Homo sapiens
  source 1..198: mol_type = other DNA
atggcagtagacccaggcctgccttctgtgaagcaagagccacctgacccagaggaggacaaggaggagaacaaggatgattctgcctccaaattggccccagaggaagaggcaggaggggctggcacacccgtgatcacggagattttcagcctgggtggaacccgcttccgagatacagcagtctggttgccataa

SEQ ID NO: 48 - DNA, 192 bp, Homo sapiens
  source 1..192: mol_type = other DNA
gcagtagacccaggcctgccttctgtgaagcaagagccacctgacccagaggaggacaaggaggagaacaaggatgattctgcctccaaattggccccagaggaagaggcaggaggggctggcacacccgtgatcacggagattttcagcctgggtggaacccgcttccgagatacagcagtctggttgcca

SEQ ID NO: 49 - DNA, 174 bp, Homo sapiens
  source 1..174: mol_type = other DNA
atggatgtctactacttcagtccaagtggtaagaagttcagaagcaagcctcagttggcaaggtacctgggaaatactgttgatctcagcagttttgacttcagaactggaaagatgatgcctagtaaattacagaagaacaaacagagactgcgaaacgatcctctcaattaa

SEQ ID NO: 50 - DNA, 168 bp, Homo sapiens
  source 1..168: mol_type = other DNA
gatgtctactacttcagtccaagtggtaagaagttcagaagcaagcctcagttggcaaggtacctgggaaatactgttgatctcagcagttttgacttcagaactggaaagatgatgcctagtaaattacagaagaacaaacagagactgcgaaacgatcctctcaat

SEQ ID NO: 51 - DNA, 249 bp, Homo sapiens
  source 1..249: mol_type = other DNA
atgggtggagaggatgatgaactcaagggcttactggggcttccagaggaggaaactgagcttgataacctgacagagttcaacactgcccacaacaagcggatttctacccttaccattgaggagggaaatctggacattcaaagaccaaagaggaagaggaagaactcacgggtgacattcagtgaggatgatgagatcatcaacccagaggatgtggatccctcagttggtcgattcaggaactaa

SEQ ID NO: 52 - DNA, 246 bp, Homo sapiens
  source 1..246: mol_type = other DNA
atgggtggagaggatgatgaactcaagggcttactggggcttccagaggaggaaactgagcttgataacctgacagagttcaacactgcccacaacaagcggatttctacccttaccattgaggagggaaatctggacattcaaagaccaaagaggaagaggaagaactcacgggtgacattcagtgaggatgatgagatcatcaacccagaggatgtggatccctcagttggtcgattcaggaac

SEQ ID NO: 53 - DNA, 192 bp, Homo sapiens
  source 1..192: mol_type = other DNA
atgcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctaa

SEQ ID NO: 54 - DNA, 186 bp, Homo sapiens
  source 1..186: mol_type = other DNA
cggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 55 - DNA, 933 bp, Homo sapiens
  source 1..933: mol_type = other DNA
ccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtg

SEQ ID NO: 56 - DNA, 327 bp, Homo sapiens
  source 1..327: mol_type = other DNA
atgtcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggagtaa

SEQ ID NO: 57 - DNA, 321 bp, Homo sapiens
  source 1..321: mol_type = other DNA
tcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggag

SEQ ID NO: 58 - DNA, 536 bp, synthetic construct
  source 1..536: mol_type = other DNA
atgcggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggataa

SEQ ID NO: 59 - DNA, 531 bp, synthetic construct
  source 1..531: mol_type = other DNA
cggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggag

SEQ ID NO: 60 - DNA, 933 bp, Homo sapiens; identical to SEQ ID NO: 55
  source 1..933: mol_type = other DNA

SEQ ID NO: 61 - DNA, 933 bp, Homo sapiens; identical to SEQ ID NO: 55
  source 1..933: mol_type = other DNA

SEQ ID NO: 62 - DNA, 5109 bp, synthetic construct
  source 1..5109: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtg

SEQ ID NO: 63 - protein, 1702 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKLEGGGGSGSPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFAC

SEQ ID NO: 64 - protein, 1688 aa, synthetic construct
MAPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLAGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGDGSPKKKVLEGGGGSGSPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 65 - DNA, 9316 bp, Homo sapiens
  source 1..9316: mol_type = other DNA
atttccgcctctggcgaatggctcgtctgtagtgcacgccgcgggcccagctgcgaccccggccccgcccccgggaccccggccatggacggtgaggtcgccctctgaccccgcggggtggcatcgccggggccgccgcggctgtggcgggagcccccgcgccgctttccgccccttcctgcgccgacgggcgctgcgcagggagcggtcggcctgccggatggtggcgcggggcggggcaggcggggaggggtctgactcagtttcccctctgggtggagggggtggcccttgactcagcatcctccctgggtgggggagcagagggaaccttgactcagtttccctccacaccgtccatgggggcagctctggcttcgtttccctattaggggagaacgcatctgattcagtttcctctctggagggaggtggggcgtgccctccctcaattttccttaagaaagtacaacaggccctgattcagcttccctctggagaagaaggattccttaccccgttttccctcaagaagcaggtgtttctggttcctttcctccaccccttagtttcaccgcaggttctagggagcaggtcctgactcagttcccctgggagggagcctggcttctcctctccgtggagagcagattcctccttgtgagaatgggcattgggtgtgtgctgaccctgatctccctcccctttcagaactgttccccctcatcttcccggcaggtaagtggcccccgcggcggtcggggaggtgctccctggggtagggcagtgggggcagccgcctaatggggctgcggtgtcccctggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaaggtcagcactgcgccagggtgtgcggggaagggactgtggaaccccagccctctgtgtttgggagtcaggtttggatgtctgggcctctgtactttggacagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacaggtgaggtccctgcaggctaggaggcccagcatgaggggacaggaggcgggctatgaagtagccgctacagtgttactcctttctggacccagggcagaattcctcactccttgccctgactcagctctgcaagtaaactgcctttgtcttgaaattatctatttgtacgtttactctagcagatgagttccaggggatacggatgggtccctaatcacttctgaattcctgacccctgtagctggaaggacaagtccctcagagtcacgtctcttgtttgaggaagaaaaccattgaaccagcactctcccctggagttgacattgcagttgacagagacaggctgtaaaaaaatatacaggccgggcatagtggctcatgcttataatcctagcacttcgggaggccaaggcaggaagatctcttgagcctaggaattcaagactagccaaggcaacatagtgagaccccatgtctacaaaaaataagaaattagctgggtatggtggcatgcgcctatagtcccagggaggctgaggtgggaggatcgcctgagcccaggaggtcactgctgcggcaagctatgattgcaccactgcacttgggtggcagggcgataccttgtctttttttttttttttttttttttaagaaagagtcttgctctgtccccagactggagtgcagtggcgcgatctcagctcactgcaacctccaccttccgggttcatgccattctcctgcctcagcttcccgaatagctgggactacaggtgcctgccaccacgcctggctaattttttgtattttttagtagagacagggtttcaccgtgttagccaggatggtctcaatctcctgaccttgtgatctgcctgtcttggcctcccaaagtgctgggattacaggcttgagccaccgcacccggcccgacactgtgtctcttaaaaataaataaatacacagataaataaacaaggtgctgtcagagtaacagtttcagtcctgaaaataaaacgaggtcataggatagaaggataacctggggtgggggagttaggggagctggtgtgtgatgactttagattgggaggtcagggaaggcctccctcagctaagacttgaatggtgagaaggaggagagaagtaggaaaagaacattccaagccgaccaaacaagtgcaaaggccttgaggtgggaatgagtgtcctgtgtttagagagtggaaaaggagccccggtggctgtaagcagggagcccacagggaggctggcaggggcaggggcacggaggtgcttgtcagctgagtgaagggacagggttcgttgcagcacagtaggaagccactggagagggttaggcatggagcgatgatctaggggtgggcagagtaggaaggagcaggctgggcaaggcaggaaggtctgggctctgtgagagacagtgggacagacgactgggggcgctcagtttccagaacctgggaatccagtgtgtgaagaagcgggacctggagcaggctatcagtcagcgcatccagaccaacaacaaccccttccaaggtgagggcaaacctgcctgccacacccgcaccctctgtcaccctccccagcccgcctttcctgcatctccctcactggccaagacacagtaaagctggaatcttggtgcccattcagtactctcccactttaccactgggaaactgagtcccaggaggggacatgacattcaggccacagtggagtcagtgctgggttctgcctcctctaggacttgtgtttcacacacgcaccagcggccctcccagcgcagagctccctgcccccactgatagcacctatatccccacttcccctgctctcctgatcgcctgttttcttggcagttcctatagaagagcagcgtggggactacgacctgaatgctgtgcggctctgcttccaggtgacagtgcgggacccatcaggcaggcccctccgcctgccgcctgtcctttctcatcccatctttgacaatcgtgagtagcgagggagacgcaggaaggggagagggtgggcctgaataggaggaagggggttggggtgagtgtggcctgcaagaggcgctctggcttcattcaatcatcttgcgtatttgtctactcttctctgcccagctctttgttggctggggagagagggcaaagaagacccagagctgtccttgaggtgtagctctggcctggcaggaagtctagcagagagtctgatgggagatgggcattttcagtattgtgtgatgagaaggtggtgtgcccacaactgcgataatgactctaggaaatgaactcccaggaacaagatagtgttcgagaatgatgattcagtgatggaagcccacagatgaattgggctgactgatcagctgcagtctttagttaagtaaccagcaggatgaatggaaataaaacgaatctggagccccaaggatgggaaaccaggataaactgggggtcaggagggaagggctcattgccaaggtgggtatggactggccttaaagattgctttggtcaggtggagaaagagtgtgatcagattggcacccactggactataagctccgggagagcagagaggtttgggttttgtgtgtggtcgtattcttgcttagatcagtgttgagtgcacagtaggcaaatacttgttgaatcggttggggtgaggagctaataagaaggtctgactttggctacaaaggtgtaggtggcaagctctgggcaaccttgactctgggctgggcagggaagttgcatctcctgcaggccaccctggggacctctcgtggctcaggtcatcctcccttgttttcactgcctcaggtgcccccaacactgccgagctcaagatctgccgagtgaaccgaaactctggcagctgcctcggtggggatgagatcttcctactgtgtgacaaggtgcagaaaggtatacatcaggaggcaggggtgggctcttgggagcaaggggtgaagctgagcagagaagtggagtgtcaggtaactggctttggatacttcctcctgtgcctcgggggcctcaggtgggccgaagggtgatgtatcagaagacaagcatcagaagttgagggggagaccatggagggttagacacactgacccacactgccaccgcatctcctgcagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacaggtacacagctggggtggcagcatcagggcagctgggctctgtgccgtgagagagaggccaagactgcatgtctggggctgtggttgagggccagaagctggagtcagagcttgggatccaagaaactggaatccatttcttgcctcatttaataataatgatacctcagacattgagctagtactacacatctgcccctattccaggcacttcactggattacctgattttaatctcatgacccttttgaagtaggagatagtgtccaacttgcctgggttcacgtggctagtaagtagcagagtctagcagagtcagcgttggaacccaggccatctggcttcaggatccttttgccacactggttgtcttgtcactaccatacagcccagccacattttggtcgaggtttgagtgtccacttggtaccagggcctgtgccaggtattgtgaatacaaaaaatgagtaagacatgctccctgcccttcagagtctcagtcacttgaggaagacaaatgtgcaaaacagatccttaagtcacggaatgacatgtgaaatggaggtgtgcgcagagaagcacgggggcacagtggcctccggggagctggagacagctgcatggagaaggtggcattgaagctggagaaagacacagaagtgtgttctccaggcaggtggcccagcacctggaagggaaggggtgttggcaactgcacggcctattcagggccaccttcgtggtttgagctgatgttgggcagtgagcaggctcagagattggagggcctggttaagctgaaggcctctaagcagagaggtgacaggagcagatttggaagttgactcaggaggccacgtgtagaagatcagagacagacagactcaaagcagggaggccattctggaaatgttgctgtagtccaggaagagaagatgcggcacaaaccttacagaaggaatagcagagcaagatcacatctccaccactgtcctcagttacccttcagccctgcagccgttttgcgtcttacagaccaagctcagtctcacctcaggacctgtgtctagctgttgcctctgcttggaatgcccaaggttttaatgcaagtggctcatgccagtccttccctgacctctaaattagcccttccctctacatttagactgtcctgacctcacttccagtattgttacatcaccatatcgtccctggaccaccgtcacttgagattttctggtttggttttgtttgtttgtttgagacagagtctcactcttgtcgcccaggctggagtgcagtggtgcaatctcggctcactgcaacctccacctcccgggttcaagtgattctccctgcctcagcctcccgagtagctgggactacaggcgcccgccaccacatccggctaattttttgtatttttagtagagacggggttttcaccatgttggccagggtggtctcgatctcttgacctcgtgatccacccgcctcgacctcccaaagtgctgggattacaggcgtgagccaccacgcctggccgagattttctggtttgtatatatgattgtgtccctctcactttttcactaggatgccaactttttgggagcagacaccttgtgtgcatactcacggccacatccccagtgtctagaagggaggctggcacatagtaggtgcttgataaatatatgtcaagttactggcggggagtgagagaaaagaagataatcattccaaggtttctagctttggtgacagggcagatggcggatggtcagggaatgcaggagaaagaacctagtttttgagggatgatgaggttgtctttgaattgatggaattgaagtgctttggagctactgaaataccactgtaattagaaagacagttctgaaagctctgactaaaatatgaaaccattagagataagaataagagtctctgctgaagtcttaaggcagttgtgcctcagctgccccagagtgccaagaaattctcgccaggcctgggtggaggctgacaatgagccaggatgtgagcagtgcaggaaggtgtgagctggaggctggggccccccaggtctcctctttgcccagagcatggcccctgtactcggcctcaccatgtggtcttagaggggcagattcagccaacagaggcctccaaaagctgtgggattctcagaactgctgagtgtcacacatcccttctcattggcagagaaggttgggagaatgggtcttgggaaagcctggctcctccccttcctcctgagggacctttgagagcgtcggggacccacctgaggagagcaagtcccacttctcccccttactttcccagacgatcgtcaccggattgaggagaaacgtaaaaggacatatgagaccttcaagagcatcatgaagaagagtcctttcagcggtgagatggggactgggaaagccagagaggaaggcagcgggcagggaggggacaccctggggctgagaactgacctagcccttgccctacaggacccaccgacccccggcctccacctcgacgcattgctgtgccttcccgcagctcagcttctgtccccaagccaggtaaggatttccttttgtcccactggaacgacaggttcagctctgttcagctgaggagcagtggagatgaagactcctgggccccattacagacctactgaatcagaggctttgaaaggatggcgcaggaaccagcgacttcctgggatgctaatgcacactgaagcctgggaaccaccgtgccacttaaattctatattgggtatcttgggaatcagaagttaaaaatgcagaatctctgctctcatctctcacttgaattgggggaacaaaaatcacctctaaattagcaagatgtgtgtgcctgtgcgaggctcaaaaactgacaagctctgcacttcaggccgctgctcacttggtgctccatgggctcattgcctctcaggacttagtttttcagctgtaaactgaggagtccagcttatctctccaagtgtcccttcctctaaaaggctggaagagtttcgtgggcttgagtagtcagggaggactccaaggtggaggtgtggctagaactggaccctacaggctgggtcagatggggtaagaggaaggagatagggtgttggcagtgactccctagagagggattgagcctgagcaccatgaagaggggctggggtacagaggacggggtggatctctagggctttctctgacccctgcctctctgtctctctcctccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctgg

SEQ ID NO: 66 - DNA, 2507 bp, Homo sapiens
  source 1..2507: mol_type = other DNA
atttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggcctccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctgg

SEQ ID NO: 67 - protein, 548 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 68 - DNA, 3215 bp, synthetic construct
  source 1..3215: mol_type = other DNA
gagctcctcgacctcaccaccattcgcgtgacggtctgcgagggcaagaacctcctgcagcgcgcgaacgagttggtgaatcccgacgccggcgcaggacgtcgacgcgaccgcggccgcccggggcccgcccgcgggggctgccgccgcgaccgcacgggcccccgcccgctcggcttcccgtccccgccgccccctcgagtagcgaccccggccgcgttcagacgacagacaataaagcatcacggtcgatttaaccacttcgctgtcagcacgcgtttgtggcgaggggcgggaggggcgggaggggcgggaggggcgggaggggcgggaggggcgggaggggcgggaggggcgggaggggcgggaggggcgggaggggcgggaggggcgggaggggcgggaggggcgggaggggcgggaggggcgggaggggcgggaggggcgggaggggcgggaggggcgggaggggcgggagaaggacacgcctcccttccgagcgcgggggacgggccgcccggagcggtggggaagtcacgaggtttggggcggcactggggtgatacttaaatgcggggtggtggacgcgagatgtcagtcctcggggacgcacggcacccccggcgattcccttcgcgagggccccggcctttttctgtcgcgggtcccggatccctccccccctctccgccgccgggcgctcgggcacgtctcattcgcctctcgagatcgttattcccggacccaaccgcccccatggacctgttggtcgacgatctgtttgcggacgcggacggggtttcgccaccgccccccaggccagccgggggtcccaagaacaccccagccgcccctccgctgtacgccaccggtcggctgagtcaggcccagctgatgccctcgccgcccatgcccgtcccccccgcggccctgtttaaccgtctcctcgacgatctgggcttcagcgcgggtcccgcgctgtgtaccatgctagatacctggaacgaggacctgttctctgggttcccgaccaacgccgacatgtaccgggagtgcaagtttctgtcgacgctgcccagcgacgtgatcgactggggggatgcgcacgtccccgagcgctccccgatcgacattcgcgcccacggcgacgtggcgttccccaccctgcccgccacccgcgacgagctgccttcgtactacgaggccatggcgcagtttttccgcggtgagctgcgggcgcgggaggagagctaccggaccgtgttggcaaatttttgctcggccctgtaccggtacctgcgcgccagcgttcggcagctacaccgccaggcacacatgcggggccgcaaccgcgacctgcgggagatgctgcgcaccacgatcgcggacaggtactaccgcgagaccgcgcgcctggcgcgcgtcctgtttctgcatctatacctctttctgagccgcgagatcctatgggccgcgtacgccgagcagatgatgcggcccgatctgttcgacggcctctgctgcgacctggagagctggcgccagttggcgtgtctgtttcagcccctgatgtttatcaacggatcgctcaccgtgcggggagttcccgtggaggcccggcgactgcgggagctaaaccacattcgcgagcacctgaacctcccgctggtgcgaagtgcggcggcggaggaacccggggcgcccctcacgaccccgcccgtcctgcagggcaaccaggcccgctcctctgggtactttatgctgctgatccgggccaagttggactcgtactccagcgtcgcgacctcggagggcgagtccgtcatgcgggagcacgcgtatagccgcgggcggaccagaaacaattacggatcgacaatcgagggcctgctcgacctcccggacgacgatgacgctcccgcggaggccgggctggtggcgccgcgcatgtcgtttctctccgcgggacaacgcccccgcagactgtccaccaccgcccccattaccgacgtcagcctgggagacgaactccgcctggacggcgaggaggtggatatgacgcccgccgacgccctggacgacttcgacttggagatgctgggggacgtggagtccccctccccgggaatgacccacgaccccgtctcgtatggggctttggacgtggacgattttgagtttgaacagatgtttaccgatgccctgggcattgacgactttggggggtaggatgtgcgaccgggcggcgcgccccccccaccgccgccccgcctcacctccgtctgtatcgcgagggttcgcaaccacagcaataaacattggcaagcaactcatcatacgcggcgtgcgttggctgtttattacgggaccatgaaagaaatggggttacgcgcggggtggggggtgtgtgccgttgggttgggcgttagtcgcgcctacgagcccgcggtcgtgtagattcgcgtcacagaacggctcgtggtgctggggtccgcgtataaaggcaggcgcgcgggtcccgttctcgcatttgcccgcgggtctgcgtggggacgaggcccaccccccccacccttgttggagcggtcgcgttttctctgttcccgtcgtgccggttcctaccccccgctccctgggaccgccccctaccccccacctccccgtttgggcctccccccccccctcgcaccacccctttcctcgtccgtctgcggggagggcgtgtgtaaaaaatcgggcctccggccaccatgtccgtgcgcgggcatgccgtacgccggagggccgcctccacccggtcccatgccccgtccgcgcatcgcgccgactcgcccgtggaggacgagcccgagggcggtggagtcgggttaatggggtacctgcgggcggtgtttaacgtggacgacgacagcgaggtcgaggccgcgggggagatggcgagcgaagagccgcccccgcgccgtcgccgggaggcccgcggtcaccccgggtcccgacgcgcgtccgaggcccgggcggcggcgcccccccgccgggcgtcctttccgcgccccaggtccgttacggccaggagccagtccgttcgcggacgccgggacagcgccatcacgcgggccccgcggggaggctacctgggcccgatggacccacgcgacgttttggggcgggtgggcggttcgcgggtggtgccctcgccgctgttcctggacgagctc

SEQ ID NO: 69 - DNA, 2211 bp, synthetic construct
  source 1..2211: mol_type = other DNA
ggatccctccccccctctccgccgccgggcgctcgggcacgtctcattcgcctctcgagatcgttattcccggacccaaccgcccccatggacctgttggtcgacgatctgtttgcggaccgggacggggtttcgccaccgccccccaggccagccgggggtcccaagaacaccccagccgcccctccgctgtacgccaccggtcggctgagtcaggcccagctgatgccctcgccgcccatgcccgtcccccccgcggccctgtttaaccgtctcctcgacgatctgggcttcagcgcgggtcccgcgctgtgtaccatgctagatacctggaacgaggacctgttctctgggttcccgaccaacgccgacatgtaccgggagtgcaagtttctgtcgacgctgcccagcgacgtgatcgactggggggatgcgcacgtccccgagcgctccccgatcgacattcgcgcccacggcgacgtggcgttccccaccctgcccgccacccgcgacgagctgccttcgtactacgaggccatggcgcagtttttccgcggtgagctgcgggcgcgggaggagagctaccggaccgtgttggcaaatttttgctcggccctgtaccggtacctgcgcgccagcgttcggcagctacaccgccaggcacacatgcggggccgcaaccgcgacctgcgggagatgctgcgcaccacgatcgcggacaggtactaccgcgagaccgcgcgcctggcgcgcgtcctgtttctgcatctatacctctttctgagccgcgagatcctatgggccgcgtacgccgagcagatgatgcggcccgatctgttcgacggcctctgctgcgacctggagagctggcgccagttggcgtgtctgtttcagcccctgatgtttatcaacggatcgctcaccgtgcggggagttcccgtggaggcccggcgactgcgggagctaaaccacattcgcgagcacctgaacctcccgctggtgcgaagtgcggcggcggaggaacccggggcgcccctcacgaccccgcccgtcctgcagggcaaccaggcccgctcctctgggtactttatgctgctgatccgggccaagttggactcgtactccagcgtcgcgacctcggagggcgagtccgtcatgcgggagcacgcgtatagccgcgggcggaccagaaacaattacggatcgacaatcgagggcctgctcgacctcccggacgacgatgacgctcctgcggaggccgggctggtggcgccgcgcatgtcgtttctctccgcgggacaacgcccccgcagactgtccaccaccgcccccattaccgacgtcagcctgggagacgaactccgcctggacggcgaggaggtggatatgacgcccgccgacgccctggacgacttcgacttggagatgctgggggacgtggagtccccctccccgggaatgacccacgaccccgtctcgtatggggctttggacgtggacgattttgagtttgaacagatgtttaccgatgccatgggcattgacgactttggggggtaggatgtgcgaccgggcggcgcgccccccccccaccaccgccccgcctcacctccgtctgtatcgcgatagagggttcgcaaccacagcaataaacattggcaagcaactcatcatacgcggcgtgcgttggctgtttattacgggaccatgaaagaaatggggttacgcgcggggtggggggtgtgtgccgttgggttgggcgttagtcgcgcctacgagcccgcggtcgtgtagattcgcgtcacagaacggctcgtggtgctggggtccgcgtataaaggcaggcgcgcgggtcccgttctcgcatttgcccgcgggtctgcgtggggacgaggcccacccccccacccttgttggagcggtcgcgttttctctgttcccgtcgtgccggttcctaccccccgctccctgggaccgccccctaccccccacctccccgtttgggcctcccccctcgcaccacccctttcctcgtccgtctgcggggagggcgtgtgtaaaaaatcgggcctccggccaccatgtccgtgcgcgggcatgccgtacgccggaggcgcgcctccacccggtcccatgccccgtccgcgcatcgcgccgactcgcccgtggaggacgagcccgagggcggtggagtcgggttaatggggtacc

SEQ ID NO: 70 - protein, 490 aa, synthetic construct
MDLLVDDLFADADGVSPPPPRPAGGPKNTPAAPPLYATGRLSQAQLMPSPPMPVPPAALFNRLLDDLGFSAGPALCTMLDTWNEDLFSGFPTNADMYRECKFLSTLPSDVIDWGDAHVPERSPIDIRAHGDVAFPTLPATRDELPSYYEAMAQFFRGELRAREESYRTVLANFCSALYRYLRASVRQLHRQAHMRGRNRDLREMLRTTIADRYYRETARLARVLFLHLYLFLSREILWAAYAEQMMRPDLFDGLCCDLESWRQLACLFQPLMFINGSLTVRGVPVEARRLRELNHIREHLNLPLVRSAAAEEPGAPLTTPPVLQGNQARSSGYFMLLIRAKLDSYSSVATSEGESVMREHAYSRGRTRNNYGSTIEGLLDLPDDDDAPAEAGLVAPRMSFLSAGQRPRRLSTTAPITDVSLGDELRLDGEEVDMTPADALDDFDLEMLGDVESPSPGMTHDPVSYGALDVDDFEFEQMFTDALGIDDFGG

SEQ ID NO: 71 - DNA, 1485 bp, Homo sapiens
  source 1..1485: mol_type = other DNA
gtatctggccagctctatctgcatgtcgccgaaaaggggcaccacctgcagctgcctggagagagggagccggtgtcctacgtcagagccgccgccgccgccggagccgccgccggggaggagcagcccgtgcccccaggactgggcccttagggaggaggaggcgagaagatggcggacgaccccagtgctgccgacaggaacgtggagatctggaagatcaagaagctcattaagagcttggaggcggcccgcggcaatggcaccagcatgatatcattgatcattcctcccaaagaccagatttcacgagtggcaaaaatgttagcggatgagtttggaactgcatctaacattaagtcacgagtaaaccgcctttcagtcctgggagccattacatctgtacaacaaaggctcaaactttataacaaagtacctccaaatggtctggttgtatactgtggaacaattgtaacagaagaaggaaaggaaaagaaagtcaacattgactttgaacctttcaaaccaattaatacgtcattgtatttgtgtgacaacaaattccatacagaggctcttacagcactactttcagatgatagcaagtttggattcattgtaatagatggtagtggtgcactttttggcacactccaaggaaacacaagagaagtcctgcacaaattcactgtggatctcccaaagaaacacggtagaggaggtcagtcagccttgcgttttgcccgtttaagaatggaaaagcgacataactatgttcggaaagtagcagagactgctgtgcagctgtttatttctggggacaaagtgaatgtggctggtctagttttagctggatccgctgactttaaaactgaactaagtcaatctgatatgtttgatcagaggttacaatcaaaagttttaaaattagttgatatatcctatggtggtgaaaatggattcaaccaagctattgagttatctactgaagtcctctccaacgtgaaattcattcaagagaagaaattaataggacgatactttgatgaaatcagccaggacacgggcaagtactgttttggcgttgaagatacactaaaggctttggaaatgggagctgtagaaattctaatagtctatgaaaatctggatataatgagatatgttcttcattgccaaggcacagaagaggagaaaattctctatctaactccagagcaagaaaaggataaatctcatttcacagacaaagagaccggacaggaacatgagcttatcgagagcatgcccctgttggaatggtttgctaacaactataaaaaatttggagctacgttggaaattgtcacagataaatcacaagaagggtctcagtttgtgaaaggatttggtggaattggaggtatcttgcggtaccgagtagatttccagggaatggaataccaaggaggagacgatgaattttttgaccttgatgactactag

SEQ ID NO: 72 - protein, 437 aa, Homo sapiens
MADDPSAADRNVEIWKIKKLIKSLEAARGNGTSMISLIIPPKDQISRVAKMLADEFGTASNIKSRVNRLSVLGAITSVQQRLKLYNKVPPNGLVVYCGTIVTEEGKEKKVNIDFEPFKPINTSLYLCDNKFHTEALTALLSDDSKFGFIVIDGSGALFGTLQGNTREVLHKFTVDLPKKHGRGGQSALRFARLRMEKRHNYVRKVAETAVQLFISGDKVNVAGLVLAGSADFKTELSQSDMFDQRLQSKVLKLVDISYGGENGFNQAIELSTEVLSNVKFIQEKKLIGRYFDEISQDTGKYCFGVEDTLKALEMGAVEILIVYENLDIMRYVLHCQGTEEEKILYLTPEQEKDKSHFTDKETGQEHELIESMPLLEWFANNYKKFGATLEIVTDKSQEGSQFVKGFGGIGGILRYRVDFQGMEYQGGDDEFFDLDDY

SEQ ID NO: 73 - DNA, 21 bp, synthetic construct
  source 1..21: mol_type = other DNA
gtggatagcggtttgactcac

SEQ ID NO: 74 - DNA, 26 bp, synthetic construct
  source 1..26: mol_type = other DNA
gtggatagcggtttgactcacgggga

SEQ ID NO: 75 - DNA, 20 bp, synthetic construct
  source 1..20: mol_type = other DNA
gcccatttgcgtcaatgggg

SEQ ID NO: 76 - DNA, 25 bp, synthetic construct
  source 1..25: mol_type = other DNA
gcccatttgcgtcaatggggcggag

SEQ ID NO: 77 - DNA, 22 bp, synthetic construct
  source 1..22: mol_type = other DNA
attgacgtcaatgggagtttgt

SEQ ID NO: 78 - DNA, 30 bp, synthetic construct
  source 1..30: mol_type = other DNA
attgacgtcaatgggagtttgttttggcac

SEQ ID NO: 79 - DNA, 22 bp, synthetic construct
  source 1..22: mol_type = other DNA
cattgacgcaaatgggcggtag

SEQ ID NO: 80 - DNA, 30 bp, synthetic construct
  source 1..30: mol_type = other DNA
cattgacgcaaatgggcggtaggcgtgtac

SEQ ID NO: 81 - DNA, 22 bp, synthetic construct
  source 1..22: mol_type = other DNA
cgctgggccaggtctctgggag

SEQ ID NO: 82 - DNA, 20 bp, synthetic construct
  source 1..20: mol_type = other DNA
ctcccagagacctggcccag

SEQ ID NO: 83 - DNA, 20 bp, synthetic construct
  source 1..20: mol_type = other DNA
tcagggtagatagctgaggg

SEQ ID NO: 84 - DNA, 22 bp, synthetic construct
  source 1..22: mol_type = other DNA
ctatctaccctgagcaggcgcc

SEQ ID NO: 85 - DNA, 20 bp, synthetic construct
  source 1..20: mol_type = other DNA
gacagggggagccctataat

SEQ ID NO: 86 - DNA, 20 bp, synthetic construct
  source 1..20: mol_type = other DNA
actgggatgtaagccatagc

SEQ ID NO: 87 - DNA, 20 bp, synthetic construct
  source 1..20: mol_type = other DNA
gttggagcttagaatgtgaa

SEQ ID NO: 88 - DNA, 20 bp, synthetic construct
  source 1..20: mol_type = other DNA
gccctatccctgggggaggg

SEQ ID NO: 89 - DNA, 20 bp, synthetic construct
  source 1..20: mol_type = other DNA
tcgggcttggggagaggagg

SEQ ID NO: 90 - DNA, 20 bp, synthetic construct
  source 1..20: mol_type = other DNA
ctctccccaccccaccttct

SEQ ID NO: 91 - DNA, 20 bp, synthetic construct
  source 1..20: mol_type = other DNA
tgtgaagggagaatgaggaa

SEQ ID NO: 92 - DNA, 20 bp, synthetic construct
  source 1..20: mol_type = other DNA
tcaggagagctactcggggt

SEQ ID NO: 93 - DNA, 6443 bp, Homo sapiens
  source 1..6443: mol_type = other DNA
ggaacttgatgctcagagaggacaagtcatttgcccaaggtcacacagctggcaactggcagagccaggattcacgccctggcaatttgactccagaatcctaaccttaacccagaagcacggcttcaagcccctggaaaccacaatacctgtggcagccagggggaggtgctggaatctcatttcacatgtggggagggggctcccctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtgtgaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtcttgattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgatttcctctaagccccagcctcagtttctctttctgcccacatactggccacacaattctcagccccctcctctccatctgtgtctgtgtgtatctttctctctgcccttttttttttttttagacggagtctggctctgtcacccaggctagagtgcagtggcacgatcttggctcactgcaacctctgcctcttgggttcaagcgattctgctgcctcagtagctgggattacaggctcacaccaccacacccggctaatttttgtatttttagtagagacgagctttcaccatgttggccaggcaggtctcaaactcctgaccaagtgatccacccgccggcctcccaaagtgctgagattacaggcctgagccaccatgcccggcctctgcccctctttcttttttagggggcagggaaaggtctcaccctgtcacccgccatcacagctcactgcagcctccacctcctggactcaagtgataagtgatcctcccgcctcagcctttccagtagctgagactacaggcgcataccactaggattaatttgggggggggggtggtgtgtgtggagatggggtctggctttgttggccaggctgatgtggaattcctgggctcaagcgatactcccaccttggcctcctgagtagctgagactactggctagcaccaccacacccagctttttattattatttgtagagacaaggtctcaatatgttgcccaggctagtctcaaacccctgggctcaagagatcctccgccatcggcctcccaaagtgctgggattccaggcatggggctccgagcccggcctgcccaacttaataatacttgttcctcagagttgcaactccaaatgacctgagattggtgcctttattctaagctattttcattttttttctgctgtcattattctcccccttctctcctccagtcttatctgatatctgcctccttcccacccaccctgcaccccatcccacccctctgtctctccctgttctcctcaggagactctggcttcctgttttcctccacttctatcttttatctctccctcctacggtttcttttctttctccccggcctgcttgtttctcccccaacccccttcatctggatttcttcttctgccattcagtttggtttgagctctctgcttctccggttccctctgagctagctgtcccttcacccactgtgaactgggtttccctgcccaaccctcattctctttctttctttctttttttttttttttttttttttttttttttgagacagagtcttgctctgttgcccagcctggagtgcagtggtgcaatcttggttcactgcaacctccacttcccagattcaagcaattctcctgcctcagcctccagagtagctgggattacaggcgtgtcccaccacacccgactaatttttgtatttttggtagagacaaggcttcggcattgttggccaggcaggtctcgaactcctgacctcaagtaatctgcctgcctcaccctcccaaagtgctgggattacaggcatgagccacctcacccggaccatccctcattctccatcctttcctccagttgtgatgtctacccctcatgtttcccaacaagcctactgggtgctgaatccaggctgggaagagaagggagcggctcttctgtcggagtctgcaccaggcccatgctgagacgagagctggcgctcagagaggggaagcttggatggaagcccaggagccgccggcactctcttctcctcccaccccctcagttctcagagacggggaggagggttcccaccaacgggggacaggctgagacttgagcttgtatctcctgggccagctgcaacatctgcttgtccctctgcccatcttggctcctgcacaccctgaacttggtgctttccctggcactgctctgatcacccacgtggaggcagcacccctcccct

SEQ ID NO: 94 - DNA, 954 bp, Homo sapiens
  source 1..954: mol_type = other DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 95 - DNA, 954 bp, Homo sapiens
  source 1..954: mol_type = other DNA
atgaaggttctgtgggctgcgttgctggtcacattcctggcaggatgccaggccaaagtcgaacaggctgtcgaaactgaacccgaaccggagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagcttcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctacaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 96 - DNA, 954 bp, Homo sapiens
  source 1..954: mol_type = other DNA
atgaaggttctgtgggctgcgttgctggtcacattcctggcaggatgccaggccaaagtcgaacaggctgtcgaaactgaacccgaaccggagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagcttcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctacaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 97-98 - skipped (2 entries)

SEQ ID NO: 99 - DNA, 130 bp, Adeno-associated dependoparvovirus B
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 100 - DNA, 141 bp, Adeno-associated dependoparvovirus B
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 101 - DNA, 141 bp, Adeno-associated dependoparvovirus B
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 102 - DNA, 130 bp, Adeno-associated dependoparvovirus B
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 103 - DNA, 49 bp, synthetic construct
  source 1..49: mol_type = other DNA
aataaaacatctttgttttcattagatctgtgtgttggttttttgtgtg

SEQ ID NO: 104 - DNA, 49 bp, synthetic construct
  source 1..49: mol_type = other DNA
aataaaagatctttgttttcattagatctgtgtgttggttttttgtgtg

SEQ ID NO: 105 - DNA, 48 bp, synthetic construct
  source 1..48: mol_type = other DNA
aaaaggccggcggccacgaaaaaggccggccaggcaaaaaagaaaaag

SEQ ID NO: 106 - DNA, 21 bp, synthetic construct
  source 1..21: mol_type = other DNA
ccaaagaaaaagaggaaagtc

SEQ ID NO: 107 - DNA, 21 bp, synthetic construct
  source 1..21: mol_type = other DNA
ccaaagaagaaacggaaggtg

SEQ ID NO: 108 - DNA, 21 bp, synthetic construct
  source 1..21: mol_type = other DNA
cccaagaagaagagaaaggtg

SEQ ID NO: 109 - DNA, 21 bp, synthetic construct
  source 1..21: mol_type = other DNA
ccgaagaaaaagcgcaaggtc

SEQ ID NO: 110 - DNA, 21 bp, synthetic construct
  source 1..21: mol_type = other DNA
cctaaaaagaaacgaaaggtt

SEQ ID NO: 111 - DNA, 21 bp, synthetic construct
  source 1..21: mol_type = other DNA
ccaaagaagaagcggaaggtc

SEQ ID NO: 112 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 113 - DNA, 212 bp, synthetic construct
  source 1..212: mol_type = other DNA
gggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 114 - DNA, 241 bp, synthetic construct; identical to SEQ ID NO: 112
  source 1..241: mol_type = other DNA

SEQ ID NO: 115 - DNA, 73 bp, synthetic construct
  source 1..73: mol_type = other DNA
gttttagtccctgaaaagggactaaaataaagagtttgcgggactctgcggggttacaatcccctaaaaccgc

SEQ ID NO: 116 - DNA, 76 bp, synthetic construct
  source 1..76: mol_type = other DNA
gttttagtactctggaaacagaatctactaaaacaaggcaaaatgccgtgtttatctcgtcaacttgttggcgaga

SEQ ID NO: 117 - DNA, 10102 bp, synthetic construct
  source 1..10102: mol_type = other DNA
gcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatttaattaacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgcgggcgtctttggaaatcctggagaacgccggatgggagacgaatggtcgtgggcaccgggagggggtggtgctgccatgaggacccgctgggccaggtctctgggaggtgagtacttgtccctttggggagcctaaggaaagagacttgacctggctttcgtcctgcttctgatattcccttctccacaagggctgagagattaggctgcttctccgggatccgcttttccccgggaaacgcgaggatgctccatggagcgtgagcatccaacttttctctcacataaaatctgtctgcccgctctcttggtttttctctgtaaagtaagcaagctgcgtttggcaaataatgaaatggaagtgcaaggaggccaagtcaacaggtggtaacgggttaacaagtgctggcgcggggtccgctagggtggaggctgagaacgccccctcgggtggctggcgcggggttggagacggcccgcgagtgtgagcggcgcctgctcagggtagatagctgagggcggatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagaggaggagagtgacttaattaaaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtggaggagggcggctcgagagccacgaacttctctctgttaaagcaagcaggagacgtggaagaaaaccccggtcccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagtcgaccatgcatctagacccagcggcgcgcccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgggcgcgccgaattcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtg

SEQ ID NO: 118 - DNA, 13039 bp, synthetic construct
  source 1..13039: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgctcagggtagatagctggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 119 - DNA, 13812 bp, synthetic construct
  source 1..13812: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgtgtacacgtctctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggggccggcccggatccggcgcaacaaacttctctctgctgaaacaagccggagatgtcgaagagaatcctggaccgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtgccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 120 - DNA, 14092 bp, synthetic construct
  source 1..14092: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgtgtacacgtctctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggacaggccaaaaagaaaaagctcgagggcggaggcgggagcggatccccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtgttcatgaatgagaaagaggacatcttatggtgcactgaaatggaaagggtatttggtttcccagtccactatactgacgtgtccaacatgagccgcttggcgaggcagagactgctgggccggtcatggagcgtgccagtcatccgccacctcttcgctccgctgaaggagtattttgcgtgtgtgtccggccggcccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 121 - DNA, 9158 bp, synthetic construct
  source 1..9158: mol_type = other DNA
gcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatccatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtggaggagggcggctcgagagccacgaacttctctctgttaaagcaagcaggagacgtggaagaaaaccccggtcccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagtcgacatgcatctagacccagcggcgcgcccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgggcgcgccgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtg

SEQ ID NO: 122 - DNA, 12952 bp, synthetic construct
  source 1..12952: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcggtactgcaccaggcggcgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatccccaaagaagaaacggaaggtgggtggaggaagtggcgggtcaggtggctctagacggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggagggcgcgcccaagaagaagaggaaagtctccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 123 - DNA, 12952 bp, synthetic construct
  source 1..12952: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggcgcggacatggaggacggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatccccaaagaagaaacggaaggtgggtggaggaagtggcgggtcaggtggctctagacggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggagggcgcgcccaagaagaagaggaaagtctccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 124 - DNA, 12950 bp, synthetic construct
  source 1..12950: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaatgggcgggacgttaacggggcggaacggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgtgtacacgtctctgttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgacggatccccaaagaagaaacggaaggtgggtggaggaagtggcgggtcaggtggctctagacggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctcgggaggtggttcgggaggtggttcggagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagacagtactccccatcaagaagcgcaagacccgggagggcgcgcccaagaagaagaggaaagtctccggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctccctaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctga

SEQ ID NO: 125 - DNA, 19 bp, synthetic construct
  source 1..19: mol_type = other DNA
ggcgaggagctgttcaccg

SEQ ID NO: 126 - DNA, 20 bp, synthetic construct
  source 1..20: mol_type = other DNA
gccacaagttcagcgtgtcc

SEQ ID NO: 127 - DNA, 3177 bp, Homo sapiens
  source 1..3177: mol_type = other DNA
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 128 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 129-130 - skipped (2 entries)

SEQ ID NO: 131 - DNA, 30 bp, synthetic construct
  source 1..30: mol_type = other DNA
cgctgggccaggtctctgggaggtgagtac

SEQ ID NO: 132 - DNA, 30 bp, synthetic construct
  source 1..30: mol_type = other DNA
ctatctaccctgagcaggcgccgctcacac

SEQ ID NO: 133 - DNA, 25 bp, synthetic construct
  source 1..25: mol_type = other DNA
ctcccagagacctggcccagcgggt

SEQ ID NO: 134 - DNA, 25 bp, synthetic construct
  source 1..25: mol_type = other DNA
tcagggtagatagctgagggcggat

SEQ ID NO: 135 - DNA, 21 bp, synthetic construct
  source 1..21: mol_type = other DNA
gcctatttcccatgattcctt

SEQ ID NO: 136 - DNA, 22 bp, synthetic construct
  source 1..22: mol_type = other DNA
aaaactgcaaactacccaagaa

(19) United States
(12) Patent Application Publication
(10) Pub. No.: US 2026/0218148 A1
(43) Pub. Date: Jul. 30, 2026

(54) COMPOSITIONS AND METHODS RELATING TO EPIGENETIC MODULATION

(71) Applicant: Duke University, Durham, NC (US)

(72) Inventors: Boris Kantor, Durham, NC (US); Ornit Chiba-Falek, Durham, NC (US)

(21) Appl. No.: 18/701,982

(22) PCT Filed: Oct. 18, 2022
(86) PCT No.: PCT/US2022/078260

§ 371 Date: Apr. 17, 2024

Related U.S. Application Data
(60) Provisional application No. 63/256,754, filed on Oct. 18, 2021.

(51) Int. Cl. C12N 9/22; A61K 31/4353; A61K 31/519; A61K 31/7088; A61K 38/00; A61K 48/00; A61P 25/28; C07K 14/47; C07K 16/28; C12N 15/113; C12N 15/52; C12N 15/864

(52) U.S. Cl. CPC C12N 9/226; A61K 31/4353; A61K 31/519; A61K 31/7088; A61K 38/00; A61K 48/0058; A61P 25/28; C07K 14/4702; C07K 16/2887; C12N 15/113; C12N 15/52; C12N 15/8645; C07K 2319/00; C12N 2310/20; C12N 2750/14143; C12N 2830/34

(57) ABSTRACT

Disclosed herein are methods of effecting precision epigenetic modulation of one or more genes of interest. Disclosed herein are isolated nucleic acid molecules, viral vectors, rAAV vectors, pharmaceutical formulations, host cells, guide RNAs, and plasmids for use in the disclosed methods.

### I. CROSS-REFERENCE TO RELATED APPLICATIONS

This application claims the benefit of priority to U.S. Provisional Patent Application No. 63/256,754 filed 18 Oct. 2021, which is incorporated by reference herein in its entirety.

### II. REFERENCE TO THE SEQUENCE LISTING

The Sequence Listing submitted 18 Oct. 2022 as an XML file named “22_2057_WO_Sequence_Listing”, created on 18 Oct. 2022 and having a size of 414 kilobytes is hereby incorporated by reference pursuant to 37 C.F.R. § 1.52(e)(5).

### III. BACKGROUND

Alzheimer's disease (AD) is sixth leading cause of death in the US and the most common cause of dementia in aging. With a rapidly growing aging population the number of AD cases is growing fast and projected to rise drastically over the next three decades. Today, more than 5 million people are living with AD in the United States alone, and by 2050, this number is projected to reach 14 million. Therefore, AD poses a huge economic burden on society placing overwhelming strain on the healthcare system. In 2020, the cost of AD to the US was $301 billion, including $206 billion in Medicare and Medicaid payments, while the caregivers provided $244 billion' worth of care (Alzheimer's Association, Alzheimer's Impact Movement: Factsheet 2020). These trends will only worsen with time because there are no therapies to halt or prevent AD (i.e., projected to cost more than $1.1 trillion annually by 2050). Despite all the research effort, money and commitment, clinical trials to identify disease modifying therapies (DMT) for AD have repeatedly failed. To date, there is no cure and no DMT for AD and there are no methods to delay the onset and/or progression of the disease. Most available treatments are palliative and aimed at relieving symptom (Sharma K. (2019) Mol Med Rep. 20:1479-1487; Olivares D, et al. (2012) Curr Alzheimer Res. 9:746-758).

Thus, there remains an unmet medical need for developing methods of effecting precision epigenetic modulation of one or more genes implicated in a disease or disorder.

### IV. BRIEF DESCRIPTION OF THE FIGURES

FIG. 1 shows the schematics of CRISPR/Cas9 activity.

FIG. 2 shows the schematic of transcriptional repression utilizing CRISPR/dCas9 platforms.

FIG. 3 shows the schematics different repressors utilized in this application to establish all-in-one AAV—fusion system for targeted gene silencing.

FIG. 4 shows the construction of all-in-one AAV vectors harboring dCas9-repressor effectors.

FIG. 5 shows the development of the reporter cell line for evaluation of all-in-one dCas9-CRISPR/Cas9-effector systems.

FIG. 6 shows the establishment and analysis of luciferase-based reporter assay used herein for screening and evaluating the efficiency of the CRISPR/Cas9 tools for epigenome-based repression.

FIG. 7 shows the design of gRNA targeting the promoter region of CMV gene.

FIG. 8 shows the design of gRNA targeting the promoter region of CMV gene with SaCas9 and CjCas9 endonuclease fused with different disclosed repressors.

FIG. 9A shows the HP1a-dSaCas9 map, FIG. 9B shows the HP1b-dSaCas9 map, and FIG. 9C shows the MBD1-dSaCas9 map.

FIG. 10A shows the MBD2-dSaCas9 map, FIG. 10B shows the NIPP1-dSaCas9 map, and FIG. 10C shows the KRAB-dSaCas9 map.

FIG. 11A shows the MeCP2-dSaCas9 map, FIG. 11B shows the KRAB-MeCP2-dSaCas9 map, and FIG. 11C shows the DNMT3A-dSaCas9 map.

FIG. 12A shows the HP1a-dCjCas9 map, FIG. 12B shows the HP1b-dCjCas9 map, and FIG. 12C shows the MBD1-dCjCas9 map.

FIG. 13A shows the MBP2-dCjCas9 map, FIG. 13B shows the NIPP1-dCjCas9 map, and FIG. 13C shows the KRAB-dCjCas9 map.

FIG. 14A shows the MeCP2-dCjCas9 map, FIG. 14B shows the KRAB-MeCP2-dCjCas9 map, and FIG. 14C shows the DNMT3A-dCjCas9 map.

FIG. 15 is a schematic of the advantages of an all-in-one AAV vector systems over the two-component counterparts.

FIG. 16 shows the screening and validation of the repressor systems using CMV-driven reporter cell line.

FIG. 17 shows the measurement of the physical titer of all-in-one systems using real-time PCR data on the viral particles.

FIG. 18 shows the measurement of the repression capacity of the vectors developed herein. Briefly, normalized luciferase measured on day 6 after AAV transduction. The data represents the percentage of luciferase for each treatment group normalized by the control group (CRISPR/dCas9 with no sgRNA and no effector). unpaired two-tail t-test with equal variance (α=0.05) was conducted to find significant gene repression (*p<0.01, **p<0.001, ***p<0.0001).

FIG. 19 shows the map of a vector using a gRNA targeting intron 1 of SNCA.

FIG. 20 shows the location of the gRNA 1 and gRNA2 selected for evaluation and screening relative to the intron 1—SNCA-GFP-Luc reporter construct.

FIG. 21 shows the gRNA1 and gRNA2 cloned into AAV vectors carrying dCjCas9 (SEQ ID NO:131 and SEQ ID NO:132) and dSaCas9 (SEQ ID NO:133-SEQ ID NO:134) with KRAB-MeCP2 transgenes. FIG. 21 also shows the schematic image of the pCL35-reporter cell line constructed to overexpress CAG-intron 1 of SNCA gene.

FIG. 22A shows gRNA1 and gRNA2 cloned into AAV-dCjCas9 and AAV-dSaCas9 vectors carrying either KRAB-MeCP2 or HP1A for targeted repression of intron 1 of SCNA. Plasmids and AAV vectors have been evaluated by transfection and transduction into the reported cell line. Briefly, normalized luciferase measured on days 4 and 6 after plasmid transfection as well as days 4, 6, 9, 12, 14, 17, and 21 after AAV transduction. The percentage of luciferase for the dCjCas9-HP1A treatment group (FIG. 22B), the dSaCas9-HP1A treatment group (FIG. 22C), the dCjCas9-KRAB-MeCP2 treatment group (FIG. 22D), and the dSaCas9-KRAP-MeCP2 treatment group (FIG. 22D) was normalized by the control group (CRISPR/dCas9 with no sgRNA and no effector). Unpaired two-tail t-test with equal variance (α=0.05) was conducted to find significant gene repression (*p<0.01, **p<0.001, ***p<0.0001).

FIG. 23A is the evaluation of the repression capacity of dCjCas9-KRAB-MeCP2 AAV vectors on the reporter HEK293T-CAG-intron 1-SNCA-GFP-LUC cells by western blotting. FIG. 23B is the evaluation of the repression capacity of dSaCas9-KRAB-MeCP2 AAV vectors on the reporter HEK293T-CAG-intron 1-SNCA-GFP-LUC cells by western blotting.

FIG. 24A shows the Cas-repressor vector screening strategy for the selection of the lead AAV/dCas9-repressor vector. FIG. 24B shows the measurement of the repression capacity of the developed vectors. Briefly, normalized luciferase measured on day 6 after AAV transduction. The data represent the percentage of luciferase for each treatment group normalized by the control group (CRISPR/dCas with no sgRNA and no effector). Unpaired two-tail t-test with equal variance (α=0.05) was conducted to find significant gene repression (*p<0.01, **p<0.001, ***p<0.0001).

FIG. 25A-FIG. 25E show the in vivo validation of the AAV/dCas9-repressor platform in mouse hippocampus using the GFP-reporter gene. FIG. 25A provide the schematic presentation of the workflow of a reporter gene assay. FIG. 25B shows the schematic of the experimental design. Bilateral brain surgery to administrate the investigational (left) and the control (right) AAV/dCas9 vectors into the mouse hippocampus. FIG. 25C shows representative example images of the mouse brain coronal sections from mature adult (4 months, upper panel) and middle age (8 months, lower panel) mice. A strong intense signal (green) observed in the right hippocampus compared to the weak signal on the left side. FIG. 25D shows the statistical analysis of the two experimental cohorts—mature adult (4 months, n=11) and middle age (8 months, n=10) mice. The signals were quantified using ImageJ Threshold function. Each open circle represents the quantified signal hippocampus left/right for a mouse. Values represent mean±SEM. * p<0.05 ** p<0.01; Mann-Whitney U Test. FIG. 25E shows the daily weights post-surgery of mature adult (6 males, 5 females; red) and 10 middle age (5 males, 5 females; blue) mice. There was no significant effect on mouse weights following injection (Day 0).

FIG. 26A-FIG. 26D show the In vitro validation of the efficacy and specificity of the dCas9-repressor viral vector system in hiPSC-derived models. FIG. 26A shows a schematic diagram of the APOE-targeted epigenome therapy system. Left, a representation of the viral-delivered epigenome editing platform; Middle, the expressed platform targeted the APOE gene, red circle designated the e4-SNP; Right, leading to specific reduction in APOE-e4 expression. FIG. 26B shows a validation study of the APOE e4-specific dCas9-repressor platform using human induced pluripotent stem cell (hiPSC) systems—APOE e4 and e3 hiPSC while FIG. 26C shows the same study in derived organoids transduced with gRNA/dCas-repressor or control (no-gRNA) viruses harboring gRNAs complementary to APOE locus. The naïve e4-hiPSC cells used as an additional control. APOE-mRNA levels were quantified by qRT-PCR. The platform resulted in decreased APOE-mRNA expression levels that was specific to the APOE e4 cell-line (FIG. 26B) and organoids (FIG. 26C), whereas no effect of was observed for the control APOEe3 cells (FIG. 26B) and organoids (FIG. 26C). FIG. 26D shows hiPSC-derived cholinergic neurons homozygote to the APOE e4 allele (APOE e4/4) were stably transduced with virus vector carrying gRNAs 1-4 paired with dCas9-repressor or a dCas9-repressor vector with no-gRNA. gRNAs 1-4 designed to target APOE regulatory sequences. The naïve e4-hiPSC cells used as an additional control. Following transduction and selection, the level of APOE-mRNA reduction was assessed using qRT-PCR. gRNA1 and 2 showed the strongest effects. * Significant reduction in APOE-mRNA expression compared to controls.

FIG. 27A shows the cloning strategy employed for an AAV expression cassette having a dual reporting system. Here, two or four copies of the Sp1 and NF-kB (AAV-2×Sp1 or AAV-2×NF-kB, and AAV-4×Sp1 or AAV-4×NF-kB, respectively) and 2 copies of the Sp1 and NF-kB (AAV-2×Sp1—2×NF-kB) were cloned into an AAV expression cassette carrying a dual reporter system comprised from eGFP and NLuc. The transcription binding sites were inserted upstream of the core version of EF-1a-promoter, namely EFS-NC, harboring neither Sp1, nor NF-kB sites. The complete EF-1a-promoter bared multiple Sp1 and NF-kB sites was used as a control. The strong, but bulky (~1500 bps) EF-1a-promoter has obvious restrain for the use with AAV vectors. Conversely, miniature EFS-NC promoter (212 bps long) is highly suitable for the delivery with the AAV systems. The designed vectors were produced in the non-concentrated format and tittered by real-time PCR. FIG. 27B shows that there was no significant difference in the physical titers of the vectors has been detected. Next, the produced vectors were used for the transduction into HEK293T cells at the MOI=10,000. FIG. 27C shows that the vectors carrying Sp1 and NF-kB binding sites demonstrated higher level of NLuc expression compared to the naïve counterpart. The vectors carrying four repeats showed higher level of expression compared to those carried two repeats of the transcription factor binding sites. Furthermore, the vector carried a repeat of both Sp1 and NF-kB cloned upstream of EFS-NC promoter in tandem shown slightly higher level of the reporter expression, nearly reaching the capacity of the vector expressed NLuc from EF-1a promoter.

FIG. 28A shows two gRNA targeting different regions of CMV promoter that were used to test the repression capacity of the vectors. The initial assessment of the repression capacity of the constructs was done by the visualization of the dGFP expressions. FIG. 28B shows the quantification of the data using the NLuc testing.

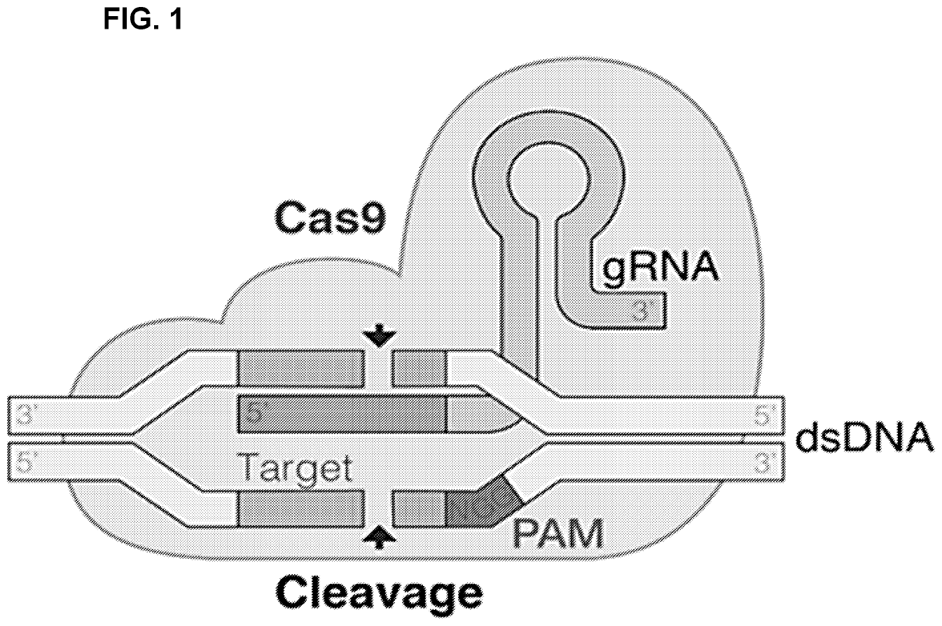

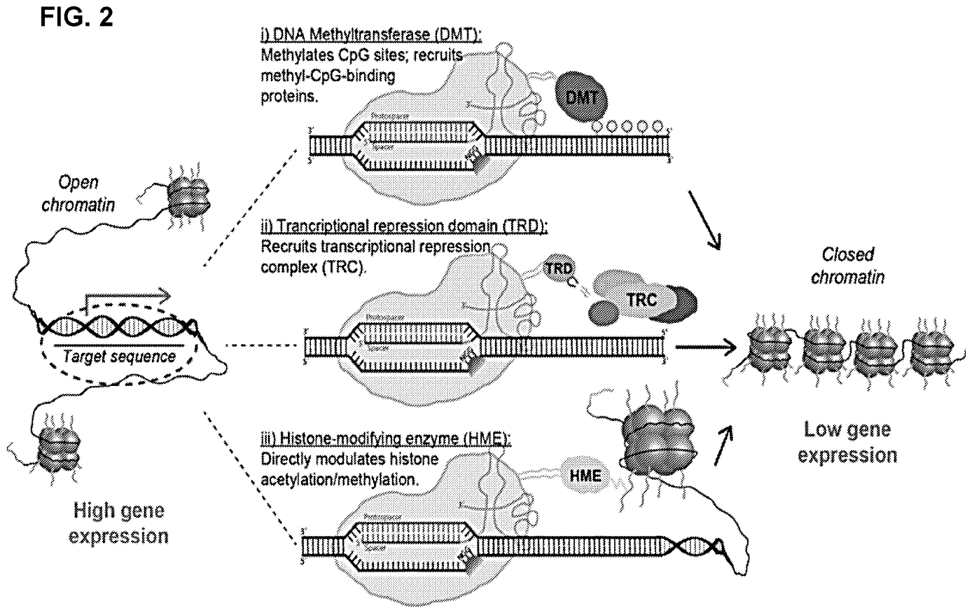

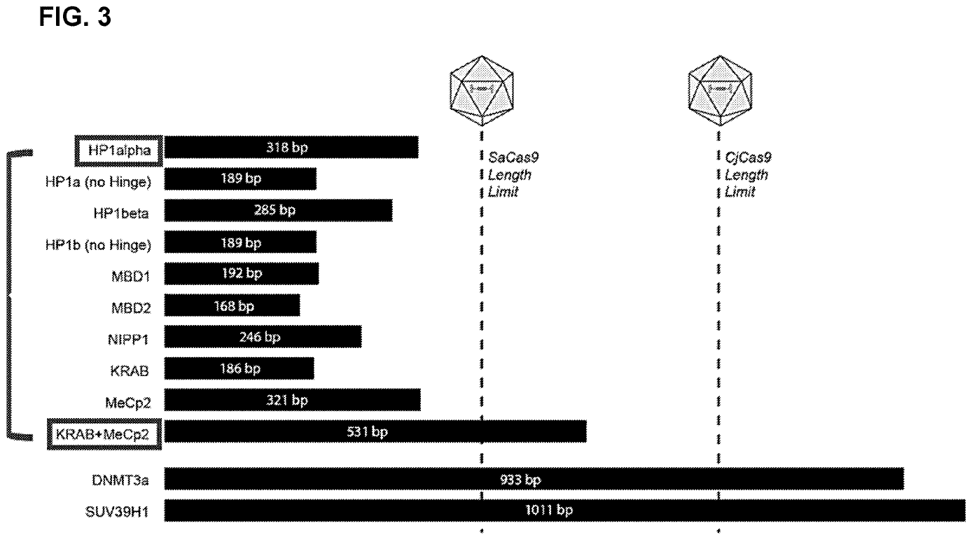

### V. BRIEF SUMMARY

Disclosed herein is an isolated nucleic acid molecule, comprising: a nucleic acid sequence encoding (i) a deactivated Cas (dCas) endonuclease, (ii) at least one polypeptide having an enzymatic activity, and (iii) at least one guide RNA targeting a gene of interest or portion thereof.

Disclosed herein is an isolated nucleic acid molecule, comprising: a nucleic acid sequence encoding (i) a deactivated Cas (dCas) endonuclease, (ii) a fusion protein having one or more enzymatic activities, and (iii) at least one guide RNA targeting a gene of interest or portion thereof

Disclosed herein is an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof.

Disclosed herein is an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA.

Disclosed herein is an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof.

Disclosed herein is an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA.

Disclosed herein is a viral vector comprising a disclosed isolated nucleic acid molecule. Disclosed herein is a viral vector comprising a disclosed nucleic acid molecule. Disclosed herein is a viral vector comprising a nucleic acid sequence encoding (i) a deactivated Cas (dCas) endonuclease, (ii) at least one polypeptide having an enzymatic activity, and (iii) at least one guide RNA targeting a gene of interest or portion thereof. Disclosed herein is a viral vector comprising a nucleic acid sequence encoding (i) a deactivated Cas (dCas) endonuclease, (ii) a fusion protein having one or more enzymatic activities, and (iii) at least one guide RNA targeting a gene of interest or portion thereof.

Disclosed herein is a recombinant AAV vector comprising a disclosed isolated nucleic acid molecule. Disclosed herein is a recombinant AAV vector comprising a disclosed nucleic acid molecule. Disclosed herein is a recombinant AAV vector comprising a nucleic acid sequence encoding (i) a deactivated Cas (dCas) endonuclease, (ii) at least one polypeptide having an enzymatic activity, and (iii) at least one guide RNA targeting a gene of interest or portion thereof. Disclosed herein is a recombinant AAV vector comprising a nucleic acid sequence encoding (i) a deactivated Cas (dCas) endonuclease, (ii) a fusion protein having one or more enzymatic activities, and (iii) at least one guide RNA targeting a gene of interest or portion thereof.

Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof.

Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA.

Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof.

Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA.

Disclosed herein is a method of effecting precision epigenetic modulation, the method comprising contacting one or more cells with a therapeutically effective amount of a disclosed isolated nucleic acid molecule, wherein the expression and/or activity of one or more genes of interest in the one or more cells is modulated. Disclosed herein is a method of effecting precision epigenetic modulation, the method comprising contacting one or more cells with a therapeutically effective amount of a disclosed isolated nucleic acid molecule, wherein the expression and/or activity of one or more genes of interest in the one or more cells is reduced or decreased.

Disclosed herein is a method of effecting precision epigenetic modulation, the method comprising contacting one or more cells with a therapeutically effective amount of a disclosed isolated nucleic acid molecule, wherein the expression and/or activity of one or more genes of interest in the one or more cells is augment or increased. Disclosed herein is a method of effecting precision epigenetic modulation, the method comprising contacting one or more cells with a therapeutically effective amount of a disclosed isolated nucleic acid molecule, wherein the expression and/or activity of APOE e4 in the one or more cells is reduced or decreased.

Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, (ii) at least one polypeptide having an enzymatic activity, and (iii) at least one guide RNA targeting a gene of interest or portion thereof, wherein the expression and/or activity the gene of interest in the one or more cells is modulated. Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, (ii) a fusion protein having one or more enzymatic activities, and (iii) at least one guide RNA targeting a gene of interest or portion thereof, wherein the expression and/or activity the gene of interest in the one or more cells is modulated.

Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof, wherein the expression and/or activity the gene of interest in the one or more cells is modulated.

Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA, wherein the expression and/or activity the gene of interest in the one or more cells is modulated.

Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof, wherein the expression and/or activity the gene of interest in the one or more cells is modulated.

Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA, wherein the expression and/or activity the gene of interest in the one or more cells is modulated.

Disclosed herein is a method of effecting precision epigenetic modulation, the method comprising contacting one or more cells in a subject with a therapeutically effective amount of a disclosed viral vector or disclosed rAAV vector, wherein the expression and/or activity of one or more genes of interest in the one or more cells is modulated.

Disclosed herein is a method of effecting precision epigenetic modulation, the method comprising contacting one or more cells in a subject with a therapeutically effective amount of a disclosed viral vector or disclosed rAAV vector, wherein the expression and/or activity of one or more genes of interest in the one or more cells is reduced or decreased. Disclosed herein is a method of effecting precision epigenetic modulation, the method comprising contacting one or more cells in a subject with a therapeutically effective amount of a disclosed viral vector or disclosed rAAV vector, wherein the expression and/or activity of one or more genes of interest in the one or more cells is augment or increased. Disclosed herein is a method of effecting precision epigenetic modulation, the method comprising contacting one or more cells in a subject with a therapeutically effective amount of a disclosed viral vector or disclosed rAAV vector, wherein the expression and/or activity of APOE e4 in the one or more cells is reduced or decreased.

Disclosed herein is a method of effecting precision epigenetic modulation, the method comprising contacting one or more cells in a subject with a therapeutically effective amount of a viral vector comprising a nucleic acid sequence encoding (i) a deactivated Cas (dCas) endonuclease, (ii) at least one polypeptide having an enzymatic activity, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a viral vector comprising a nucleic acid sequence encoding (i) a deactivated Cas (dCas) endonuclease, (ii) a fusion protein having one or more enzymatic activities, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a recombinant AAV vector comprising a nucleic acid sequence encoding (i) a deactivated Cas (dCas) endonuclease, (ii) at least one polypeptide having an enzymatic activity, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a recombinant AAV vector comprising a nucleic acid sequence encoding (i) a deactivated Cas (dCas) endonuclease, (ii) a fusion protein having one or more enzymatic activities, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA, wherein the expression and/or activity of one or more genes of interest in the one or more cells is modulated.

Disclosed herein is a method of effecting precision epigenetic modulation, the method comprising contacting one or more cells in a subject with a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA, wherein the expression and/or activity of one or more genes of interest in the one or more cells is modulated.

Disclosed herein is a method of treating and/or preventing Alzheimer's disease progression in a subject, the method comprising administering to a subject a therapeutically effective amount of a disclosed isolated nucleic acid molecule, thereby reducing the pathological phenotype associated with Alzheimer's disease.

Disclosed herein is a method of treating and/or preventing Alzheimer's disease progression in a subject, the method comprising administering to a subject a therapeutically effective amount of an isolated nucleic acid molecule, comprising: a nucleic acid sequence encoding (i) a deactivated Cas (dCas) endonuclease, (ii) at least one polypeptide having an enzymatic activity, and (iii) at least one guide RNA targeting the APOE gene or a portion of the APOE gene, thereby reducing the pathological phenotype associated with Alzheimer's disease.

Disclosed herein is a method of treating and/or preventing Parkinson's disease progression in a subject, the method comprising administering to a subject a therapeutically effective amount of a disclosed isolated nucleic acid molecule, thereby reducing the pathological phenotype associated with Parkinson's disease.

Disclosed herein is a method of treating and/or preventing Parkinson's disease progression in a subject, the method comprising administering to a subject a therapeutically effective amount of an isolated nucleic acid molecule, comprising: a nucleic acid sequence encoding (i) a deactivated Cas (dCas) endonuclease, (ii) at least one polypeptide having an enzymatic activity, and (iii) at least one guide RNA targeting the SNCA gene or a portion of the SNCA gene, thereby reducing the pathological phenotype associated with Parkinson's disease.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Alzheimer's Disease

Alzheimer's disease (AD) is the sixth leading cause of death in the United States and the most common cause of dementia in aging. With a rapidly growing aging population, the number of AD cases is growing fast and is projected to rise drastically over the next three decades. In the U.S. alone today, more than 5 million people are living with AD. By 2050, this number is projected to reach 14 million cases. Therefore, AD poses a huge economic burden on society placing overwhelming strain on the healthcare system. Currently, the cost of AD to the US is $301 billion, including $206 billion in Medicare and Medicaid payments. It is estimated that caregivers provide 244 billion dollars' worth of care (Costs of Alzheimer's to Medicare and Medicaid. (2020); Alzheimer's Association, Alzheimer's Impact Movement: Fact Sheet 2020). In the absence of a treatment that halts or prevents AD, the cost of AD to society is projected to be more than $1.1 trillion annually by 2050. Mainstream research for LOAD therapies continues to focus on the Amyloid Cascade Hypothesis, and as such Aβ has been for decades the leading molecular target (Mawuenyega K G, et al. (2013). Anal Biochem 440:56-62). Despite all the research effort, money, and commitment, numerous clinical trials aimed at identifying disease modifying therapies (DMT) for AD have failed. Current treatments are mostly palliative and aimed at relieving symptoms management (Sharma K. (2019). Mol Med Rep 20:1479-1487; Olivares D, et al. (2012). Curr Alzheimer Res 9:746-758). Thus, AD remains an unmet medical need underscoring the urgent need for a paradigm shift in AD drug discovery research.

### B. Late Onset Alzheimer's Disease (LOAD)

LOAD is a heterogenous disease with various genetic etiologies (Lo M T, et al. (2019). Neurobiol Aging 84:243 e1-243 e9; Nacmias B, et al. (2018). J Alzheimers Dis 62:903-911). A major reason for the failure to identify an effective treatment is likely the inaccurate consideration of LOAD as a homogeneous disease. In this respect, mounting evidence demonstrates the heterogeneity in the underlying pathophysiologic processes of LOAD and shows variability in the genetic risk and molecular profiles amongst AD patients (Reitz C. (2016). Ann Transl Med 4:107; Chiba-Falek O, et al. (2017). Expert Rev Precis Med Drug Dev 2:47-55). Advancement in LOAD therapy requires the development and validation of new therapeutic targets including those tailored to a specific sub-group of patients with a specific set of risk factors. APOE has emerged as potential promising targets for LOAD treatment (Huynh T V, et al. (2017). Neuron 96:1013-1023 e4; Brody D L, et al. (2008). Annu Rev Neurosci 31:175-193; Kim J, et al. (2012). J Exp Med 209:2149-2156).

### C. Apolipoprotein E (ApoE)

Apoliporotein E is encoded by the APOE gene positioned on chromosome 19q13.32 (GRCh 38: chr19:44,905,795-44,909,392). Two common coding SNPs in exon 4 give rise to three allelic variants (APOE e2, APOE e3, and APOE e4), encoding three corresponding protein isoforms that differ at two amino acid positions 112 and 158. The e4 allele of the apolipoprotein E gene (APOE e4) is the first, strongest, and most firmly established genetic risk factor for LOAD (Corder E H, et al. (1993). Science 261:921-923; Liu N, et al. (2008). Adv Genet 60:335-405; Schmechel D E, et al. (1993). Proc Natl Acad Sci USA 90:9649-9653; Saunders A M, et al. (1993). Neurology 43:1467-1472).

The initial discovery was made nearly 30 years ago by linkage analysis of pedigrees (Corder E H, et al. (1993). Science 261:921-923). Over the ensuing years, APOE e4 has become the most highly replicated genetic risk factor for LOAD (Corder E H, et al. (1993). Science 261:921-923; Liu N, et al. (2008). Adv Genet 60:335-405; Schmechel D E, et al. (1993). Proc Natl Acad Sci USA 90:9649-9653; Saunders A M, et al. (1993). Neurology 43:1467-1472). Subsequent LOAD genome-wide association studies (GWAS) have confirmed strong associations with the APOE genomic region, and no other LOAD-association remotely approached the same level of significance (Harold D, et al. (2009). Nat Genet 41:1088-1093; Lambert J C, et al. (2009). Nat Genet 41:1094-1099; Heinzen E L, et al. (2009). J Alzheimers Dis; Kamboh M I, et al. (2012). Mol Psychiatry 17:1340-1346; Kamboh M I, et al. (2012). Transl Psychiatry 2:e117; Seshadri S, et al. (2010). Jama 303:1832-1840; Kunkle B W, et al. (2019). Nat Genet 51:414-430; Lambert J C, et al. (2013). Nat Genet 45:1452-1458; Coon K D, et al. (2007). J Clin Psychiatry 68:613-618).

Carrying the APOE e4 variant significantly increases the lifetime risk for LOAD, whereas the number of e4 copies affects the level of risk and is associated with lower age of clinical disease onset (Corder E H, et al. (1993). Science 261:921-923; Farrer L A, et al. (1997). Jama 278:1349-1356). APOE e3 is natural and APOE e2 confers a protective effect (Saunders A M, et al. (1993). Neurology 43:1467-1472; Farrer L A, et al. (1997). Jama 278:1349-1356; Reiman E M, et al. (2020). Nat Commun 11:667).

About 40-65% of LOAD patients carry the e4 allele as compared to 10-15% in the general population. Data indicates that ApoE e4 acquired hyperfunction (gain of toxic effects) (Farrer L A, et al. (1997). Jama 278:1349-1356). Data implicates several cellular pathways through which ApoE e4 may exert toxicity associated with LOAD pathologic phenotypes (Huang Y A, et al. (2017). Cell 168:427-441 e21; Sen A, et al. (2015). J Neurosci 35:7538-7551; Theendakara V, et al. (2013). Proc Natl Acad Sci USA 110:18303-18308; Theendakara V, et al. (2016). J Neurosci 36:685-700; Min S W, et al. (2010). Neuron 67:953-966; Tambini M D, et al. (2016). EMBO Rep 17:27-36; Hatters D M, et al. (2006). J Mol Biol 361:932-944). Thus, decreasing the levels of ApoE e4 has a therapeutic implication. However, ApoE e4 as a target for LOAD remains significantly understudied, despite the few recent studies that have begun to pave the way.

### D. Relevant Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have Alzheimer's disease, be suspected of having Alzheimer's disease, or be at risk of developing and/or acquiring Alzheimer's disease. In an aspect, a subject can have Parkinson's disease, be suspected of having Parkinson's disease, or be at risk of developing and/or acquiring Parkinson's disease.

As used herein, the term “diagnosed” means having been subjected to an examination by a person of skill, for example, a physician, and found to have a condition that can be diagnosed or treated by one or more of the disclosed agents, disclosed therapeutic agents, disclosed pharmaceutical formulations, or a combination thereof, or by one or more of the disclosed methods. For example, “diagnosed with Alzheimer's disease” or “diagnosed with Parkinson's disease” means having been subjected to an examination by a person of skill, for example, a physician, and found to have a condition that can be treated by one or more of the disclosed isolated nucleic acid molecules, disclosed viral vectors, disclosed rAAV vectors, disclosed pharmaceutical formulations, disclosed host cells, disclosed gRNAs, disclosed plasmids, or any combination thereof, or by one or more of the disclosed methods. For example, “suspected of having Alzheimer's disease” or “suspected of having Parkinson's disease” can mean having been subjected to an examination by a person of skill, for example, a physician, and found to have a condition that can likely be treated by one or more of the disclosed isolated nucleic acid molecules, disclosed viral vectors, disclosed rAAV vectors, disclosed pharmaceutical formulations, disclosed host cells, disclosed gRNAs, disclosed plasmids, or any combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” can refer to a subject that has been diagnosed with or is suspected of having Alzheimer's disease (AD) or Parkinson's disease. In an aspect, a patient can refer to a subject that has been diagnosed with or is suspected of having AD or PD and is seeking treatment or receiving treatment for AD or PD.

As used herein, the phrase “identified to be in need of treatment for a disorder,” or the like, refers to selection of a subject based upon need for treatment of the disorder. For example, a subject can be identified as having a need for treatment of a disorder (e.g., such as Alzheimer's disease or Parkinson's disease) based upon an earlier diagnosis by a person of skill and thereafter subjected to treatment for the disorder (e.g., AD or Parkinson's disease). In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, expression, condition, severity, disease, or other biological parameter. This can include, but is not limited to, the complete ablation of the activity, level, response, expression, condition, severity, disease, or other biological parameter. This can also include, for example, a 10% inhibition or reduction in the activity, level, response, condition, severity, disease, or other biological parameter as compared to the native or control level (e.g., a subject not having Alzheimer's disease or Parkinson's disease). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as Alzheimer's disease or Parkinson's disease). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating Alzheimer's disease or Parkinson's disease can reduce the severity of an established disease in a subject by 1%-100% as compared to a control (such as, for example, an individual not having AD or PD). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of AD or PD. For example, treating Alzheimer's disease can reduce one or more symptoms of AD or PD in a subject by 1%-100% as compared to a control (such as, for example, an individual not having AD or PD). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established AD or PD. It is understood that treatment does not necessarily refer to a cure or complete ablation or eradication of AD or PD. However, in an aspect, treatment can refer to a cure or complete ablation or eradication of AD or PD.

As used herein, a “biomarker” refers to a defined characteristic that is measured as an indicator of normal biological processes, pathogenic processes, or response to an exposure of intervention. In an aspect, a biomarker can be diagnostic (i.e., detects or classifies a pathological condition), prognostic (i.e., predicts the probability of disease occurrence or progression), pharmacodynamic/responsive (i.e., identifies a change in response to a therapeutic intervention), predictive (i.e., predicts how an individual or subject might respond to a particular intervention or event). In an aspect, a biomarker can be diagnostic, prognostic, pharmacodynamic/responsive, and/or predictive at the same time. In an aspect, a biomarker can be diagnostic, prognostic, pharmacodynamic/responsive, and/or predictive at different times (e.g., first a biomarker can be diagnostic and then later, the same biomarker can be prognostic, pharmacodynamic/responsive, and/or predictive). A biomarker can be an objective measure that can be linked to a clinical outcome assessment. A biomarker can be used by the skilled person to make a clinical decision based on its context of use.

As used herein, “operably linked” means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, a “regulatory element” can refer to promoters, enhancers, internal ribosomal entry sites (IRES), and other expression control elements (e.g., transcription termination signals, such as polyadenylation signals and poly-U sequences). Regulatory elements are discussed infra and can include those that direct constitutive expression of a nucleotide sequence in many types of host cells and those that direct expression of the nucleotide sequence only in certain host cells (e.g., tissue-specific regulatory sequences).

As known to the art, RNA binding proteins consist of multiple repetitive sequences that contain only a few specific basic domains. Structurally, common RNA-binding domains mainly include RNA-recognition motif (RRM), K homology (KH) domain, double-stranded RBD (dsRBD), cold-shock domain (CSD), arginine-glycine-glycine (RGG) motif, tyrosine-rich domain, and zinc fingers (ZnF) of the CCHC, CCCH, ZZ type etc. According to the different functions of RBPs in cells, RBPs can be divided into epithelial splicing regulatory proteins (ESRP1), cytoplasmic polyadenylation element binding protein family (CPEB1/2), Hu-antigen R (HuR), heterogeneous nuclear ribonucleoprotein family members (hnRNP A/D/H/K/M/E/L), insulin-like growth factor 2 mRNA family members (IMP1/2/3), zfh family of transcription factors (ZEB1/2), KH-type splicing regulatory protein (KHSRP), La ribonucleoprotein domain family members (LARP1/6/7), Lin-28 homolog proteins (Lin28), Musashi protein family (MSI1/2), Pumilio protein family (PUM1/2), Quaking (QK), RNA-binding motif protein family (4/10/38/47), Src-associated substrate during mitosis of 68 kDa (SAM68), serine and arginine rich splicing factor (SRSF1/3), T cell intracellular antigens (TIA1/TIAR), and Upstream of N-Ras (UNR).

As used herein, “immune tolerance,” “immunological tolerance,” and “immunotolerance” refers to a state of unresponsiveness or blunted response of the immune system to substances (e.g., a disclosed isolated nucleic acid molecule, a disclosed vector, a disclosed transgene product, a disclosed pharmaceutical formulation, a disclosed therapeutic agent, etc.) that have the capacity to elicit an immune response in a subject. Immune tolerance is induced by prior exposure to a specific antigen. Immune tolerance can be determined in a subject by measuring antibodies against a particular antigen or by liver-restricted transgene expression with a viral vector (such as, for example, AAV). Low or absent antibody titers over time is an indicator of immune tolerance. For example, in an aspect, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, a missing, deficient, and/or mutant protein or enzyme).

As used herein, “peptide,” “polypeptide,” and “protein” are used interchangeably, and refer to a compound comprised of amino acid residues covalently linked by peptide bonds. A protein must contain at least two amino acids and there is no limitation on the maximum number of amino acids that can comprise a protein's sequence. The term “peptide” can refer to a short chain of amino acids including, for example, natural peptides, recombinant peptides, synthetic peptides, or any combination thereof. Proteins and peptides can include, for example, biologically active fragments, substantially homologous polypeptides, oligopeptides, homodimers, heterodimers, variants of polypeptides, modified polypeptides, derivatives, analogs, and fusion proteins, among others.

“Nucleic acid” or “oligonucleotide” or “polynucleotide” as used herein means at least two nucleotides covalently linked together. The depiction of a single strand can also define the sequence of the complementary strand. Thus, a nucleic acid can encompass the complementary strand of a depicted single strand. Many variants of a nucleic acid can be used for the same purpose as a given nucleic acid. Thus, a nucleic acid can encompass substantially identical nucleic acids and complements thereof. A single strand can provide a probe that can hybridize to a target sequence under stringent hybridization conditions. Thus, a nucleic acid can encompass a probe that hybridizes under stringent hybridization conditions. A nucleic acid can be single-stranded, or double-stranded, or can contain portions of both double-stranded and single-stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. Also as used herein, the terms “nucleic acid,” “nucleic acid molecule,” “nucleic acid construct,” “nucleotide sequence”, and “polynucleotide” can refer to RNA or DNA that is linear or branched, single or double stranded, or a hybrid thereof. The term can encompass RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. A “synthetic” nucleic acid or polynucleotide, as used herein, refers to a nucleic acid or polynucleotide that is not found in nature but is constructed by the hand of man and therefore is not a product of nature.

A “polynucleotide” is a sequence of nucleotide bases, and may be RNA, DNA, or DNA-RNA hybrid sequences (including both naturally occurring and non-naturally occurring nucleotides).

A “fragment” or “portion” of a nucleotide sequence can be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of, or consisting of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the disclosure can be, where appropriate, included in a larger polynucleotide of which it is a constituent. In an aspect, a fragment or portion of a nucleotide sequence or nucleic acid sequence can comprise the sequence encoding an exon having one or more mutations. In an aspect, a fragment or portion of a nucleotide sequence or nucleic acid sequence can comprise a target of interest.

A “fragment” or “portion” of an amino acid sequence can be understood to mean an amino acid sequence of reduced length relative (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, or more amino acids) to a reference amino acid sequence and comprising, consisting essentially of, or consisting of an amino acid sequence of contiguous amino acids identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference amino acid sequence. Such an amino acid fragment or portion according to the disclosure can be, where appropriate, included in a larger amino acid sequence of which it is a constituent.

A “heterologous” or a “recombinant” nucleotide or amino acid sequence as used interchangeably herein can refer to a nucleotide or an amino acid sequence not naturally associated with a host cell into which it is introduced, including non-naturally occurring multiple copies of a naturally occurring nucleotide or amino acid sequence.

Different nucleic acids or proteins having homology can be referred to as “homologues”. The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the disclosed compositions and disclosed methods can comprise homologues to the disclosed nucleotide sequences and/or disclosed polypeptide sequences.

“Orthologous,” as used herein, can refer to homologous nucleotide sequences and/or amino acid sequences in different species that arose from a common ancestral gene during speciation. A homologue of a disclosed nucleotide sequence or a disclosed polypeptide can have substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to a disclosed nucleotide sequence or a disclosed polypeptide.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

Ubiquitous/constitutive promoters” are known to the art and include, but are not limited to, a CMV major immediate-early enhancer/chicken beta-actin promoter, a cytomegalovirus (CMV) major immediate-early promoter, an Elongation Factor 1-α (EF1-α) promoter, a simian vacuolating virus 40 (SV40) promoter, an AmpR promoter, a P7K promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a 3-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

As used herein, the term “serotype” is a distinction used to refer to an AAV having a capsid that is serologically distinct from other AAV serotypes. Serologic distinctiveness can be determined by the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes).

As used herein, “tropism” refers to the specificity of an AAV capsid protein present in an AAV viral particle, for infecting a particular type of cell or tissue. The tropism of an AAV capsid for a particular type of cell or tissue may be determined by measuring the ability of AAV vector particles comprising the hybrid AAV capsid protein to infect or to transduce a particular type of cell or tissue, using standard assays that are well-known in the art such as those disclosed in the examples of the present application. As used herein, the term “liver tropism” or “hepatic tropism” refers to the tropism for liver or hepatic tissue and cells, including hepatocytes.

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, “expression cassette” or “transgene cassette” can refer to a distinct component of vector DNA comprising a transgene and one or more regulatory sequences to be expressed by a transfected cell. Generally, an expression cassette or transgene cassette can comprise a promoter sequence, an open reading frame (i.e., the transgene), and a 3′ untranslated region (e.g., in eukaryotes a polyadenylation site).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing progression of Alzheimer's disease (AD) or Parkinson's disease (PD) is intended. The words “prevent” and “preventing” and “prevention” also refer to prophylactic or preventative measures for protecting or precluding a subject (e.g., an individual) not having AD or PD or an AD-related or PD-related complication from progressing to that complication. In an aspect, preventing or reducing APOE expression and/or activity is intended. In an aspect, preventing or reducing SCNA expression and/or activity is intended.

As used herein, the terms “administering” and “administration” refer to any method of providing one or more of the disclosed isolated nucleic acid molecules, disclosed pharmaceutical formulations, disclosed vectors, or any combination thereof to a subject. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed therapeutic agent, a disclosed pharmaceutical composition, or a combination thereof can comprise administration directly into the CNS (e.g., intraparenchymal, intracerebroventriular, inthrathecal cisternal, intrathecal (lumbar), deep gray matter delivery, convection-enhanced delivery to deep gray matter) or the PNS. Administration can be continuous or intermittent.

In an aspect, a “therapeutic agent” can be a “biologically active agent” or “biologic active agent” or “bioactive agent”, which refers to an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

In an aspect, a “therapeutic agent” can be any agent that effects a desired clinical outcome in a subject having AD or PD, suspected of having AD or PD, and/or likely to develop or acquire AD or PD. In an aspect, a disclosed therapeutic agent can be an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable.

By “determining the amount” is meant both an absolute quantification of a particular analyte (e.g., an mRNA sequence) or a determination of the relative abundance of a particular analyte (e.g., an amount as compared to a mRNA sequence). The phrase includes both direct or indirect measurements of abundance (e.g., individual mRNA transcripts may be quantified or the amount of amplification of an mRNA sequence under certain conditions for a certain period may be used a surrogate for individual transcript quantification) or both.

As used herein, “modifying the method” can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, by changing the duration of time one or more of the disclosed isolated nucleic acid molecules, disclosed vectors, disclosed pharmaceutical formulations, or a combination are administered to a subject, or by substituting for one or more of the disclosed components and/or reagents with a similar or equivalent component and/or reagent. The same applies to all disclosed therapeutic agents, immune modulators, immunosuppressive agents, proteosome inhibitors, etc.

In an aspect, a therapeutic agent can be a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. Examples include but are not limited to a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of glycogen branching enzymes and amylases. Sequences with substantial sequence identity do not necessarily have the same length and may differ in length. For example, sequences that have the same nucleotide sequence but of which one has additional nucleotides on the 3′- and/or 5′-side are 100% identical.

In an aspect, the skilled person can determine an efficacious dose, an efficacious schedule, and an efficacious route of administration for one or more of the disclosed isolated nucleic acid molecules, disclosed pharmaceutical formulations, disclosed vectors, or any combination thereof so as to treat or prevent AD or PD. In an aspect, the skilled person can also alter, change, or modify an aspect of an administering step to improve efficacy of one or more of the disclosed isolated nucleic acid molecules, disclosed pharmaceutical formulations, disclosed vectors, or any combination thereof. In an aspect, the skilled person can determine an efficacious dose, an efficacious schedule, and an efficacious route of administration for any disclosed isolated nucleic acid molecule, disclosed pharmaceutical formulation, disclosed vector, disclosed therapeutic agent, or any combination thereof.

As used herein, “modifying the method” can comprise modifying or changing one or more features or aspects of one or more steps of a disclosed method. For example, in an aspect, a method can be altered by changing the amount of one or more of the disclosed isolated nucleic acid molecules, disclosed viral vectors, disclosed rAAV vectors, disclosed pharmaceutical formulations, disclosed host cells, disclosed gRNAs, disclosed plasmids, or any combination thereof, or administered to a subject, or by changing the frequency of administration of one or more of the disclosed isolated nucleic acid molecules, disclosed viral vectors, disclosed rAAV vectors, disclosed pharmaceutical formulations, disclosed host cells, disclosed gRNAs, disclosed plasmids, or any combination thereof, or by changing the duration of time that the one or more of the disclosed isolated nucleic acid molecules, disclosed viral vectors, disclosed lentiviral vectors, disclosed pharmaceutical formulations, disclosed host cells, disclosed gRNAs, disclosed plasmids, or any combination thereof, or are administered to a subject.

As used herein, “isolated” refers to a nucleic acid molecule or a nucleic acid sequence that has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated proteins or nucleic acids, or cells containing such, in some examples are at least 50% pure, such as at least 75%, at least 80%, at least 90%, at least 95%, at least 98%, or at least 100% pure.

As used herein, “concurrently” means (1) simultaneously in time, or (2) at different times during the course of a common treatment schedule.

The term “contacting” as used herein refers to bringing one or more of disclosed isolated nucleic acid molecules, disclosed pharmaceutical formulations, disclosed vectors, or any combination thereof together with a target area or intended target area in such a manner that the one or more of the disclosed isolated nucleic acid molecules, disclosed pharmaceutical formulations, disclosed vectors, or any combination thereof exert an effect on the intended target or targeted area either directly or indirectly. A target area or intended target area can be one or more of a subject's organs (e.g., lungs, heart, liver, kidney, brain, etc.). In an aspect, a target area or intended target area can be any cell or any organ infected by AD or PD (such as cholinergic neurons). In an aspect, a target area or intended target area can be the brain or various neuronal populations. In an aspect, a target area or intended target area can be any cell or any organ infected by an over-expression or an under-expression of one or more genes.

As used herein, “determining” can refer to measuring or ascertaining the presence and severity of AD or PD. Methods and techniques used to determine the presence and/or severity of AD or PD are typically known to the medical arts. For example, the art is familiar with the ways to identify and/or diagnose the presence, severity, or both of AD or PD. In an aspect, “determining” can also refer to measuring or ascertaining the level of one or more proteins or peptides in a biosample, or measuring or ascertaining the level or one or more RNAs or miRNAs in a biosample. Methods and techniques for determining the expression and/or activity level of relevant proteins, peptides, mRNA, DNA, or any combination thereof known to the art and are disclosed herein.

As used herein, “effective amount” and “amount effective” can refer to an amount that is sufficient to achieve the desired result such as, for example, the treatment and/or prevention of AD or PD. As used herein, the terms “effective amount” and “amount effective” can refer to an amount that is sufficient to achieve the desired an effect on an undesired condition (e.g., a AD or PD). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed isolated nucleic acid molecule, a disclosed pharmaceutical formulation, a disclosed vector, or any combination thereof that (i) treats the particular disease, condition, or disorder (e.g., AD or PD), (ii) attenuates, ameliorates, or eliminates one or more symptoms of the particular disease, condition, or disorder (e.g., AD or PD), or (iii) delays the onset of one or more symptoms of the particular disease, condition, or disorder described herein (e.g., AD or PD). The specific therapeutically effective dose level for any particular patient will depend upon a variety of factors including the disorder being treated and the severity of the disorder; the isolated nucleic acid molecules, disclosed pharmaceutical formulations, disclosed vectors, or any combination thereof employed; the disclosed methods employed; the age, body weight, general health, sex and diet of the patient; the time of administration; the route of administration; the rate of excretion of the disclosed isolated nucleic acid molecules, disclosed pharmaceutical formulations, disclosed vectors, or any combination thereof employed; the duration of the treatment; drugs used in combination or coincidental with the disclosed isolated nucleic acid molecules, disclosed pharmaceutical formulations, disclosed vectors, or any combination thereof employed, and other like factors well known in the medical arts. For example, it is well within the skill of the art to start doses of the disclosed isolated nucleic acid molecules, disclosed pharmaceutical formulations, disclosed vectors, or any combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecules, disclosed pharmaceutical formulations, disclosed vectors, or any combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, AD or PD

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of one or more of the disclosed agents, disclosed therapeutic agents, disclosed nucleic acid molecules, disclosed viral vectors, disclosed rAAV vectors, disclosed pharmaceutical formulations or a combination thereof includes the use of more than one therapy (e.g., additional therapeutic agents). Administration “in combination with” one or more additional therapeutic agents includes simultaneous (e.g., concurrent) and consecutive administration in any order. The use of the term “in combination” does not restrict the order in which therapies are administered to a subject. By way of non-limiting example, a first therapy (e.g., one or more of the disclosed isolated nucleic acid molecules, disclosed pharmaceutical formulations, disclosed vectors, or any combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., one or more of the disclosed isolated nucleic acid molecules, disclosed pharmaceutical formulations, disclosed vectors, or any combination thereof or one or more additional therapeutic agents) to a subject having or diagnosed with AD or PD.

As used herein, “CRISPR or clustered regularly interspaced short palindromic repeat” is an ideal tool for correction of genetic abnormalities associated with diseases such as Alzheimer's disease or PD. The system can be designed to target genomic DNA directly. A CRISPR system involves two main components: a Cas9 enzyme and a guide (gRNA). The gRNA contains a targeting sequence for DNA binding (at, for example, the APOE promoter region) and a scaffold sequence for Cas9 binding. Cas9 nuclease is often used to “knockout” target genes such as for example, the APOE e4 allele. Also, multiple gRNAs can be employed to suppress or activate multiple genes simultaneously, hence increasing the treatment efficacy and reducing resistance potentially caused by new mutations in the target genes.

As used herein, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RNAs to cleave different target nucleic acid sequences. CRISPR-based endonucleases are RNA-guided endonucleases derived from CRISPR/Cas systems.

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Cszl, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Campylobacter jejuni, Oscillatoria sp., Petrotoga mobilis, Thermosipho africanus, or Acaryochloris marina. In an aspect, the CRISPR-based nuclease can be derived from a Cas9 protein from Streptococcus pyogenes. In an aspect, the CRISPR-based nuclease can comprise the sequence set forth in any one of SEQ ID NO:22-SEQ ID NO:27.

As used herein, “CRISPRa” refers to CRISPR Activation, which is using a dCas9 or dCas9-activator with a gRNA to increase transcription of a target gene.

As used herein, “CRISPRi” refers to CRISPR Interference, which is using a dCas9 or dCas9-repressor with a gRNA to repress/decrease transcription of a target gene.

As used herein, “dCas9” refers to enzymatically inactive form of Cas9, which can bind, but cannot cleave, DNA.

As used herein, “Protospacer Adjacent Motif” or “PAM” refers to a sequence adjacent to the target sequence that is necessary for Cas enzymes to bind target DNA.

Disclosed are the components to be used to prepare the disclosed isolated nucleic acid molecules, disclosed pharmaceutical formulations, disclosed vectors, or any combination thereof as well the disclosed isolated nucleic acid molecules, disclosed pharmaceutical formulations, disclosed vectors, or any combination thereof used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspects or combination of aspects of the disclosed methods.

### E. Compositions

### 1. Isolated Nucleic Acid Molecules

Disclosed herein is an isolated nucleic acid molecule, comprising: a nucleic acid sequence encoding (i) a deactivated Cas (dCas) endonuclease, (ii) at least one polypeptide having an enzymatic activity, and (iii) at least one guide RNA targeting a gene of interest or portion thereof.

Disclosed herein is an isolated nucleic acid molecule, comprising: a nucleic acid sequence encoding (i) a deactivated Cas (dCas) endonuclease, (ii) a fusion protein having one or more enzymatic activities, and (iii) at least one guide RNA targeting a gene of interest or portion thereof.

In an aspect, a disclosed dCas endonuclease can comprise a dCas9 endonuclease.

In an aspect, a disclosed dCas9 endonuclease can comprise a deactivated Staphylococcus aureus Cas9 (dSaCas9), a deactivated Streptococcus pyogenes Cas9 (dSpCas9), a deactivated Campylobacter jejuni Cas9 (dCjCas9), or a variant dCas9 endonuclease. In an aspect, a disclosed variant dCas9 can comprise a variant dSaCas9, a variant dSpCas9, or a variant dCjCa9. In an aspect, a disclosed variant dSpCas9 can comprise dVQR, dEQR, or dVRER.

In an aspect, a disclosed dSpCas9 can comprise the sequence set forth in SEQ ID NO:22, SEQ ID NO:23, or a fragment thereof. In an aspect, a disclosed dSpCas9 can comprise a sequence having at least 60%, at least 65%, at least 70%, at least 75%, at least 80%, at least 85%, at least 90%, or at least 95% identity to the sequence set forth in SEQ ID NO:22, SEQ ID NO:23, or a fragment thereof. In an aspect, a disclosed encoded dSpCas9 can comprise the sequence set forth in SEQ ID NO:19 or a fragment thereof. In an aspect, a disclosed encoded dSpCas9 can comprise a sequence having at least 60%, at least 65%, at least 70%, at least 75%, at least 80%, at least 85%, at least 90%, or at least 95% identity to the sequence set forth in SEQ ID NO:19 or a fragment thereof.

In an aspect, a disclosed dSaCas9 can comprise the sequence set forth in SEQ ID NO:24 or a fragment thereof. In an aspect, a disclosed dSaCas9 can comprise a sequence having at least 60%, at least 65%, at least 70%, at least 75%, at least 80%, at least 85%, at least 90%, or at least 95% identity to the sequence set forth in SEQ ID NO:24 or a fragment thereof. In an aspect, a disclosed encoded dSaCas9 can comprise the sequence set forth in SEQ ID NO:20 or a fragment thereof. In an aspect, a disclosed encoded dSaCas9 can comprise a sequence having at least 60%, at least 65%, at least 70%, at least 75%, at least 80%, at least 85%, at least 90%, or at least 95% identity to the sequence set forth in SEQ ID NO:20 or a fragment thereof.

In an aspect, a disclosed dCjCas9 can comprise the sequence set forth in SEQ ID NO:25, SEQ ID NO:26, or a fragment thereof. In an aspect, a disclosed dCjCas9 can comprise a sequence having at least 60%, at least 65%, at least 70%, at least 75%, at least 80%, at least 85%, at least 90%, or at least 95% identity to the sequence set forth in SEQ ID NO:25, SEQ ID NO:26, or a fragment thereof. In an aspect, a disclosed encoded dCjCas9 can comprise the sequence set forth in SEQ ID NO:21 or a fragment thereof. In an aspect, a disclosed encoded dCjCas9 can comprise a sequence having at least 60%, at least 65%, at least 70%, at least 75%, at least 80%, at least 85%, at least 90%, or at least 95% identity to the sequence set forth in SEQ ID NO:21 or a fragment thereof.

In an aspect, a disclosed dVQR can comprise D1135V, R1335Q, and T1337R. In an aspect, a disclosed dEQR can comprise D1135E, R1335Q, and T1337R. In an aspect, a disclosed dVRER can comprise D1135V, G1218R, R1335E, and T1337R. In an aspect, a disclosed dVRER can comprise the sequence set forth in SEQ ID NO:27 or a fragment thereof.

In an aspect, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof.

In an aspect, a disclosed encoded polypeptide can comprise HP1a, HP1b, MBD1, MBD2, Krüppel-Associated Box (KRAB), NIPP1, the Transcription Repression Domain (TRD) of Methyl-CpG Binding Protein 2 (MeCP2), DNMT3A, any combination thereof, or any fusion thereof. In an aspect, a disclosed fusion or disclosed fusion protein can comprise HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect, a disclosed HP1a can comprise the sequence set forth in SEQ ID NO:43, SEQ ID NO:44, or fragment thereof. In an aspect, a disclosed HP1a can comprise a sequence having at least 60%, at least 65%, at least 70%, at least 75%, at least 80%, at least 85%, at least 90%, or at least 95% identity to the sequence set forth in SEQ ID NO:43, SEQ ID NO:44, or a fragment thereof. In an aspect, a disclosed encoded HP1a can comprise the sequence set forth in SEQ ID NO:28, SEQ ID NO:29, or a fragment thereof. In an aspect, a disclosed encoded HP1a can comprise a sequence having at least 60%, at least 65%, at least 70%, at least 75%, at least 80%, at least 85%, at least 90%, or at least 95% identity to the sequence set forth in SEQ ID NO:28, SEQ ID NO:29, or a fragment thereof.

In an aspect, a disclosed HP1b can comprise the sequence set forth in SEQ ID NO:45, SEQ ID NO:46, or a fragment thereof. In an aspect, a disclosed HP1b can comprise a sequence having at least 60%, at least 65%, at least 70%, at least 75%, at least 80%, at least 85%, at least 90%, or at least 95% identity to the sequence set forth in SEQ ID NO:45, SEQ ID NO:46, or a fragment thereof. In an aspect, a disclosed encoded HP1b can comprise the sequence set forth in SEQ ID NO:30, SEQ ID NO:31, or a fragment thereof. In an aspect, a disclosed encoded HP1a can comprise a sequence having at least 60%, at least 65%, at least 70%, at least 75%, at least 80%, at least 85%, at least 90%, or at least 95% identity to the sequence set forth in SEQ ID NO:30, SEQ ID NO:31, or a fragment thereof.

In an aspect, a disclosed MBD1 can comprise the sequence set forth in SEQ ID NO:47, SEQ ID NO:48, or a fragment thereof. In an aspect, a disclosed MBD1 can comprise a sequence having at least 60%, at least 65%, at least 70%, at least 75%, at least 80%, at least 85%, at least 90%, or at least 95% identity to the sequence set forth in SEQ ID NO:47, SEQ ID NO:48, or a fragment thereof. In an aspect, a disclosed encoded MBD1 can comprise the sequence set forth in SEQ ID NO:32, SEQ ID NO:33, SEQ ID NO:34, or a fragment thereof. In an aspect, a disclosed encoded MBD1 can comprise a sequence having at least 60%, at least 65%, at least 70%, at least 75%, at least 80%, at least 85%, at least 90%, or at least 95% identity to the sequence set forth in SEQ ID NO:32, SEQ ID NO:33, SEQ ID NO:34, or a fragment thereof.

In an aspect, a disclosed MBD2 can comprise the sequence set forth in SEQ ID NO:49, SEQ ID NO:50, or a fragment thereof. In an aspect, a disclosed MBD2 can comprise a sequence having at least 60%, at least 65%, at least 70%, at least 75%, at least 80%, at least 85%, at least 90%, or at least 95% identity to the sequence set forth in SEQ ID NO:49, SEQ ID NO:50, or a fragment thereof. In an aspect, a disclosed encoded MBD2 can comprise the sequence set forth in SEQ ID NO:35, SEQ ID NO:36, or a fragment thereof. In an aspect, a disclosed encoded MBD2 can comprise a sequence having at least 60%, at least 65%, at least 70%, at least 75%, at least 80%, at least 85%, at least 90%, or at least 95% identity to the sequence set forth in SEQ ID NO:35, SEQ ID NO:36, or a fragment thereof.

In an aspect, a disclosed KRAB can comprise the sequence set forth in SEQ ID NO:53, SEQ ID NO:54, SEQ ID NO:55, or a fragment thereof. In an aspect, a disclosed KRAB can comprise a sequence having at least 60%, at least 65%, at least 70%, at least 75%, at least 80%, at least 85%, at least 90%, or at least 95% identity to the sequence set forth in SEQ ID NO:53, SEQ ID NO:54, SEQ ID NO:55, or a fragment thereof. In an aspect, a disclosed encoded KRAB can comprise the sequence set forth in SEQ ID NO:39 or a fragment thereof. In an aspect, a disclosed encoded KRAB can comprise a sequence having at least 60%, at least 65%, at least 70%, at least 75%, at least 80%, at least 85%, at least 90%, or at least 95% identity to the sequence set forth in SEQ ID NO:39 or a fragment thereof.

In an aspect, a disclosed NIPP1 can comprise the sequence set forth in SEQ ID NO:51, SEQ ID NO:52, or a fragment thereof. In an aspect, a disclosed NIPP1 can comprise a sequence having at least 60%, at least 65%, at least 70%, at least 75%, at least 80%, at least 85%, at least 90%, or at least 95% identity to the sequence set forth in SEQ ID NO:51, SEQ ID NO:52, or a fragment thereof. In an aspect, a disclosed encoded NIPP1 can comprise the sequence set forth in SEQ ID NO:37, SEQ ID NO:38, or a fragment thereof. In an aspect, a disclosed encoded NIPP1 can comprise a sequence having at least 60%, at least 65%, at least 70%, at least 75%, at least 80%, at least 85%, at least 90%, or at least 95% identity to the sequence set forth in SEQ ID NO:37, SEQ ID NO:38, or a fragment thereof.

In an aspect, a disclosed MeCP2 can comprise the sequence set forth in SEQ ID NO:56, SEQ ID NO:57, or a fragment thereof. In an aspect, a disclosed MeCP2 can comprise a sequence having at least 60%, at least 65%, at least 70%, at least 75%, at least 80%, at least 85%, at least 90%, or at least 95% identity to the sequence set forth in SEQ ID NO:56, SEQ ID NO:57, or a fragment thereof. In an aspect, a disclosed encoded MeCP2 can comprise the sequence set forth in SEQ ID NO:40 or a fragment thereof. In an aspect, a disclosed encoded MeCP2 can comprise a sequence having at least 60%, at least 65%, at least 70%, at least 75%, at least 80%, at least 85%, at least 90%, or at least 95% identity to the sequence set forth in SEQ ID NO:40 or a fragment thereof.

In an aspect, a disclosed KRAB-MeCP2 can comprise the sequence set forth in SEQ ID NO:58, SEQ ID NO:59, or a fragment thereof. In an aspect, a disclosed KRAB-MeCP2 can comprise a sequence having at least 60%, at least 65%, at least 70%, at least 75%, at least 80%, at least 85%, at least 90%, or at least 95% identity to the sequence set forth in SEQ ID NO:58, SEQ ID NO:59, or a fragment thereof. In an aspect, a disclosed encoded KRAB-MeCP2 can comprise the sequence set forth in SEQ ID NO:41 or a fragment thereof. In an aspect, a disclosed encoded KRAB-MeCP2 can comprise a sequence having at least 60%, at least 65%, at least 70%, at least 75%, at least 80%, at least 85%, at least 90%, or at least 95% identity to the sequence set forth in SEQ ID NO:41 or a fragment thereof.

In an aspect, a disclosed DNMT3A can comprise the sequence set forth in SEQ ID NO:60, SEQ ID NO:61, or a fragment thereof. In an aspect, a disclosed DNMT3A can comprise a sequence having at least 60%, at least 65%, at least 70%, at least 75%, at least 80%, at least 85%, at least 90%, or at least 95% identity to the sequence set forth in SEQ ID NO:60, SEQ ID NO:61, or a fragment thereof. In an aspect, a disclosed encoded DNMT3A can comprise the sequence set forth in SEQ ID NO:42 or a fragment thereof. In an aspect, a disclosed encoded DNMT3A can comprise a sequence having at least 60%, at least 65%, at least 70%, at least 75%, at least 80%, at least 85%, at least 90%, or at least 95% identity to the sequence set forth in SEQ ID NO:42 or a fragment thereof.

In an aspect, a disclosed encoded polypeptide can comprise transcription activation activity. In an aspect, a disclosed encoded polypeptide comprising transcription activation activity can comprise a transactivation domain. In an aspect, a disclosed transactivation domain can comprise a VP16 protein, a series of linked VP16 proteins, a p65 domain of NFκB, or any combination thereof.

In an aspect, a disclosed VP16 can comprise the sequence set forth in SEQ ID NO:68, SEQ ID NO:69, or a fragment thereof. In an aspect, a disclosed VP16 can comprise a sequence having at least 60%, at least 65%, at least 70%, at least 75%, at least 80%, at least 85%, at least 90%, or at least 95% identity to the sequence set forth in SEQ ID NO:68, SEQ ID NO:69, or a fragment thereof. In an aspect, a disclosed encoded VP16 can comprise the sequence set forth in SEQ ID NO:70 or a fragment thereof. In an aspect, a disclosed encoded VP16 can comprise a sequence having at least 60%, at least 65%, at least 70%, at least 75%, at least 80%, at least 85%, at least 90%, or at least 95% identity to the sequence set forth in SEQ ID NO:70 or a fragment thereof.

In an aspect, a disclosed p65 domain of NFκB or RELA can comprise the sequence set forth in SEQ ID NO:65, SEQ ID NO:66, or a fragment thereof. In an aspect, a disclosed p65 domain of NFκB or RELA can comprise a sequence having at least 60%, at least 65%, at least 70%, at least 75%, at least 80%, at least 85%, at least 90%, or at least 95% identity to the sequence set forth in SEQ ID NO:65, SEQ ID NO:66, or a fragment thereof. In an aspect, a disclosed encoded p65 domain of NFκB or RELA can comprise the sequence set forth in SEQ ID NO:67 or a fragment thereof. In an aspect, a disclosed encoded p65 domain of NFκB or RELA can comprise a sequence having at least 60%, at least 65%, at least 70%, at least 75%, at least 80%, at least 85%, at least 90%, or at least 95% identity to the sequence set forth in SEQ ID NO:67 or a fragment thereof.

In an aspect, a disclosed encoded polypeptide can comprise transcription repression activity. In an aspect, a disclosed encoded polypeptide comprising transcription repression activity can comprise a Krüppel associated box domain, an ERF repressor domain, a MXI1 repressor domain, a SID4x repressor domain, a fused KRAB-MeCP2 domain, a MeCP2 TRD domain, a MAS-SID repressor domain, a TATA box binding protein activity, or any combination thereof. In an aspect, a disclosed encoded polypeptide comprising transcription repression activity can comprise HP1 repressor activity, MeCP2 repressor activity, MBD1 repressor activity, MBD2 repressor activity, MBD3 repressor activity, MBD4 repressor activity, KRAB repressor activity, SUV39H1 repressor activity, SUV39H2 repressor activity, CTCF insulator-repressor activity, LSD-1 histone-demethylase repressor activity, or any combination thereof.

In an aspect, a disclosed encoded polypeptide can comprise transcription release factor activity. In an aspect, a disclosed encoded polypeptide comprising transcription release factor activity can comprise Eukaryotic Release Factor 1 (ERF1) activity or Eukaryotic Release Factor 3 (ERF3) activity. In an aspect, a disclosed ERF1 can comprise the sequence set forth in SEQ ID NO:71 or a fragment thereof. In an aspect, a disclosed encoded ERF1 can comprise a sequence having at least 60%, at least 65%, at least 70%, at least 75%, at least 80%, at least 85%, at least 90%, or at least 95% identity to the sequence set forth in SEQ ID NO:72 or a fragment thereof.

In an aspect, a disclosed encoded polypeptide can comprise histone modification activity. In an aspect, a disclosed encoded polypeptide comprising histone modification activity can comprise histone acetyltransferase, histone deacetylase, histone demethylase, histone methyltransferase activity, or any combination thereof.

In an aspect, a disclosed encoded polypeptide can comprise nucleic acid association activity. In an aspect, a disclosed encoded polypeptide comprising nucleic acid association activity can comprise a helix-turn-helix region, a leucine zipper region, a winged helix region, a winged helix-turn-helix region, a helix-loop-helix region, an immunoglobulin fold, a B3 domain, a zinc finger, a HMG-box, a Wor3 domain, a TAL effector DNA-binding domain, or any combination thereof.

In an aspect, a disclosed encoded polypeptide can comprise methyltransferase activity. In an aspect, a disclosed encoded polypeptide comprising methyltransferase activity can comprise DNA (cytosine-5)-methyltransferase 3a (DNMT3A). In an aspect, a disclosed encoded polypeptide can comprise demethylase activity. In an aspect, a disclosed encoded polypeptide comprising demethylase activity can comprise ten-eleven translocation methylcytosine dioxygenase 1 (TET1) or lysine-specific histone demethylase 1 (LSD1). In an aspect, a disclosed encoded polypeptide can comprise acetyltransferase activity. In an aspect, a disclosed encoded polypeptide comprising acetyltransferase activity can comprise histone acetyltransferase. In an aspect, a disclosed encoded polypeptide can comprise deacetylase activity. In an aspect, a disclosed encoded polypeptide comprising deacetylase activity can comprise histone deacetylase.

In an aspect, a disclosed encoded dCas endonuclease is fused to the at least one encoded polypeptide having an enzymatic activity. In an aspect, a disclosed dCas endonuclease can comprise dSpCas9 and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dSaCas9 and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dCjCas9 and a disclosed polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof.

In an aspect, a disclosed dCas endonuclease can comprise dVQR, dEQR, or dVRER and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dSpCas9, dSaCas9, or dCjCas9 and a disclosed encoded polypeptide can comprise KRAB-MeCP2. In an aspect, a disclosed dCas endonuclease can comprise dSpCas9, dSaCas9, or dCjCas9, and a disclosed fusion can comprise HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect, a disclosed gene of interest or portion thereof can demonstrate a reduced expression and/or activity level when compared to wild-type or control expression level. In an aspect, a disclosed gRNA can targes a gene of interest or portion thereof that demonstrates a reduced expression and/or activity level when compared to wild-type or control expression level. In an aspect, a disclosed gene of interest or portion thereof can demonstrate an increased expression and/or activity level when compared to wild-type or control expression level.

In an aspect, a disclosed gene of interest can comprise ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARID1B, ARID2, ASH1L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANEl, CPS1, CPSF1, CRB1, CREBBP, CUBN, CUL7, CUX1, DCC, DCHS1, DEPDC5, DICER1, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNC1H1, DYNC2H1, DYSF, EIF2AK4, EP300, EPG5, ERCC6, ERCC6L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREBIL, GRIN2A, GRIN2B, GRIN2D, HCFC1, HECW2, HERC1, HERC2, HFM1, HIVEP1, HIVEP2, HMCN1, HSPG2, HTT, HUWEl, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12L, MED13, MED13L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MYO18B, MYO3A, MYO5A, MYO5B, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAIl, RALGAPA1, RANBP2, RB1CC1, RELN, RERE, REV3L, RIC1, RIMS1, RIMS2, RNF213, ROBO1, ROBO2, ROBO3, ROS1, RP1, RP1L1, RTTN, RUSC2, RYR1, RYR2, SACS, SAMD9, SAMD9L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAM1, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or any variant thereof, or any fragment thereof, or any portion thereof.

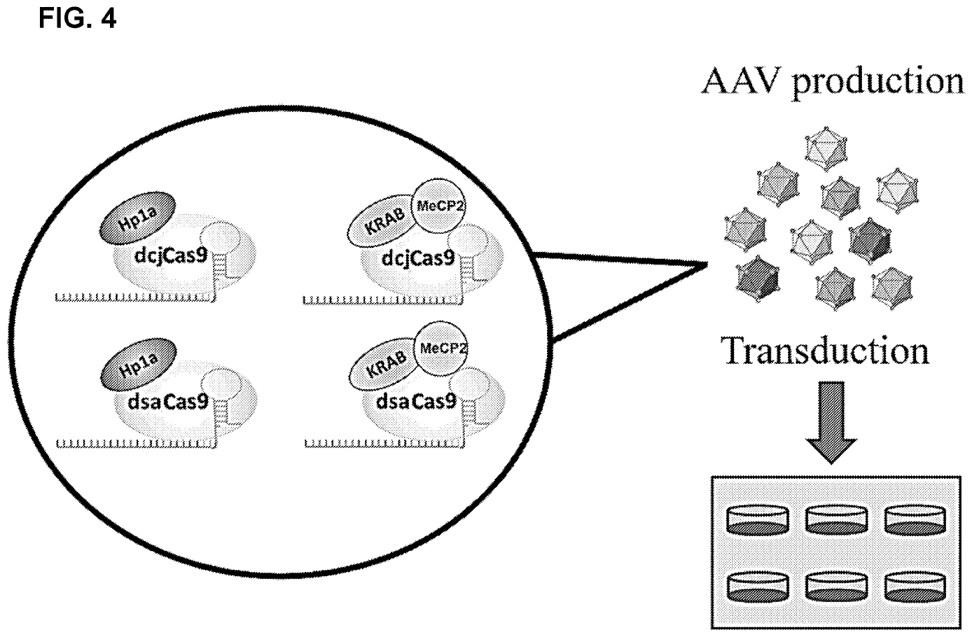

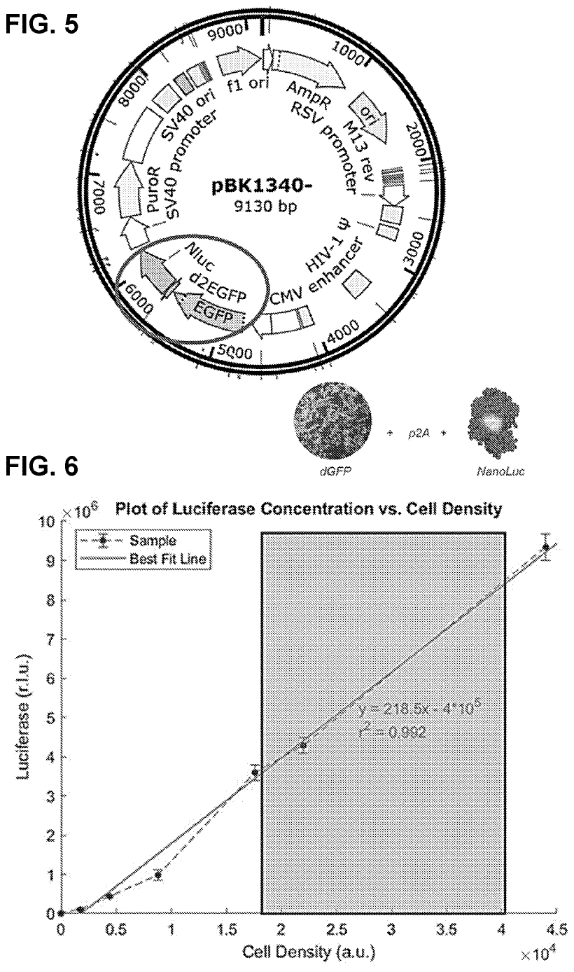

In an aspect, a disclosed gene of interest can comprise APOE (UniProt ID: P02649), APP (UniProt ID: P05067), ATXN2 (UniProt ID: Q99700), CHMP2B (UniProt ID: Q9UQN3), DCTN1 (UniProt ID: Q14203), FIG. 4 (UniProt ID: Q92562), FUS (UniProt ID: P35637), GBA (UniProt ID: P04062), GRN (UniProt ID: P28799), HNRNPA1 (UniProt ID: P09651), HTT (UniProt ID: P42858), LRRK2 (UniProt ID: Q5S007), MATR3 (UniProt ID: P43243), OPTN (UniProt ID: Q96CV9), PARK7 (UniProt ID: Q99497), PFN1 (UniProt ID: P07737), PRPH (UniProt ID: P41219), PSEN1 (UniProt ID: P49768), SETX (UniProt ID: Q7Z333), SIGMAR1 (UniProt ID: Q99720), SNCA (UniProt ID: P37840), SOD1 (UniProt ID: P00441), SPG11 (UniProt ID: Q96JI7), SQSTM1 (UniProt ID: Q13501), TARDBP (UniProt ID: Q13148), TBK1 (UniProt ID: Q9UHD2), TBP (UniProt ID: P20226), TRPM7 (UniProt ID: Q96QT4), TUBA4A (UniProt ID: P68366), UBQLN2 (UniProt ID: Q9UHD9), UCHL1 (UniProt ID: P09936), VAPB (UniProt ID: 095292), VCP (UniProt ID: P55072), VPS35 (UniProt ID: Q96QK1), or any variant thereof, or any fragment thereof, or any portion thereof.

In an aspect, a disclosed gRNA can target a gene of interest or portion thereof that demonstrates an increased expression level when compared to wild-type or control expression level. In an aspect, a disclosed gRNA targeting a gene of interest or portion thereof can comprise at least two gRNAs. In an aspect, disclosed gRNAs can target the same gene of interest or portion thereof. In an aspect, disclosed gRNAs can target different genes of interest or portions thereof. In an aspect, a disclosed gRNA can target a gene or portion thereof that demonstrates a loss of function. In an aspect, a disclosed gRNA can target a gene or portion thereof that demonstrates a gain of function.

In an aspect, a disclosed gRNA can target the APOE gene. In an aspect, a disclosed gRNA can target the promoter of the APOE gene. In an aspect, a disclosed gRNA can target exon 4 of the APOE gene. In an aspect, a disclosed gRNA can target a protospacer-adjacent motif (PAM) created by a SNP rs429358 in exon 4 of the APOE gene. In an aspect, a disclosed APOE gene can comprise the sequence set forth in SEQ ID NO:93-SEQ ID NO:96 or a fragment thereof. In an aspect, a disclosed gRNA targeting the APOE gene and/or the promoter of the APOE gene can comprise the sequence set forth in SEQ ID NO:85-SEQ ID NO:92.

In an aspect, a disclosed gRNA can target the SNCA gene. In an aspect, a disclosed gRNA can target the promoter of the SNCA gene. In an aspect, a disclosed gRNA can target exon 1 of the SNCA gene. In an aspect, a disclosed gRNA targeting the SCNA gene and/or the promoter of the SNCA gene can comprise the sequence set forth in SEQ ID NO:81-SEQ ID NO:84.

In an aspect, a disclosed gRNA can target a CMV promoter. In an aspect, a disclosed gRNA targeting the CMV promoter can comprise the sequence set forth in SEQ ID NO:73-SEQ ID NO:80 or a fragment thereof. In an aspect, a disclosed gRNA can target a GFP gene. In an aspect, a disclosed gRNA targeting the GFP gene can comprise the sequence set forth in SEQ ID NO:125, SEQ ID NO:126, or a fragment thereof.

In an aspect, a disclosed isolated nucleic acid molecule can further comprise a nucleic acid sequencing encoding one or more regulatory elements. In an aspect, a disclosed regulatory element can comprise a promoter, an enhancer, a promoter/enhancer, an internal ribosomal entry site, a transcription terminal signal, a polyadenylation signal, a Sp1 and/or NF-kB transcriptional factor binding site, a p2A signal, a woodchuck hepatitis virus post-transcriptional regulatory element, a Phi signal-packaging signal, a rev responsive element, a 5′-LTR, a 3′-LTR, an inverted terminal repeat, a nuclear localization signal (NLS), or any combination thereof.

In an aspect, a disclosed NLS can comprise the sequence set forth in SEQ ID NO:105-SEQ ID NO:111 or a fragment thereof. In an aspect, a disclosed PolyA sequence can comprise the sequence set forth in SEQ ID NO:103, SEQ ID NO:104, or a fragment thereof. In an aspect, a disclosed ITR can comprise the sequence set forth in SEQ ID NO:99-SEQ ID NO:102 or a fragment thereof.

In an aspect, a disclosed isolated nucleic acid molecule can further a nucleic acid sequence encoding one or more promoters. In an aspect, a disclosed promoter can comprise a U6 promoter, a chicken 3-actin promoter, an EF-1α, a CMV promoter, a CMV promoter/enhancer, a fragment thereof, or any combination thereof. In an aspect, a disclosed U6 promoter can comprise the sequence set forth in SEQ ID NO:114 or a fragment thereof. In an aspect, a disclosed U6 promoter can comprise a sequence having at least 60%, at least 65%, at least 70%, at least 75%, at least 80%, at least 85%, at least 90%, at least 95%, or greater than 95% identity to the sequence set forth in SEQ ID NO:114 or a fragment thereof. In an aspect, a disclosed EF-1α promoter can comprise the sequence set forth in SEQ ID NO:112, SEQ ID NO:113, or a fragment thereof. In an aspect, a disclosed EF-1α promoter can comprise a sequence having at least 60%, at least 65%, at least 70%, at least 75%, at least 80%, at least 85%, at least 90%, at least 95%, or greater than 95% identity to the sequence set forth in SEQ ID NO:112, SEQ ID NO:113, or a fragment thereof. In an aspect, a promoter can be a short EF1alpha (EFS-NF) promoter.

In an aspect, a disclosed promoter can be operably linked to the dCas endonuclease. In an aspect, a disclosed promoter operably linked to the dCas endonuclease can comprise an EF-1α promoter. In an aspect, a disclosed promoter operably linked to the dCas endonuclease can be operably linked to the at least one polypeptide having enzymatic activity. In an aspect, a disclosed promoter can be operably linked to the dCas endonuclease and the at least one polypeptide having enzymatic activity. In an aspect, a disclosed promoter can be operably linked to the fusion protein having one or more enzymatic activities. In an aspect, a disclosed promoter can be operably linked to the dCas endonuclease and the fusion protein having one or more enzymatic activities. In an aspect, a disclosed promoter operably can be linked to the at least one guide RNA targeting a gene of interest or portion thereof. In an aspect, a disclosed promoter operably linked to the at least one guide RNA can comprise a U6 promoter.

In an aspect, a disclosed isolated nucleic acid molecule can further comprise a gRNA scaffold. In an aspect, a disclosed gRNA scaffold can comprise the sequence set forth in SEQ ID NO: 115, SEQ ID NO: 116, or a fragment thereof.

In an aspect, a disclosed isolated nucleic acid molecule can further comprise a nucleic acid sequence encoding one or more promoters, wherein a first promoter can be operably linked to the dCas endonuclease, and wherein a second promoter can be operably linked to the at least one guide RNA targeting a gene of interest or portion thereof. In an aspect, a disclosed isolated nucleic acid molecule can further comprise a nucleic acid sequence encoding one or more promoters, wherein a first promoter can be operably linked to the dCas endonuclease and the at least one polypeptide having an enzymatic activity, and wherein a second promoter can be operably linked to the at least one guide RNA targeting a gene of interest thereof.

In an aspect, a disclosed encoded polypeptide can comprise HP1a, HP1b, MBD1, MBD2, Krüppel-Associated Box (KRAB), NIPP1, the Transcription Repression Domain (TRD) of Methyl-CpG Binding Protein 2 (MeCP2), DNMT3A, any combination thereof, or any fusion thereof.

In an aspect, a disclosed nucleic acid sequence can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art. In an aspect, any disclosed Cas9 endonuclease, a disclosed polypeptide having enzymatic activities, a disclosed fusion product or a disclosed fusion protein, or any combination thereof can be codon-optimized.

In an aspect, a disclosed isolated nucleic acid molecule can comprise a sequence that is about 4.5 kilobases or less than about 4.5 kilobases.

Disclosed herein is an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof.

Disclosed herein is an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA.

Disclosed herein is an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof.

Disclosed herein is an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA.

In an aspect, a disclosed isolated nucleic acid molecule can treat and/or prevent Alzheimer's disease progression. In an aspect, a disclosed isolated nucleic acid molecule can treat and/or prevent Parkinson's disease progression.

### 2. Fusion Proteins

Disclosed herein is a fusion protein comprising a first component having one or more enzymatic activities and a second component comprising a dCas9.

In an aspect of a disclosed fusion protein, a disclosed dCas endonuclease can comprise a dCas9 endonuclease. In an aspect, a disclosed dCas9 endonuclease can comprise a deactivated Staphylococcus aureus Cas9 (dSaCas9), a deactivated Streptococcus pyogenes Cas9 (dSpCas9), a deactivated Campylobacter jejuni Cas9 (dCjCas9), or a variant dCas9 endonuclease. In an aspect, a disclosed variant dCas9 can comprise a variant dSaCas9, a variant dSpCas9, or a variant dCjCa9. In an aspect, a disclosed variant dSpCas9 can comprise dVQR, dEQR, or dVRER.

In an aspect, a disclosed dSpCas9 can comprise the sequence set forth in SEQ ID NO:22, SEQ ID NO:23, or a fragment thereof. In an aspect, a disclosed dSpCas9 can comprise a sequence having at least 60%, at least 65%, at least 70%, at least 75%, at least 80%, at least 85%, at least 90%, or at least 95% identity to the sequence set forth in SEQ ID NO:22, SEQ ID NO:23, or a fragment thereof. In an aspect, a disclosed encoded dSpCas9 can comprise the sequence set forth in SEQ ID NO:19 or a fragment thereof. In an aspect, a disclosed encoded dSpCas9 can comprise a sequence having at least 60%, at least 65%, at least 70%, at least 75%, at least 80%, at least 85%, at least 90%, or at least 95% identity to the sequence set forth in SEQ ID NO:19 or a fragment thereof.

In an aspect, a disclosed dSaCas9 can comprise the sequence set forth in SEQ ID NO:24 or a fragment thereof. In an aspect, a disclosed dSaCas9 can comprise a sequence having at least 60%, at least 65%, at least 70%, at least 75%, at least 80%, at least 85%, at least 90%, or at least 95% identity to the sequence set forth in SEQ ID NO:24 or a fragment thereof. In an aspect, a disclosed encoded dSaCas9 can comprise the sequence set forth in SEQ ID NO:20 or a fragment thereof. In an aspect, a disclosed encoded dSaCas9 can comprise a sequence having at least 60%, at least 65%, at least 70%, at least 75%, at least 80%, at least 85%, at least 90%, or at least 95% identity to the sequence set forth in SEQ ID NO:20 or a fragment thereof.

In an aspect, a disclosed dCjCas9 can comprise the sequence set forth in SEQ ID NO:25, SEQ ID NO:26, or a fragment thereof. In an aspect, a disclosed dCjCas9 can comprise a sequence having at least 60%, at least 65%, at least 70%, at least 75%, at least 80%, at least 85%, at least 90%, or at least 95% identity to the sequence set forth in SEQ ID NO:25, SEQ ID NO:26, or a fragment thereof. In an aspect, a disclosed encoded dCjCas9 can comprise the sequence set forth in SEQ ID NO:21 or a fragment thereof. In an aspect, a disclosed encoded dCjCas9 can comprise a sequence having at least 60%, at least 65%, at least 70%, at least 75%, at least 80%, at least 85%, at least 90%, or at least 95% identity to the sequence set forth in SEQ ID NO:21 or a fragment thereof.

In an aspect, a disclosed dVQR can comprise D1135V, R1335Q, and T1337R. In an aspect, a disclosed dEQR can comprise D1135E, R1335Q, and T1337R. In an aspect, a disclosed dVRER can comprise D1135V, G1218R, R1335E, and T1337R. In an aspect, a disclosed dVRER can comprise the sequence set forth in SEQ ID NO:27 or a fragment thereof.

In an aspect, a disclosed first component can comprise transcription activation activity, transcription repression activity, transcription release factor activity, histone modification activity, nucleic acid association activity, methyltransferase activity, demethylase activity, acetyltransferase activity, deacetylase activity, or any combination thereof. In an aspect, a disclosed first component can comprise HP1a, HP1b, MBD1, MBD2, Krüppel-Associated Box (KRAB), NIPP1, the Transcription Repression Domain (TRD) of Methyl-CpG Binding Protein 2 (MeCP2), DNMT3A, any combination thereof, or any fusion thereof. In an aspect, a disclosed first component can comprise HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect, a disclosed HP1a can comprise the sequence set forth in SEQ ID NO:43, SEQ ID NO:44, or fragment thereof. In an aspect, a disclosed HP1a can comprise a sequence having at least 60%, at least 65%, at least 70%, at least 75%, at least 80%, at least 85%, at least 90%, or at least 95% identity to the sequence set forth in SEQ ID NO:43, SEQ ID NO:44, or a fragment thereof. In an aspect, a disclosed encoded HP1a can comprise the sequence set forth in SEQ ID NO:28, SEQ ID NO:29, or a fragment thereof. In an aspect, a disclosed encoded HP1a can comprise a sequence having at least 60%, at least 65%, at least 70%, at least 75%, at least 80%, at least 85%, at least 90%, or at least 95% identity to the sequence set forth in SEQ ID NO:28, SEQ ID NO:29, or a fragment thereof.

In an aspect, a disclosed HP1b can comprise the sequence set forth in SEQ ID NO:45, SEQ ID NO:46, or a fragment thereof. In an aspect, a disclosed HP1b can comprise a sequence having at least 60%, at least 65%, at least 70%, at least 75%, at least 80%, at least 85%, at least 90%, or at least 95% identity to the sequence set forth in SEQ ID NO:45, SEQ ID NO:46, or a fragment thereof. In an aspect, a disclosed encoded HP1b can comprise the sequence set forth in SEQ ID NO:30, SEQ ID NO:31, or a fragment thereof. In an aspect, a disclosed encoded HP1a can comprise a sequence having at least 60%, at least 65%, at least 70%, at least 75%, at least 80%, at least 85%, at least 90%, or at least 95% identity to the sequence set forth in SEQ ID NO:30, SEQ ID NO:31, or a fragment thereof.

In an aspect, a disclosed MBD1 can comprise the sequence set forth in SEQ ID NO:47, SEQ ID NO:48, or a fragment thereof. In an aspect, a disclosed MBD1 can comprise a sequence having at least 60%, at least 65%, at least 70%, at least 75%, at least 80%, at least 85%, at least 90%, or at least 95% identity to the sequence set forth in SEQ ID NO:47, SEQ ID NO:48, or a fragment thereof. In an aspect, a disclosed encoded MBD1 can comprise the sequence set forth in SEQ ID NO:32, SEQ ID NO:33, SEQ ID NO:34, or a fragment thereof. In an aspect, a disclosed encoded MBD1 can comprise a sequence having at least 60%, at least 65%, at least 70%, at least 75%, at least 80%, at least 85%, at least 90%, or at least 95% identity to the sequence set forth in SEQ ID NO:32, SEQ ID NO:33, SEQ ID NO:34, or a fragment thereof.

In an aspect, a disclosed MBD2 can comprise the sequence set forth in SEQ ID NO:49, SEQ ID NO:50, or a fragment thereof. In an aspect, a disclosed MBD2 can comprise a sequence having at least 60%, at least 65%, at least 70%, at least 75%, at least 80%, at least 85%, at least 90%, or at least 95% identity to the sequence set forth in SEQ ID NO:49, SEQ ID NO:50, or a fragment thereof. In an aspect, a disclosed encoded MBD2 can comprise the sequence set forth in SEQ ID NO:35, SEQ ID NO:36, or a fragment thereof. In an aspect, a disclosed encoded MBD2 can comprise a sequence having at least 60%, at least 65%, at least 70%, at least 75%, at least 80%, at least 85%, at least 90%, or at least 95% identity to the sequence set forth in SEQ ID NO:35, SEQ ID NO:36, or a fragment thereof.

In an aspect, a disclosed KRAB can comprise the sequence set forth in SEQ ID NO:53, SEQ ID NO:54, SEQ ID NO:55, or a fragment thereof. In an aspect, a disclosed KRAB can comprise a sequence having at least 60%, at least 65%, at least 70%, at least 75%, at least 80%, at least 85%, at least 90%, or at least 95% identity to the sequence set forth in SEQ ID NO:53, SEQ ID NO:54, SEQ ID NO:55, or a fragment thereof. In an aspect, a disclosed encoded KRAB can comprise the sequence set forth in SEQ ID NO:39 or a fragment thereof. In an aspect, a disclosed encoded KRAB can comprise a sequence having at least 60%, at least 65%, at least 70%, at least 75%, at least 80%, at least 85%, at least 90%, or at least 95% identity to the sequence set forth in SEQ ID NO:39 or a fragment thereof.

In an aspect, a disclosed NIPP1 can comprise the sequence set forth in SEQ ID NO:51, SEQ ID NO:52, or a fragment thereof. In an aspect, a disclosed NIPP1 can comprise a sequence having at least 60%, at least 65%, at least 70%, at least 75%, at least 80%, at least 85%, at least 90%, or at least 95% identity to the sequence set forth in SEQ ID NO:51, SEQ ID NO:52, or a fragment thereof. In an aspect, a disclosed encoded NIPP1 can comprise the sequence set forth in SEQ ID NO:37, SEQ ID NO:38, or a fragment thereof. In an aspect, a disclosed encoded NIPP1 can comprise a sequence having at least 60%, at least 65%, at least 70%, at least 75%, at least 80%, at least 85%, at least 90%, or at least 95% identity to the sequence set forth in SEQ ID NO:37, SEQ ID NO:38, or a fragment thereof.

In an aspect, a disclosed MeCP2 can comprise the sequence set forth in SEQ ID NO:56, SEQ ID NO:57, or a fragment thereof. In an aspect, a disclosed MeCP2 can comprise a sequence having at least 60%, at least 65%, at least 70%, at least 75%, at least 80%, at least 85%, at least 90%, or at least 95% identity to the sequence set forth in SEQ ID NO:56, SEQ ID NO:57, or a fragment thereof. In an aspect, a disclosed encoded MeCP2 can comprise the sequence set forth in SEQ ID NO:40 or a fragment thereof. In an aspect, a disclosed encoded MeCP2 can comprise a sequence having at least 60%, at least 65%, at least 70%, at least 75%, at least 80%, at least 85%, at least 90%, or at least 95% identity to the sequence set forth in SEQ ID NO:40 or a fragment thereof.

In an aspect, a disclosed KRAB-MeCP2 can comprise the sequence set forth in SEQ ID NO:58, SEQ ID NO:59, or a fragment thereof. In an aspect, a disclosed KRAB-MeCP2 can comprise a sequence having at least 60%, at least 65%, at least 70%, at least 75%, at least 80%, at least 85%, at least 90%, or at least 95% identity to the sequence set forth in SEQ ID NO:58, SEQ ID NO:59, or a fragment thereof. In an aspect, a disclosed encoded KRAB-MeCP2 can comprise the sequence set forth in SEQ ID NO:41 or a fragment thereof. In an aspect, a disclosed encoded KRAB-MeCP2 can comprise a sequence having at least 60%, at least 65%, at least 70%, at least 75%, at least 80%, at least 85%, at least 90%, or at least 95% identity to the sequence set forth in SEQ ID NO:41 or a fragment thereof.

In an aspect, a disclosed DNMT3A can comprise the sequence set forth in SEQ ID NO:60, SEQ ID NO:61, or a fragment thereof. In an aspect, a disclosed DNMT3A can comprise a sequence having at least 60%, at least 65%, at least 70%, at least 75%, at least 80%, at least 85%, at least 90%, or at least 95% identity to the sequence set forth in SEQ ID NO:60, SEQ ID NO:61, or a fragment thereof. In an aspect, a disclosed encoded DNMT3A can comprise the sequence set forth in SEQ ID NO:42 or a fragment thereof. In an aspect, a disclosed encoded DNMT3A can comprise a sequence having at least 60%, at least 65%, at least 70%, at least 75%, at least 80%, at least 85%, at least 90%, or at least 95% identity to the sequence set forth in SEQ ID NO:42 or a fragment thereof.

In an aspect, a disclosed first component can comprise transcription activation activity. In an aspect, a disclosed first component comprising transcription activation activity can comprise a transactivation domain. In an aspect, a disclosed transactivation domain can comprise a VP16 protein, a series of linked VP16 proteins, a p65 domain of NFκB, or any combination thereof. In an aspect, a disclosed VP16 can comprise the sequence set forth in SEQ ID NO:68, SEQ ID NO:69, or a fragment thereof. In an aspect, a disclosed VP16 can comprise a sequence having at least 60%, at least 65%, at least 70%, at least 75%, at least 80%, at least 85%, at least 90%, or at least 95% identity to the sequence set forth in SEQ ID NO:68, SEQ ID NO:69, or a fragment thereof. In an aspect, a disclosed encoded VP16 can comprise the sequence set forth in SEQ ID NO:70 or a fragment thereof. In an aspect, a disclosed encoded VP16 can comprise a sequence having at least 60%, at least 65%, at least 70%, at least 75%, at least 80%, at least 85%, at least 90%, or at least 95% identity to the sequence set forth in SEQ ID NO:70 or a fragment thereof. In an aspect, a disclosed p65 domain of NFκB or RELA can comprise the sequence set forth in SEQ ID NO:65, SEQ ID NO:66, or a fragment thereof. In an aspect, a disclosed p65 domain of NFκB or RELA can comprise a sequence having at least 60%, at least 65%, at least 70%, at least 75%, at least 80%, at least 85%, at least 90%, or at least 95% identity to the sequence set forth in SEQ ID NO:65, SEQ ID NO:66, or a fragment thereof. In an aspect, a disclosed encoded p65 domain of NFκB or RELA can comprise the sequence set forth in SEQ ID NO:67 or a fragment thereof. In an aspect, a disclosed encoded p65 domain of NFκB or RELA can comprise a sequence having at least 60%, at least 65%, at least 70%, at least 75%, at least 80%, at least 85%, at least 90%, or at least 95% identity to the sequence set forth in SEQ ID NO:67 or a fragment thereof.

In an aspect, a disclosed first component can comprise transcription repression activity. In an aspect, a disclosed first component comprising transcription repression activity can comprise a Krüppel associated box domain, an ERF repressor domain, a MXI1 repressor domain, a SID4x repressor domain, a fused KRAB-MeCP2 domain, a MeCP2 TRD domain, a MAS-SID repressor domain, a TATA box binding protein activity, or any combination thereof. In an aspect, a disclosed first component comprising transcription repression activity can comprise HP1 repressor activity, MeCP2 repressor activity, MBD1 repressor activity, MBD2 repressor activity, MBD3 repressor activity, MBD4 repressor activity, KRAB repressor activity, SUV39H1 repressor activity, SUV39H2 repressor activity, CTCF insulator-repressor activity, LSD-1 histone-demethylase repressor activity, or any combination thereof.

In an aspect, a disclosed first component can comprise transcription release factor activity. In an aspect, a disclosed encoded polypeptide comprising transcription release factor activity can comprise Eukaryotic Release Factor 1 (ERF1) activity or Eukaryotic Release Factor 3 (ERF3) activity. In an aspect, a disclosed ERF1 can comprise the sequence set forth in SEQ ID NO:71 or a fragment thereof. In an aspect, a disclosed encoded ERF1 can comprise a sequence having at least 60%, at least 65%, at least 70%, at least 75%, at least 80%, at least 85%, at least 90%, or at least 95% identity to the sequence set forth in SEQ ID NO:72 or a fragment thereof.

In an aspect, a disclosed first component can comprise histone modification activity. In an aspect, a disclosed first component comprising histone modification activity can comprise histone acetyltransferase, histone deacetylase, histone demethylase, histone methyltransferase activity, or any combination thereof. In an aspect, a disclosed first component can comprise nucleic acid association activity. In an aspect, a disclosed first component comprising nucleic acid association activity can comprise a helix-turn-helix region, a leucine zipper region, a winged helix region, a winged helix-turn-helix region, a helix-loop-helix region, an immunoglobulin fold, a B3 domain, a zinc finger, a HMG-box, a Wor3 domain, a TAL effector DNA-binding domain, or any combination thereof. In an aspect, a disclosed first component can comprise methyltransferase activity. In an aspect, a disclosed first component comprising methyltransferase activity can comprise DNA (cytosine-5)-methyltransferase 3a (DNMT3A). In an aspect, a disclosed first component can comprise demethylase activity. In an aspect, a disclosed first component comprising demethylase activity can comprise ten-eleven translocation methylcytosine dioxygenase 1 (TET1) or lysine-specific histone demethylase 1 (LSD1). In an aspect, a disclosed first component can comprise acetyltransferase activity. In an aspect, a disclosed first component comprising acetyltransferase activity can comprise histone acetyltransferase. In an aspect, a disclosed first component can comprise deacetylase activity. In an aspect, a disclosed first component comprising deacetylase activity can comprise histone deacetylase.

In an aspect, a disclosed fusion protein can comprise dSpCas9 and HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed fusion protein can comprise dSaCas9 and HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed fusion protein can comprise dCjCas9 and HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dVQR, dEQR, or dVRER and a disclosed first component can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed fusion protein can comprise dSpCas9, dSaCas9, or dCjCas9 and KRAB-MeCP2.

In an aspect, a disclosed fusion protein can comprise dSpCas9, dSaCas9, or dCjCas9, and HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect, a disclosed fusion protein can reduce expression and/or activity level of a target gene or gene of interest when compared to wild-type or control expression level. In an aspect, a disclosed fusion protein can increase expression and/or activity level of a target gene or gene of interest when compared to wild-type or control expression level.

In an aspect, a disclosed target gene or gene of interest can comprise ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARID1B, ARID2, ASH1L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANEl, CPS1, CPSF1, CRB1, CREBBP, CUBN, CUL7, CUX1, DCC, DCHS1, DEPDC5, DICER1, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNC1H1, DYNC2H1, DYSF, EIF2AK4, EP300, EPG5, ERCC6, ERCC6L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREBIL, GRIN2A, GRIN2B, GRIN2D, HCFC1, HECW2, HERC1, HERC2, HFM1, HIVEP1, HIVEP2, HMCN1, HSPG2, HTT, HUWEl, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12L, MED13, MED13L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MYO18B, MYO3A, MYO5A, MYO5B, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAIl, RALGAPA1, RANBP2, RB1CC1, RELN, RERE, REV3L, RIC1, RIMS1, RIMS2, RNF213, ROBO1, ROBO2, ROBO3, ROS1, RP1, RP1L1, RTTN, RUSC2, RYR1, RYR2, SACS, SAMD9, SAMD9L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAM1, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or any variant thereof, or any fragment thereof, or any portion thereof.

In an aspect, a disclosed gene of interest can comprise APOE (UniProt ID: P02649), APP (UniProt ID: P05067), ATXN2 (UniProt ID: Q99700), CHMP2B (UniProt ID: Q9UQN3), DCTN1 (UniProt ID: Q14203), FIG. 4 (UniProt ID: Q92562), FUS (UniProt ID: P35637), GBA (UniProt ID: P04062), GRN (UniProt ID: P28799), HNRNPA1 (UniProt ID: P09651), HTT (UniProt ID: P42858), LRRK2 (UniProt ID: Q5S007), MATR3 (UniProt ID: P43243), OPTN (UniProt ID: Q96CV9), PARK7 (UniProt ID: Q99497), PFN1 (UniProt ID: P07737), PRPH (UniProt ID: P41219), PSEN1 (UniProt ID: P49768), SETX (UniProt ID: Q7Z333), SIGMAR1 (UniProt ID: Q99720), SNCA (UniProt ID: P37840), SOD1 (UniProt ID: P00441), SPG11 (UniProt ID: Q96JI7), SQSTM1 (UniProt ID: Q13501), TARDBP (UniProt ID: Q13148), TBK1 (UniProt ID: Q9UHD2), TBP (UniProt ID: P20226), TRPM7 (UniProt ID: Q96QT4), TUBA4A (UniProt ID: P68366), UBQLN2 (UniProt ID: Q9UHD9), UCHL1 (UniProt ID: P09936), VAPB (UniProt ID: 095292), VCP (UniProt ID: P55072), VPS35 (UniProt ID: Q96QK1), or any variant thereof, or any fragment thereof, or any portion thereof.

In an aspect, a disclosed fusion protein can treat and/or prevent Alzheimer's disease progression. In an aspect, a disclosed fusion protein can treat and/or prevent Parkinson's disease progression.

### 3. Viral Vectors

Disclosed herein is a viral vector comprising a disclosed isolated nucleic acid molecule. Disclosed herein is a viral vector comprising a disclosed nucleic acid molecule. Disclosed herein is a viral vector comprising a nucleic acid sequence encoding (i) a deactivated Cas (dCas) endonuclease, (ii) at least one polypeptide having an enzymatic activity, and (iii) at least one guide RNA targeting a gene of interest or portion thereof. Disclosed herein is a viral vector comprising a nucleic acid sequence encoding (i) a deactivated Cas (dCas) endonuclease, (ii) a fusion protein having one or more enzymatic activities, and (iii) at least one guide RNA targeting a gene of interest or portion thereof.

Disclosed herein is a recombinant AAV vector comprising a disclosed isolated nucleic acid molecule. Disclosed herein is a recombinant AAV vector comprising a disclosed nucleic acid molecule. Disclosed herein is a recombinant AAV vector comprising a nucleic acid sequence encoding (i) a deactivated Cas (dCas) endonuclease, (ii) at least one polypeptide having an enzymatic activity, and (iii) at least one guide RNA targeting a gene of interest or portion thereof. Disclosed herein is a recombinant AAV vector comprising a nucleic acid sequence encoding (i) a deactivated Cas (dCas) endonuclease, (ii) a fusion protein having one or more enzymatic activities, and (iii) at least one guide RNA targeting a gene of interest or portion thereof.

In an aspect of a disclosed viral vector or disclosed recombinant AAV vector, a disclosed dCas endonuclease can comprise a dCas9 endonuclease. In an aspect, a disclosed dCas9 endonuclease can comprise a deactivated Staphylococcus aureus Cas9 (dSaCas9), a deactivated Streptococcus pyogenes Cas9 (dSpCas9), a deactivated Campylobacter jejuni Cas9 (dCjCas9), or a variant dCas9 endonuclease. In an aspect, a disclosed variant dCas9 can comprise a variant dSaCas9, a variant dSpCas9, or a variant dCjCa9. In an aspect, a disclosed variant dSpCas9 can comprise dVQR, dEQR, or dVRER.

In an aspect of a disclosed viral vector or disclosed recombinant AAV vector, a disclosed dSpCas9 can comprise the sequence set forth in SEQ ID NO:22, SEQ ID NO:23, or a fragment thereof. In an aspect, a disclosed dSpCas9 can comprise a sequence having at least 60%, at least 65%, at least 70%, at least 75%, at least 80%, at least 85%, at least 90%, or at least 95% identity to the sequence set forth in SEQ ID NO:22, SEQ ID NO:23, or a fragment thereof. In an aspect, a disclosed encoded dSpCas9 can comprise the sequence set forth in SEQ ID NO:19 or a fragment thereof. In an aspect, a disclosed encoded dSpCas9 can comprise a sequence having at least 60%, at least 65%, at least 70%, at least 75%, at least 80%, at least 85%, at least 90%, or at least 95% identity to the sequence set forth in SEQ ID NO:19 or a fragment thereof.

In an aspect of a disclosed viral vector or disclosed recombinant AAV vector, a disclosed dSaCas9 can comprise the sequence set forth in SEQ ID NO:24 or a fragment thereof. In an aspect, a disclosed dSaCas9 can comprise a sequence having at least 60%, at least 65%, at least 70%, at least 75%, at least 80%, at least 85%, at least 90%, or at least 95% identity to the sequence set forth in SEQ ID NO:24 or a fragment thereof. In an aspect, a disclosed encoded dSaCas9 can comprise the sequence set forth in SEQ ID NO:20 or a fragment thereof. In an aspect, a disclosed encoded dSaCas9 can comprise a sequence having at least 60%, at least 65%, at least 70%, at least 75%, at least 80%, at least 85%, at least 90%, or at least 95% identity to the sequence set forth in SEQ ID NO:20 or a fragment thereof.

In an aspect of a disclosed viral vector or disclosed recombinant AAV vector, a disclosed dCjCas9 can comprise the sequence set forth in SEQ ID NO:25, SEQ ID NO:26, or a fragment thereof. In an aspect, a disclosed dCjCas9 can comprise a sequence having at least 60%, at least 65%, at least 70%, at least 75%, at least 80%, at least 85%, at least 90%, or at least 95% identity to the sequence set forth in SEQ ID NO:25, SEQ ID NO:26, or a fragment thereof. In an aspect, a disclosed encoded dCjCas9 can comprise the sequence set forth in SEQ ID NO:21 or a fragment thereof. In an aspect, a disclosed encoded dCjCas9 can comprise a sequence having at least 60%, at least 65%, at least 70%, at least 75%, at least 80%, at least 85%, at least 90%, or at least 95% identity to the sequence set forth in SEQ ID NO:21 or a fragment thereof.

In an aspect, a disclosed dVQR can comprise D1135V, R1335Q, and T1337R. In an aspect, a disclosed dEQR can comprise D1135E, R1335Q, and T1337R. In an aspect, a disclosed dVRER can comprise D1135V, G1218R, R1335E, and T1337R. In an aspect, a disclosed dVRER can comprise the sequence set forth in SEQ ID NO:27 or a fragment thereof.

In an aspect of a disclosed viral vector or disclosed recombinant AAV vector, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof.

In an aspect of a disclosed viral vector or disclosed recombinant AAV vector, a disclosed encoded polypeptide can comprise HP1a, HP1b, MBD1, MBD2, Krüppel-Associated Box (KRAB), NIPP1, the Transcription Repression Domain (TRD) of Methyl-CpG Binding Protein 2 (MeCP2), DNMT3A, any combination thereof, or any fusion thereof. In an aspect of a disclosed viral vector or disclosed recombinant AAV vector, a disclosed fusion or disclosed fusion protein can comprise HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect, a disclosed HP1a can comprise the sequence set forth in SEQ ID NO:43, SEQ ID NO:44, or fragment thereof. In an aspect, a disclosed HP1a can comprise a sequence having at least 60%, at least 65%, at least 70%, at least 75%, at least 80%, at least 85%, at least 90%, or at least 95% identity to the sequence set forth in SEQ ID NO:43, SEQ ID NO:44, or a fragment thereof. In an aspect, a disclosed encoded HP1a can comprise the sequence set forth in SEQ ID NO:28, SEQ ID NO:29, or a fragment thereof. In an aspect, a disclosed encoded HP1a can comprise a sequence having at least 60%, at least 65%, at least 70%, at least 75%, at least 80%, at least 85%, at least 90%, or at least 95% identity to the sequence set forth in SEQ ID NO:28, SEQ ID NO:29, or a fragment thereof.

In an aspect, a disclosed HP1b can comprise the sequence set forth in SEQ ID NO:45, SEQ ID NO:46, or a fragment thereof. In an aspect, a disclosed HP1b can comprise a sequence having at least 60%, at least 65%, at least 70%, at least 75%, at least 80%, at least 85%, at least 90%, or at least 95% identity to the sequence set forth in SEQ ID NO:45, SEQ ID NO:46, or a fragment thereof. In an aspect, a disclosed encoded HP1b can comprise the sequence set forth in SEQ ID NO:30, SEQ ID NO:31, or a fragment thereof. In an aspect, a disclosed encoded HP1a can comprise a sequence having at least 60%, at least 65%, at least 70%, at least 75%, at least 80%, at least 85%, at least 90%, or at least 95% identity to the sequence set forth in SEQ ID NO:30, SEQ ID NO:31, or a fragment thereof.

In an aspect, a disclosed MBD1 can comprise the sequence set forth in SEQ ID NO:47, SEQ ID NO:48, or a fragment thereof. In an aspect, a disclosed MBD1 can comprise a sequence having at least 60%, at least 65%, at least 70%, at least 75%, at least 80%, at least 85%, at least 90%, or at least 95% identity to the sequence set forth in SEQ ID NO:47, SEQ ID NO:48, or a fragment thereof. In an aspect, a disclosed encoded MBD1 can comprise the sequence set forth in SEQ ID NO:32, SEQ ID NO:33, SEQ ID NO:34, or a fragment thereof. In an aspect, a disclosed encoded MBD1 can comprise a sequence having at least 60%, at least 65%, at least 70%, at least 75%, at least 80%, at least 85%, at least 90%, or at least 95% identity to the sequence set forth in SEQ ID NO:32, SEQ ID NO:33, SEQ ID NO:34, or a fragment thereof.

In an aspect, a disclosed MBD2 can comprise the sequence set forth in SEQ ID NO:49, SEQ ID NO:50, or a fragment thereof. In an aspect, a disclosed MBD2 can comprise a sequence having at least 60%, at least 65%, at least 70%, at least 75%, at least 80%, at least 85%, at least 90%, or at least 95% identity to the sequence set forth in SEQ ID NO:49, SEQ ID NO:50, or a fragment thereof. In an aspect, a disclosed encoded MBD2 can comprise the sequence set forth in SEQ ID NO:35, SEQ ID NO:36, or a fragment thereof. In an aspect, a disclosed encoded MBD2 can comprise a sequence having at least 60%, at least 65%, at least 70%, at least 75%, at least 80%, at least 85%, at least 90%, or at least 95% identity to the sequence set forth in SEQ ID NO:35, SEQ ID NO:36, or a fragment thereof.

In an aspect, a disclosed KRAB can comprise the sequence set forth in SEQ ID NO:53, SEQ ID NO:54, SEQ ID NO:55, or a fragment thereof. In an aspect, a disclosed KRAB can comprise a sequence having at least 60%, at least 65%, at least 70%, at least 75%, at least 80%, at least 85%, at least 90%, or at least 95% identity to the sequence set forth in SEQ ID NO:53, SEQ ID NO:54, SEQ ID NO:55, or a fragment thereof. In an aspect, a disclosed encoded KRAB can comprise the sequence set forth in SEQ ID NO:39 or a fragment thereof. In an aspect, a disclosed encoded KRAB can comprise a sequence having at least 60%, at least 65%, at least 70%, at least 75%, at least 80%, at least 85%, at least 90%, or at least 95% identity to the sequence set forth in SEQ ID NO:39 or a fragment thereof.

In an aspect, a disclosed NIPP1 can comprise the sequence set forth in SEQ ID NO:51, SEQ ID NO:52, or a fragment thereof. In an aspect, a disclosed NIPP1 can comprise a sequence having at least 60%, at least 65%, at least 70%, at least 75%, at least 80%, at least 85%, at least 90%, or at least 95% identity to the sequence set forth in SEQ ID NO:51, SEQ ID NO:52, or a fragment thereof. In an aspect, a disclosed encoded NIPP1 can comprise the sequence set forth in SEQ ID NO:37, SEQ ID NO:38, or a fragment thereof. In an aspect, a disclosed encoded NIPP1 can comprise a sequence having at least 60%, at least 65%, at least 70%, at least 75%, at least 80%, at least 85%, at least 90%, or at least 95% identity to the sequence set forth in SEQ ID NO:37, SEQ ID NO:38, or a fragment thereof.

In an aspect, a disclosed MeCP2 can comprise the sequence set forth in SEQ ID NO:56, SEQ ID NO:57, or a fragment thereof. In an aspect, a disclosed MeCP2 can comprise a sequence having at least 60%, at least 65%, at least 70%, at least 75%, at least 80%, at least 85%, at least 90%, or at least 95% identity to the sequence set forth in SEQ ID NO:56, SEQ ID NO:57, or a fragment thereof. In an aspect, a disclosed encoded MeCP2 can comprise the sequence set forth in SEQ ID NO:40 or a fragment thereof. In an aspect, a disclosed encoded MeCP2 can comprise a sequence having at least 60%, at least 65%, at least 70%, at least 75%, at least 80%, at least 85%, at least 90%, or at least 95% identity to the sequence set forth in SEQ ID NO:40 or a fragment thereof.

In an aspect, a disclosed KRAB-MeCP2 can comprise the sequence set forth in SEQ ID NO:58, SEQ ID NO:59, or a fragment thereof. In an aspect, a disclosed KRAB-MeCP2 can comprise a sequence having at least 60%, at least 65%, at least 70%, at least 75%, at least 80%, at least 85%, at least 90%, or at least 95% identity to the sequence set forth in SEQ ID NO:58, SEQ ID NO:59, or a fragment thereof. In an aspect, a disclosed encoded KRAB-MeCP2 can comprise the sequence set forth in SEQ ID NO:41 or a fragment thereof. In an aspect, a disclosed encoded KRAB-MeCP2 can comprise a sequence having at least 60%, at least 65%, at least 70%, at least 75%, at least 80%, at least 85%, at least 90%, or at least 95% identity to the sequence set forth in SEQ ID NO:41 or a fragment thereof.

In an aspect, a disclosed DNMT3A can comprise the sequence set forth in SEQ ID NO:60, SEQ ID NO:61, or a fragment thereof. In an aspect, a disclosed DNMT3A can comprise a sequence having at least 60%, at least 65%, at least 70%, at least 75%, at least 80%, at least 85%, at least 90%, or at least 95% identity to the sequence set forth in SEQ ID NO:60, SEQ ID NO:61, or a fragment thereof. In an aspect, a disclosed encoded DNMT3A can comprise the sequence set forth in SEQ ID NO:42 or a fragment thereof. In an aspect, a disclosed encoded DNMT3A can comprise a sequence having at least 60%, at least 65%, at least 70%, at least 75%, at least 80%, at least 85%, at least 90%, or at least 95% identity to the sequence set forth in SEQ ID NO:42 or a fragment thereof.

In an aspect, a disclosed encoded polypeptide can comprise transcription activation activity. A disclosed encoded polypeptide comprising transcription activation activity can comprise a transactivation domain. In an aspect, a disclosed transactivation domain can comprise a VP16 protein, a series of linked VP16 proteins, a p65 domain of NFκB, or any combination thereof.

In an aspect, a disclosed VP16 can comprise the sequence set forth in SEQ ID NO:68, SEQ ID NO:69, or a fragment thereof. In an aspect, a disclosed VP16 can comprise a sequence having at least 60%, at least 65%, at least 70%, at least 75%, at least 80%, at least 85%, at least 90%, or at least 95% identity to the sequence set forth in SEQ ID NO:68, SEQ ID NO:69, or a fragment thereof. In an aspect, a disclosed encoded VP16 can comprise the sequence set forth in SEQ ID NO:70 or a fragment thereof. In an aspect, a disclosed encoded VP16 can comprise a sequence having at least 60%, at least 65%, at least 70%, at least 75%, at least 80%, at least 85%, at least 90%, or at least 95% identity to the sequence set forth in SEQ ID NO:70 or a fragment thereof.

In an aspect, a disclosed p65 domain of NFκB or RELA can comprise the sequence set forth in SEQ ID NO:65, SEQ ID NO:66, or a fragment thereof. In an aspect, a disclosed p65 domain of NFκB or RELA can comprise a sequence having at least 60%, at least 65%, at least 70%, at least 75%, at least 80%, at least 85%, at least 90%, or at least 95% identity to the sequence set forth in SEQ ID NO:65, SEQ ID NO:66, or a fragment thereof. In an aspect, a disclosed encoded p65 domain of NFκB or RELA can comprise the sequence set forth in SEQ ID NO:67 or a fragment thereof. In an aspect, a disclosed encoded p65 domain of NFκB or RELA can comprise a sequence having at least 60%, at least 65%, at least 70%, at least 75%, at least 80%, at least 85%, at least 90%, or at least 95% identity to the sequence set forth in SEQ ID NO:67 or a fragment thereof.

In an aspect, a disclosed encoded polypeptide can comprise transcription repression activity. A disclosed encoded polypeptide comprising transcription repression activity can comprise a Krüppel associated box domain, an ERF repressor domain, a MXI1 repressor domain, a SID4x repressor domain, a fused KRAB-MeCP2 domain, a MeCP2 TRD domain, a MAS-SID repressor domain, a TATA box binding protein activity, or any combination thereof. In an aspect, a disclosed encoded polypeptide comprising transcription repression activity can comprise HP1 repressor activity, MeCP2 repressor activity, MBD1 repressor activity, MBD2 repressor activity, MBD3 repressor activity, MBD4 repressor activity, KRAB repressor activity, SUV39H1 repressor activity, SUV39H2 repressor activity, CTCF insulator-repressor activity, LSD-1 histone-demethylase repressor activity, or any combination thereof.

In an aspect, a disclosed encoded polypeptide can comprise transcription release factor activity. A disclosed encoded polypeptide comprising transcription release factor activity can comprise Eukaryotic Release Factor 1 (ERF1) activity or Eukaryotic Release Factor 3 (ERF3) activity. In an aspect, a disclosed ERF1 can comprise the sequence set forth in SEQ ID NO:71 or a fragment thereof. In an aspect, a disclosed encoded ERF1 can comprise a sequence having at least 60%, at least 65%, at least 70%, at least 75%, at least 80%, at least 85%, at least 90%, or at least 95% identity to the sequence set forth in SEQ ID NO:72 or a fragment thereof.

In an aspect, a disclosed encoded polypeptide can comprise histone modification activity. A disclosed encoded polypeptide comprising histone modification activity can comprise histone acetyltransferase, histone deacetylase, histone demethylase, histone methyltransferase activity, or any combination thereof.

In an aspect, a disclosed encoded polypeptide can comprise nucleic acid association activity. A disclosed encoded polypeptide comprising nucleic acid association activity can comprise a helix-turn-helix region, a leucine zipper region, a winged helix region, a winged helix-turn-helix region, a helix-loop-helix region, an immunoglobulin fold, a B3 domain, a zinc finger, a HMG-box, a Wor3 domain, a TAL effector DNA-binding domain, or any combination thereof.

In an aspect, a disclosed encoded polypeptide can comprise methyltransferase activity. In an aspect, a disclosed encoded polypeptide comprising methyltransferase activity can comprise DNA (cytosine-5)-methyltransferase 3a (DNMT3A). In an aspect, a disclosed encoded polypeptide can comprise demethylase activity. In an aspect, a disclosed encoded polypeptide comprising demethylase activity can comprise ten-eleven translocation methylcytosine dioxygenase 1 (TET1) or lysine-specific histone demethylase 1 (LSD1). In an aspect, a disclosed encoded polypeptide can comprise acetyltransferase activity. In an aspect, a disclosed encoded polypeptide comprising acetyltransferase activity can comprise histone acetyltransferase. In an aspect, a disclosed encoded polypeptide can comprise deacetylase activity. A disclosed encoded polypeptide comprising deacetylase activity can comprise histone deacetylase.

In an aspect, a disclosed encoded dCas endonuclease is fused to the at least one encoded polypeptide having an enzymatic activity. In an aspect, a disclosed dCas endonuclease can comprise dSpCas9 and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dSaCas9 and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dCjCas9 and a disclosed polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof.

In an aspect, a disclosed dCas endonuclease can comprise dVQR, dEQR, or dVRER and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dSpCas9, dSaCas9, or dCjCas9 and a disclosed encoded polypeptide can comprise KRAB-MeCP2.

In an aspect, a disclosed dCas endonuclease can comprise dSpCas9, dSaCas9, or dCjCas9, and a disclosed fusion can comprise HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect of a disclosed viral vector or disclosed recombinant AAV vector, a disclosed gene of interest or portion thereof can demonstrate a reduced expression level when compared to wild-type or control expression level. In an aspect of a disclosed viral vector or disclosed recombinant AAV vector, a disclosed gRNA can targes a gene of interest or portion thereof that demonstrates a reduced expression level when compared to wild-type or control expression level.

In an aspect of a disclosed viral vector or disclosed recombinant AAV vector, a disclosed gene of interest or portion thereof can demonstrate an increased expression level when compared to wild-type or control expression level. In an aspect of a disclosed viral vector or disclosed recombinant AAV vector, a disclosed gRNA can target a gene of interest or portion thereof that demonstrates an increased expression level when compared to wild-type or control expression level.

In an aspect, a disclosed gRNA targeting a gene of interest or portion thereof can comprise at least two gRNAs. In an aspect, disclosed gRNAs can target the same gene of interest or portion thereof. In an aspect, disclosed gRNAs can target different genes of interest or portions thereof.

In an aspect, a disclosed gRNA can target a gene or portion thereof that demonstrates a loss of function. In an aspect, a disclosed gRNA can target a gene or portion thereof that demonstrates a gain of function. Disclosed target genes and disclosed genes of interest are discussed supra.

In an aspect of a disclosed viral vector or disclosed recombinant AAV vector, a disclosed gRNA can target the APOE gene. In an aspect, a disclosed gRNA can target the promoter of the APOE gene. In an aspect, a disclosed gRNA can target exon 4 of the APOE gene. In an aspect, a disclosed gRNA can target a protospacer-adjacent motif (PAM) created by a SNP rs429358 in exon 4 of the APOE gene. In an aspect, a disclosed APOE gene can comprise the sequence set forth in SEQ ID NO:93-SEQ ID NO:96 or a fragment thereof. In an aspect of a disclosed viral vector or disclosed recombinant AAV vector, a disclosed gRNA targeting the APOE gene and/or the promoter of the APOE gene can comprise the sequence set forth in SEQ ID NO:85-SEQ ID NO:92.

In an aspect of a disclosed viral vector or disclosed recombinant AAV vector, a disclosed gRNA can target the SNCA gene. In an aspect, a disclosed gRNA can target the promoter of the SNCA gene. In an aspect, a disclosed gRNA can target exon 1 of the SNCA gene. In an aspect, a disclosed gRNA targeting the SCNA gene and/or the promoter of the SNCA gene can comprise the sequence set forth in SEQ ID NO:81-SEQ ID NO:84.

In an aspect of a disclosed viral vector or disclosed recombinant AAV vector, a disclosed gRNA can target a CMV promoter. In an aspect, a disclosed gRNA targeting the CMV promoter can comprise the sequence set forth in SEQ ID NO:73-SEQ ID NO:80 or a fragment thereof. In an aspect of a disclosed viral vector or disclosed recombinant AAV vector, a disclosed gRNA can target a GFP gene. In an aspect, a disclosed gRNA targeting the GFP gene can comprise the sequence set forth in SEQ ID NO:125, SEQ ID NO:126, or a fragment thereof.

In an aspect, a disclosed viral vector or disclosed recombinant AAV vector can further comprise a nucleic acid sequencing encoding one or more regulatory elements. In an aspect, a disclosed regulatory element can comprise a promoter, an enhancer, a promoter/enhancer, an internal ribosomal entry site, a transcription terminal signal, a polyadenylation signal, a Sp1 and/or NF-kB transcriptional factor binding site, a p2A signal, a woodchuck hepatitis virus post-transcriptional regulatory element, a Phi signal-packaging signal, a rev responsive element, a 5′-LTR, a 3′-LTR, an inverted terminal repeat, a nuclear localization signal (NLS), or any combination thereof.

In an aspect, a disclosed NLS can comprise the sequence set forth in SEQ ID NO:105-SEQ ID NO:111 or a fragment thereof. In an aspect, a disclosed PolyA sequence can comprise the sequence set forth in SEQ ID NO:103, SEQ ID NO:104, or a fragment thereof. In an aspect, a disclosed ITR can comprise the sequence set forth in SEQ ID NO:99-SEQ ID NO:102 or a fragment thereof.

In an aspect, a disclosed viral vector or disclosed recombinant AAV vector can further a nucleic acid sequence encoding one or more promoters. In an aspect, a disclosed promoter can comprise a U6 promoter, a chicken 3-actin promoter, an EF-1α, a CMV promoter, a CMV promoter/enhancer, a fragment thereof, or any combination thereof. In an aspect, a disclosed U6 promoter can comprise the sequence set forth in SEQ ID NO:114 or a fragment thereof. In an aspect, a disclosed U6 promoter can comprise a sequence having at least 60%, at least 65%, at least 70%, at least 75%, at least 80%, at least 85%, at least 90%, at least 95%, or greater than 95% identity to the sequence set forth in SEQ ID NO:114 or a fragment thereof. In an aspect, a disclosed EF-1α promoter can comprise the sequence set forth in SEQ ID NO:112, SEQ ID NO:113, or a fragment thereof. In an aspect, a disclosed EF-1α promoter can comprise a sequence having at least 60%, at least 65%, at least 70%, at least 75%, at least 80%, at least 85%, at least 90%, at least 95%, or greater than 95% identity to the sequence set forth in SEQ ID NO:112, SEQ ID NO: 113, or a fragment thereof. In an aspect, a promoter can be a short EF-1α (EFS-NF) promoter.

In an aspect of a disclosed viral vector or disclosed recombinant AAV vector, a disclosed promoter can be operably linked to the dCas endonuclease. In an aspect, a disclosed promoter operably linked to the dCas endonuclease can comprise an EF-1α or EFS-NC promoter. In an aspect, a disclosed promoter operably linked to the dCas endonuclease can be operably linked to the at least one polypeptide having enzymatic activity. In an aspect, a disclosed promoter can be operably linked to the dCas endonuclease and the at least one polypeptide having enzymatic activity. In an aspect, a disclosed promoter can be operably linked to the fusion protein having one or more enzymatic activities. In an aspect, a disclosed promoter can be operably linked to the dCas endonuclease and the fusion protein having one or more enzymatic activities.

In an aspect, a disclosed promoter operably can be linked to the at least one guide RNA targeting a gene of interest or portion thereof. In an aspect, a disclosed promoter operably linked to the at least one guide RNA can comprise a U6 promoter.

In an aspect, a disclosed viral vector or disclosed recombinant AAV vector can further comprise a gRNA scaffold. In an aspect, a disclosed gRNA scaffold can comprise the sequence set forth in SEQ ID NO:115, SEQ ID NO:116, or a fragment thereof.

In an aspect, a disclosed viral vector or disclosed recombinant AAV vector can further comprise one or more promoters, wherein a first promoter can be operably linked to the dCas endonuclease, and wherein a second promoter can be operably linked to the at least one guide RNA targeting a gene of interest or portion thereof.

In an aspect, a disclosed viral vector or disclosed recombinant AAV vector can further comprise one or more promoters, wherein a first promoter can be operably linked to the dCas endonuclease and the at least one polypeptide having an enzymatic activity, and wherein a second promoter can be operably linked to the at least one guide RNA targeting a gene of interest thereof.

In an aspect, a disclosed viral vector or disclosed recombinant AAV vector, a disclosed encoded polypeptide can comprise HP1a, HP1b, MBD1, MBD2, Krüppel-Associated Box (KRAB), NIPP1, the Transcription Repression Domain (TRD) of Methyl-CpG Binding Protein 2 (MeCP2), DNMT3A, any combination thereof, or any fusion thereof.

In an aspect, a disclosed viral vector or disclosed recombinant AAV vector can comprise a nucleic acid that is CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art. In an aspect, any disclosed Cas9 endonuclease, a disclosed polypeptide having enzymatic activities, a disclosed fusion product or a disclosed fusion protein, or any combination thereof can be codon-optimized.

In an aspect, a disclosed viral vector or disclosed recombinant AAV vector can comprise a sequence that is about 4.5 kilobases or less than about 4.5 kilobases.

Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof.

Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA.

Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof.

Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA.

In an aspect, a disclosed recombinant AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV 11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1). In an aspect, a disclosed AAV vector can be AAV8. In an aspect, a disclosed AAV vector can be AAVhum.8. In an aspect, a disclosed AAV vector can be AAV9.

In an aspect, a disclosed viral vector or disclosed recombinant AAV vector can comprise the sequence set forth in any of SEQ ID NO:01-SEQ ID NO:18, or a variant thereof, or a fragment thereof, or a portion thereof. In an aspect, a disclosed viral vector or disclosed recombinant AAV vector can comprise a sequence having at least 60%, at least 65%, at least 70%, at least 75%, at least 80%, at least 85%, at least 90%, or at least 95% identity to the sequence set forth in SEQ ID NO:01-SEQ ID NO:18, or a variant thereof, or a fragment thereof, or a portion thereof.

Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof. Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA. Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof. Disclosed herein is a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA.

In an aspect, a disclosed viral vector or a disclosed recombinant AAV vector can treat and/or prevent Alzheimer's disease progression. In an aspect, a disclosed viral vector or a disclosed recombinant AAV vector can treat and/or prevent Parkinson's disease progression.

### 4. Pharmaceutical Formulations

Disclosed herein is pharmaceutical formulation comprising a disclosed isolated nucleic acid molecule and a pharmaceutically acceptable carrier. Disclosed herein is pharmaceutical formulation comprising an isolated nucleic acid molecule comprising a nucleic acid sequence encoding (i) a deactivated Cas (dCas) endonuclease, (ii) at least one polypeptide having an enzymatic activity, and (iii) at least one guide RNA targeting a gene of interest or portion thereof, and a pharmaceutically acceptable carrier.

Disclosed herein is pharmaceutical formulation comprising a nucleic acid sequence encoding (i) a deactivated Cas (dCas) endonuclease, (ii) a fusion protein having one or more enzymatic activities, and (iii) at least one guide RNA targeting a gene of interest or portion thereof, and a pharmaceutically acceptable carrier.

Disclosed herein is pharmaceutical formulation comprising an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof, and a pharmaceutically acceptable carrier.

Disclosed herein is pharmaceutical formulation comprising an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA, and a pharmaceutically acceptable carrier.

Disclosed herein is pharmaceutical formulation comprising an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof, and a pharmaceutically acceptable carrier.

Disclosed herein is pharmaceutical formulation comprising an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA, and a pharmaceutically acceptable carrier.

Disclosed herein is pharmaceutical formulation comprising a viral vector comprising a nucleic acid sequence encoding (i) a deactivated Cas (dCas) endonuclease, (ii) at least one polypeptide having an enzymatic activity, and (iii) at least one guide RNA targeting a gene of interest or portion thereof, and a pharmaceutically acceptable carrier. Disclosed herein is pharmaceutical formulation comprising a nucleic acid sequence encoding (i) a deactivated Cas (dCas) endonuclease, (ii) at least one polypeptide having an enzymatic activity, and (iii) at least one guide RNA targeting a gene of interest or portion thereof, and a pharmaceutically acceptable carrier. Disclosed herein is pharmaceutical formulation comprising a nucleic acid sequence encoding (i) a deactivated Cas (dCas) endonuclease, (ii) a fusion protein having one or more enzymatic activities, and (iii) at least one guide RNA targeting a gene of interest or portion thereof, and a pharmaceutically acceptable carrier.

Disclosed herein is pharmaceutical formulation comprising a recombinant AAV vector comprising a disclosed isolated nucleic acid molecule, and a pharmaceutically acceptable carrier. Disclosed herein is pharmaceutical formulation comprising a recombinant AAV vector comprising a disclosed nucleic acid molecule, and a pharmaceutically acceptable carrier. Disclosed herein is pharmaceutical formulation comprising a recombinant AAV vector comprising a nucleic acid sequence encoding (i) a deactivated Cas (dCas) endonuclease, (ii) at least one polypeptide having an enzymatic activity, and (iii) at least one guide RNA targeting a gene of interest or portion thereof, and a pharmaceutically acceptable carrier. Disclosed herein is pharmaceutical formulation comprising a recombinant AAV vector comprising a nucleic acid sequence encoding (i) a deactivated Cas (dCas) endonuclease, (ii) a fusion protein having one or more enzymatic activities, and (iii) at least one guide RNA targeting a gene of interest or portion thereof, and a pharmaceutically acceptable carrier.

Disclosed herein is pharmaceutical formulation comprising a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof, and a pharmaceutically acceptable carrier. Disclosed herein is pharmaceutical formulation comprising a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA, and a pharmaceutically acceptable carrier. Disclosed herein is pharmaceutical formulation comprising a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof, and a pharmaceutically acceptable carrier. Disclosed herein is pharmaceutical formulation comprising a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA, and a pharmaceutically acceptable carrier.

In an aspect, a disclosed formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed composition can comprise one or more proteasome inhibitors. In an aspect, a disclosed composition can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed formulation can comprise a RNA therapeutic. An RNA therapeutic can comprise RNA-mediated interference (RNAi) and/or antisense oligonucleotides (ASO). In an aspect, a disclosed formulation can comprise a disclosed small molecule.

In an aspect, a disclosed pharmaceutical formulation can treat and/or prevent Alzheimer's disease progression. In an aspect, a disclosed pharmaceutical formulation can treat and/or prevent Parkinson's disease progression.

### 5. Host Cells

Disclosed herein is a host cell or a host cell line comprising an isolated nucleic acid molecule comprising a nucleic acid sequence encoding (i) a deactivated Cas (dCas) endonuclease, (ii) at least one polypeptide having an enzymatic activity, and (iii) at least one guide RNA targeting a gene of interest or portion thereof. Disclosed herein is a host cell or a host cell line comprising an isolated nucleic acid molecule comprising a nucleic acid sequence encoding (i) a deactivated Cas (dCas) endonuclease, (ii) a fusion protein having one or more enzymatic activities, and (iii) at least one guide RNA targeting a gene of interest or portion thereof. Disclosed herein is a host cell or a host cell line comprising an isolated nucleic acid molecule comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof. Disclosed herein is a host cell or a host cell line comprising an isolated nucleic acid molecule comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA.

Disclosed herein is a host cell or a host cell line comprising an isolated nucleic acid molecule comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof.

Disclosed herein is a host cell or a host cell line comprising an isolated nucleic acid molecule comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA.

Disclosed herein is a host cell or a host cell line transduced by a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof.

Disclosed herein is a host cell or a host cell line transduced by a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA.

Disclosed herein is a host cell or a host cell line transduced by a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof.

Disclosed herein is a host cell or a host cell line transduced by a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA.

Disclosed herein is a host cell or a host cell line transfected by a plasmid comprising the sequence set forth in any one of SEQ ID NO:01-SEQ ID NO:18 or SEQ ID NO:117-SEQ ID NO:124.

6. Guide RNAs (gRNAs)

Disclosed herein is a guide RNA or gRNA comprising the sequence set forth in any one of SEQ ID NO:73-SEQ ID NO:92 or SEQ ID NO:125-SEQ ID NO:126. Disclosed herein is a guide RNA or gRNA targeting a disclosed gene of interest or portion thereof. Disclosed gRNAs are listed below.

| Identifier | Description |
|---|---|
| SEQ ID NO: 73 | gRNA targeting promoter region of CMV |
| SEQ ID NO: 74 | gRNA targeting promoter region of CMV |
| SEQ ID NO: 75 | gRNA targeting promoter region of CMV |
| SEQ ID NO: 76 | gRNA targeting promoter region of CMV |
| SEQ ID NO: 77 | gRNA targeting promoter region of CMV |
| SEQ ID NO: 78 | gRNA targeting promoter region of CMV |
| SEQ ID NO: 79 | gRNA targeting promoter region of CMV |
| SEQ ID NO: 80 | gRNA targeting promoter region of CMV |
| SEQ ID NO: 81 | gRNA targeting promoter region of CAG-INTRON1 |
| SEQ ID NO: 82 | gRNA targeting promoter region of CAG-INTRON1 |
| SEQ ID NO: 83 | gRNA targeting promoter region of CAG-INTRON1 |
| SEQ ID NO: 84 | gRNA targeting promoter region of CAG-INTRON1 |
| SEQ ID NO: 85 | gRNA targeting promoter region of APOE |
| SEQ ID NO: 86 | gRNA targeting promoter region of APOE |
| SEQ ID NO: 87 | gRNA targeting promoter region of APOE |
| SEQ ID NO: 88 | gRNA targeting promoter region of APOE |
| SEQ ID NO: 89 | gRNA targeting promoter region of APOE |
| SEQ ID NO: 90 | gRNA targeting promoter region of APOE |
| SEQ ID NO: 91 | gRNA targeting promoter region of APOE |
| SEQ ID NO: 92 | gRNA targeting promoter region of APOE |
| SEQ ID NO: 125 | gRNA targeting GFP ORF |
| SEQ ID NO: 126 | gRNA targeting GFP ORF |

As known to the art, a gRNA provides the targeting of a CRISPR/Cas9-based epigenome modifying system. A guide RNA is a specific RNA sequence that recognizes the target DNA region of interest (such as, for example, APOE e4 allele) and directs the Cas endonuclease there for editing. The gRNA is made up of two parts: crispr RNA (crRNA), a 17-20 nucleotide sequence complementary to the target DNA, and a tracr RNA, which serves as a binding scaffold for the Cas nuclease. In an aspect, a disclosed gRNA scaffold can comprise the sequence set forth in SEQ ID NO:115 or SEQ ID NO:116.

In an aspect, a disclosed gRNA can serve to direct a disclosed deactivated Cas9 endonuclease (e.g., dSpCas9, dSaCas9, or dCjCas9) or a disclosed fusion product having a deactivated endonuclease to a target area of interest (such as, for example, the promoter of the APOE gene or the APOE e4 allele or a targeted gene of interest as disclosed above). In an aspect, a disclosed gRNA can serve to direct a disclosed deactivated Cas9 endonuclease (e.g., dSpCas9, dSaCas9, or dCjCas9) or a disclosed fusion product having a disclosed deactivated endonuclease to a target area of interest (such as, for example, a gene having a loss of function).

### 7. Plasmids

Disclosed herein is a plasmid comprising the sequence set forth in any of SEQ ID NO:1-SEQ ID NO:18. Disclosed herein is a plasmid comprising the sequence set forth in any of SEQ ID NO: 117-SEQ ID NO: 124. Plasmids disclosed herein include but are not limited to those listed below.

| Identifier | Plasmid Description |
|---|---|
| SEQ ID NO: 120 | pBK546 carrying dCas9-DNMT3A fused transgene linked to |
| | puromycin reporter via p2A cleavage signal |
| SEQ ID NO: 119 | pBK539 carrying dCas9-DNMT3A fused transgene linked to GFP |
| | reporter via p2A cleavage signal |
| SEQ ID NO: 118 | pBK500 carrying all-in-one lentiviral vector containing fusion |
| | protein and gRNA4 |
| SEQ ID NO: 122 | pBK1531 carrying a Lentiviral vector with dCas9-VRER-MeCP2 |
| | Transcription Repression Domain (TRD) and gRNA1 |
| SEQ ID NO: 123 | pBK1532 carrying a Lentiviral vector with dCas9-VRER-MeCP2 |
| | Transcription Repression Domain (TRD) and gRNA2 |
| SEQ ID NO: 124 | pBK1536 carrying a Lentiviral vector with dCas9-VRER-MeCP2 |
| | Transcription Repression Domain (TRD) without gRNA |
| SEQ ID NO: 117 | pCL35 carrying a Lentiviral vector carrying reporter system |
| | comprised of CAGp-SNCA-INTRON1-SNCA-ORF-dGFP-Nano- |
| | Luc-WPRE-CV40-Puro |
| SEQ ID NO: 121 | pBK1340 carrying a Lentiviral vector carrying reporter system |
| | comprised of CMVp-dGFP-Nano-Luc-WPRE-CV40-Puro |

### F. Methods of Effecting Precision Epigenetic Modulation

Disclosed herein is a method of effecting precision epigenetic modulation, the method comprising contacting one or more cells with a therapeutically effective amount of a disclosed isolated nucleic acid molecule, wherein the expression and/or activity of one or more genes of interest in the one or more cells is modulated. Disclosed herein is a method of effecting precision epigenetic modulation, the method comprising contacting one or more cells with a therapeutically effective amount of a disclosed isolated nucleic acid molecule, wherein the expression and/or activity of one or more genes of interest in the one or more cells is reduced or decreased.

Disclosed herein is a method of effecting precision epigenetic modulation, the method comprising contacting one or more cells with a therapeutically effective amount of a disclosed isolated nucleic acid molecule, wherein the expression and/or activity of one or more genes of interest in the one or more cells is augment or increased. Disclosed herein is a method of effecting precision epigenetic modulation, the method comprising contacting one or more cells with a therapeutically effective amount of a disclosed isolated nucleic acid molecule, wherein the expression and/or activity of APOE e4 in the one or more cells is reduced or decreased.

Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, (ii) at least one polypeptide having an enzymatic activity, and (iii) at least one guide RNA targeting a gene of interest or portion thereof, wherein the expression and/or activity the gene of interest in the one or more cells is modulated. Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, (ii) a fusion protein having one or more enzymatic activities, and (iii) at least one guide RNA targeting a gene of interest or portion thereof, wherein the expression and/or activity the gene of interest in the one or more cells is modulated.

Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof, wherein the expression and/or activity the gene of interest in the one or more cells is modulated.

Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA, wherein the expression and/or activity the gene of interest in the one or more cells is modulated.

Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof, wherein the expression and/or activity the gene of interest in the one or more cells is modulated.

Disclosed herein is a method of effecting precision epigenetic modulation, the method comprising contacting one or more cells with a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA, wherein the expression and/or activity the gene of interest in the one or more cells is modulated.

Disclosed herein is a method of effecting precision epigenetic modulation, the method comprising contacting one or more cells in a subject with a therapeutically effective amount of a disclosed viral vector or disclosed rAAV vector, wherein the expression and/or activity of one or more genes of interest in the one or more cells is modulated.

Disclosed herein is a method of effecting precision epigenetic modulation, the method comprising contacting one or more cells in a subject with a therapeutically effective amount of a disclosed viral vector or disclosed rAAV vector, wherein the expression and/or activity of one or more genes of interest in the one or more cells is reduced or decreased. Disclosed herein is a method of effecting precision epigenetic modulation, the method comprising contacting one or more cells in a subject with a therapeutically effective amount of a disclosed viral vector or disclosed rAAV vector, wherein the expression and/or activity of one or more genes of interest in the one or more cells is augment or increased. Disclosed herein is a method of effecting precision epigenetic modulation, the method comprising contacting one or more cells in a subject with a therapeutically effective amount of a disclosed viral vector or disclosed rAAV vector, wherein the expression and/or activity of APOE e4 in the one or more cells is reduced or decreased.

Disclosed herein is a method of effecting precision epigenetic modulation, the method comprising contacting one or more cells in a subject with a therapeutically effective amount of a viral vector comprising a nucleic acid sequence encoding (i) a deactivated Cas (dCas) endonuclease, (ii) at least one polypeptide having an enzymatic activity, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a viral vector comprising a nucleic acid sequence encoding (i) a deactivated Cas (dCas) endonuclease, (ii) a fusion protein having one or more enzymatic activities, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a recombinant AAV vector comprising a nucleic acid sequence encoding (i) a deactivated Cas (dCas) endonuclease, (ii) at least one polypeptide having an enzymatic activity, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a recombinant AAV vector comprising a nucleic acid sequence encoding (i) a deactivated Cas (dCas) endonuclease, (ii) a fusion protein having one or more enzymatic activities, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the fusion protein, and (v) a promoter operably linked to the at least one guide RNA, wherein the expression and/or activity of one or more genes of interest in the one or more cells is modulated.

Disclosed herein is a method of effecting precision epigenetic modulation, the method comprising contacting one or more cells in a subject with a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting a gene of interest or portion thereof, wherein the expression and/or activity of one or more genes of interest in the one or more cells is modulated.

Disclosed herein is a method of effecting precision epigenetic modulation, the method comprising contacting one or more cells in a subject with a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting a gene of interest or portion thereof, (iv) a promoter operably linked to the dCas endonuclease and the at least one polypeptide, and (v) a promoter operably linked to the at least one guide RNA, wherein the expression and/or activity of one or more genes of interest in the one or more cells is modulated.

In an aspect, a disclosed gene of interest or portion thereof can demonstrate a reduced expression and/or activity level when compared to wild-type or control expression level. In an aspect, a disclosed gene of interest or portion thereof can demonstrate an increased expression and/or activity level when compared to wild-type or control expression level. In an aspect, a disclosed gRNA can target a gene of interest or portion thereof that demonstrates an increased expression level when compared to wild-type or control expression level. In an aspect, a disclosed gRNA can target a gene of interest or portion thereof that demonstrates a reduced expression and/or activity level when compared to wild-type or control expression level.

In an aspect, a disclosed method of effecting precision epigenetic modulation comprises measuring the level of expression and/or the level of activity of the disclosed gene of interest. In an aspect, a disclosed method can comprise repeating the measuring step one or more times.

In an aspect, a disclosed gRNA targeting a gene of interest or portion thereof can comprise at least two gRNAs. In an aspect, disclosed gRNAs can target the same gene of interest or portion thereof. In an aspect, disclosed gRNAs can target different genes of interest or portions thereof. In an aspect, a disclosed gRNA can target a gene or portion thereof that demonstrates a loss of function. In an aspect, a disclosed gRNA can target a gene or portion thereof that demonstrates a gain of function.

In an aspect, a disclosed gene of interest can comprise ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARID1B, ARID2, ASH1L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANEl, CPS1, CPSF1, CRB1, CREBBP, CUBN, CUL7, CUX1, DCC, DCHS1, DEPDC5, DICER1, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNC1H1, DYNC2H1, DYSF, EIF2AK4, EP300, EPG5, ERCC6, ERCC6L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREBIL, GRIN2A, GRIN2B, GRIN2D, HCFC1, HECW2, HERC1, HERC2, HFM1, HIVEP1, HIVEP2, HMCN1, HSPG2, HTT, HUWEl, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12L, MED13, MED13L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MYO18B, MYO3A, MYO5A, MYO5B, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAIl, RALGAPA1, RANBP2, RB1CC1, RELN, RERE, REV3L, RIC1, RIMS1, RIMS2, RNF213, ROBO1, ROBO2, ROBO3, ROS1, RP1, RP1L1, RTTN, RUSC2, RYR1, RYR2, SACS, SAMD9, SAMD9L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAM1, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or any variant thereof, or any fragment thereof, or any portion thereof.

In an aspect, a disclosed gene of interest can comprise APOE (UniProt ID: P02649), APP (UniProt ID: P05067), ATXN2 (UniProt ID: Q99700), CHMP2B (UniProt ID: Q9UQN3), DCTN1 (UniProt ID: Q14203), FIG. 4 (UniProt ID: Q92562), FUS (UniProt ID: P35637), GBA (UniProt ID: P04062), GRN (UniProt ID: P28799), HNRNPA1 (UniProt ID: P09651), HTT (UniProt ID: P42858), LRRK2 (UniProt ID: Q5S007), MATR3 (UniProt ID: P43243), OPTN (UniProt ID: Q96CV9), PARK7 (UniProt ID: Q99497), PFN1 (UniProt ID: P07737), PRPH (UniProt ID: P41219), PSEN1 (UniProt ID: P49768), SETX (UniProt ID: Q7Z333), SIGMAR1 (UniProt ID: Q99720), SNCA (UniProt ID: P37840), SOD1 (UniProt ID: P00441), SPG11 (UniProt ID: Q96JI7), SQSTM1 (UniProt ID: Q13501), TARDBP (UniProt ID: Q13148), TBK1 (UniProt ID: Q9UHD2), TBP (UniProt ID: P20226), TRPM7 (UniProt ID: Q96QT4), TUBA4A (UniProt ID: P68366), UBQLN2 (UniProt ID: Q9UHD9), UCHL1 (UniProt ID: P09936), VAPB (UniProt ID: 095292), VCP (UniProt ID: P55072), VPS35 (UniProt ID: Q96QK1), or any variant thereof, or any fragment thereof, or any portion thereof.

In an aspect of a disclosed method, a disclosed dCas endonuclease can comprise a dCas9 endonuclease. In an aspect, a disclosed dCas9 endonuclease can comprise a deactivated Staphylococcus aureus Cas9 (dSaCas9), a deactivated Streptococcus pyogenes Cas9 (dSpCas9), a deactivated Campylobacter jejuni Cas9 (dCjCas9), or a variant dCas9 endonuclease. In an aspect, a disclosed variant dCas9 can comprise a variant dSaCas9, a variant dSpCas9, or a variant dCjCa9. In an aspect, a disclosed variant dSpCas9 can comprise dVQR, dEQR, or dVRER.

In an aspect of a disclosed method, a disclosed dSpCas9 can comprise the sequence set forth in SEQ ID NO:22, SEQ ID NO:23, or a fragment thereof. In an aspect, a disclosed dSpCas9 can comprise a sequence having at least 60%, at least 65%, at least 70%, at least 75%, at least 80%, at least 85%, at least 90%, or at least 95% identity to the sequence set forth in SEQ ID NO:22, SEQ ID NO:23, or a fragment thereof. In an aspect, a disclosed encoded dSpCas9 can comprise the sequence set forth in SEQ ID NO:19 or a fragment thereof. In an aspect, a disclosed encoded dSpCas9 can comprise a sequence having at least 60%, at least 65%, at least 70%, at least 75%, at least 80%, at least 85%, at least 90%, or at least 95% identity to the sequence set forth in SEQ ID NO:19 or a fragment thereof.

In an aspect of a disclosed method, a disclosed dSaCas9 can comprise the sequence set forth in SEQ ID NO:24 or a fragment thereof. In an aspect, a disclosed dSaCas9 can comprise a sequence having at least 60%, at least 65%, at least 70%, at least 75%, at least 80%, at least 85%, at least 90%, or at least 95% identity to the sequence set forth in SEQ ID NO:24 or a fragment thereof. In an aspect, a disclosed encoded dSaCas9 can comprise the sequence set forth in SEQ ID NO:20 or a fragment thereof. In an aspect, a disclosed encoded dSaCas9 can comprise a sequence having at least 60%, at least 65%, at least 70%, at least 75%, at least 80%, at least 85%, at least 90%, or at least 95% identity to the sequence set forth in SEQ ID NO:20 or a fragment thereof.

In an aspect of a disclosed method, a disclosed dCjCas9 can comprise the sequence set forth in SEQ ID NO:25, SEQ ID NO:26, or a fragment thereof. In an aspect, a disclosed dCjCas9 can comprise a sequence having at least 60%, at least 65%, at least 70%, at least 75%, at least 80%, at least 85%, at least 90%, or at least 95% identity to the sequence set forth in SEQ ID NO:25, SEQ ID NO:26, or a fragment thereof. In an aspect, a disclosed encoded dCjCas9 can comprise the sequence set forth in SEQ ID NO:21 or a fragment thereof. In an aspect, a disclosed encoded dCjCas9 can comprise a sequence having at least 60%, at least 65%, at least 70%, at least 75%, at least 80%, at least 85%, at least 90%, or at least 95% identity to the sequence set forth in SEQ ID NO:21 or a fragment thereof.

In an aspect, a disclosed dVQR can comprise D1135V, R1335Q, and T1337R. In an aspect, a disclosed dEQR can comprise D1135E, R1335Q, and T1337R. In an aspect, a disclosed dVRER can comprise D1135V, G1218R, R1335E, and T1337R. In an aspect, a disclosed dVRER can comprise the sequence set forth in SEQ ID NO:27 or a fragment thereof.

In an aspect of a disclosed method, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof.

In an aspect of a disclosed method, a disclosed encoded polypeptide can comprise HP1a, HP1b, MBD1, MBD2, Krüppel-Associated Box (KRAB), NIPP1, the Transcription Repression Domain (TRD) of Methyl-CpG Binding Protein 2 (MeCP2), DNMT3A, any combination thereof, or any fusion thereof.

In an aspect of a disclosed method, a disclosed fusion or disclosed fusion protein can comprise HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect of a disclosed method, a disclosed HP1a can comprise the sequence set forth in SEQ ID NO:43, SEQ ID NO:44, or fragment thereof. In an aspect, a disclosed HP1a can comprise a sequence having at least 60%, at least 65%, at least 70%, at least 75%, at least 80%, at least 85%, at least 90%, or at least 95% identity to the sequence set forth in SEQ ID NO:43, SEQ ID NO:44, or a fragment thereof. In an aspect, a disclosed encoded HP1a can comprise the sequence set forth in SEQ ID NO:28, SEQ ID NO:29, or a fragment thereof. In an aspect, a disclosed encoded HP1a can comprise a sequence having at least 60%, at least 65%, at least 70%, at least 75%, at least 80%, at least 85%, at least 90%, or at least 95% identity to the sequence set forth in SEQ ID NO:28, SEQ ID NO:29, or a fragment thereof.

In an aspect of a disclosed method, a disclosed HP1b can comprise the sequence set forth in SEQ ID NO:45, SEQ ID NO:46, or a fragment thereof. In an aspect, a disclosed HP1b can comprise a sequence having at least 60%, at least 65%, at least 70%, at least 75%, at least 80%, at least 85%, at least 90%, or at least 95% identity to the sequence set forth in SEQ ID NO:45, SEQ ID NO:46, or a fragment thereof. In an aspect, a disclosed encoded HP1b can comprise the sequence set forth in SEQ ID NO:30, SEQ ID NO:31, or a fragment thereof. In an aspect, a disclosed encoded HIP1a can comprise a sequence having at least 60%, at least 65%, at least 70%, at least 75%, at least 80%, at least 85%, at least 90%, or at least 95% identity to the sequence set forth in SEQ ID NO:30, SEQ ID NO:31, or a fragment thereof.

In an aspect of a disclosed method, a disclosed MBD1 can comprise the sequence set forth in SEQ ID NO:47, SEQ ID NO:48, or a fragment thereof. In an aspect, a disclosed MBD1 can comprise a sequence having at least 60%, at least 65%, at least 70%, at least 75%, at least 80%, at least 85%, at least 90%, or at least 95% identity to the sequence set forth in SEQ ID NO:47, SEQ ID NO:48, or a fragment thereof. In an aspect, a disclosed encoded MBD1 can comprise the sequence set forth in SEQ ID NO:32, SEQ ID NO:33, SEQ ID NO:34, or a fragment thereof. In an aspect, a disclosed encoded MBD1 can comprise a sequence having at least 60%, at least 65%, at least 70%, at least 75%, at least 80%, at least 85%, at least 90%, or at least 95% identity to the sequence set forth in SEQ ID NO:32, SEQ ID NO:33, SEQ ID NO:34, or a fragment thereof.

In an aspect of a disclosed method, a disclosed MBD2 can comprise the sequence set forth in SEQ ID NO:49, SEQ ID NO:50, or a fragment thereof. In an aspect, a disclosed MBD2 can comprise a sequence having at least 60%, at least 65%, at least 70%, at least 75%, at least 80%, at least 85%, at least 90%, or at least 95% identity to the sequence set forth in SEQ ID NO:49, SEQ ID NO:50, or a fragment thereof. In an aspect, a disclosed encoded MBD2 can comprise the sequence set forth in SEQ ID NO:35, SEQ ID NO:36, or a fragment thereof. In an aspect, a disclosed encoded MBD2 can comprise a sequence having at least 60%, at least 65%, at least 70%, at least 75%, at least 80%, at least 85%, at least 90%, or at least 95% identity to the sequence set forth in SEQ ID NO:35, SEQ ID NO:36, or a fragment thereof.

In an aspect of a disclosed method, a disclosed KRAB can comprise the sequence set forth in SEQ ID NO:53, SEQ ID NO:54, SEQ ID NO:55, or a fragment thereof. In an aspect, a disclosed KRAB can comprise a sequence having at least 60%, at least 65%, at least 70%, at least 75%, at least 80%, at least 85%, at least 90%, or at least 95% identity to the sequence set forth in SEQ ID NO:53, SEQ ID NO:54, SEQ ID NO:55, or a fragment thereof. In an aspect, a disclosed encoded KRAB can comprise the sequence set forth in SEQ ID NO:39 or a fragment thereof. In an aspect, a disclosed encoded KRAB can comprise a sequence having at least 60%, at least 65%, at least 70%, at least 75%, at least 80%, at least 85%, at least 90%, or at least 95% identity to the sequence set forth in SEQ ID NO:39 or a fragment thereof.

In an aspect of a disclosed method, a disclosed NIPP1 can comprise the sequence set forth in SEQ ID NO:51, SEQ ID NO:52, or a fragment thereof. In an aspect, a disclosed NIPP1 can comprise a sequence having at least 60%, at least 65%, at least 70%, at least 75%, at least 80%, at least 85%, at least 90%, or at least 95% identity to the sequence set forth in SEQ ID NO:51, SEQ ID NO:52, or a fragment thereof. In an aspect, a disclosed encoded NIPP1 can comprise the sequence set forth in SEQ ID NO:37, SEQ ID NO:38, or a fragment thereof. In an aspect, a disclosed encoded NIPP1 can comprise a sequence having at least 60%, at least 65%, at least 70%, at least 75%, at least 80%, at least 85%, at least 90%, or at least 95% identity to the sequence set forth in SEQ ID NO:37, SEQ ID NO:38, or a fragment thereof.

In an aspect of a disclosed method, a disclosed MeCP2 can comprise the sequence set forth in SEQ ID NO:56, SEQ ID NO:57, or a fragment thereof. In an aspect, a disclosed MeCP2 can comprise a sequence having at least 60%, at least 65%, at least 70%, at least 75%, at least 80%, at least 85%, at least 90%, or at least 95% identity to the sequence set forth in SEQ ID NO:56, SEQ ID NO:57, or a fragment thereof. In an aspect, a disclosed encoded MeCP2 can comprise the sequence set forth in SEQ ID NO:40 or a fragment thereof. In an aspect, a disclosed encoded MeCP2 can comprise a sequence having at least 60%, at least 65%, at least 70%, at least 75%, at least 80%, at least 85%, at least 90%, or at least 95% identity to the sequence set forth in SEQ ID NO:40 or a fragment thereof.

In an aspect of a disclosed method, a disclosed KRAB-MeCP2 can comprise the sequence set forth in SEQ ID NO:58, SEQ ID NO:59, or a fragment thereof. In an aspect, a disclosed KRAB-MeCP2 can comprise a sequence having at least 60%, at least 65%, at least 70%, at least 75%, at least 80%, at least 85%, at least 90%, or at least 95% identity to the sequence set forth in SEQ ID NO:58, SEQ ID NO:59, or a fragment thereof. In an aspect, a disclosed encoded KRAB-MeCP2 can comprise the sequence set forth in SEQ ID NO:41 or a fragment thereof. In an aspect, a disclosed encoded KRAB-MeCP2 can comprise a sequence having at least 60%, at least 65%, at least 70%, at least 75%, at least 80%, at least 85%, at least 90%, or at least 95% identity to the sequence set forth in SEQ ID NO:41 or a fragment thereof.

In an aspect of a disclosed method, a disclosed DNMT3A can comprise the sequence set forth in SEQ ID NO:60, SEQ ID NO:61, or a fragment thereof. In an aspect, a disclosed DNMT3A can comprise a sequence having at least 60%, at least 65%, at least 70%, at least 75%, at least 80%, at least 85%, at least 90%, or at least 95% identity to the sequence set forth in SEQ ID NO:60, SEQ ID NO:61, or a fragment thereof. In an aspect, a disclosed encoded DNMT3A can comprise the sequence set forth in SEQ ID NO:42 or a fragment thereof. In an aspect, a disclosed encoded DNMT3A can comprise a sequence having at least 60%, at least 65%, at least 70%, at least 75%, at least 80%, at least 85%, at least 90%, or at least 95% identity to the sequence set forth in SEQ ID NO:42 or a fragment thereof.

In an aspect of a disclosed method, a disclosed encoded polypeptide can comprise transcription activation activity. A disclosed encoded polypeptide comprising transcription activation activity can comprise a transactivation domain. In an aspect, a disclosed transactivation domain can comprise a VP16 protein, a series of linked VP16 proteins, a p65 domain of NFκB, or any combination thereof.

In an aspect of a disclosed method, a disclosed VP16 can comprise the sequence set forth in SEQ ID NO:68, SEQ ID NO:69, or a fragment thereof. In an aspect, a disclosed VP16 can comprise a sequence having at least 60%, at least 65%, at least 70%, at least 75%, at least 80%, at least 85%, at least 90%, or at least 95% identity to the sequence set forth in SEQ ID NO:68, SEQ ID NO:69, or a fragment thereof. In an aspect, a disclosed encoded VP16 can comprise the sequence set forth in SEQ ID NO:70 or a fragment thereof. In an aspect, a disclosed encoded VP16 can comprise a sequence having at least 60%, at least 65%, at least 70%, at least 75%, at least 80%, at least 85%, at least 90%, or at least 95% identity to the sequence set forth in SEQ ID NO:70 or a fragment thereof.

In an aspect of a disclosed method, a disclosed p65 domain of NFκB or RELA can comprise the sequence set forth in SEQ ID NO:65, SEQ ID NO:66, or a fragment thereof. In an aspect, a disclosed p65 domain of NFκB or RELA can comprise a sequence having at least 60%, at least 65%, at least 70%, at least 75%, at least 80%, at least 85%, at least 90%, or at least 95% identity to the sequence set forth in SEQ ID NO:65, SEQ ID NO:66, or a fragment thereof. In an aspect, a disclosed encoded p65 domain of NFκB or RELA can comprise the sequence set forth in SEQ ID NO:67 or a fragment thereof. In an aspect, a disclosed encoded p65 domain of NFκB or RELA can comprise a sequence having at least 60%, at least 65%, at least 70%, at least 75%, at least 80%, at least 85%, at least 90%, or at least 95% identity to the sequence set forth in SEQ ID NO:67 or a fragment thereof.

In an aspect of a disclosed method, a disclosed encoded polypeptide can comprise transcription repression activity. A disclosed encoded polypeptide comprising transcription repression activity can comprise a Krüppel associated box domain, an ERF repressor domain, a MXI1 repressor domain, a SID4x repressor domain, a fused KRAB-MeCP2 domain, a MeCP2 TRD domain, a MAS-SID repressor domain, a TATA box binding protein activity, or any combination thereof. In an aspect, a disclosed encoded polypeptide comprising transcription repression activity can comprise HP1 repressor activity, MeCP2 repressor activity, MBD1 repressor activity, MBD2 repressor activity, MBD3 repressor activity, MBD4 repressor activity, KRAB repressor activity, SUV39H1 repressor activity, SUV39H2 repressor activity, CTCF insulator-repressor activity, LSD-1 histone-demethylase repressor activity, or any combination thereof. In an aspect of a disclosed method, a disclosed encoded polypeptide can comprise transcription release factor activity. A disclosed encoded polypeptide comprising transcription release factor activity can comprise Eukaryotic Release Factor 1 (ERF1) activity or Eukaryotic Release Factor 3 (ERF3) activity. In an aspect, a disclosed ERF1 can comprise the sequence set forth in SEQ ID NO:71 or a fragment thereof. In an aspect, a disclosed encoded ERF1 can comprise a sequence having at least 60%, at least 65%, at least 70%, at least 75%, at least 80%, at least 85%, at least 90%, or at least 95% identity to the sequence set forth in SEQ ID NO:72 or a fragment thereof.

In an aspect of a disclosed method, a disclosed encoded polypeptide can comprise histone modification activity. A disclosed encoded polypeptide comprising histone modification activity can comprise histone acetyltransferase, histone deacetylase, histone demethylase, histone methyltransferase activity, or any combination thereof.

In an aspect of a disclosed method, a disclosed encoded polypeptide can comprise nucleic acid association activity. A disclosed encoded polypeptide comprising nucleic acid association activity can comprise a helix-turn-helix region, a leucine zipper region, a winged helix region, a winged helix-turn-helix region, a helix-loop-helix region, an immunoglobulin fold, a B3 domain, a zinc finger, a HMG-box, a Wor3 domain, a TAL effector DNA-binding domain, or any combination thereof. In an aspect of a disclosed method, a disclosed encoded polypeptide can comprise methyltransferase activity. A disclosed encoded polypeptide comprising methyltransferase activity can comprise DNA (cytosine-5)-methyltransferase 3a (DNMT3A).

In an aspect of a disclosed method, a disclosed encoded polypeptide can comprise demethylase activity. A disclosed encoded polypeptide comprising demethylase activity can comprise ten-eleven translocation methylcytosine dioxygenase 1 (TET1) or lysine-specific histone demethylase 1 (LSD1).

In an aspect of a disclosed method, a disclosed encoded polypeptide can comprise acetyltransferase activity. A disclosed encoded polypeptide comprising acetyltransferase activity can comprise histone acetyltransferase. In an aspect of a disclosed method, a disclosed encoded polypeptide can comprise deacetylase activity. A disclosed encoded polypeptide comprising deacetylase activity can comprise histone deacetylase.

In an aspect of a disclosed method, a disclosed encoded dCas endonuclease is fused to the at least one encoded polypeptide having an enzymatic activity. In an aspect, a disclosed dCas endonuclease can comprise dSpCas9 and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dSaCas9 and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dCjCas9 and a disclosed polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof.

In an aspect of a disclosed method, a disclosed dCas endonuclease can comprise dVQR, dEQR, or dVRER and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof.

In an aspect of a disclosed method, a disclosed dCas endonuclease can comprise dSpCas9, dSaCas9, or dCjCas9 and a disclosed encoded polypeptide can comprise KRAB-MeCP2.

In an aspect of a disclosed method, a disclosed dCas endonuclease can comprise dSpCas9, dSaCas9, or dCjCas9, and a disclosed fusion can comprise HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect of a disclosed method, a disclosed gene of interest or portion thereof can demonstrate a reduced expression level when compared to wild-type or control expression level. In an aspect of a disclosed method, a disclosed gRNA can targes a gene of interest or portion thereof that demonstrates a reduced expression level when compared to wild-type or control expression level. In an aspect of a disclosed method, a disclosed gene of interest or portion thereof can demonstrate an increased expression level when compared to wild-type or control expression level. In an aspect of a disclosed method, a disclosed gRNA can target a gene of interest or portion thereof that demonstrates an increased expression level when compared to wild-type or control expression level.

In an aspect of a disclosed method, a disclosed gRNA targeting a gene of interest or portion thereof can comprise at least two gRNAs. In an aspect, disclosed gRNAs can target the same gene of interest or portion thereof. In an aspect, disclosed gRNAs can target different genes of interest or portions thereof. In an aspect of a disclosed method, a disclosed gRNA can target a gene or portion thereof that demonstrates a loss of function. In an aspect, a disclosed gRNA can target a gene or portion thereof that demonstrates a gain of function. Disclosed target genes and disclosed genes of interest are discussed supra.

In an aspect of a disclosed method, a disclosed gRNA can target the APOE gene. In an aspect, a disclosed gRNA can target the promoter of the APOE gene. In an aspect, a disclosed gRNA can target exon 4 of the APOE gene. In an aspect, a disclosed gRNA can target a protospacer-adjacent motif (PAM) created by a SNP rs429358 in exon 4 of the APOE gene. In an aspect, a disclosed APOE gene can comprise the sequence set forth in SEQ ID NO:93-SEQ ID NO:96 or a fragment thereof. In an aspect of a disclosed viral vector or disclosed recombinant AAV vector, a disclosed gRNA targeting the APOE gene and/or the promoter of the APOE gene can comprise the sequence set forth in SEQ ID NO:85-SEQ ID NO:92.

In an aspect of a disclosed method, a disclosed gRNA can target the SNCA gene. In an aspect, a disclosed gRNA can target the promoter of the SNCA gene. In an aspect, a disclosed gRNA can target exon 1 of the SNCA gene. In an aspect, a disclosed gRNA targeting the SCNA gene and/or the promoter of the SNCA gene can comprise the sequence set forth in SEQ ID NO:81-SEQ ID NO:84.

In an aspect of a disclosed method, a disclosed gRNA can target a CMV promoter. In an aspect, a disclosed gRNA targeting the CMV promoter can comprise the sequence set forth in SEQ ID NO:73-SEQ ID NO:80 or a fragment thereof. In an aspect of a disclosed method, a disclosed gRNA can target a GFP gene. In an aspect, a disclosed gRNA targeting the GFP gene can comprise the sequence set forth in SEQ ID NO:125, SEQ ID NO:126, or a fragment thereof.

In an aspect of a disclosed method, a disclosed viral vector or disclosed recombinant AAV vector can further comprise a nucleic acid sequencing encoding one or more regulatory elements. In an aspect, a disclosed regulatory element can comprise a promoter, an enhancer, a promoter/enhancer, an internal ribosomal entry site, a transcription terminal signal, a polyadenylation signal, a Sp1 and/or NF-kB transcriptional factor binding site, a p2A signal, a woodchuck hepatitis virus post-transcriptional regulatory element, a Phi signal-packaging signal, a rev responsive element, a 5′-LTR, a 3′-LTR, an inverted terminal repeat, a nuclear localization signal (NLS), or any combination thereof.

In an aspect, a disclosed NLS can comprise the sequence set forth in SEQ ID NO:105-SEQ ID NO:111 or a fragment thereof. In an aspect, a disclosed PolyA sequence can comprise the sequence set forth in SEQ ID NO:103, SEQ ID NO:104, or a fragment thereof. In an aspect, a disclosed ITR can comprise the sequence set forth in SEQ ID NO:99-SEQ ID NO:102 or a fragment thereof.

In an aspect of a disclosed method, a disclosed viral vector or disclosed recombinant AAV vector can further a nucleic acid sequence encoding one or more promoters. In an aspect, a disclosed promoter can comprise a U6 promoter, a chicken β-actin promoter, an EF-1α, a CMV promoter, a CMV promoter/enhancer, a fragment thereof, or any combination thereof. In an aspect, a disclosed U6 promoter can comprise the sequence set forth in SEQ ID NO:114 or a fragment thereof. In an aspect, a disclosed U6 promoter can comprise a sequence having at least 60%, at least 65%, at least 70%, at least 75%, at least 80%, at least 85%, at least 90%, at least 95%, or greater than 95% identity to the sequence set forth in SEQ ID NO:114 or a fragment thereof. In an aspect, a disclosed EF-1α promoter can comprise the sequence set forth in SEQ ID NO:112, SEQ ID NO:113, or a fragment thereof. In an aspect, a disclosed EF-1α promoter can comprise a sequence having at least 60%, at least 65%, at least 70%, at least 75%, at least 80%, at least 85%, at least 90%, at least 95%, or greater than 95% identity to the sequence set forth in SEQ ID NO:112, SEQ ID NO:113, or a fragment thereof. In an aspect, a promoter can be a short EF-1α (EFS-NF) promoter.

In an aspect of a disclosed method, a disclosed promoter can be operably linked to the dCas endonuclease. In an aspect, a disclosed promoter operably linked to the dCas endonuclease can comprise an EF-1α or EFS-NC promoter. In an aspect, a disclosed promoter operably linked to the dCas endonuclease can be operably linked to the at least one polypeptide having enzymatic activity. In an aspect, a disclosed promoter can be operably linked to the dCas endonuclease and the at least one polypeptide having enzymatic activity. In an aspect, a disclosed promoter can be operably linked to the fusion protein having one or more enzymatic activities. In an aspect, a disclosed promoter can be operably linked to the dCas endonuclease and the fusion protein having one or more enzymatic activities.

In an aspect, a disclosed promoter operably can be linked to the at least one guide RNA targeting a gene of interest or portion thereof. In an aspect, a disclosed promoter operably linked to the at least one guide RNA can comprise a U6 promoter.

In an aspect of a disclosed method, a disclosed viral vector or disclosed recombinant AAV vector can further comprise a gRNA scaffold. In an aspect, a disclosed gRNA scaffold can comprise the sequence set forth in SEQ ID NO:115, SEQ ID NO:116, or a fragment thereof.

In an aspect of a disclosed method, a disclosed viral vector or disclosed recombinant AAV vector can further comprise one or more promoters, wherein a first promoter can be operably linked to the dCas endonuclease, and wherein a second promoter can be operably linked to the at least one guide RNA targeting a gene of interest or portion thereof.

In an aspect of a disclosed method, a disclosed viral vector or disclosed recombinant AAV vector can further comprise one or more promoters, wherein a first promoter can be operably linked to the dCas endonuclease and the at least one polypeptide having an enzymatic activity, and wherein a second promoter can be operably linked to the at least one guide RNA targeting a gene of interest thereof.

In an aspect of a disclosed method, a disclosed viral vector or disclosed recombinant AAV vector, a disclosed encoded polypeptide can comprise HP1a, HP1b, MBD1, MBD2, Krüppel-Associated Box (KRAB), NIPP1, the Transcription Repression Domain (TRD) of Methyl-CpG Binding Protein 2 (MeCP2), DNMT3A, any combination thereof, or any fusion thereof.

In an aspect of a disclosed method, a disclosed viral vector or disclosed recombinant AAV vector can comprise a nucleic acid that is CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art. In an aspect, any disclosed Cas9 endonuclease, a disclosed polypeptide having enzymatic activities, a disclosed fusion product or a disclosed fusion protein, or any combination thereof can be codon-optimized.

In an aspect of a disclosed method, a disclosed viral vector or disclosed recombinant AAV vector can comprise a sequence that is about 4.5 kilobases or less than about 4.5 kilobases.

In an aspect of a disclosed method of effecting precision epigenetic modulation, modulating can comprise increasing expression and/or activity of one or more genes of interest. In an aspect of a disclosed method of effecting precision epigenetic modulation, modulating can comprise decreasing expression and/or activity of one or more genes of interest.

In an aspect, a disclosed method of effecting precision epigenetic modulation can comprise reducing expression and/or activity of APOE e4. In an aspect, a disclosed method can comprise reducing expression and/or activity of APOE regardless of the subject's genotype. In an aspect, a disclosed method can comprise reducing expression and/or activity of SNCA.

In an aspect, the disclosed cells can be neurons (e.g., cholinergic neurons, dopaminergic neurons, etc.). In an aspect, the disclosed cells can be cells affected by an overexpression level and/or activity level of one or more genes of interest or one or more target genes.

In an aspect, the disclosed cells can be in a subject. In an aspect of a disclosed method, a subject can be a human. In an aspect, a subject can be suspected of having or can be diagnosed with having Alzheimer's disease (such as, for example, PD). In an aspect, a subject can be suspected of having or can be diagnosed with having Parkinson's disease or Lewy Body Dementia. In an aspect, a disclosed subject can be symptomatic or asymptomatic.

In an aspect, a disclosed method can comprise reducing the pathological phenotype associated with Alzheimer's disease. In an aspect, reducing the pathological phenotype associated with Alzheimer's disease can comprise reducing the Aβ42/40 ratio and reducing the level of Tau. In an aspect, a disclosed method can comprise diagnosing the subject with Alzheimer's disease.

In an aspect, a disclosed method can comprise reducing the pathological phenotype associated with Parkinson's disease or Lewy Body Dementia. In an aspect, reducing the pathological phenotype associated with Alzheimer's disease can comprise reducing the number and/or amount of amyloid plaques. In an aspect, a disclosed method can comprise diagnosing the subject with Parkinson's disease or Lewy Body Dementia.

In an aspect of a disclosed method of effecting precision epigenetic modulation, by knowing what disease or disorder is affecting the subject, the skilled person can identify the relevant gene or genes. In an aspect, a subject can be a subject having a disease or disorder. In an aspect, a disease or disorder can be any disease or disorder disclosed herein, for example, characterized by an overexpression and/or over-activity or characterized by a loss of function.

In an aspect, a subject can be a subject in need of treatment of a disclosed disease or disorder (e.g., a genetic disease or disorder).

In an aspect, a disclosed method of effecting precision epigenetic modulation can restore the functionality and/or structural integrity of a missing, deficient, and/or mutant protein or enzyme. In an aspect, a disclosed method of effecting precision epigenetic modulation can comprise restoring one or more aspects of cellular homeostasis and/or cellular functionality and/or metabolic dysregulation.

In an aspect, restoring one or more aspects of cellular homeostasis and/or cellular functionality and/or metabolic dysregulation can comprise reducing the expression and/or activity level of at least one gene of interest or at least one target gene. In an aspect, a gene of interest can be a gene that demonstrates over expression and/or over activity.

In an aspect, restoring one or more aspects of cellular homeostasis and/or cellular functionality and/or metabolic dysregulation can comprise increasing the expression and/or activity level of at least one gene of interest or at least one target gene. In an aspect, a gene of interest can be a gene that demonstrates a loss of function or reduced expression and/or activity level.

In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof.

In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme). In an aspect, restoration can be a partial or incomplete restoration. In an aspect, restoration can be complete or near complete restoration such that the level of expression, activity, and/or functionality is like that of a wild-type or control level.

In an aspect, restoring the activity and/or functionality of a gene of interest can comprise increasing or enhancing the expression and/or activity level of that gene. In an aspect, increasing or enhancing can comprise an elevation of at least about 5%, 10%, 15%, 20%, 25%, 35%, 50%, 75%, 80%, 85%, 90%, 95%, 97%, 98%, 99%, 100%, 200%, 300%, 400%, 500%, or more as compared to a control (such as a pre-treatment level). In an aspect, restoring the activity and/or functionality of a gene of interest can comprise decreasing or reducing the expression and/or activity level of that gene. In an aspect, decreasing or reducing can comprise a decrease of at least about 5%, 10%, 15%, 20%, 25%, 35%, 50%, 75%, 80%, 85%, 90%, 95%, 97%, 98%, 99%, 100%, 200%, 300%, 400%, 500%, or more as compared to a control (such as a pre-treatment level).

In an aspect of a disclosed method of effecting precision epigenetic modulation, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect of a disclosed method of effecting precision epigenetic modulation, administering can comprise intravenous administration, intracerebral administration, intra-CSF administration, intracerebroventricular (ICV) administration, intraventricular administration, intra-cisterna magna (ICM) administration, intraparenchymal administration, intrathecal (lumbar, cisternal, or both) administration, intrahepatic administration, hepatic intra-arterial administration, hepatic portal vein (HPV) administration, or any combination thereof. In an aspect, a disclosed vector can be administered via LNP administration.

In an aspect, a disclosed method of effecting precision epigenetic modulation can employ multiple routes of administration to the subject. In an aspect, a disclosed method can employ a first route of administration that can be the same or different as a second and/or subsequent routes of administration. In an aspect, a disclosed nucleic acid molecule, a disclosed vector, and/or a disclosed pharmaceutical formulation can be concurrently and/or serially administered to a subject via multiple routes of administration. For example, in an aspect, administering a disclosed nucleic acid molecule, a disclosed vector, and/or a disclosed pharmaceutical formulation can comprise intravenous administration and intra-cistern magna (ICM) administration. In an aspect, administering a disclosed nucleic acid molecule, a disclosed vector, and/or a disclosed pharmaceutical formulation can comprise IV administration and intrathecal (ITH) administration.

In an aspect of a disclosed method of effecting precision epigenetic modulation, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ vg/kg to about 8×10¹³ vg/kg or about 1×10¹² vg/kg to about 8×10¹³ vg/kg. In an aspect, a disclosed vector can be administered at a dose of about 1×10¹³ vg/kg to about 6×10¹³ vg/kg. In an aspect, a disclosed vector can be administered at a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a disclosed vector can be administered at a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method of effecting precision epigenetic modulation can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method of effecting precision epigenetic modulation can further comprise administering to the subject a therapeutically effective amount of a therapeutic agent. A therapeutic agent can be any disclosed agent that effects a desired clinical outcome.

In an aspect, a disclosed method of effecting precision epigenetic modulation can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method of effecting precision epigenetic modulation can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of effecting precision epigenetic modulation can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method of effecting precision epigenetic modulation can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of effecting precision epigenetic modulation can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of effecting precision epigenetic modulation can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect of a disclosed method, a subject can be a human. In an aspect, a subject can be suspected of having or can be diagnosed with having Alzheimer's disease (such as, for example, PD). In an aspect, a disclosed subject can be symptomatic or asymptomatic.

In an aspect, a disclosed method of effecting precision epigenetic modulation can comprise reducing the pathological phenotype associated with Alzheimer's disease. In an aspect, reducing the pathological phenotype associated with Alzheimer's disease can comprise reducing the Aβ42/40 ratio and reducing the level of Tau. In an aspect, a disclosed method can comprise diagnosing the subject with Alzheimer's disease.

In an aspect, a disclosed method can comprise repeating one or more steps of the method and/or modifying one or more steps of the method (such as, for example, an administering step).

In an aspect, a disclosed method of effecting precision epigenetic modulation can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, a disclosed method of effecting precision epigenetic modulation can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In an aspect, a disclosed method of effecting precision epigenetic modulation can further comprise generating and/or validating one or more of the disclosed isolated nucleic acid molecules, one or more of the disclosed vectors, one or more of the disclosed pharmaceutical formulations, or any combination thereof.

In an aspect, a disclosed method of effecting precision epigenetic modulation can further comprise contacting the cells with a second disclosed isolated nucleic acid molecule, a second disclosed vector, a second disclosed pharmaceutical formulations, or any combination thereof. In an aspect, a disclosed method of effecting precision epigenetic modulation can further comprise contacting the cells with additional disclosed isolated nucleic acid molecules, additional disclosed vectors, additional disclosed pharmaceutical formulations, or any combination thereof.

### G. Methods of Treating and/or Preventing Alzheimer's Disease Progression

Disclosed herein is a method of treating and/or preventing Alzheimer's disease progression in a subject, the method comprising administering to a subject a therapeutically effective amount of a disclosed isolated nucleic acid molecule, thereby reducing the pathological phenotype associated with Alzheimer's disease.

Disclosed herein is a method of treating and/or preventing Alzheimer's disease progression in a subject, the method comprising administering to a subject a therapeutically effective amount of an isolated nucleic acid molecule, comprising: a nucleic acid sequence encoding (i) a deactivated Cas (dCas) endonuclease, (ii) at least one polypeptide having an enzymatic activity, and (iii) at least one guide RNA targeting the APOE gene or a portion of the APOE gene, thereby reducing the pathological phenotype associated with Alzheimer's disease.

Disclosed herein is a method of treating and/or preventing Alzheimer's disease progression in a subject, the method comprising administering to a subject a therapeutically effective amount of an isolated nucleic acid molecule, comprising: a nucleic acid sequence encoding (i) a deactivated Cas (dCas) endonuclease, (ii) a fusion protein having one or more enzymatic activities, and (iii) at least one guide RNA targeting the APOE gene or a portion of the APOE gene, thereby reducing the pathological phenotype associated with Alzheimer's disease.

Disclosed herein is a method of treating and/or preventing Alzheimer's disease progression in a subject, the method comprising administering to a subject a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting the APOE gene or a portion of the APOE gene, thereby reducing the pathological phenotype associated with Alzheimer's disease.

Disclosed herein is a method of treating and/or preventing Alzheimer's disease progression in a subject, the method comprising administering to a subject a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting the APOE gene or a portion of the APOE gene, (iv) a promoter operably linked to the dCas endonuclease and the fusion protein, and (v) a promoter operably linked to the at least one guide RNA, thereby reducing the pathological phenotype associated with Alzheimer's disease.

Disclosed herein is a method of treating and/or preventing Alzheimer's disease progression in a subject, the method comprising administering to a subject a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting the APOE gene or a portion of the APOE gene, thereby reducing the pathological phenotype associated with Alzheimer's disease.

Disclosed herein is a method of treating and/or preventing Alzheimer's disease progression in a subject, the method comprising administering to a subject a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting the APOE gene or a portion of the APOE gene, (iv) a promoter operably linked to the dCas endonuclease and the at least one polypeptide, and (v) a promoter operably linked to the at least one guide RNA, thereby reducing the pathological phenotype associated with Alzheimer's disease.

Disclosed herein is a method of treating and/or preventing Alzheimer's disease progression in a subject, the method comprising administering to a subject a therapeutically effective amount of a disclosed viral vector or disclosed rAAV vector, thereby reducing the pathological phenotype associated with Alzheimer's disease. Disclosed herein is a method of treating and/or preventing Alzheimer's disease progression in a subject, the method comprising administering to a subject a therapeutically effective amount of a disclosed viral vector or disclosed rAAV vector, thereby reducing the pathological phenotype associated with Alzheimer's disease.

Disclosed herein is a method of treating and/or preventing Alzheimer's disease progression in a subject, the method comprising administering to a subject a therapeutically effective amount of a disclosed viral vector or disclosed rAAV vector, thereby reducing the pathological phenotype associated with Alzheimer's disease. Disclosed herein is a method of treating and/or preventing Alzheimer's disease progression in a subject, the method comprising administering to a subject a therapeutically effective amount of a disclosed viral vector or disclosed rAAV vector, thereby reducing the pathological phenotype associated with Alzheimer's disease.

Disclosed herein is a method of treating and/or preventing Alzheimer's disease progression in a subject, the method comprising administering to a subject a therapeutically effective amount of a viral vector comprising a nucleic acid sequence encoding (i) a deactivated Cas (dCas) endonuclease, (ii) at least one polypeptide having an enzymatic activity, and (iii) at least one guide RNA targeting the APOE gene or a portion of the APOE gene, thereby reducing the pathological phenotype associated with Alzheimer's disease. Disclosed herein is a method of treating and/or preventing Alzheimer's disease progression in a subject, the method comprising administering to a subject a therapeutically effective amount of a viral vector comprising a nucleic acid sequence encoding (i) a deactivated Cas (dCas) endonuclease, (ii) a fusion protein having one or more enzymatic activities, and (iii) at least one guide RNA targeting the APOE gene or a portion of the APOE gene, thereby reducing the pathological phenotype associated with Alzheimer's disease.

Disclosed herein is a method of treating and/or preventing Alzheimer's disease progression in a subject, the method comprising administering to a subject a therapeutically effective amount of a recombinant AAV vector comprising a nucleic acid sequence encoding (i) a deactivated Cas (dCas) endonuclease, (ii) at least one polypeptide having an enzymatic activity, and (iii) at least one guide RNA targeting the APOE gene or a portion of the APOE gene, thereby reducing the pathological phenotype associated with Alzheimer's disease.

Disclosed herein is a method of treating and/or preventing Alzheimer's disease progression in a subject, the method comprising administering to a subject a therapeutically effective amount of a recombinant AAV vector comprising a nucleic acid sequence encoding (i) a deactivated Cas (dCas) endonuclease, (ii) a fusion protein having one or more enzymatic activities, and (iii) at least one guide RNA targeting the APOE gene or a portion of the APOE gene, thereby reducing the pathological phenotype associated with Alzheimer's disease.

Disclosed herein is a method of treating and/or preventing Alzheimer's disease progression in a subject, the method comprising administering to a subject a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting the APOE gene or a portion of the APOE gene, thereby reducing the pathological phenotype associated with Alzheimer's disease.

Disclosed herein is a method of treating and/or preventing Alzheimer's disease progression in a subject, the method comprising administering to a subject a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting the APOE gene or a portion of the APOE gene, (iv) a promoter operably linked to the dCas endonuclease and the fusion protein, and (v) a promoter operably linked to the at least one guide RNA, thereby reducing the pathological phenotype associated with Alzheimer's disease.

Disclosed herein is a method of treating and/or preventing Alzheimer's disease progression in a subject, the method comprising administering to a subject a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting the APOE gene or a portion of the APOE gene, thereby reducing the pathological phenotype associated with Alzheimer's disease.

Disclosed herein is a method of treating and/or preventing Alzheimer's disease progression in a subject, the method comprising administering to a subject a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting the APOE gene or a portion of the APOE gene, (iv) a promoter operably linked to the dCas endonuclease and the at least one polypeptide, and (v) a promoter operably linked to the at least one guide RNA, thereby reducing the pathological phenotype associated with Alzheimer's disease.

In an aspect, a disclosed method of treating and/or preventing Alzheimer's disease progression can reduce and/or decrease the expression and/or activity of APOE and/or APOE e4. In an aspect of a disclosed method of treating and/or preventing Alzheimer's disease progression, the expression and/or activity of APOE and/or APOE e4 is reduced and/or decreased.

In an aspect of a disclosed method of treating and/or preventing Alzheimer's disease progression, reducing the pathological phenotype associated with Alzheimer's disease can comprise reducing the Aβ42/40 ratio and reducing the level of Tau. In an aspect, a disclosed method can comprise diagnosing the subject with Alzheimer's disease.

In an aspect, prior to the administering step, the expression and/or activity level of APOE or APOE e4 can be elevated and/or increased when compared to wild-type or control expression level. In an aspect, following the administering step, the expression and/or activity level of APOE or APOE e4 can be reduced and/or decreased when compared to the pre-administering step level.

In an aspect, a disclosed method of effecting precision epigenetic modulation comprises measuring the level of expression and/or the level of activity of the disclosed gene of interest. In an aspect, a disclosed method can comprise repeating the measuring step one or more times.

In an aspect, a disclosed gRNA targeting the APOE gene or a portion of the APOE gene can comprise at least two gRNAs. In an aspect, disclosed gRNAs can target the same portion of the APOE gene or different portions of the APOE gene. In an aspect, a disclosed gRNA can target the promoter of the APOE gene. In an aspect, a disclosed gRNA can target exon 4 of the APOE gene. In an aspect, a disclosed gRNA can target a protospacer-adjacent motif (PAM) created by a SNP rs429358 in exon 4 of the APOE gene. In an aspect, a disclosed APOE gene can comprise the sequence set forth in SEQ ID NO:93-SEQ ID NO:96 or a fragment thereof. In an aspect, a disclosed gRNA targeting the APOE gene and/or the promoter of the APOE gene can comprise the sequence set forth in SEQ ID NO:85-SEQ ID NO:92.

In an aspect of a disclosed method, a disclosed dCas endonuclease can comprise a dCas9 endonuclease. In an aspect, a disclosed dCas9 endonuclease can comprise a deactivated Staphylococcus aureus Cas9 (dSaCas9), a deactivated Streptococcus pyogenes Cas9 (dSpCas9), a deactivated Campylobacter jejuni Cas9 (dCjCas9), or a variant dCas9 endonuclease. In an aspect, a disclosed variant dCas9 can comprise a variant dSaCas9, a variant dSpCas9, or a variant dCjCa9. In an aspect, a disclosed variant dSpCas9 can comprise dVQR, dEQR, or dVRER.

In an aspect of a disclosed method, a disclosed dSpCas9 can comprise the sequence set forth in SEQ ID NO:22, SEQ ID NO:23, or a fragment thereof. In an aspect, a disclosed dSpCas9 can comprise a sequence having at least 60%, at least 65%, at least 70%, at least 75%, at least 80%, at least 85%, at least 90%, or at least 95% identity to the sequence set forth in SEQ ID NO:22, SEQ ID NO:23, or a fragment thereof. In an aspect, a disclosed encoded dSpCas9 can comprise the sequence set forth in SEQ ID NO:19 or a fragment thereof. In an aspect, a disclosed encoded dSpCas9 can comprise a sequence having at least 60%, at least 65%, at least 70%, at least 75%, at least 80%, at least 85%, at least 90%, or at least 95% identity to the sequence set forth in SEQ ID NO:19 or a fragment thereof.

In an aspect of a disclosed method, a disclosed dSaCas9 can comprise the sequence set forth in SEQ ID NO:24 or a fragment thereof. In an aspect, a disclosed dSaCas9 can comprise a sequence having at least 60%, at least 65%, at least 70%, at least 75%, at least 80%, at least 85%, at least 90%, or at least 95% identity to the sequence set forth in SEQ ID NO:24 or a fragment thereof. In an aspect, a disclosed encoded dSaCas9 can comprise the sequence set forth in SEQ ID NO:20 or a fragment thereof. In an aspect, a disclosed encoded dSaCas9 can comprise a sequence having at least 60%, at least 65%, at least 70%, at least 75%, at least 80%, at least 85%, at least 90%, or at least 95% identity to the sequence set forth in SEQ ID NO:20 or a fragment thereof.

In an aspect of a disclosed method, a disclosed dCjCas9 can comprise the sequence set forth in SEQ ID NO:25, SEQ ID NO:26, or a fragment thereof. In an aspect, a disclosed dCjCas9 can comprise a sequence having at least 60%, at least 65%, at least 70%, at least 75%, at least 80%, at least 85%, at least 90%, or at least 95% identity to the sequence set forth in SEQ ID NO:25, SEQ ID NO:26, or a fragment thereof. In an aspect, a disclosed encoded dCjCas9 can comprise the sequence set forth in SEQ ID NO:21 or a fragment thereof. In an aspect, a disclosed encoded dCjCas9 can comprise a sequence having at least 60%, at least 65%, at least 70%, at least 75%, at least 80%, at least 85%, at least 90%, or at least 95% identity to the sequence set forth in SEQ ID NO:21 or a fragment thereof.

In an aspect, a disclosed dVQR can comprise D1135V, R1335Q, and T1337R. In an aspect, a disclosed dEQR can comprise D1135E, R1335Q, and T1337R. In an aspect, a disclosed dVRER can comprise D1135V, G1218R, R1335E, and T1337R. In an aspect, a disclosed dVRER can comprise the sequence set forth in SEQ ID NO:27 or a fragment thereof.

In an aspect of a disclosed method, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof.

In an aspect of a disclosed method, a disclosed encoded polypeptide can comprise HP1a, HP1b, MBD1, MBD2, Krüppel-Associated Box (KRAB), NIPP1, the Transcription Repression Domain (TRD) of Methyl-CpG Binding Protein 2 (MeCP2), DNMT3A, any combination thereof, or any fusion thereof.

In an aspect of a disclosed method, a disclosed fusion or disclosed fusion protein can comprise HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect of a disclosed method, a disclosed HP1a can comprise the sequence set forth in SEQ ID NO:43, SEQ ID NO:44, or fragment thereof. In an aspect, a disclosed HP1a can comprise a sequence having at least 60%, at least 65%, at least 70%, at least 75%, at least 80%, at least 85%, at least 90%, or at least 95% identity to the sequence set forth in SEQ ID NO:43, SEQ ID NO:44, or a fragment thereof. In an aspect, a disclosed encoded HP1a can comprise the sequence set forth in SEQ ID NO:28, SEQ ID NO:29, or a fragment thereof. In an aspect, a disclosed encoded HP1a can comprise a sequence having at least 60%, at least 65%, at least 70%, at least 75%, at least 80%, at least 85%, at least 90%, or at least 95% identity to the sequence set forth in SEQ ID NO:28, SEQ ID NO:29, or a fragment thereof.

In an aspect of a disclosed method, a disclosed HP1b can comprise the sequence set forth in SEQ ID NO:45, SEQ ID NO:46, or a fragment thereof. In an aspect, a disclosed HP1b can comprise a sequence having at least 60%, at least 65%, at least 70%, at least 75%, at least 80%, at least 85%, at least 90%, or at least 95% identity to the sequence set forth in SEQ ID NO:45, SEQ ID NO:46, or a fragment thereof. In an aspect, a disclosed encoded HP1b can comprise the sequence set forth in SEQ ID NO:30, SEQ ID NO:31, or a fragment thereof. In an aspect, a disclosed encoded HIP1a can comprise a sequence having at least 60%, at least 65%, at least 70%, at least 75%, at least 80%, at least 85%, at least 90%, or at least 95% identity to the sequence set forth in SEQ ID NO:30, SEQ ID NO:31, or a fragment thereof.

In an aspect of a disclosed method, a disclosed MBD1 can comprise the sequence set forth in SEQ ID NO:47, SEQ ID NO:48, or a fragment thereof. In an aspect, a disclosed MBD1 can comprise a sequence having at least 60%, at least 65%, at least 70%, at least 75%, at least 80%, at least 85%, at least 90%, or at least 95% identity to the sequence set forth in SEQ ID NO:47, SEQ ID NO:48, or a fragment thereof. In an aspect, a disclosed encoded MBD1 can comprise the sequence set forth in SEQ ID NO:32, SEQ ID NO:33, SEQ ID NO:34, or a fragment thereof. In an aspect, a disclosed encoded MBD1 can comprise a sequence having at least 60%, at least 65%, at least 70%, at least 75%, at least 80%, at least 85%, at least 90%, or at least 95% identity to the sequence set forth in SEQ ID NO:32, SEQ ID NO:33, SEQ ID NO:34, or a fragment thereof.

In an aspect of a disclosed method, a disclosed MBD2 can comprise the sequence set forth in SEQ ID NO:49, SEQ ID NO:50, or a fragment thereof. In an aspect, a disclosed MBD2 can comprise a sequence having at least 60%, at least 65%, at least 70%, at least 75%, at least 80%, at least 85%, at least 90%, or at least 95% identity to the sequence set forth in SEQ ID NO:49, SEQ ID NO:50, or a fragment thereof. In an aspect, a disclosed encoded MBD2 can comprise the sequence set forth in SEQ ID NO:35, SEQ ID NO:36, or a fragment thereof. In an aspect, a disclosed encoded MBD2 can comprise a sequence having at least 60%, at least 65%, at least 70%, at least 75%, at least 80%, at least 85%, at least 90%, or at least 95% identity to the sequence set forth in SEQ ID NO:35, SEQ ID NO:36, or a fragment thereof.

In an aspect of a disclosed method, a disclosed KRAB can comprise the sequence set forth in SEQ ID NO:53, SEQ ID NO:54, SEQ ID NO:55, or a fragment thereof. In an aspect, a disclosed KRAB can comprise a sequence having at least 60%, at least 65%, at least 70%, at least 75%, at least 80%, at least 85%, at least 90%, or at least 95% identity to the sequence set forth in SEQ ID NO:53, SEQ ID NO:54, SEQ ID NO:55, or a fragment thereof. In an aspect, a disclosed encoded KRAB can comprise the sequence set forth in SEQ ID NO:39 or a fragment thereof. In an aspect, a disclosed encoded KRAB can comprise a sequence having at least 60%, at least 65%, at least 70%, at least 75%, at least 80%, at least 85%, at least 90%, or at least 95% identity to the sequence set forth in SEQ ID NO:39 or a fragment thereof.

In an aspect of a disclosed method, a disclosed NIPP1 can comprise the sequence set forth in SEQ ID NO:51, SEQ ID NO:52, or a fragment thereof. In an aspect, a disclosed NIPP1 can comprise a sequence having at least 60%, at least 65%, at least 70%, at least 75%, at least 80%, at least 85%, at least 90%, or at least 95% identity to the sequence set forth in SEQ ID NO:51, SEQ ID NO:52, or a fragment thereof. In an aspect, a disclosed encoded NIPP1 can comprise the sequence set forth in SEQ ID NO:37, SEQ ID NO:38, or a fragment thereof. In an aspect, a disclosed encoded NIPP1 can comprise a sequence having at least 60%, at least 65%, at least 70%, at least 75%, at least 80%, at least 85%, at least 90%, or at least 95% identity to the sequence set forth in SEQ ID NO:37, SEQ ID NO:38, or a fragment thereof.

In an aspect of a disclosed method, a disclosed MeCP2 can comprise the sequence set forth in SEQ ID NO:56, SEQ ID NO:57, or a fragment thereof. In an aspect, a disclosed MeCP2 can comprise a sequence having at least 60%, at least 65%, at least 70%, at least 75%, at least 80%, at least 85%, at least 90%, or at least 95% identity to the sequence set forth in SEQ ID NO:56, SEQ ID NO:57, or a fragment thereof. In an aspect, a disclosed encoded MeCP2 can comprise the sequence set forth in SEQ ID NO:40 or a fragment thereof. In an aspect, a disclosed encoded MeCP2 can comprise a sequence having at least 60%, at least 65%, at least 70%, at least 75%, at least 80%, at least 85%, at least 90%, or at least 95% identity to the sequence set forth in SEQ ID NO:40 or a fragment thereof.

In an aspect of a disclosed method, a disclosed KRAB-MeCP2 can comprise the sequence set forth in SEQ ID NO:58, SEQ ID NO:59, or a fragment thereof. In an aspect, a disclosed KRAB-MeCP2 can comprise a sequence having at least 60%, at least 65%, at least 70%, at least 75%, at least 80%, at least 85%, at least 90%, or at least 95% identity to the sequence set forth in SEQ ID NO:58, SEQ ID NO:59, or a fragment thereof. In an aspect, a disclosed encoded KRAB-MeCP2 can comprise the sequence set forth in SEQ ID NO:41 or a fragment thereof. In an aspect, a disclosed encoded KRAB-MeCP2 can comprise a sequence having at least 60%, at least 65%, at least 70%, at least 75%, at least 80%, at least 85%, at least 90%, or at least 95% identity to the sequence set forth in SEQ ID NO:41 or a fragment thereof.

In an aspect of a disclosed method, a disclosed DNMT3A can comprise the sequence set forth in SEQ ID NO:60, SEQ ID NO:61, or a fragment thereof. In an aspect, a disclosed DNMT3A can comprise a sequence having at least 60%, at least 65%, at least 70%, at least 75%, at least 80%, at least 85%, at least 90%, or at least 95% identity to the sequence set forth in SEQ ID NO:60, SEQ ID NO:61, or a fragment thereof. In an aspect, a disclosed encoded DNMT3A can comprise the sequence set forth in SEQ ID NO:42 or a fragment thereof. In an aspect, a disclosed encoded DNMT3A can comprise a sequence having at least 60%, at least 65%, at least 70%, at least 75%, at least 80%, at least 85%, at least 90%, or at least 95% identity to the sequence set forth in SEQ ID NO:42 or a fragment thereof.

In an aspect of a disclosed method, a disclosed encoded polypeptide can comprise transcription repression activity. A disclosed encoded polypeptide comprising transcription repression activity can comprise a Krüppel associated box domain, an ERF repressor domain, a MXI1 repressor domain, a SID4x repressor domain, a fused KRAB-MeCP2 domain, a MeCP2 TRD domain, a MAS-SID repressor domain, a TATA box binding protein activity, or any combination thereof. In an aspect, a disclosed encoded polypeptide comprising transcription repression activity can comprise HP1 repressor activity, MeCP2 repressor activity, MBD1 repressor activity, MBD2 repressor activity, MBD3 repressor activity, MBD4 repressor activity, KRAB repressor activity, SUV39H1 repressor activity, SUV39H2 repressor activity, CTCF insulator-repressor activity, LSD-1 histone-demethylase repressor activity, or any combination thereof.

In an aspect of a disclosed method, a disclosed encoded polypeptide can comprise transcription release factor activity. A disclosed encoded polypeptide comprising transcription release factor activity can comprise Eukaryotic Release Factor 1 (ERF1) activity or Eukaryotic Release Factor 3 (ERF3) activity. In an aspect, a disclosed ERF1 can comprise the sequence set forth in SEQ ID NO:71 or a fragment thereof. In an aspect, a disclosed encoded ERF1 can comprise a sequence having at least 60%, at least 65%, at least 70%, at least 75%, at least 80%, at least 85%, at least 90%, or at least 95% identity to the sequence set forth in SEQ ID NO:72 or a fragment thereof.

In an aspect of a disclosed method, a disclosed encoded polypeptide can comprise histone modification activity. A disclosed encoded polypeptide comprising histone modification activity can comprise histone acetyltransferase, histone deacetylase, histone demethylase, histone methyltransferase activity, or any combination thereof.

In an aspect of a disclosed method, a disclosed encoded polypeptide can comprise nucleic acid association activity. A disclosed encoded polypeptide comprising nucleic acid association activity can comprise a helix-turn-helix region, a leucine zipper region, a winged helix region, a winged helix-turn-helix region, a helix-loop-helix region, an immunoglobulin fold, a B3 domain, a zinc finger, a HMG-box, a Wor3 domain, a TAL effector DNA-binding domain, or any combination thereof.

In an aspect of a disclosed method, a disclosed encoded polypeptide can comprise methyltransferase activity. A disclosed encoded polypeptide comprising methyltransferase activity can comprise DNA (cytosine-5)-methyltransferase 3a (DNMT3A). In an aspect of a disclosed method, a disclosed encoded polypeptide can comprise demethylase activity. A disclosed encoded polypeptide comprising demethylase activity can comprise ten-eleven translocation methylcytosine dioxygenase 1 (TET1) or lysine-specific histone demethylase 1 (LSD1). In an aspect of a disclosed method, a disclosed encoded polypeptide can comprise acetyltransferase activity. A disclosed encoded polypeptide comprising acetyltransferase activity can comprise histone acetyltransferase. In an aspect of a disclosed method, a disclosed encoded polypeptide can comprise deacetylase activity. A disclosed encoded polypeptide comprising deacetylase activity can comprise histone deacetylase.

In an aspect of a disclosed method, a disclosed encoded dCas endonuclease is fused to the at least one encoded polypeptide having an enzymatic activity. In an aspect, a disclosed dCas endonuclease can comprise dSpCas9 and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dSaCas9 and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dCjCas9 and a disclosed polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof.

In an aspect of a disclosed method, a disclosed dCas endonuclease can comprise dVQR, dEQR, or dVRER and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect of a disclosed method, a disclosed dCas endonuclease can comprise dSpCas9, dSaCas9, or dCjCas9 and a disclosed encoded polypeptide can comprise KRAB-MeCP2.

In an aspect of a disclosed method, a disclosed dCas endonuclease can comprise dSpCas9, dSaCas9, or dCjCas9, and a disclosed fusion can comprise HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect of a disclosed method, a disclosed viral vector or disclosed recombinant AAV vector can further comprise a nucleic acid sequencing encoding one or more regulatory elements. In an aspect, a disclosed regulatory element can comprise a promoter, an enhancer, a promoter/enhancer, an internal ribosomal entry site, a transcription terminal signal, a polyadenylation signal, a Sp1 and/or NF-kB transcriptional factor binding site, a p2A signal, a woodchuck hepatitis virus post-transcriptional regulatory element, a Phi signal-packaging signal, a rev responsive element, a 5′-LTR, a 3′-LTR, an inverted terminal repeat, a nuclear localization signal (NLS), or any combination thereof.

In an aspect, a disclosed NLS can comprise the sequence set forth in SEQ ID NO:105-SEQ ID NO:111 or a fragment thereof. In an aspect, a disclosed PolyA sequence can comprise the sequence set forth in SEQ ID NO:103, SEQ ID NO:104, or a fragment thereof. In an aspect, a disclosed ITR can comprise the sequence set forth in SEQ ID NO:99-SEQ ID NO:102 or a fragment thereof.

In an aspect of a disclosed method, a disclosed viral vector or disclosed recombinant AAV vector can further a nucleic acid sequence encoding one or more promoters. In an aspect, a disclosed promoter can comprise a U6 promoter, a chicken β-actin promoter, an EF-1a, a CMV promoter, a CMV promoter/enhancer, a fragment thereof, or any combination thereof. In an aspect, a disclosed U6 promoter can comprise the sequence set forth in SEQ ID NO:114 or a fragment thereof. In an aspect, a disclosed U6 promoter can comprise a sequence having at least 60%, at least 65%, at least 70%, at least 75%, at least 80%, at least 85%, at least 90%, at least 95%, or greater than 95% identity to the sequence set forth in SEQ ID NO:114 or a fragment thereof. In an aspect, a disclosed EF-1α promoter can comprise the sequence set forth in SEQ ID NO:112, SEQ ID NO:113, or a fragment thereof. In an aspect, a disclosed EF-1α promoter can comprise a sequence having at least 60%, at least 65%, at least 70%, at least 75%, at least 80%, at least 85%, at least 90%, at least 95%, or greater than 95% identity to the sequence set forth in SEQ ID NO:112, SEQ ID NO:113, or a fragment thereof. In an aspect, a promoter can be a short EF-1α (EFS-NF) promoter.

In an aspect of a disclosed method, a disclosed promoter can be operably linked to the dCas endonuclease. In an aspect, a disclosed promoter operably linked to the dCas endonuclease can comprise an EF-1α or EFS-NC promoter. In an aspect, a disclosed promoter operably linked to the dCas endonuclease can be operably linked to the at least one polypeptide having enzymatic activity. In an aspect, a disclosed promoter can be operably linked to the dCas endonuclease and the at least one polypeptide having enzymatic activity. In an aspect, a disclosed promoter can be operably linked to the fusion protein having one or more enzymatic activities. In an aspect, a disclosed promoter can be operably linked to the dCas endonuclease and the fusion protein having one or more enzymatic activities.

In an aspect, a disclosed promoter operably can be linked to the at least one guide RNA targeting the APOE gene or a portion of the APOE gene. In an aspect, a disclosed promoter operably linked to the at least one guide RNA can comprise a U6 promoter.

In an aspect of a disclosed method, a disclosed viral vector or disclosed recombinant AAV vector can further comprise a gRNA scaffold. In an aspect, a disclosed gRNA scaffold can comprise the sequence set forth in SEQ ID NO:115, SEQ ID NO:116, or a fragment thereof.

In an aspect of a disclosed method, a disclosed viral vector or disclosed recombinant AAV vector can further comprise one or more promoters, wherein a first promoter can be operably linked to the dCas endonuclease, and wherein a second promoter can be operably linked to the at least one guide RNA targeting the APOE gene or a portion of the APOE gene. In an aspect of a disclosed method, a disclosed viral vector or disclosed recombinant AAV vector can further comprise one or more promoters, wherein a first promoter can be operably linked to the dCas endonuclease and the at least one polypeptide having an enzymatic activity, and wherein a second promoter can be operably linked to the at least one guide RNA the APOE gene or a portion of the APOE gene.

In an aspect of a disclosed method, a disclosed viral vector or disclosed recombinant AAV vector, a disclosed encoded polypeptide can comprise HP1a, HP1b, MBD1, MBD2, Krüppel-Associated Box (KRAB), NIPP1, the Transcription Repression Domain (TRD) of Methyl-CpG Binding Protein 2 (MeCP2), DNMT3A, any combination thereof, or any fusion thereof.

In an aspect of a disclosed method, a disclosed nucleic acid sequence can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art. In an aspect, any disclosed dCas9 endonuclease, a disclosed polypeptide having enzymatic activities, a disclosed fusion product or a disclosed fusion protein, or any combination thereof can be codon-optimized.

In an aspect of a disclosed method, a disclosed viral vector or disclosed recombinant AAV vector can comprise a sequence that is about 4.5 kilobases or less than about 4.5 kilobases.

In an aspect, a disclosed method can further comprise effecting precision epigenetic modulation of the APOE gene or a portion of the APOE gene. In an aspect, modulating can comprise decreasing and/or reducing expression and/or activity of the APOE gene or a portion of the APOE gene. In an aspect, modulating can comprise decreasing and/or reducing expression and/or activity of the APOE e4 gene or a portion of the APOE e4 gene. In an aspect, the disclosed cells can be neurons (e.g., cholinergic neurons, dopaminergic neurons, etc.). In an aspect, the disclosed cells can be cells affected by an overexpression level and/or activity level of the APOE gene or a portion of the APOE gene.

In an aspect, the disclosed cells can be in a subject. In an aspect of a disclosed method, a subject can be a human. In an aspect, a subject can be suspected of having or can be diagnosed with having Alzheimer's disease (such as, for example, PD).

In an aspect, a disclosed method can comprise reducing the pathological phenotype associated with Alzheimer's disease. In an aspect, reducing the pathological phenotype associated with Alzheimer's disease can comprise reducing the Aβ42/40 ratio and reducing the level of Tau. In an aspect, a disclosed method can comprise diagnosing the subject with Alzheimer's disease.

In an aspect, a subject can be a subject in need of treatment of Alzheimer's disease.

In an aspect, a disclosed method of treating and/or preventing Alzheimer's disease progression can comprise restoring one or more aspects of cellular homeostasis and/or cellular functionality and/or metabolic dysregulation.

In an aspect, restoring one or more aspects of cellular homeostasis and/or cellular functionality and/or metabolic dysregulation can comprise reducing the expression and/or activity level of the APOE gene or a portion of the APOE gene.

In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof.

In an aspect, restoring one or more aspects of cellular homeostasis can comprise improving, enhancing, restoring, and/or preserving one or more aspects of cellular structural and/or functional integrity. In an aspect, restoring the activity and/or functionality of a missing, deficient, and/or mutant protein or enzyme can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme). In an aspect, restoration can be a partial or incomplete restoration. In an aspect, restoration can be complete or near complete restoration such that the level of expression, activity, and/or functionality is like that of a wild-type or control level.

In an aspect, restoring the activity and/or functionality of the APOE gene or a portion of the APOE gene can comprise decreasing or reducing the expression and/or activity level of the APOE gene or a portion of the APOE gene. In an aspect, decreasing or reducing can comprise a decrease of at least about 5%, 10%, 15%, 20%, 25%, 35%, 50%, 75%, 80%, 85%, 90%, 95%, 97%, 98%, 99%, 100%, 200%, 300%, 400%, 500%, or more as compared to a control (such as a pre-treatment level).

In an aspect of a disclosed method of treating and/or preventing Alzheimer's disease progression, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect of a disclosed method of treating and/or preventing Alzheimer's disease progression, administering can comprise intravenous administration, intracerebral administration, intra-CSF administration, intracerebroventricular (ICV) administration, intraventricular administration, intra-cisterna magna (ICM) administration, intraparenchymal administration, intrathecal (lumbar, cisternal, or both) administration, intrahepatic administration, hepatic intra-arterial administration, hepatic portal vein (HPV) administration, or any combination thereof. In an aspect, a disclosed vector can be administered via LNP administration.

In an aspect, a disclosed method of treating and/or preventing Alzheimer's disease progression can employ multiple routes of administration to the subject. In an aspect, a disclosed method can employ a first route of administration that can be the same or different as a second and/or subsequent routes of administration. In an aspect, a disclosed nucleic acid molecule, a disclosed vector, and/or a disclosed pharmaceutical formulation can be concurrently and/or serially administered to a subject via multiple routes of administration. For example, in an aspect, administering a disclosed nucleic acid molecule, a disclosed vector, and/or a disclosed pharmaceutical formulation can comprise intravenous administration and intra-cistern magna (ICM) administration. In an aspect, administering a disclosed nucleic acid molecule, a disclosed vector, and/or a disclosed pharmaceutical formulation can comprise IV administration and intrathecal (ITH) administration.

In an aspect of a disclosed method of treating and/or preventing Alzheimer's disease progression, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ vg/kg to about 8×10¹³ vg/kg or about 1×10¹² vg/kg to about 8×10¹³ vg/kg. In an aspect, a disclosed vector can be administered at a dose of about 1×10¹³ vg/kg to about 6×10¹³ vg/kg. In an aspect, a disclosed vector can be administered at a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a disclosed vector can be administered at a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method of treating and/or preventing Alzheimer's disease progression can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method of treating and/or preventing Alzheimer's disease progression can further comprise administering to the subject a therapeutically effective amount of a therapeutic agent. A therapeutic agent can be any disclosed agent that effects a desired clinical outcome.

In an aspect, a disclosed method of treating and/or preventing Alzheimer's disease progression can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method of treating and/or preventing Alzheimer's disease progression can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of treating and/or preventing Alzheimer's disease progression can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method of treating and/or preventing Alzheimer's disease progression can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of treating and/or preventing Alzheimer's disease progression can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of treating and/or preventing Alzheimer's disease progression can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect of a disclosed method, a subject can be a human. In an aspect, a subject can be suspected of having or can be diagnosed with having Alzheimer's disease (such as, for example, PD). In an aspect, a disclosed subject can be symptomatic or asymptomatic.

In an aspect, a disclosed method can comprise repeating one or more steps of the method and/or modifying one or more steps of the method (such as, for example, an administering step).

In an aspect, a disclosed method of treating and/or preventing Alzheimer's disease progression can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, a disclosed method of treating and/or preventing Alzheimer's disease progression can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In an aspect, a disclosed method of treating and/or preventing Alzheimer's disease progression can further comprise generating and/or validating one or more of the disclosed isolated nucleic acid molecules, one or more of the disclosed vectors, one or more of the disclosed pharmaceutical formulations, or any combination thereof.

In an aspect, a disclosed method of treating and/or preventing Alzheimer's disease progression can further comprise administering to the subject a second disclosed isolated nucleic acid molecule, a second disclosed vector, a second disclosed pharmaceutical formulations, or any combination thereof. In an aspect, a disclosed method of treating and/or preventing Alzheimer's disease progression can further comprise administering to the subject additional disclosed isolated nucleic acid molecules, additional disclosed vectors, additional disclosed pharmaceutical formulations, or any combination thereof.

### H. Methods of Treating and/or Preventing Parkinson's Disease Progression

Disclosed herein is a method of treating and/or preventing Parkinson's disease progression in a subject, the method comprising administering to a subject a therapeutically effective amount of a disclosed isolated nucleic acid molecule, thereby reducing the pathological phenotype associated with Parkinson's disease.

Disclosed herein is a method of treating and/or preventing Parkinson's disease progression in a subject, the method comprising administering to a subject a therapeutically effective amount of an isolated nucleic acid molecule, comprising: a nucleic acid sequence encoding (i) a deactivated Cas (dCas) endonuclease, (ii) at least one polypeptide having an enzymatic activity, and (iii) at least one guide RNA targeting the SNCA gene or a portion of the SNCA gene, thereby reducing the pathological phenotype associated with Parkinson's disease.

Disclosed herein is a method of treating and/or preventing Parkinson's disease progression in a subject, the method comprising administering to a subject a therapeutically effective amount of an isolated nucleic acid molecule, comprising: a nucleic acid sequence encoding (i) a deactivated Cas (dCas) endonuclease, (ii) a fusion protein having one or more enzymatic activities, and (iii) at least one guide RNA targeting the SNCA gene or a portion of the SNCA gene, thereby reducing the pathological phenotype associated with Parkinson's disease.

Disclosed herein is a method of treating and/or preventing Parkinson's disease progression in a subject, the method comprising administering to a subject a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting the SNCA gene or a portion of the SNCA gene, thereby reducing the pathological phenotype associated with Parkinson's disease.

Disclosed herein is a method of treating and/or preventing Parkinson's disease progression in a subject, the method comprising administering to a subject a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting the SNCA gene or a portion of the SNCA gene, (iv) a promoter operably linked to the dCas endonuclease and the fusion protein, and (v) a promoter operably linked to the at least one guide RNA, thereby reducing the pathological phenotype associated with Parkinson's disease.

Disclosed herein is a method of treating and/or preventing Parkinson's disease progression in a subject, the method comprising administering to a subject a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting the SNCA gene or a portion of the SNCA gene, thereby reducing the pathological phenotype associated with Parkinson's disease.

Disclosed herein is a method of treating and/or preventing Parkinson's disease progression in a subject, the method comprising administering to a subject a therapeutically effective amount of an isolated nucleic acid molecule, comprising: a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting the SNCA gene or a portion of the SNCA gene, (iv) a promoter operably linked to the dCas endonuclease and the at least one polypeptide, and (v) a promoter operably linked to the at least one guide RNA, thereby reducing the pathological phenotype associated with Parkinson's disease.

Disclosed herein is a method of treating and/or preventing Parkinson's disease progression in a subject, the method comprising administering to a subject a therapeutically effective amount of a disclosed viral vector or disclosed rAAV vector, thereby reducing the pathological phenotype associated with Parkinson's disease. Disclosed herein is a method of treating and/or preventing Parkinson's disease progression in a subject, the method comprising administering to a subject a therapeutically effective amount of a disclosed viral vector or disclosed rAAV vector, thereby reducing the pathological phenotype associated with Parkinson's disease. Disclosed herein is a method of treating and/or preventing Parkinson's disease progression in a subject, the method comprising administering to a subject a therapeutically effective amount of a disclosed viral vector or disclosed rAAV vector, thereby reducing the pathological phenotype associated with Parkinson's disease. Disclosed herein is a method of treating and/or preventing Parkinson's disease progression in a subject, the method comprising administering to a subject a therapeutically effective amount of a disclosed viral vector or disclosed rAAV vector, thereby reducing the pathological phenotype associated with Parkinson's disease.

Disclosed herein is a method of treating and/or preventing Parkinson's disease progression in a subject, the method comprising administering to a subject a therapeutically effective amount of a viral vector comprising a nucleic acid sequence encoding (i) a deactivated Cas (dCas) endonuclease, (ii) at least one polypeptide having an enzymatic activity, and (iii) at least one guide RNA targeting the SNCA gene or a portion of the SNCA gene, thereby reducing the pathological phenotype associated with Parkinson's disease. Disclosed herein is a method of treating and/or preventing Parkinson's disease progression in a subject, the method comprising administering to a subject a therapeutically effective amount of a viral vector comprising a nucleic acid sequence encoding (i) a deactivated Cas (dCas) endonuclease, (ii) a fusion protein having one or more enzymatic activities, and (iii) at least one guide RNA targeting the SNCA gene or a portion of the SNCA gene, thereby reducing the pathological phenotype associated with Parkinson's disease.

Disclosed herein is a method of treating and/or preventing Parkinson's disease progression in a subject, the method comprising administering to a subject a therapeutically effective amount of a recombinant AAV vector comprising a nucleic acid sequence encoding (i) a deactivated Cas (dCas) endonuclease, (ii) at least one polypeptide having an enzymatic activity, and (iii) at least one guide RNA targeting the SNCA gene or a portion of the SNCA gene, thereby reducing the pathological phenotype associated with Parkinson's disease. Disclosed herein is a method of treating and/or preventing Parkinson's disease progression in a subject, the method comprising administering to a subject a therapeutically effective amount of a recombinant AAV vector comprising a nucleic acid sequence encoding (i) a deactivated Cas (dCas) endonuclease, (ii) a fusion protein having one or more enzymatic activities, and (iii) at least one guide RNA targeting the SNCA gene or a portion of the SNCA gene, thereby reducing the pathological phenotype associated with Parkinson's disease

Disclosed herein is a method of treating and/or preventing Parkinson's disease progression in a subject, the method comprising administering to a subject a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, and (iii) at least one guide RNA targeting the SNCA gene or a portion of the SNCA gene, thereby reducing the pathological phenotype associated with Parkinson's disease.

Disclosed herein is a method of treating and/or preventing Parkinson's disease progression in a subject, the method comprising administering to a subject a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion product comprises any combination of HP1a, HP1b, MBD1, MBD2, KRAB, NIPP1, MeCP2, or DNMT3A, (iii) at least one guide RNA targeting the SNCA gene or a portion of the SNCA gene, (iv) a promoter operably linked to the dCas endonuclease and the fusion protein, and (v) a promoter operably linked to the at least one guide RNA, thereby reducing the pathological phenotype associated with Parkinson's disease.

Disclosed herein is a method of treating and/or preventing Parkinson's disease progression in a subject, the method comprising administering to a subject a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, and (iii) at least one guide RNA targeting the SNCA gene or a portion of the SNCA gene, thereby reducing the pathological phenotype associated with Parkinson's disease.

Disclosed herein is a method of treating and/or preventing Parkinson's disease progression in a subject, the method comprising administering to a subject a therapeutically effective amount of a viral vector or a recombinant AAV vector comprising a nucleic acid sequence encoding (i) a deactivated Cas (dCas) endonuclease, wherein the dCas is a dSpCas9, dSaCas9, or dCjCas9, (ii) a fusion protein having one or more enzymatic activities, wherein the fusion protein comprises any combination of HP1a, HP1b, MBD1, MBD2, KRAB, NIPP1, MeCP2, or DNMT3A, (iii) at least one guide RNA targeting the SNCA gene or a portion of the SNCA gene, (iv) a promoter operably linked to the dCas endonuclease and the at least one polypeptide, and (v) a promoter operably linked to the at least one guide RNA, thereby reducing the pathological phenotype associated with Parkinson's disease.

In an aspect, a disclosed method of treating and/or preventing Parkinson's disease progression can reduce and/or decrease the expression and/or activity of SNCA. In an aspect of a disclosed method of treating and/or preventing Parkinson's disease progression, the expression and/or activity of SNCA is reduced and/or decreased. In an aspect, a disclosed method can comprise diagnosing the subject with Parkinson's disease.

In an aspect, a disclosed method of effecting precision epigenetic modulation comprises measuring the level of expression and/or the level of activity of the disclosed gene of interest. In an aspect, a disclosed method can comprise repeating the measuring step one or more times.

In an aspect, prior to the administering step, the expression and/or activity level of SNCA can be elevated and/or increased when compared to wild-type or control expression level. In an aspect, following the administering step, the expression and/or activity level of SNCA can be reduced and/or decreased when compared to the pre-administering step level. In an aspect, a disclosed gRNA targeting the APOE gene or a portion of the SNCA gene can comprise at least two gRNAs. In an aspect, disclosed gRNAs can target the same portion of the SNCA gene or different portions of the SNCA gene. In an aspect, a disclosed gRNA can target the promoter of the SNCA gene. In an aspect, a disclosed gRNA can target intron 1 of the SNCA gene. In an aspect, a disclosed SNCA gene can comprise the sequence set forth in SEQ ID NO:127. In an aspect, a disclosed gRNA targeting the SNCA gene and/or the promoter of the APOE gene can comprise the sequence set forth in SEQ ID NO:81-SEQ ID NO:84.

In an aspect of a disclosed method, a disclosed dCas endonuclease can comprise a dCas9 endonuclease. In an aspect, a disclosed dCas9 endonuclease can comprise a deactivated Staphylococcus aureus Cas9 (dSaCas9), a deactivated Streptococcus pyogenes Cas9 (dSpCas9), a deactivated Campylobacter jejuni Cas9 (dCjCas9), or a variant dCas9 endonuclease. In an aspect, a disclosed variant dCas9 can comprise a variant dSaCas9, a variant dSpCas9, or a variant dCjCa9. In an aspect, a disclosed variant dSpCas9 can comprise dVQR, dEQR, or dVRER.

In an aspect of a disclosed method, a disclosed dSpCas9 can comprise the sequence set forth in SEQ ID NO:22, SEQ ID NO:23, or a fragment thereof. In an aspect, a disclosed dSpCas9 can comprise a sequence having at least 60%, at least 65%, at least 70%, at least 75%, at least 80%, at least 85%, at least 90%, or at least 95% identity to the sequence set forth in SEQ ID NO:22, SEQ ID NO:23, or a fragment thereof. In an aspect, a disclosed encoded dSpCas9 can comprise the sequence set forth in SEQ ID NO:19 or a fragment thereof. In an aspect, a disclosed encoded dSpCas9 can comprise a sequence having at least 60%, at least 65%, at least 70%, at least 75%, at least 80%, at least 85%, at least 90%, or at least 95% identity to the sequence set forth in SEQ ID NO:19 or a fragment thereof.

In an aspect of a disclosed method, a disclosed dSaCas9 can comprise the sequence set forth in SEQ ID NO:24 or a fragment thereof. In an aspect, a disclosed dSaCas9 can comprise a sequence having at least 60%, at least 65%, at least 70%, at least 75%, at least 80%, at least 85%, at least 90%, or at least 95% identity to the sequence set forth in SEQ ID NO:24 or a fragment thereof. In an aspect, a disclosed encoded dSaCas9 can comprise the sequence set forth in SEQ ID NO:20 or a fragment thereof. In an aspect, a disclosed encoded dSaCas9 can comprise a sequence having at least 60%, at least 65%, at least 70%, at least 75%, at least 80%, at least 85%, at least 90%, or at least 95% identity to the sequence set forth in SEQ ID NO:20 or a fragment thereof.

In an aspect of a disclosed method, a disclosed dCjCas9 can comprise the sequence set forth in SEQ ID NO:25, SEQ ID NO:26, or a fragment thereof. In an aspect, a disclosed dCjCas9 can comprise a sequence having at least 60%, at least 65%, at least 70%, at least 75%, at least 80%, at least 85%, at least 90%, or at least 95% identity to the sequence set forth in SEQ ID NO:25, SEQ ID NO:26, or a fragment thereof. In an aspect, a disclosed encoded dCjCas9 can comprise the sequence set forth in SEQ ID NO:21 or a fragment thereof. In an aspect, a disclosed encoded dCjCas9 can comprise a sequence having at least 60%, at least 65%, at least 70%, at least 75%, at least 80%, at least 85%, at least 90%, or at least 95% identity to the sequence set forth in SEQ ID NO:21 or a fragment thereof.

In an aspect, a disclosed dVQR can comprise D1135V, R1335Q, and T1337R. In an aspect, a disclosed dEQR can comprise D1135E, R1335Q, and T1337R. In an aspect, a disclosed dVRER can comprise D1135V, G1218R, R1335E, and T1337R. In an aspect, a disclosed dVRER can comprise the sequence set forth in SEQ ID NO:27 or a fragment thereof.

In an aspect of a disclosed method, a disclosed encoded polypeptide can comprise transcription activation activity, transcription repression activity, transcription release factor activity, histone modification activity, nucleic acid association activity, methyltransferase activity, demethylase activity, acetyltransferase activity, deacetylase activity, or any combination thereof.

In an aspect of a disclosed method, a disclosed encoded polypeptide can comprise HP1a, HP1b, MBD1, MBD2, Krüppel-Associated Box (KRAB), NIPP1, the Transcription Repression Domain (TRD) of Methyl-CpG Binding Protein 2 (MeCP2), DNMT3A, any combination thereof, or any fusion thereof.

In an aspect of a disclosed method, a disclosed fusion or disclosed fusion protein can comprise HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect of a disclosed method, a disclosed HP1a can comprise the sequence set forth in SEQ ID NO:43, SEQ ID NO:44, or fragment thereof. In an aspect, a disclosed HP1a can comprise a sequence having at least 60%, at least 65%, at least 70%, at least 75%, at least 80%, at least 85%, at least 90%, or at least 95% identity to the sequence set forth in SEQ ID NO:43, SEQ ID NO:44, or a fragment thereof. In an aspect, a disclosed encoded HP1a can comprise the sequence set forth in SEQ ID NO:28, SEQ ID NO:29, or a fragment thereof. In an aspect, a disclosed encoded HP1a can comprise a sequence having at least 60%, at least 65%, at least 70%, at least 75%, at least 80%, at least 85%, at least 90%, or at least 95% identity to the sequence set forth in SEQ ID NO:28, SEQ ID NO:29, or a fragment thereof.

In an aspect of a disclosed method, a disclosed HP1b can comprise the sequence set forth in SEQ ID NO:45, SEQ ID NO:46, or a fragment thereof. In an aspect, a disclosed HP1b can comprise a sequence having at least 60%, at least 65%, at least 70%, at least 75%, at least 80%, at least 85%, at least 90%, or at least 95% identity to the sequence set forth in SEQ ID NO:45, SEQ ID NO:46, or a fragment thereof. In an aspect, a disclosed encoded HP1b can comprise the sequence set forth in SEQ ID NO:30, SEQ ID NO:31, or a fragment thereof. In an aspect, a disclosed encoded HIP1a can comprise a sequence having at least 60%, at least 65%, at least 70%, at least 75%, at least 80%, at least 85%, at least 90%, or at least 95% identity to the sequence set forth in SEQ ID NO:30, SEQ ID NO:31, or a fragment thereof.

In an aspect of a disclosed method, a disclosed MBD1 can comprise the sequence set forth in SEQ ID NO:47, SEQ ID NO:48, or a fragment thereof. In an aspect, a disclosed MBD1 can comprise a sequence having at least 60%, at least 65%, at least 70%, at least 75%, at least 80%, at least 85%, at least 90%, or at least 95% identity to the sequence set forth in SEQ ID NO:47, SEQ ID NO:48, or a fragment thereof. In an aspect, a disclosed encoded MBD1 can comprise the sequence set forth in SEQ ID NO:32, SEQ ID NO:33, SEQ ID NO:34, or a fragment thereof. In an aspect, a disclosed encoded MBD1 can comprise a sequence having at least 60%, at least 65%, at least 70%, at least 75%, at least 80%, at least 85%, at least 90%, or at least 95% identity to the sequence set forth in SEQ ID NO:32, SEQ ID NO:33, SEQ ID NO:34, or a fragment thereof.

In an aspect of a disclosed method, a disclosed MBD2 can comprise the sequence set forth in SEQ ID NO:49, SEQ ID NO:50, or a fragment thereof. In an aspect, a disclosed MBD2 can comprise a sequence having at least 60%, at least 65%, at least 70%, at least 75%, at least 80%, at least 85%, at least 90%, or at least 95% identity to the sequence set forth in SEQ ID NO:49, SEQ ID NO:50, or a fragment thereof. In an aspect, a disclosed encoded MBD2 can comprise the sequence set forth in SEQ ID NO:35, SEQ ID NO:36, or a fragment thereof. In an aspect, a disclosed encoded MBD2 can comprise a sequence having at least 60%, at least 65%, at least 70%, at least 75%, at least 80%, at least 85%, at least 90%, or at least 95% identity to the sequence set forth in SEQ ID NO:35, SEQ ID NO:36, or a fragment thereof.

In an aspect of a disclosed method, a disclosed KRAB can comprise the sequence set forth in SEQ ID NO:53, SEQ ID NO:54, SEQ ID NO:55, or a fragment thereof. In an aspect, a disclosed KRAB can comprise a sequence having at least 60%, at least 65%, at least 70%, at least 75%, at least 80%, at least 85%, at least 90%, or at least 95% identity to the sequence set forth in SEQ ID NO:53, SEQ ID NO:54, SEQ ID NO:55, or a fragment thereof. In an aspect, a disclosed encoded KRAB can comprise the sequence set forth in SEQ ID NO:39 or a fragment thereof. In an aspect, a disclosed encoded KRAB can comprise a sequence having at least 60%, at least 65%, at least 70%, at least 75%, at least 80%, at least 85%, at least 90%, or at least 95% identity to the sequence set forth in SEQ ID NO:39 or a fragment thereof.

In an aspect of a disclosed method, a disclosed NIPP1 can comprise the sequence set forth in SEQ ID NO:51, SEQ ID NO:52, or a fragment thereof. In an aspect, a disclosed NIPP1 can comprise a sequence having at least 60%, at least 65%, at least 70%, at least 75%, at least 80%, at least 85%, at least 90%, or at least 95% identity to the sequence set forth in SEQ ID NO:51, SEQ ID NO:52, or a fragment thereof. In an aspect, a disclosed encoded NIPP1 can comprise the sequence set forth in SEQ ID NO:37, SEQ ID NO:38, or a fragment thereof. In an aspect, a disclosed encoded NIPP1 can comprise a sequence having at least 60%, at least 65%, at least 70%, at least 75%, at least 80%, at least 85%, at least 90%, or at least 95% identity to the sequence set forth in SEQ ID NO:37, SEQ ID NO:38, or a fragment thereof.

In an aspect of a disclosed method, a disclosed MeCP2 can comprise the sequence set forth in SEQ ID NO:56, SEQ ID NO:57, or a fragment thereof. In an aspect, a disclosed MeCP2 can comprise a sequence having at least 60%, at least 65%, at least 70%, at least 75%, at least 80%, at least 85%, at least 90%, or at least 95% identity to the sequence set forth in SEQ ID NO:56, SEQ ID NO:57, or a fragment thereof. In an aspect, a disclosed encoded MeCP2 can comprise the sequence set forth in SEQ ID NO:40 or a fragment thereof. In an aspect, a disclosed encoded MeCP2 can comprise a sequence having at least 60%, at least 65%, at least 70%, at least 75%, at least 80%, at least 85%, at least 90%, or at least 95% identity to the sequence set forth in SEQ ID NO:40 or a fragment thereof.

In an aspect of a disclosed method, a disclosed KRAB-MeCP2 can comprise the sequence set forth in SEQ ID NO:58, SEQ ID NO:59, or a fragment thereof. In an aspect, a disclosed KRAB-MeCP2 can comprise a sequence having at least 60%, at least 65%, at least 70%, at least 75%, at least 80%, at least 85%, at least 90%, or at least 95% identity to the sequence set forth in SEQ ID NO:58, SEQ ID NO:59, or a fragment thereof. In an aspect, a disclosed encoded KRAB-MeCP2 can comprise the sequence set forth in SEQ ID NO:41 or a fragment thereof. In an aspect, a disclosed encoded KRAB-MeCP2 can comprise a sequence having at least 60%, at least 65%, at least 70%, at least 75%, at least 80%, at least 85%, at least 90%, or at least 95% identity to the sequence set forth in SEQ ID NO:41 or a fragment thereof.

In an aspect of a disclosed method, a disclosed DNMT3A can comprise the sequence set forth in SEQ ID NO:60, SEQ ID NO:61, or a fragment thereof. In an aspect, a disclosed DNMT3A can comprise a sequence having at least 60%, at least 65%, at least 70%, at least 75%, at least 80%, at least 85%, at least 90%, or at least 95% identity to the sequence set forth in SEQ ID NO:60, SEQ ID NO:61, or a fragment thereof. In an aspect, a disclosed encoded DNMT3A can comprise the sequence set forth in SEQ ID NO:42 or a fragment thereof. In an aspect, a disclosed encoded DNMT3A can comprise a sequence having at least 60%, at least 65%, at least 70%, at least 75%, at least 80%, at least 85%, at least 90%, or at least 95% identity to the sequence set forth in SEQ ID NO:42 or a fragment thereof.

In an aspect of a disclosed method, a disclosed encoded polypeptide can comprise transcription repression activity. A disclosed encoded polypeptide comprising transcription repression activity can comprise a Krüppel associated box domain, an ERF repressor domain, a MXI1 repressor domain, a SID4x repressor domain, a fused KRAB-MeCP2 domain, a MeCP2 TRD domain, a MAS-SID repressor domain, a TATA box binding protein activity, or any combination thereof. In an aspect, a disclosed encoded polypeptide comprising transcription repression activity can comprise HP1 repressor activity, MeCP2 repressor activity, MBD1 repressor activity, MBD2 repressor activity, MBD3 repressor activity, MBD4 repressor activity, KRAB repressor activity, SUV39H1 repressor activity, SUV39H2 repressor activity, CTCF insulator-repressor activity, LSD-1 histone-demethylase repressor activity, or any combination thereof.

In an aspect of a disclosed method, a disclosed encoded polypeptide can comprise transcription release factor activity. A disclosed encoded polypeptide comprising transcription release factor activity can comprise Eukaryotic Release Factor 1 (ERF1) activity or Eukaryotic Release Factor 3 (ERF3) activity. In an aspect, a disclosed ERF1 can comprise the sequence set forth in SEQ ID NO:71 or a fragment thereof. In an aspect, a disclosed encoded ERF1 can comprise a sequence having at least 60%, at least 65%, at least 70%, at least 75%, at least 80%, at least 85%, at least 90%, or at least 95% identity to the sequence set forth in SEQ ID NO:72 or a fragment thereof.

In an aspect of a disclosed method, a disclosed encoded polypeptide can comprise histone modification activity. A disclosed encoded polypeptide comprising histone modification activity can comprise histone acetyltransferase, histone deacetylase, histone demethylase, histone methyltransferase activity, or any combination thereof.

In an aspect of a disclosed method, a disclosed encoded polypeptide can comprise nucleic acid association activity. A disclosed encoded polypeptide comprising nucleic acid association activity can comprise a helix-turn-helix region, a leucine zipper region, a winged helix region, a winged helix-turn-helix region, a helix-loop-helix region, an immunoglobulin fold, a B3 domain, a zinc finger, a HMG-box, a Wor3 domain, a TAL effector DNA-binding domain, or any combination thereof.

In an aspect of a disclosed method, a disclosed encoded polypeptide can comprise methyltransferase activity. A disclosed encoded polypeptide comprising methyltransferase activity can comprise DNA (cytosine-5)-methyltransferase 3a (DNMT3A). In an aspect of a disclosed method, a disclosed encoded polypeptide can comprise demethylase activity. A disclosed encoded polypeptide comprising demethylase activity can comprise ten-eleven translocation methylcytosine dioxygenase 1 (TET1) or lysine-specific histone demethylase 1 (LSD1). In an aspect of a disclosed method, a disclosed encoded polypeptide can comprise acetyltransferase activity. A disclosed encoded polypeptide comprising acetyltransferase activity can comprise histone acetyltransferase. In an aspect of a disclosed method, a disclosed encoded polypeptide can comprise deacetylase activity. A disclosed encoded polypeptide comprising deacetylase activity can comprise histone deacetylase.

In an aspect of a disclosed method, a disclosed encoded dCas endonuclease is fused to the at least one encoded polypeptide having an enzymatic activity. In an aspect, a disclosed dCas endonuclease can comprise dSpCas9 and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dSaCas9 and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof. In an aspect, a disclosed dCas endonuclease can comprise dCjCas9 and a disclosed polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof.

In an aspect of a disclosed method, a disclosed dCas endonuclease can comprise dVQR, dEQR, or dVRER and a disclosed encoded polypeptide can comprise HP1a, HP1b, MBD1, MBD2, Krüppel-associated box (KRAB), NIPP1, the transcription repression domain (TRD) of Methyl-CpG Binding Protein 2 (MeCP2), DNMT3A, any combination thereof, or any fusion thereof.

In an aspect of a disclosed method, a disclosed dCas endonuclease can comprise dSpCas9, dSaCas9, or dCjCas9 and a disclosed encoded polypeptide can comprise KRAB-MeCP2.

In an aspect of a disclosed method, a disclosed dCas endonuclease can comprise dSpCas9, dSaCas9, or dCjCas9, and a disclosed fusion can comprise HP1a-HP1a, HP1a-HP1b, HP1a-MBD1, HP1a-MBD2, HP1a-KRAB, HP1a-NIPP1, HP1a-MeCP2, HP1a-DNMT3A, HP1b-HP1b, HP1b-HP1a, HP1b-MBD1, HP1b-MBD2, HP1b-KRAB, HP1b-NIPP1, HP1b-MeCP2, HP1b-DNMT3A, MBD1-MBD1, MBD1-HP1a, MBD1-HP1b, MBD1-MBD2, MBD1-KRAB, MBD1-NIPP1, MBD1-MeCP2, MBD1-DNMT3A, MBD2-MBD2, MBD2-HP1a, MBD2-HP1b, MBD2-MBD1, MBD2-KRAB, MBD2-NIPP1, MBD2-MeCP2, MBD2-DNMT3A, KRAB-KRAB, KRAB-HP1a, KRAB-HP1b, KRAB-MBD1, KRAB-MBD2, KRAB-NIPP1, KRAB-MeCP2, KRAB-DNMT3A, NIPP1-NIPP1, NIPP1-HP1a, NIPP1-HP1b, NIPP1-MBD1, NIPP1-MBD2, NIPP1-KRAB, NIPP1-MeCP2, NIPP1-DNMT3A, MeCP2-MeCP2, MeCP2-HP1a, MeCP2-HP1b, MeCP2-MBD1, MeCP2-MBD2, MeCP2-KRAB, MeCP2-NIPP1, MeCP2-DNMT3A, DNMT3A-DNMT3A, DNMT3A-HP1a, DNMT3A-HP1b, DNMT3A-MBD1, DNMT3A-MBD2, DNMT3A-KRAB, DNMT3A-NIPP1, or DNMT3A-MeCP2.

In an aspect of a disclosed method, a disclosed viral vector or disclosed recombinant AAV vector can further comprise a nucleic acid sequencing encoding one or more regulatory elements. In an aspect, a disclosed regulatory element can comprise a promoter, an enhancer, a promoter/enhancer, an internal ribosomal entry site, a transcription terminal signal, a polyadenylation signal, a Sp1 and/or NF-kB transcriptional factor binding site, a p2A signal, a woodchuck hepatitis virus post-transcriptional regulatory element, a Phi signal-packaging signal, a rev responsive element, a 5′-LTR, a 3′-LTR, an inverted terminal repeat, a nuclear localization signal (NLS), or any combination thereof.

In an aspect, a disclosed NLS can comprise the sequence set forth in SEQ ID NO:105-SEQ ID NO:111 or a fragment thereof. In an aspect, a disclosed PolyA sequence can comprise the sequence set forth in SEQ ID NO:103, SEQ ID NO:104, or a fragment thereof. In an aspect, a disclosed ITR can comprise the sequence set forth in SEQ ID NO:99-SEQ ID NO:102 or a fragment thereof.

In an aspect of a disclosed method, a disclosed viral vector or disclosed recombinant AAV vector can further a nucleic acid sequence encoding one or more promoters. In an aspect, a disclosed promoter can comprise a U6 promoter, a chicken β-actin promoter, an EF-1α, a CMV promoter, a CMV promoter/enhancer, a fragment thereof, or any combination thereof. In an aspect, a disclosed U6 promoter can comprise the sequence set forth in SEQ ID NO:114 or a fragment thereof. In an aspect, a disclosed U6 promoter can comprise a sequence having at least 60%, at least 65%, at least 70%, at least 75%, at least 80%, at least 85%, at least 90%, at least 95%, or greater than 95% identity to the sequence set forth in SEQ ID NO:114 or a fragment thereof. In an aspect, a disclosed EF-1α promoter can comprise the sequence set forth in SEQ ID NO:112, SEQ ID NO:113, or a fragment thereof. In an aspect, a disclosed EF-1α promoter can comprise a sequence having at least 60%, at least 65%, at least 70%, at least 75%, at least 80%, at least 85%, at least 90%, at least 95%, or greater than 95% identity to the sequence set forth in SEQ ID NO:112, SEQ ID NO:113, or a fragment thereof. In an aspect, a promoter can be a short EF-1α (EFS-NF) promoter.

In an aspect of a disclosed method, a disclosed promoter can be operably linked to the dCas endonuclease. In an aspect, a disclosed promoter operably linked to the dCas endonuclease can comprise an EF-1α or EFS-NC promoter. In an aspect, a disclosed promoter operably linked to the dCas endonuclease can be operably linked to the at least one polypeptide having enzymatic activity. In an aspect, a disclosed promoter can be operably linked to the dCas endonuclease and the at least one polypeptide having enzymatic activity. In an aspect, a disclosed promoter can be operably linked to the fusion protein having one or more enzymatic activities. In an aspect, a disclosed promoter can be operably linked to the dCas endonuclease and the fusion protein having one or more enzymatic activities.

In an aspect, a disclosed promoter operably can be linked to the at least one guide RNA targeting the SNCA gene or a portion of the SNCA gene. In an aspect, a disclosed promoter operably linked to the at least one guide RNA can comprise a U6 promoter.

In an aspect of a disclosed method, a disclosed viral vector or disclosed recombinant AAV vector can further comprise a gRNA scaffold. In an aspect, a disclosed gRNA scaffold can comprise the sequence set forth in SEQ ID NO:115, SEQ ID NO:116, or a fragment thereof.

In an aspect of a disclosed method, a disclosed viral vector or disclosed recombinant AAV vector can further comprise one or more promoters, wherein a first promoter can be operably linked to the dCas endonuclease, and wherein a second promoter can be operably linked to the at least one guide RNA targeting the SNCA gene or a portion of the SNCA gene.

In an aspect of a disclosed method, a disclosed viral vector or disclosed recombinant AAV vector can further comprise one or more promoters, wherein a first promoter can be operably linked to the dCas endonuclease and the at least one polypeptide having an enzymatic activity, and wherein a second promoter can be operably linked to the at least one guide RNA the SNCA gene or a portion of the SNCA gene.

In an aspect of a disclosed method, a disclosed viral vector or disclosed recombinant AAV vector, a disclosed encoded polypeptide can comprise HP1a, HP1b, MBD1, MBD2, Krüppel-Associated Box (KRAB), NIPP1, the Transcription Repression Domain (TRD) of Methyl-CpG Binding Protein 2 (MeCP2), DNMT3A, any combination thereof, or any fusion thereof.

In an aspect of a disclosed method, a disclosed nucleic acid sequence can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art. In an aspect, any disclosed dCas9 endonuclease, a disclosed polypeptide having enzymatic activities, a disclosed fusion product or a disclosed fusion protein, or any combination thereof can be codon-optimized.

In an aspect of a disclosed method, a disclosed viral vector or disclosed recombinant AAV vector can comprise a sequence that is about 4.5 kilobases or less than about 4.5 kilobases.

In an aspect, a disclosed method can further comprise effecting precision epigenetic modulation of the SNCA gene or a portion of the SNCA gene. In an aspect, modulating can comprise decreasing and/or reducing expression and/or activity of the SNCA gene or a portion of the SNCA gene.

In an aspect, the disclosed cells can be neurons (e.g., cholinergic neurons, dopaminergic neurons, etc.). In an aspect, the disclosed cells can be cells affected by an overexpression level and/or activity level of the SNCA gene or a portion of the SNCA gene.

In an aspect, the disclosed cells can be in a subject. In an aspect of a disclosed method, a subject can be a human. In an aspect, a subject can be suspected of having or can be diagnosed with having Parkinson's disease or Lewy Body Dementia.

In an aspect, a disclosed method can comprise reducing the pathological phenotype associated with Parkinson's disease. In an aspect, reducing the pathological phenotype associated with Parkinson's disease can comprise reducing the number and/or amount of amyloid plaques. In an aspect, a disclosed method can comprise diagnosing the subject with Parkinson's disease.

In an aspect, a subject can be a subject in need of treatment of Parkinson's disease. In an aspect, a disclosed method of treating and/or preventing Parkinson's disease progression can comprise restoring one or more aspects of cellular homeostasis and/or cellular functionality and/or metabolic dysregulation.

In an aspect, restoring one or more aspects of cellular homeostasis and/or cellular functionality and/or metabolic dysregulation can comprise reducing the expression and/or activity level of the SNCA gene or a portion of the SNCA gene. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof.

In an aspect, restoring one or more aspects of cellular homeostasis can comprise improving, enhancing, restoring, and/or preserving one or more aspects of cellular structural and/or functional integrity. In an aspect, restoring the activity and/or functionality of a missing, deficient, and/or mutant protein or enzyme can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme). In an aspect, restoration can be a partial or incomplete restoration. In an aspect, restoration can be complete or near complete restoration such that the level of expression, activity, and/or functionality is like that of a wild-type or control level.

In an aspect, restoring the activity and/or functionality of the SNCA gene or a portion of the SNCA gene can comprise decreasing or reducing the expression and/or activity level of the SNCA gene or a portion of the SNCA gene. In an aspect, decreasing or reducing can comprise a decrease of at least about 5%, 10%, 15%, 20%, 25%, 35%, 50%, 75%, 80%, 85%, 90%, 95%, 97%, 98%, 99%, 100%, 200%, 300%, 400%, 500%, or more as compared to a control (such as a pre-treatment level).

In an aspect of a disclosed method of treating and/or preventing Parkinson's disease progression, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect of a disclosed method of treating and/or preventing Parkinson's disease progression, administering can comprise intravenous administration, intracerebral administration, intra-CSF administration, intracerebroventricular (ICV) administration, intraventricular administration, intra-cisterna magna (ICM) administration, intraparenchymal administration, intrathecal (lumbar, cisternal, or both) administration, intrahepatic administration, hepatic intra-arterial administration, hepatic portal vein (HPV) administration, or any combination thereof. In an aspect, a disclosed vector can be administered via LNP administration.

In an aspect, a disclosed method of treating and/or preventing Parkinson's disease progression can employ multiple routes of administration to the subject. In an aspect, a disclosed method can employ a first route of administration that can be the same or different as a second and/or subsequent routes of administration. In an aspect, a disclosed nucleic acid molecule, a disclosed vector, and/or a disclosed pharmaceutical formulation can be concurrently and/or serially administered to a subject via multiple routes of administration. For example, in an aspect, administering a disclosed nucleic acid molecule, a disclosed vector, and/or a disclosed pharmaceutical formulation can comprise intravenous administration and intra-cistern magna (ICM) administration. In an aspect, administering a disclosed nucleic acid molecule, a disclosed vector, and/or a disclosed pharmaceutical formulation can comprise IV administration and intrathecal (ITH) administration.

In an aspect of a disclosed method of treating and/or preventing Parkinson's disease progression, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ vg/kg to about 8×10¹³ vg/kg or about 1×10¹² vg/kg to about 8×10¹³ vg/kg. In an aspect, a disclosed vector can be administered at a dose of about 1×10¹³ vg/kg to about 6×10¹³ vg/kg. In an aspect, a disclosed vector can be administered at a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a disclosed vector can be administered at a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method of treating and/or preventing Parkinson's disease progression can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method of treating and/or preventing Parkinson's disease progression can further comprise administering to the subject a therapeutically effective amount of a therapeutic agent. A therapeutic agent can be any disclosed agent that effects a desired clinical outcome.

In an aspect, a disclosed method of treating and/or preventing Parkinson's disease progression can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method of treating and/or preventing Parkinson's disease progression can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of treating and/or preventing Parkinson's disease progression can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method of treating and/or preventing Parkinson's disease progression can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of treating and/or preventing Parkinson's disease progression can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of treating and/or preventing Parkinson's disease progression can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect of a disclosed method, a subject can be a human. In an aspect, a subject can be suspected of having or can be diagnosed with having Parkinson's disease (such as, for example, PD). In an aspect, a disclosed subject can be symptomatic or asymptomatic.

In an aspect, a disclosed method can comprise repeating one or more steps of the method and/or modifying one or more steps of the method (such as, for example, an administering step).

In an aspect, a disclosed method of treating and/or preventing Parkinson's disease progression can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, a disclosed method of treating and/or preventing Parkinson's disease progression can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In an aspect, a disclosed method of treating and/or preventing Parkinson's disease progression can further comprise generating and/or validating one or more of the disclosed isolated nucleic acid molecules, one or more of the disclosed vectors, one or more of the disclosed pharmaceutical formulations, or any combination thereof.

In an aspect, a disclosed method of treating and/or preventing Parkinson's disease progression can further comprise administering to the subject a second disclosed isolated nucleic acid molecule, a second disclosed vector, a second disclosed pharmaceutical formulations, or any combination thereof. In an aspect, a disclosed method of treating and/or preventing Parkinson's disease progression can further comprise administering to the subject additional disclosed isolated nucleic acid molecules, additional disclosed vectors, additional disclosed pharmaceutical formulations, or any combination thereof.

### 1. Kits

Disclosed herein is a kit comprising one or more disclosed isolated nucleic acid molecules, disclosed vectors, disclosed AAV vectors, disclosed pharmaceutical formulations, disclosed host cells, disclosed guide RNAs, disclosed plasmids, or any combination thereof with or without additional therapeutic agents to effect precision epigenetic modulation. Disclosed herein is a kit comprising one or more disclosed isolated nucleic acid molecules, disclosed vectors, disclosed AAV vectors, disclosed pharmaceutical formulations, disclosed host cells, disclosed guide RNAs, disclosed plasmids, or any combination thereof with or without additional therapeutic agents to treat and/or prevent Alzheimer's disease progression. Disclosed herein is a kit comprising one or more disclosed isolated nucleic acid molecules, disclosed vectors, disclosed AAV vectors, disclosed pharmaceutical formulations, disclosed host cells, disclosed guide RNAs, disclosed plasmids, or any combination thereof with or without additional therapeutic agents to treat and/or prevent Parkinson's disease progression.

In an aspect, a disclosed kit can be used in a disclosed method to reduce expression and/or activity of APOE regardless of the subject's genotype. In an aspect, a disclosed kit can be used in a disclosed method to reduce expression and/or activity of SNCA.

In an aspect, a disclosed kit can comprise at least two components constituting the kit. Together, the components constitute a functional unit for a given purpose (such as, for example, treating a subject diagnosed with or suspected of having Alzheimer's disease or Parkinson's disease). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed pharmaceutical formulation, a disclosed therapeutic agent, a disclosed reagent, or a combination thereof, and a label or package insert with instructions for use. In an aspect, suitable containers include, for example, bottles, vials, syringes, blister pack, etc. The containers can be formed from a variety of materials such as glass or plastic. The container can hold, for example, a disclosed pharmaceutical formulation and/or a disclosed therapeutic agent and can have a sterile access port (for example the container may be an intravenous solution bag or a vial having a stopper pierceable by a hypodermic injection needle). The label or package insert can indicate that a disclosed pharmaceutical formulation and/or a disclosed therapeutic agent can be used for treating, preventing, inhibiting, and/or ameliorating Alzheimer's disease or Parkinson's disease or complications and/or symptoms associated with Alzheimer's disease or Parkinson's disease. In an aspect, a disclosed kit can comprise additional components necessary for administration such as, for example, other buffers, diluents, filters, needles, and syringes. In an aspect, a disclosed kit can comprise those components (e.g., primers) necessary to measure one or more times the level of expression and/or the level of activity of the disclosed gene of interest.

### J. Miscellaneous

Disclosed herein is a composition comprising (a)(i) a fusion protein or (ii) a nucleic acid sequence encoding a fusion protein, the fusion protein comprising two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, deacetylase activity, or combination thereof, and (b)(i) at least one guide RNA (gRNA) or (ii) a nucleic acid sequence encoding at least one guide gRNA, wherein the at least one gRNA targets the fusion protein to a target region within the APOE gene.

Disclosed herein is an isolated polynucleotide encoding the composition disclosed above.

Disclosed herein is a vector comprising the isolated polynucleotide disclosed above.

Disclosed herein is a host cell comprising the isolated polynucleotide disclosed above or the vector disclosed above. Disclosed herein is a pharmaceutical composition comprising the least one composition disclosed above, the isolated polynucleotide disclosed above, the vector disclosed above, the host cell disclosed above, or any combination thereof, and a pharmaceutically acceptable carrier and/or excipient.

Disclosed herein is a kit comprising at least one composition disclosed above, the isolated polynucleotide disclosed above, the vector disclosed above, the pharmaceutical composition disclosed above, or any combination.

Disclosed herein is a method of in vivo modulation of expression of a APOE gene in a cell or a subject, the method comprising contacting the cell or subject with at least one composition disclosed above, an isolated polynucleotide disclosed above, a vector disclosed above, a pharmaceutical composition disclosed above, or any combination thereof in an amount sufficient to modulate expression of the gene.

Disclosed herein is a method of treating a disease or disorder associated with elevated APOE expression levels in a subject and as the general approach to reduce e4-allele expression associated with the LOAD, the method comprising administering to the subject or a cell in the subject at least one composition disclosed above, an isolated polynucleotide disclosed above, a vector disclosed above, a pharmaceutical composition disclosed above, or any combination thereof such that the disease or disorder is treated.

Disclosed herein a method of in vivo modulating expression of a APOE gene in a cell or a subject, the method comprising contacting the cell or subject with: (a)(i) a fusion protein or (ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b)(i) at least one guide RNA (gRNA) that targets the fusion molecule to a target region within the APOE gene or (ii) a nucleic acid sequence encoding at least one gRNA that targets the fusion protein to a target region within the APOE gene, in an amount sufficient to modulate expression of the gene.

Disclosed herein is a method of treating a disease or disorder associated with elevated APOE expression levels in a subject and expression of e4-allele in a subject, the method comprising administering to the subject or a cell in the subject: (a)(i) a fusion protein or (ii) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b)(i) at least one guide RNA (gRNA) that targets the fusion molecule to a target region within the APOE gene or (ii) a nucleic acid sequence encoding at least one gRNA that targets the fusion molecule to a target region within the APOE gene, in an amount sufficient to modulate expression of the gene.

Disclosed herein is a viral vector system for epigenomic editing, the viral vector system comprising: (a) a nucleic acid sequence encoding a fusion protein, wherein the fusion protein comprises two heterologous polypeptide domains, wherein the first polypeptide domain comprises a Clustered Regularly Interspaced Short Palindromic Repeats associated (Cas) protein and the second polypeptide domain comprises a peptide having an activity selected from the group consisting of transcription activation activity, transcription repression activity, transcription release factor activity, histone modification activity, nucleic acid association activity, methyltransferase activity, demethylase activity, acetyltransferase activity, and deacetylase activity; and (b) a nucleic acid sequence encoding at least one guide RNA (gRNA) that targets the fusion protein to a target region within the APOE gene.

TABLE 1
Sequence Information

| SEQ | | | |
|---|---|---|---|
| ID | | | |
| NO: | Name of Sequence | Type | Source |
| 01 | Hp1a-dCjCas9 Plasmid | NT | artificial |
| 02 | Hp1b-dCjCas9 Plasmid | NT | artificial |
| 03 | MBD1-dCjCas9 Plasmid | NT | artificial |
| 04 | MBD2-dCjCas9 Plasmid | NT | artificial |
| 05 | NIPP1-dCjCas9 Plasmid | NT | artificial |
| 06 | KRAB-dCjCas9 Plasmid | NT | artificial |
| 07 | MeCP2-dCjCas9 Plasmid | NT | artificial |
| 08 | KRAB-MeCP2-dCjCas9 Plasmid | NT | artificial |
| 09 | DNMT3A-dCjCas9 Plasmid | NT | artificial |
| 10 | Hp1a-dSaCas9 Plasmid | NT | artificial |
| 11 | Hp1b-dSaCas9 Plasmid | NT | artificial |
| 12 | MBD1-dSaCas9 Plasmid | NT | artificial |
| 13 | MBD2-dSaCas9 Plasmid | NT | artificial |
| 14 | NIPP1-dSaCas9 Plasmid | NT | artificial |
| 15 | KRAB-dSaCas9 Plasmid | NT | artificial |
| 16 | MeCP2-dSaCas9 Plasmid | NT | artificial |
| 17 | KRAB-MeCP2-dSaCas9 Plasmid | NT | artificial |
| 18 | DNMT3A-dSaCas9 Plasmid | NT | artificial |
| 19 | dSpCas9 | AA | Streptococcus pyogenes |
| 20 | dSaCas9 | AA | Staphylococcus aureus |
| 21 | dCjCas9 | AA | Campylobacter jejuni |
| 22 | dSpCas9 | NT | Streptococcus pyogenes |
| 23 | dSaCas9 | NT | Staphylococcus aureus |
| 24 | dSaCas9 | NT | Staphylococcus aureus |
| 25 | dCjCas9 | NT | Campylobacter jejuni (Cj) |
| 26 | dCjCas9 | NT | Campylobacter jejuni (Cj) |
| 27 | VRER Cas9 | NT | artificial |
| 28 | HP1 alpha | AA | human |
| 29 | HP1 alpha | AA | human |
| 30 | HP1 beta | AA | human |
| 31 | HP1 beta | AA | human |
| 32 | MBD1 | AA | artificial |
| 33 | MBD1 isoform 1 | AA | human |
| 34 | MBD1 isoform 6 | AA | human |
| 35 | MBD2 | AA | human |
| 36 | MBD2 | AA | human |
| 37 | NIPP1 | AA | human |
| 38 | NIPP1 | AA | human |
| 39 | KRAB | AA | artificial |
| 40 | MeCP2 | AA | human |
| 41 | KRAB-MeCP2 | AA | artificial |
| 42 | DNMT3A sequence | AA | human |
| 43 | HP1 alpha | NT | human |
| 44 | HP1a Hinge + CSD | NT | artificial |
| 45 | HP1 beta | NT | human |
| 46 | HP1B Hinge + CSD | NT | artificial |
| 47 | MBD1 | NT | human |
| 48 | MBD1 | NT | human |
| 49 | MBD2 | NT | human |
| 50 | MBD2 | NT | human |
| 51 | NIPP1 | NT | human |
| 52 | NIPP1 | NT | human |
| 53 | KRAB | NT | human |
| 54 | KRAB | NT | human |
| 55 | KRAB | NT | human |
| 56 | MeCP2 | NT | human |
| 57 | MeCP2 | NT | human |
| 58 | KRAB-MeCP2 | NT | artificial |
| 59 | KRAB-MeCP2 | NT | artificial |
| 60 | DNMT3A NT sequence | NT | human |
| 61 | DNMT3A NT sequence | NT | human |
| 62 | dCas9-DNMT3A fusion protein | NT | artificial |
| 63 | dCas9-DNMT3A fusion protein | AA | artificial |
| 64 | VRER-DNMT3A | AA | artificial |
| 65 | NF-Kβ subunit/RELA Proto-Oncogene | NT | human |
| 66 | NF-Kβ subunit/RELA Proto-Oncogene | NT | human |
| | (mRNA) | | |
| 67 | NF-Kβ subunit/RELA Proto-Oncogene | AA | human |
| 68 | VP16 | NT | Herpes simplex virus type 2 |
| 69 | VP16 | NT | Herpes simplex virus type 2 |
| 70 | VP16 | AA | Herpes simplex virus type 2 |
| 71 | Eukaryotic Release Factor 1 | NT | human |
| 72 | Eukaryotic Release Factor 1 | AA | human |
| 73 | gRNA targeting CMV promoter region | NT | artificial |
| 74 | gRNA targeting CMV promoter region | NT | artificial |
| 75 | gRNA targeting CMV promoter region | NT | artificial |
| 76 | gRNA targeting CMV promoter region | NT | artificial |
| 77 | gRNA targeting CMV promoter region | NT | artificial |
| 78 | gRNA targeting CMV promoter region | NT | artificial |
| 79 | gRNA targeting CMV promoter region | NT | artificial |
| 80 | gRNA targeting CMV promoter region | NT | artificial |
| 81 | gRNA targeting promoter region of | NT | artificial |
| | CAG-INTRON1 | | |
| 82 | gRNA targeting promoter region of | NT | artificial |
| | CAG-INTRON1 | | |
| 83 | gRNA targeting promoter region of | NT | artificial |
| | CAG-INTRON1 | | |
| 84 | gRNA targeting promoter region of | NT | artificial |
| | CAG-INTRON1 | | |
| 85 | gRNA targeting APOE promoter region | NT | artificial |
| 86 | gRNA targeting APOE promoter region | NT | artificial |
| 87 | gRNA targeting APOE promoter region | NT | artificial |
| 88 | gRNA targeting APOE promoter region | NT | artificial |
| 89 | gRNA targeting APOE promoter region | NT | artificial |
| 90 | gRNA targeting APOE promoter region | NT | artificial |
| 91 | gRNA targeting APOE promoter region | NT | artificial |
| 92 | gRNA targeting APOE promoter region | NT | artificial |
| 93 | APOE | NT | human |
| 94 | APOE e2 | NT | human |
| 95 | APOE e3 | NT | human |
| 96 | APOE e4 | NT | human |
| 97 | PAM for CJCas9 | NT | artificial |
| 98 | PAM for CaCAS9 | NT | artificial |
| 99 | ITR | NT | AAV2 |
| 100 | ITR | NT | AAV2 |
| 101 | ITR | NT | AAV2 |
| 102 | ITR | NT | AAV2 |
| 103 | PolyA | NT | artificial |
| 104 | PolyA | NT | artificial |
| 105 | Nuclear Localization Signal | NT | artificial |
| 106 | Nuclear Localization Signal | NT | artificial |
| 107 | Nuclear Localization Signal | NT | artificial |
| 108 | Nuclear Localization Signal | NT | artificial |
| 109 | Nuclear Localization Signal | NT | artificial |
| 110 | Nuclear Localization Signal | NT | artificial |
| 111 | Nuclear Localization Signal | NT | artificial |
| 112 | EF-1a Core Promoter | NT | artificial |
| 113 | EF-1a Core Promoter | NT | artificial |
| 114 | U6 Promoter | NT | artificial |
| 115 | Cj gRNA Scaffold | NT | artificial |
| 116 | Sa gRNA Scaffold | NT | artificial |
| 117 | pCL35-Lentiviral vector carrying | NT | artificial |
| | reporter system comprised of CAGp- | | |
| | SNCA-INTRON1-SNCA-ORF-dGFP- | | |
| | Nano-Luc-WPRE-CV40-Puro | | |
| 118 | pBK500-Lentivirus construct sequence | NT | artificial |
| | containing fusion protein and gRNA | | |
| 119 | pBK539-Plasmid carrying dCas9- | NT | artificial |
| | DNMT3 A fused transgene linked to GFP | | |
| | selection gene via p2A cleavage signal | | |
| 120 | pBK546-Plasmid carrying dCas9- | NT | artificial |
| | DNMT3A fused transgene linked to | | |
| | puromycin selection gene via p2A | | |
| | cleavage signal | | |
| 121 | pBK1340-Lentiviral vector carrying | NT | artificial |
| | reporter system comprised of CMVp- | | |
| | dGFP-Nano-Luc-WPRE-CV40-Puro | | |
| 122 | pBK1531-Lentiviral vector carrying | NT | artificial |
| | dCas9-VRER-MeCP2 Transcription | | |
| | Repression Domain (TRD) and gRNA1 | | |
| 123 | pBK1532-Lentiviral vector carrying | NT | artificial |
| | dCas9-VRER-MeCP2 Transcription | | |
| | Repression Domain (TRD) and gRNA2 | | |
| 124 | pBK1536-Lentiviral vector carrying | NT | artificial |
| | dCas9-VRER-KRAB-MeCP2 | | |
| | Transcription Repression Domain (TRD) | | |
| | without gRNA | | |
| 125 | gRNA targeting GFP ORF | NT | artificial |
| 126 | gRNA targeting GFP ORF | NT | artificial |
| 127 | Alpha-synuclein | NT | human |
| 128 | Alpha-synuclein | AA | human |
| 129 | CjCas9 PAM | NT | artificial |
| 130 | SaCas9 PAM | NT | artificial |
| 131 | CjCas9 gRNA1 | NT | artificial |
| 132 | CjCas9 gRNA2 | NT | artificial |
| 133 | SaCas9 gRNA1 | NT | artificial |
| 134 | SaCas9 gRNA2 | NT | artificial |
| 135 | U6/R1 primer | NT | artificial |
| 136 | U6/L1 primer | NT | artificial |

### VII. EXAMPLES

As detailed herein, innovative epigenome editing tools to downregulate APOE expression precisely and in allele-specific manner are described and claimed.

Epigenome editing represents a novel appealing approach for gene therapy mediated by altering expression of a specific gene based on CRISPR/dCas9 technology. While genome editing approaches change the genetic code via DNA cleavage and are prone to introduce new mutations, epigenome editing as described herein uses deactivated nuclease. Thus, the DNA remains intact, and the deactivated nuclease does not change the DNA sequence.

The allelic discrimination approach described herein is innovative allowing a precise and fine-tuned downregulation of APOE e4 allele expression. The novel all-in-one vector described herein circumvents several challenges related to gene therapy. First, the AAV backbone is optimized for high packaging and delivery efficiencies of the CRISPR/Cas9 components. Second, the all-in-one vector is suitable for a broad range of cellular tropisms. Third, the all-in-one vector demonstrates low cytotoxicity and immunogenicity. Fourth, the all-in-one vector demonstrates long-term and sustainable expression to ensure the durability of the epigenetic changes.

The design and implementation of an Sp1-NF-kB harbored vector backbone carrying a compact CRISPR-dCas9-based transcriptional repression tool that can fit all-in-one within a single AAV vector to achieve efficient epigenetic editing is described herein. The development of this robust epigenetic editing tool enables novel architectures for synthetic gene circuits and potential safe gene therapy for several diseases.

### Materials and Methods

Plasmid Design and Construction: All expression cassettes of AAV vectors harboring repressor proteins were created by cloning of the above ORFs into SphI site created in previously made AAV backbone. The cloned inserts were synthesized using Gene Script's service.

AAV Vector Production: Plasmids were all packaged into AAV9. AAV vectors were generated using a triple transient transfection protocol in HEK293T (ATCC® CRL3216™) human embryonic kidney cells using polyethyleneimine (PEI). Briefly, for each virus, media for four 25 mm plates of 70-80% confluent HEK293T cells were first changed into Dulbecco's Modified Eagle Medium (DMEM) (Gibco #: 11965-092) without fetal bovine serum (FBS) (Hyclone #: SH30087.01). One hour after triple transfecting the cells with pHelper (12 μg/plate), pAAV Rep-Cap (10 μg/plate), and pAAV ITR-expression (6 μg/plate) plasmids using PEI and DMEM, the media was then changed to DMEM with 10% FBS.

After 72 hours, cells were collected, and the cell pellets underwent 4 freeze thaw cycles in −80° C. and sonification before purified using a double round cesium chloride (CsCl) gradient protocol. Fractions positive for vector genome were pooled and concentrated using an Amicon® Ultra-4 Centrifugal Filter Unit (UFC810096) against three washes of PBS at 4° C. to remove CsCl. Vector and viral stocks were aliquoted and stored at −80° C. AAV titers were determined by SYBR Green qPCR against a standard curve and primer specific to the U6 promotor of the virus.

Cell Culture. HEK293T/pBK1340 and HEK293T/pCL35 cell lines were generated through transduction of HEK293T cells (ATCC® CRL3216™) with pLenti-pBK1340 and pLenti-pCL35 vectors, respectively, and then selected through puromycin selection. These cells expressed green fluorescence protein (GFP) and luciferase downstream of a CMV promoter or a CAG promoter and α-synuclein protein. The GFP tag provided visual changes of gene repression whereas the luciferase provided higher spatial resolution identifying protein concentration changes overtime. Maintenance cells were grown in DMEM (Gibco), supplemented with 10% fetal bovine serum (Gibco), penicillin/streptomycin 1% (Thermo Fisher Scientific), 2 mM L-glutamine, 1% MEM NEAA (Gibco), and 1 mM sodium pyruvate (Gibco). Before lipofectamine 3000 transfection or AAV transduction, the cells were seeded at 2.5×10⁵ cells per 12-well plate and cultured in DMEM with 2% FBS with no supplements.

Lipofectamine 3000 Transfections. Plasmids were first introduced through liposomal transfection using Invitrogen Lipofectamine 3000 Transfection Kit (Cat #L3000-015) according to the manufacturer's instructions. All cells were plated in 12-well plates. 1 μg DNA was first added to 50 μL of Opti-MEM medium (Gibco) and 2 μL of P3000, followed by the addition of 50 μL of Opti-MEM and Lipofectamine 3000 mix (Opti-MEM: Lipofectamine 3000=50:3). The final mixture was then incubated at room temperature for 15 min and then 100 μl of the final mixture was immediately added to each well of the cells.

AAV Transduction. At 50% confluency, HEK293T/pBK1340 and HEK293T/pCL35 cell lines were transduced with AAV/dCas9-HP1A, AAV/sgRNA-dCas9-HP1A, AAV/dCas9-KRAB-MeCP2, and AAV/sgRNA-dCas9-KRAB-MeCP2 for both dCjCas9 and dSaCas9 variants at MOI of 700,000 vg/cell. At 48 hr post transduction, the cells reached 100% confluency and were split to 40% confluency. Over the course of 21 days, the cells were passaged and harvested at 70% confluency to prevent epigenetic modifications caused by over confluency.

Luciferase Reporter Assay. Cells from each 12-well plate were first harvested and washed twice with 1×PBS before being resuspended in 200 μL of 1×PBS. 50 μL of the cell and 1×PBS mixture were transferred into a 96-well plate bottom white plate (Costar Cat #3922) and lysis buffer from Nano-Glo Luciferase Assay Kit (Promega Cat #N1120) was added directly to the plates following the manufacturer's protocol. The data was obtained using a microplate spectrophotometer (Bio-Rad, Hercules, CA). Total protein concentration, which was determined by DC Protein Assay Set (Bio-Rad, Hercules, CA), was then used for data normalization.

Western Blotting. α-synuclein protein expression levels in the HEK293T/pCL35 cell lines were determined by western blotting with the α-synuclein rabbit monoclonal antibody (ab212184, Abcam; 1:1,000) and with rabbit monoclonal antibody (mAb) GAPDH (AM4302, Ambion; 1:5,000) for normalization. HEK293T/pCL35 cells were harvested from the 24-well plate and cells were lysed with NP-40 buffer: 150 mM NaCl, 1.0% IGEPAL CA-630, 50 mM Tris-HCL (pH 8.0), and 1× protease inhibitor cocktail. Total protein concentrations were measured by DC Protein Assay Set (Bio-Rad, Hercules, CA), and 3 g of each sample was run on 15-well Tris-glycine SDS-PAGE gels. The samples were transferred to nitrocellulose membranes. The blots were first blocked with 5% BSA in TBST then incubated in the primary antibody overnight at 4° C. (ab212184, Abcam; 1:1,000). Afterwards, the blots were washed three times with TBST, incubated with the secondary antibody in 5% milk in TBST for 1 hour, and washed again with TBST for three times. The densitometry was measured using ImageJ software, and α-synuclein expression was normalized to GAPDH expression in the same lane.

Genome DNA Extraction and AAV Integration Analysis. Genome DNA was extracted from each cell line every 3-4 days after transfection or transduction using DNeasy Blood and Tissue Kit (QIAGEN) per the manufacturer's instructions. qPCR was used to quantify the level of AAV integration by replicating viral dSaCas9 and dCjCas9 sequences. The following primers were used: U6/R1 (5′-gcctatttcccatgattcctt-3′ (SEQ ID NO:135)) and U6/L1 (5′-aaaactgcaaactacccaagaa-3′ (SEQ ID NO:136)).

Statistical Analysis. The significance of the differences between no sgRNA and with sgRNA groups were analyzed statistically using student's t-test (Realstats Excel). The gene expression was measured through the changes in luciferase concentration using a NanoLuc assay, while a BCA Assay was also conducted to obtain the overall amino acid concentration to normalize the data. The treated cells were then normalized using the control group of cells transfected/transduced with no sgRNA and no transcriptional repressor domain.

$$Percentage of Luciferase ( POL ) = \frac{[ Luciferase ]}{[ Total Amino Acid ]} \qquad ( 1 )$$

$$Normalized Luciferase = \frac{POL in Treatment Group}{POL in Control Group} \qquad ( 2 )$$

Equations (1-2) demonstrate how the luciferase was normalized and quantified.

### Example 1

### Summary of Experiments Described Herein

The Adeno-Associated Virus (AAV)-CRISPR/deactivated(d)Cas9-repressor platform has been established and validated in vitro. The functional packaging capacity of the AAV is small, ~4.7 kb genome size, which is a limitation to package the bulky multicomponent CRISPR/Cas system into a single AAV (Rittiner J E, et al. (2020). Front Mol Neurosci 13:148). Co-delivering the CRISPR/Cas9 system with separate AAV vectors results inefficient and inadequate delivery (Rittiner J E, et al. (2020). Front Mol Neurosci 13:148).

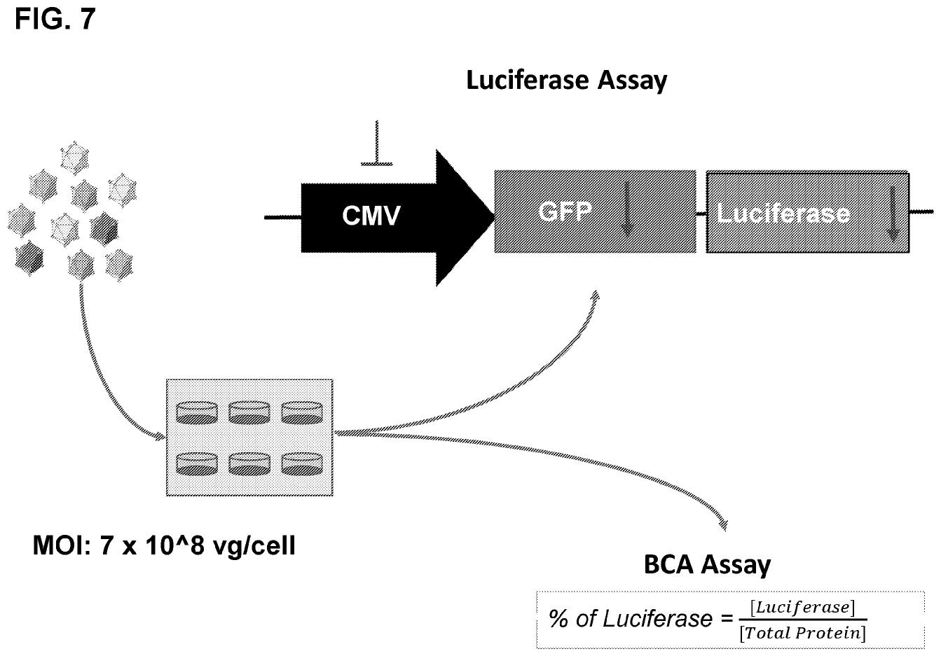

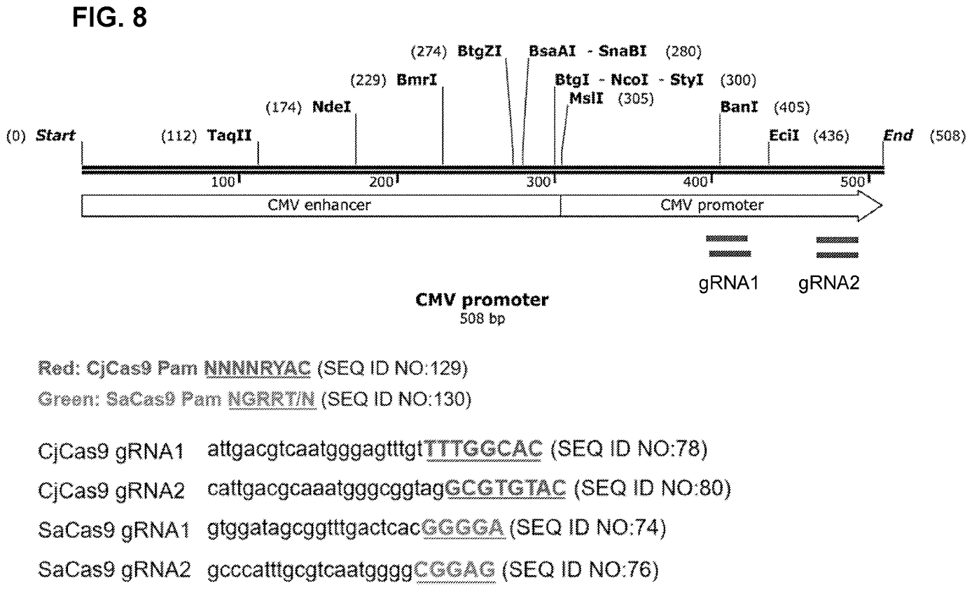

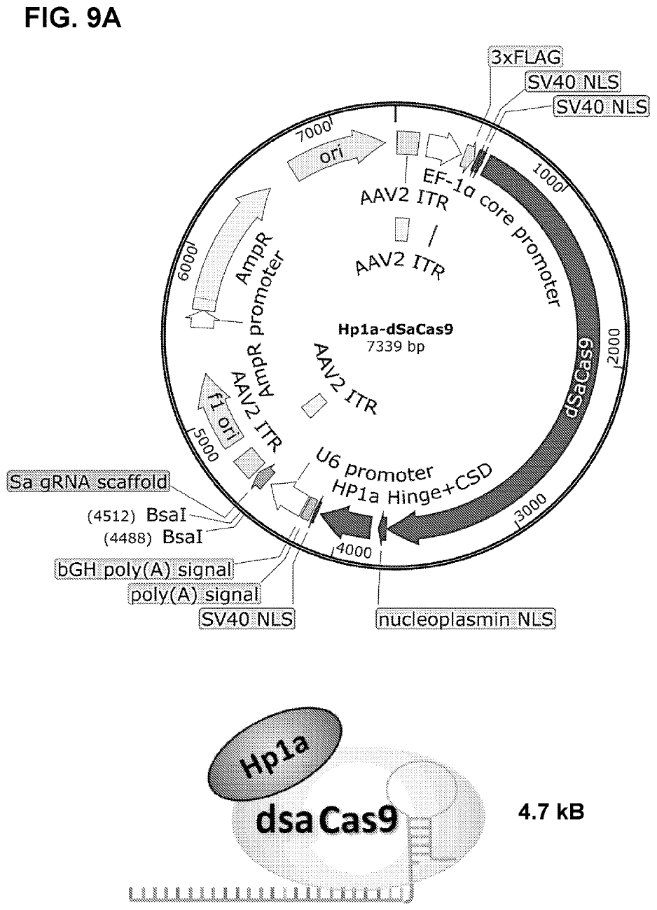

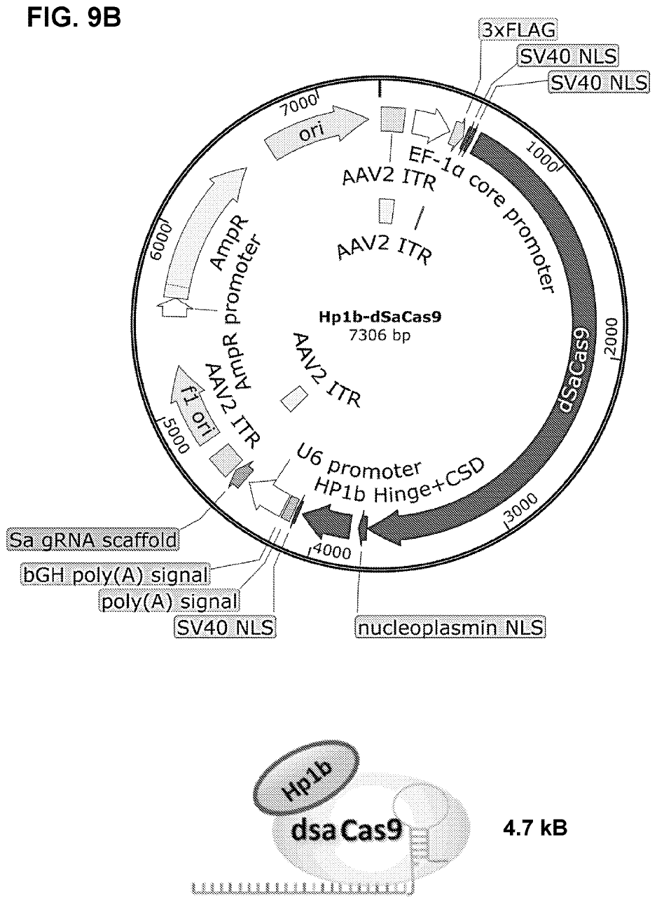

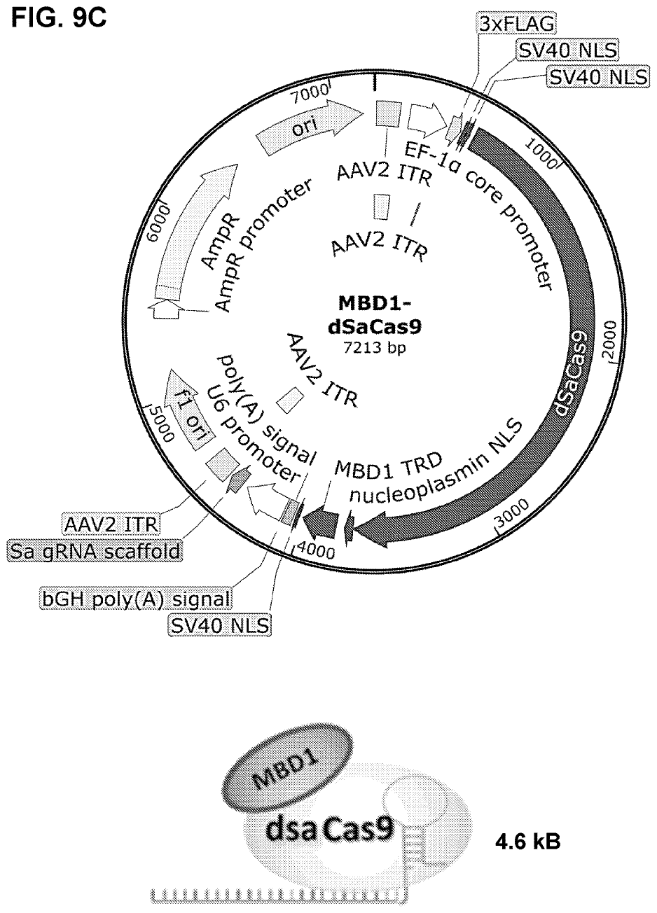

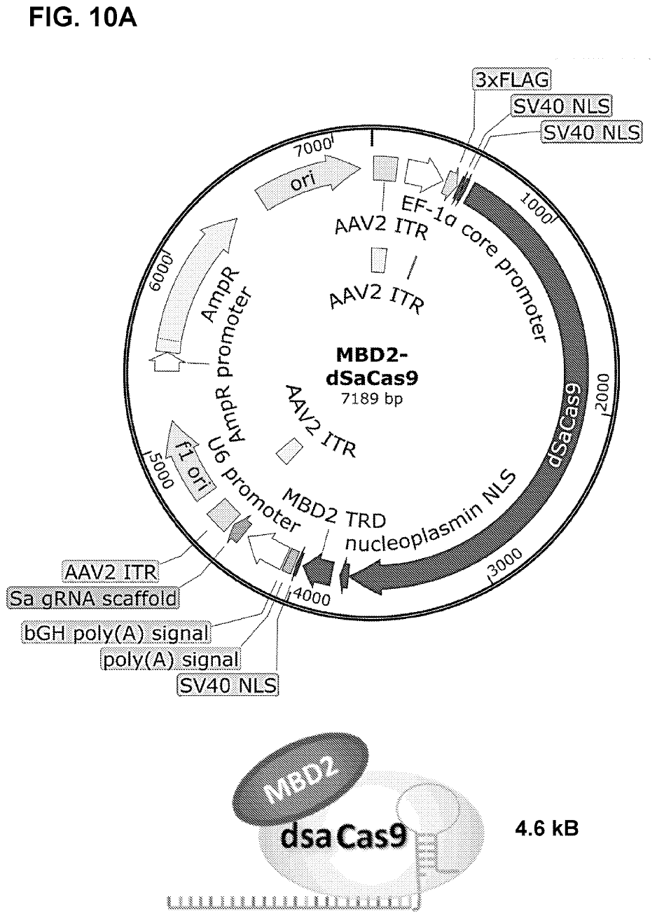

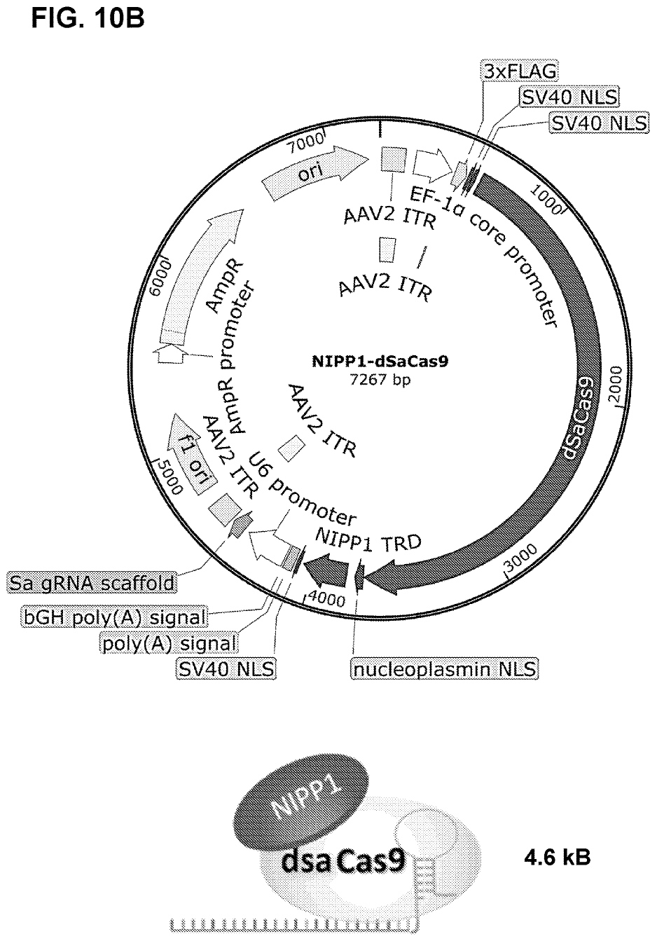

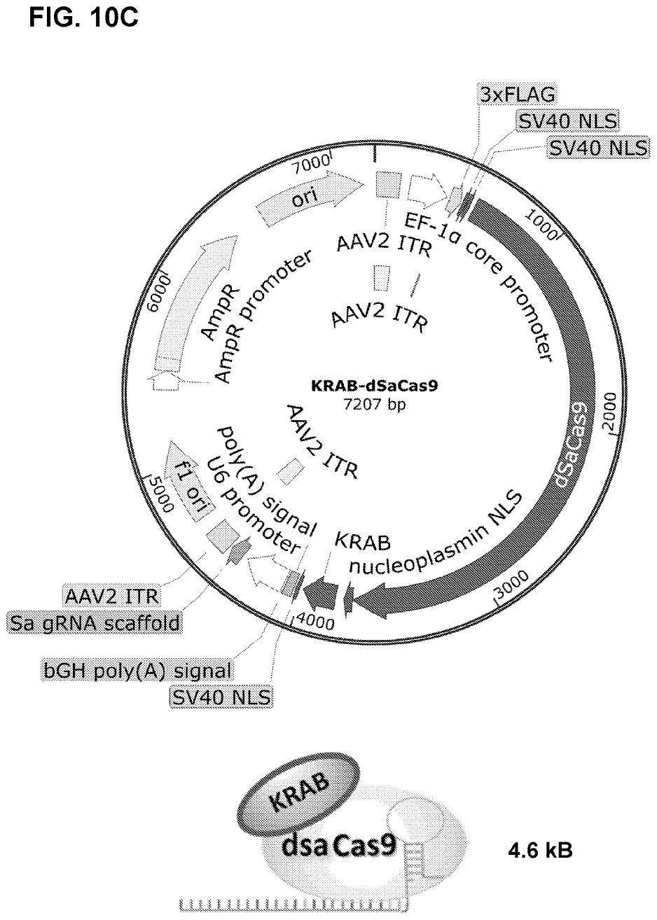

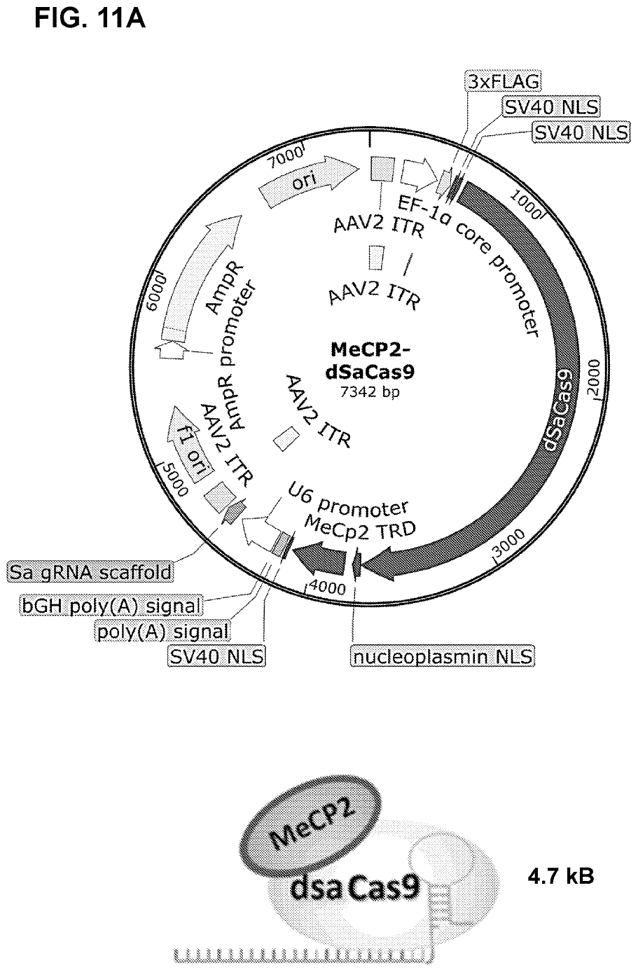

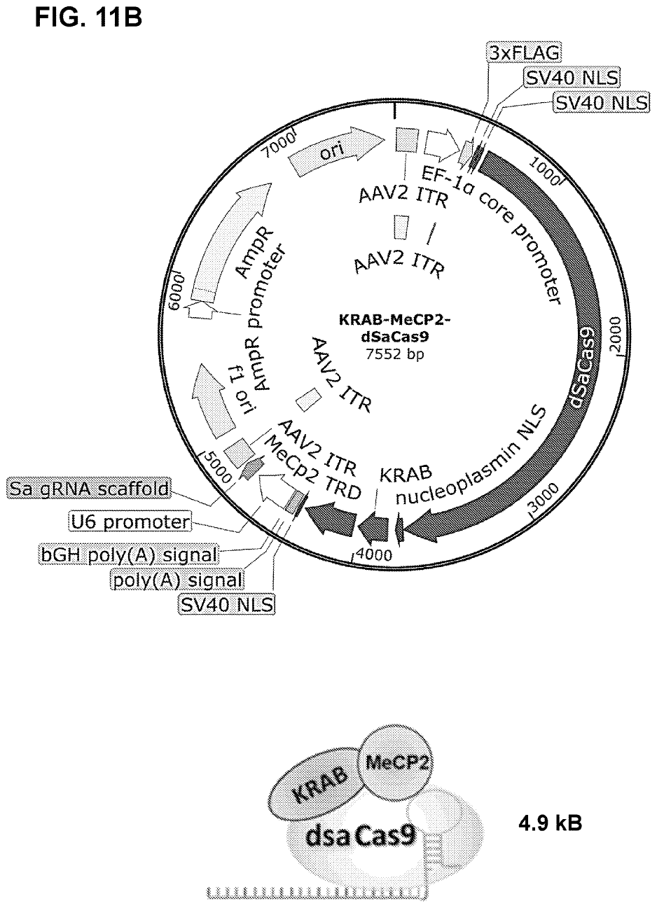

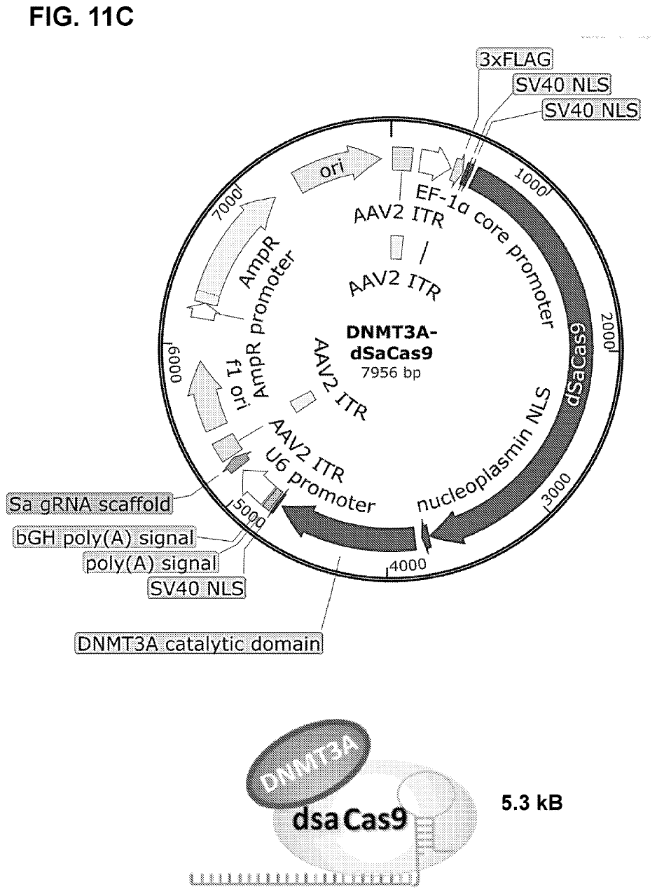

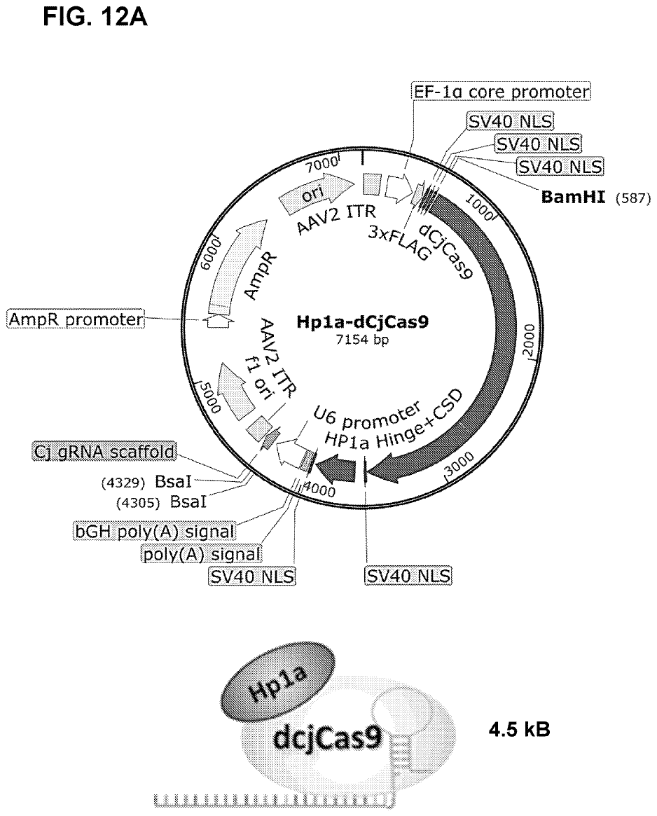

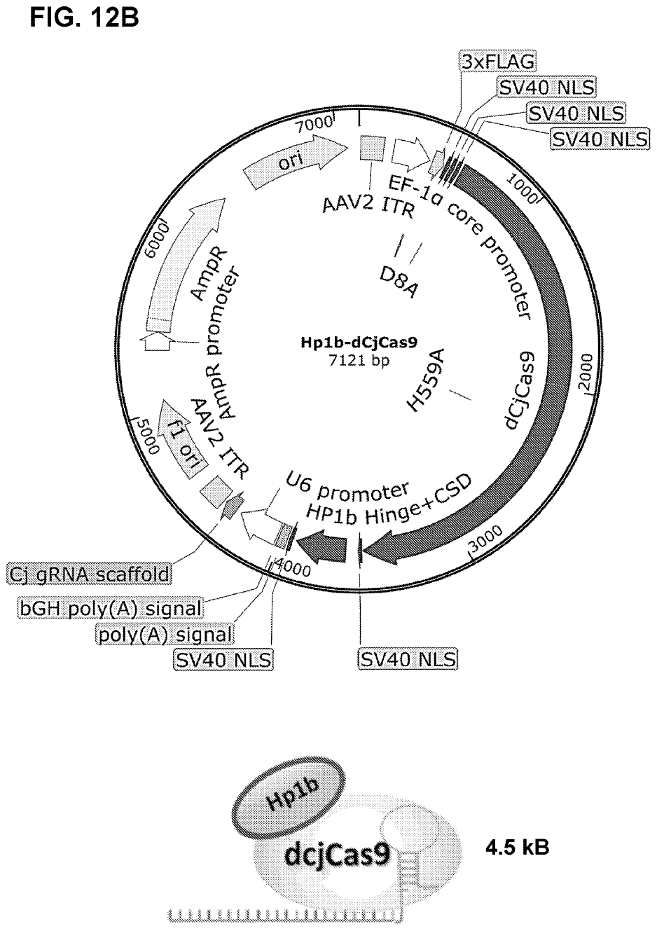

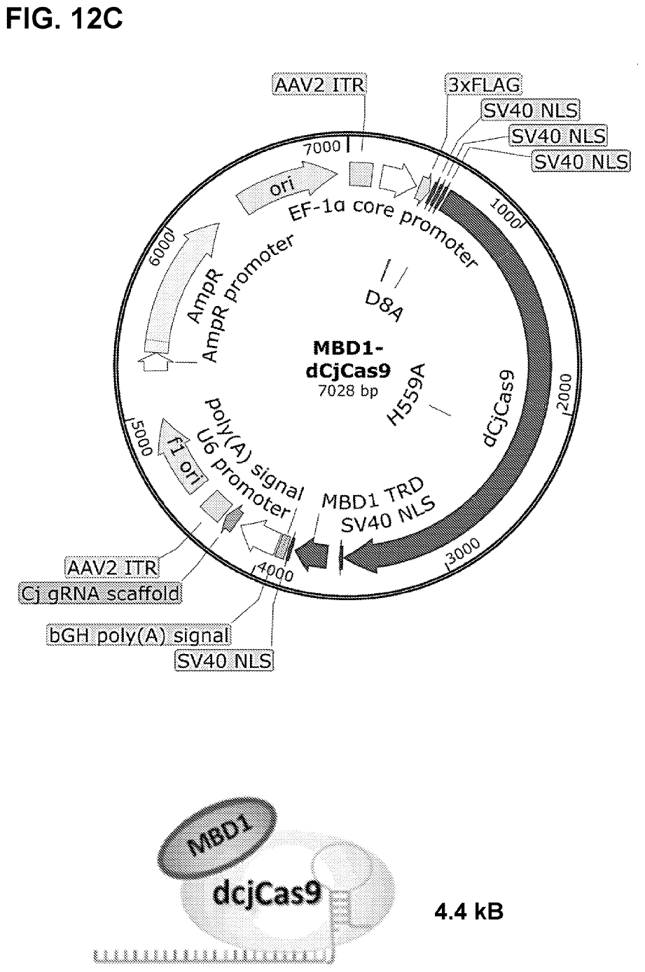

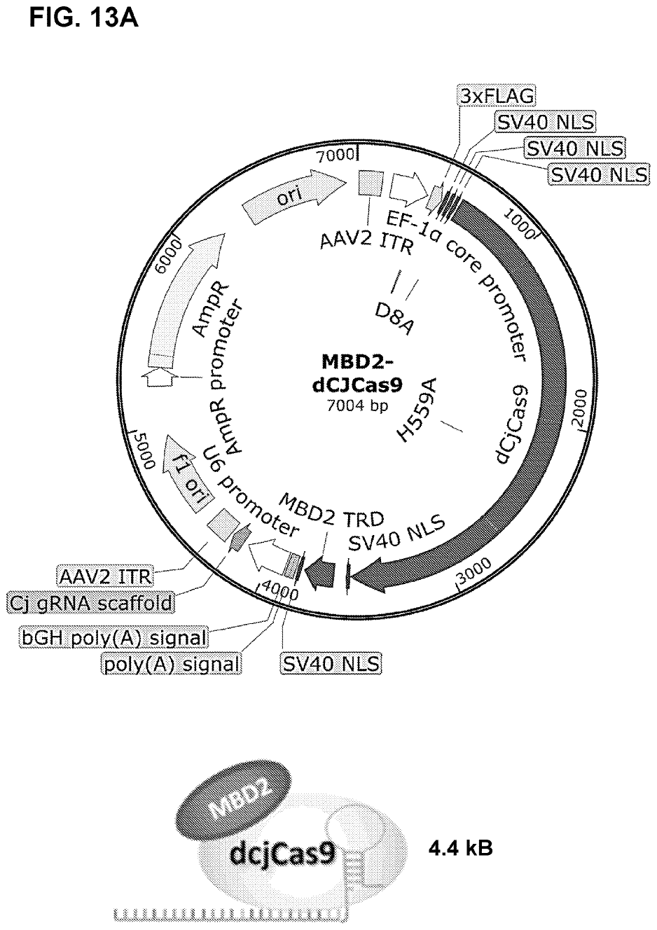

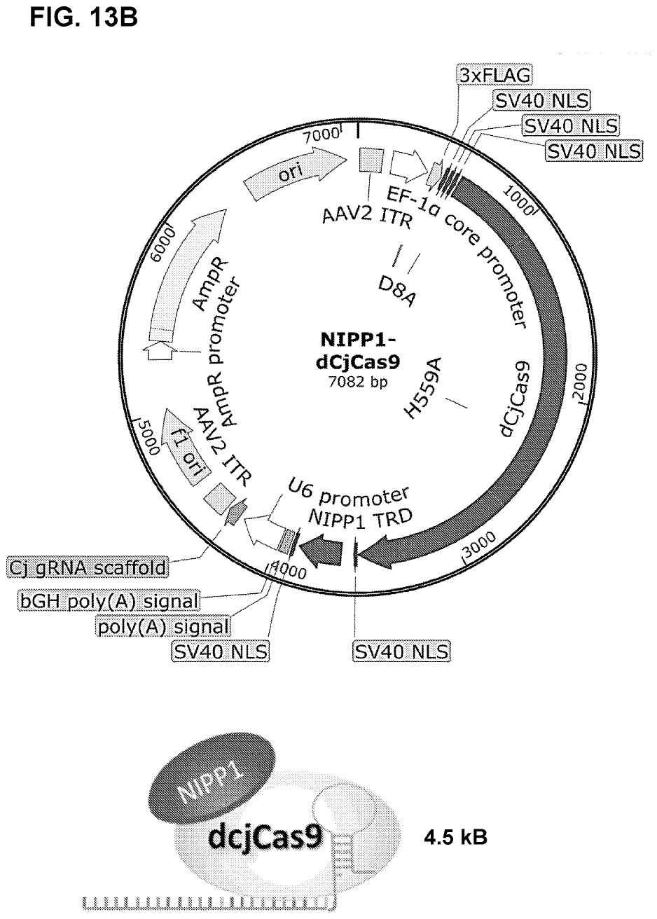

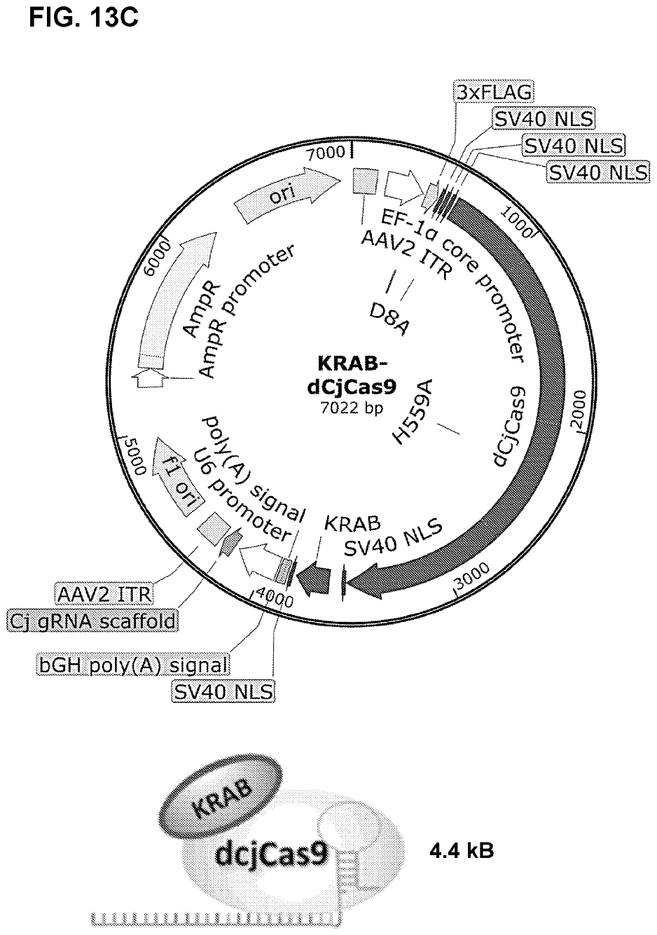

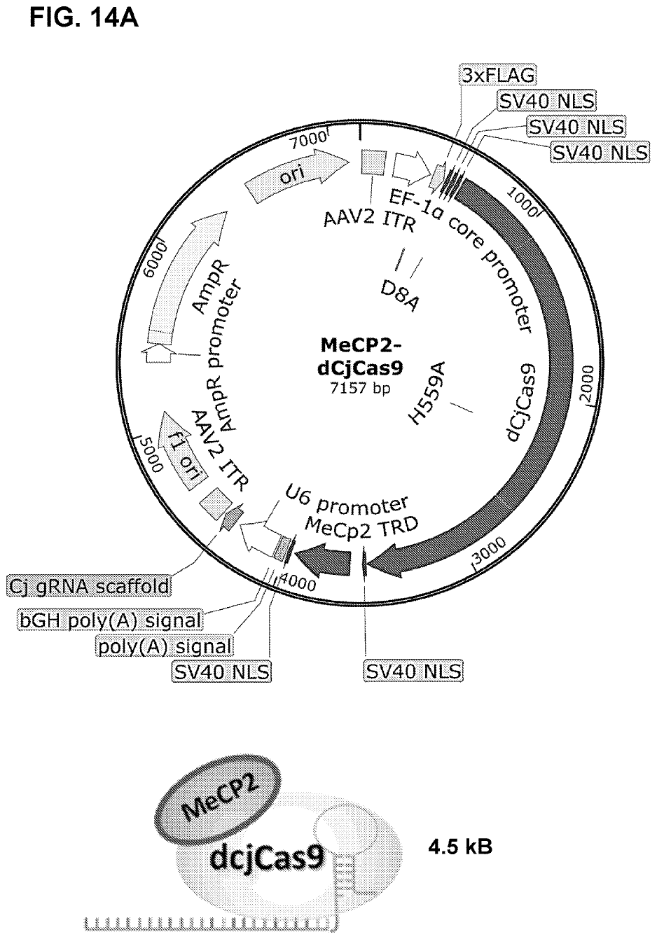

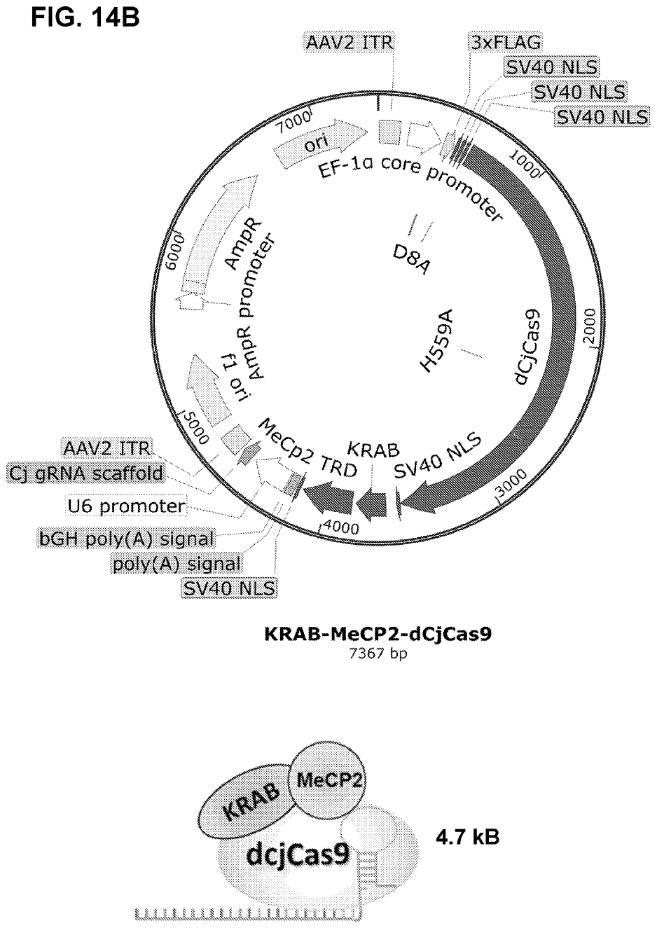

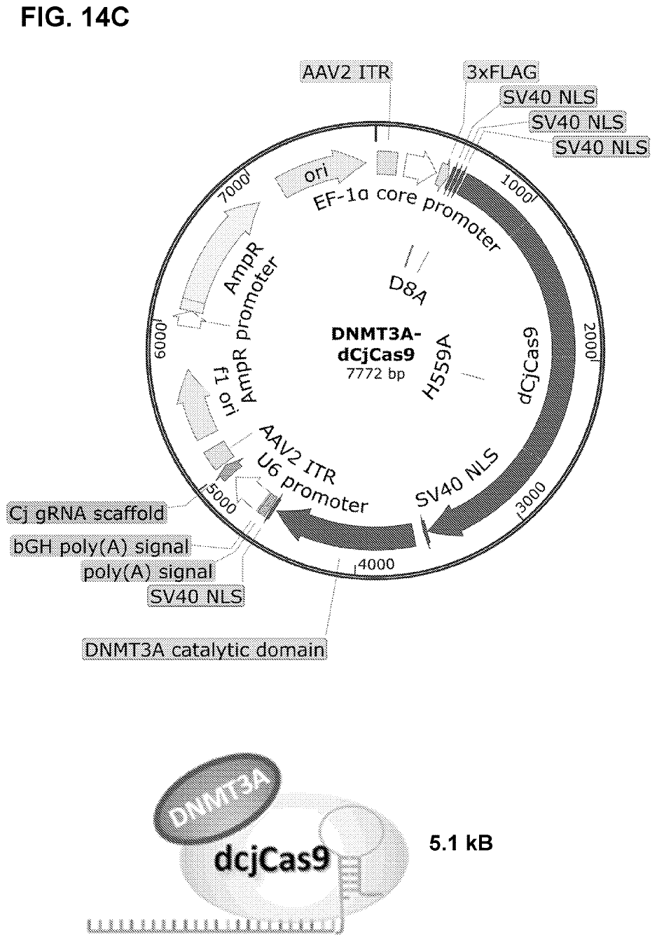

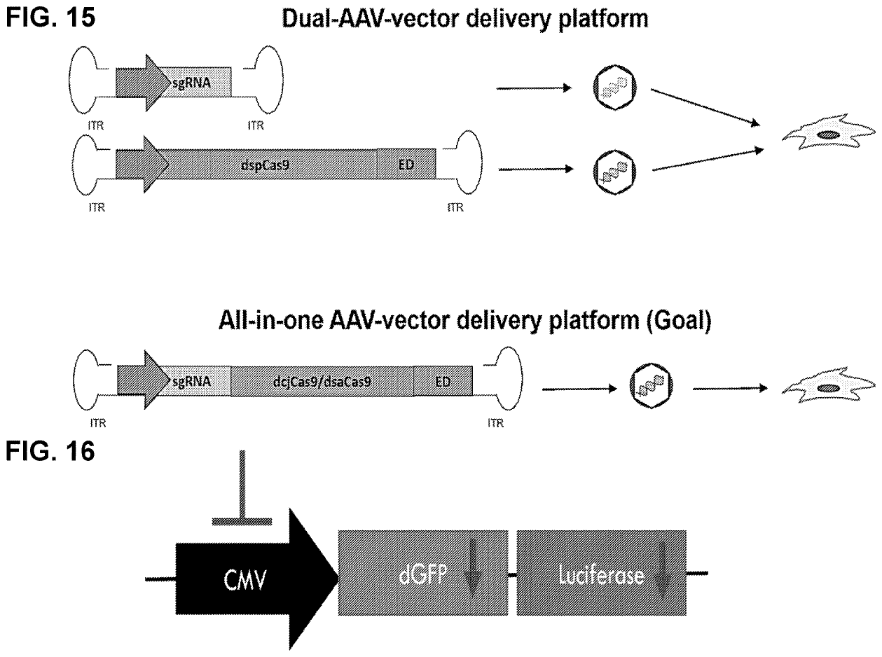

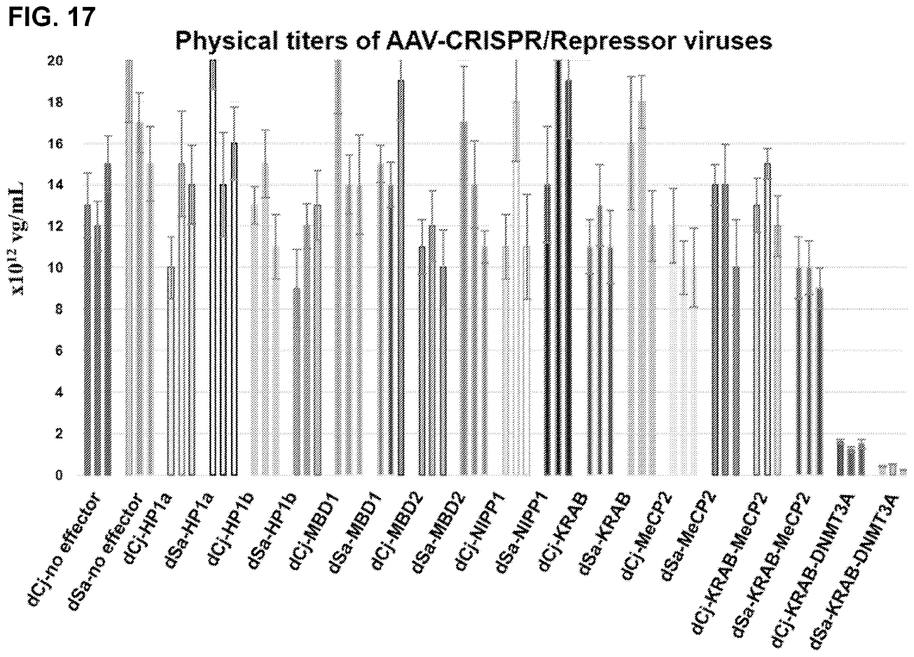

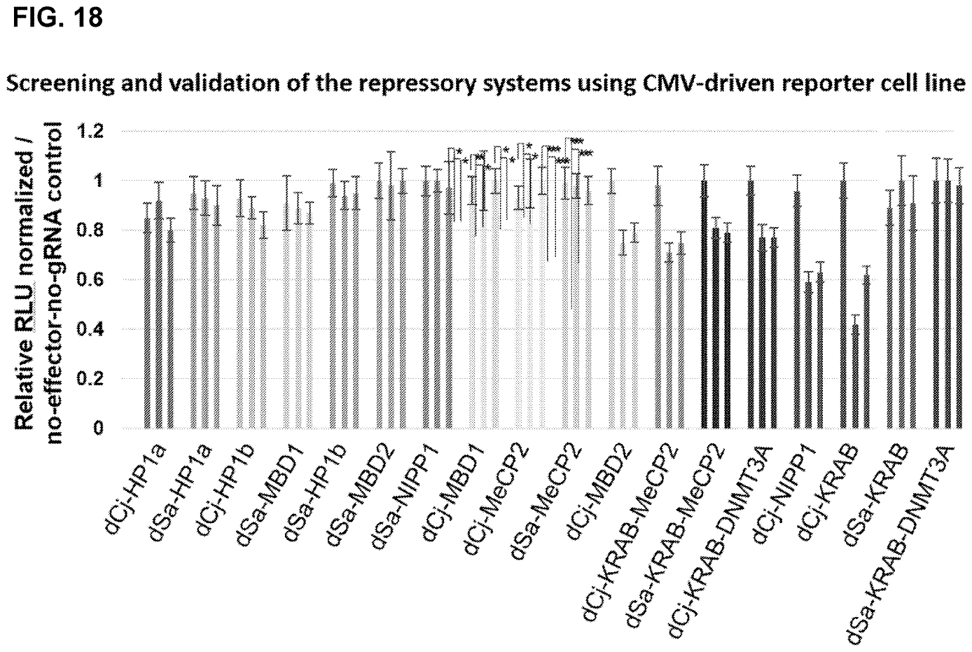

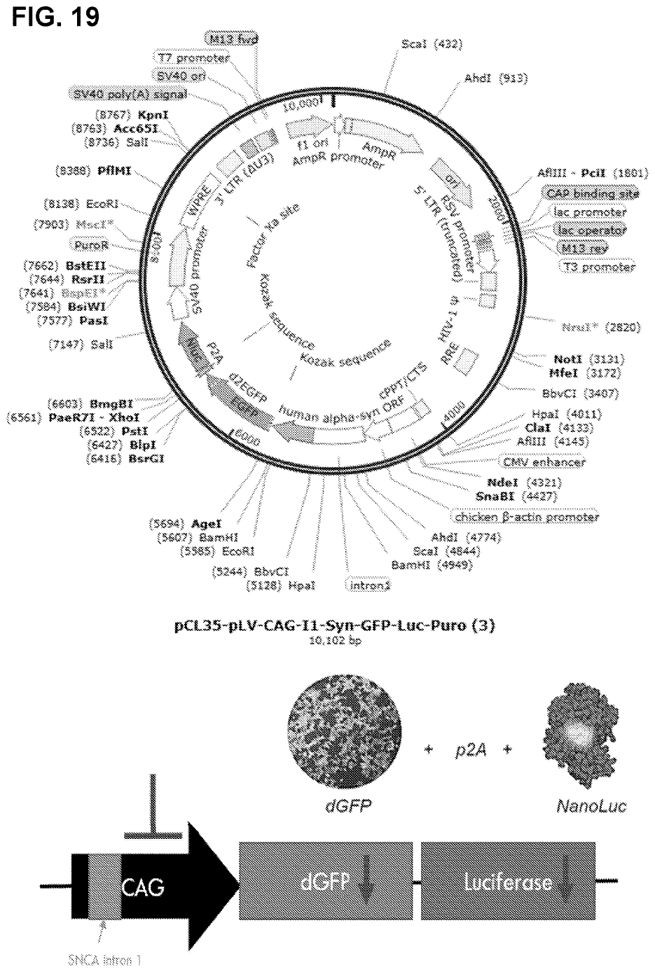

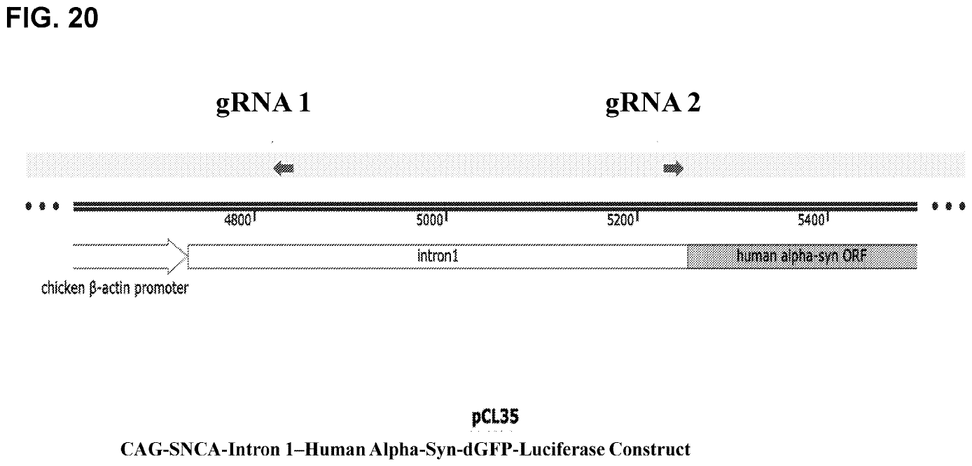

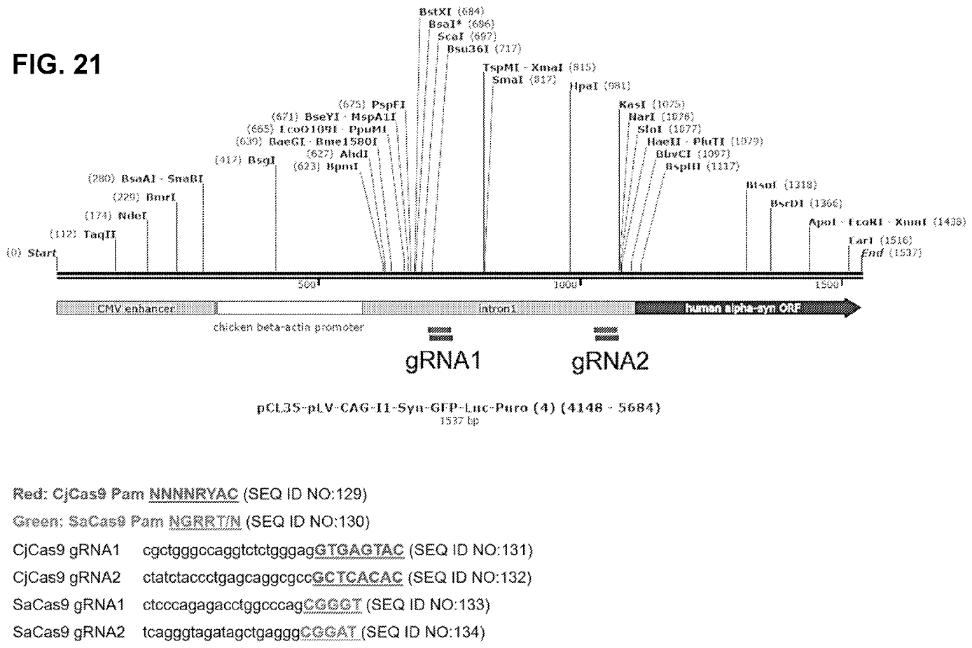

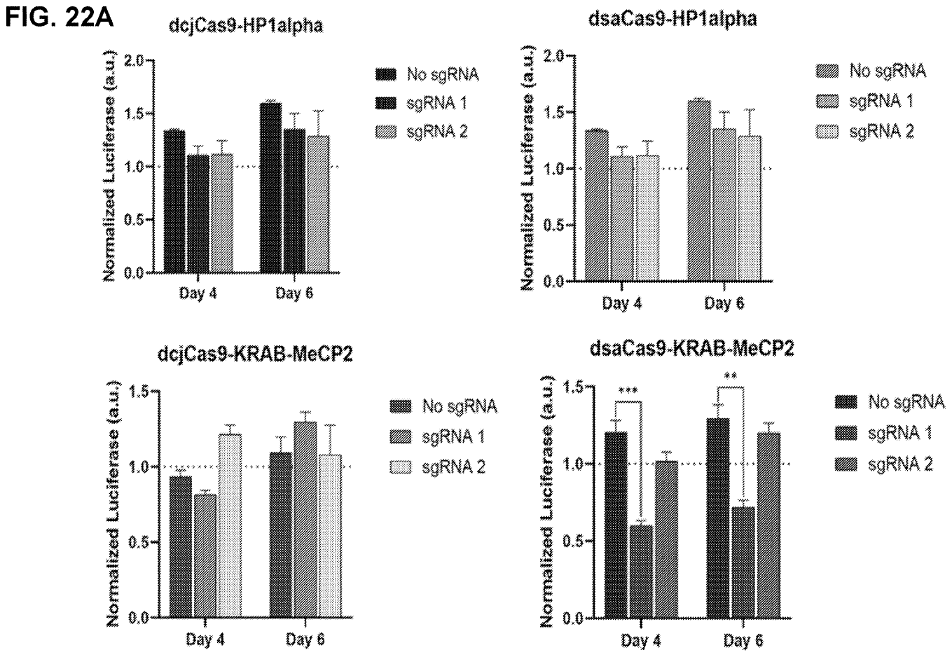

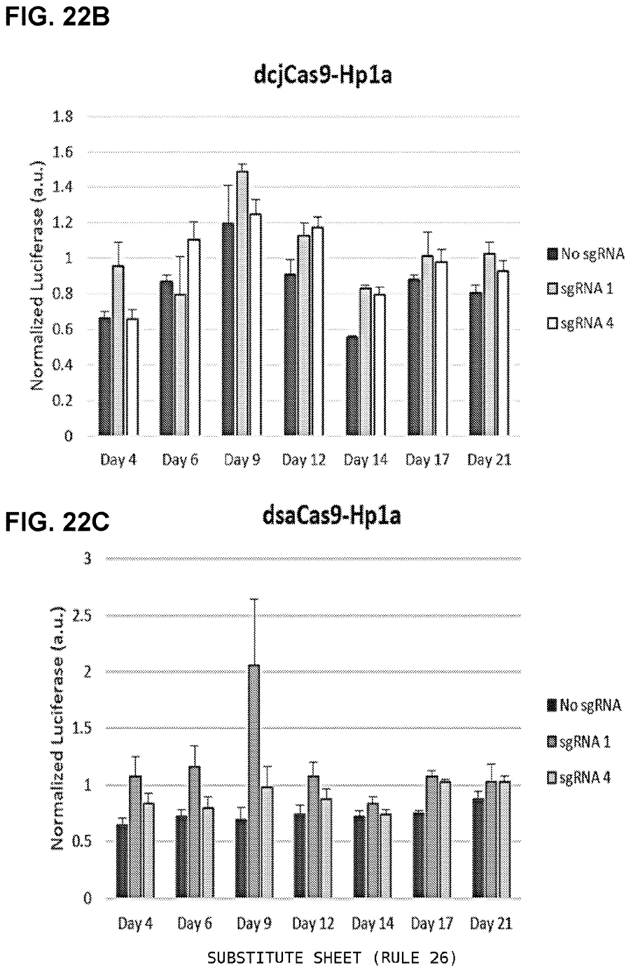

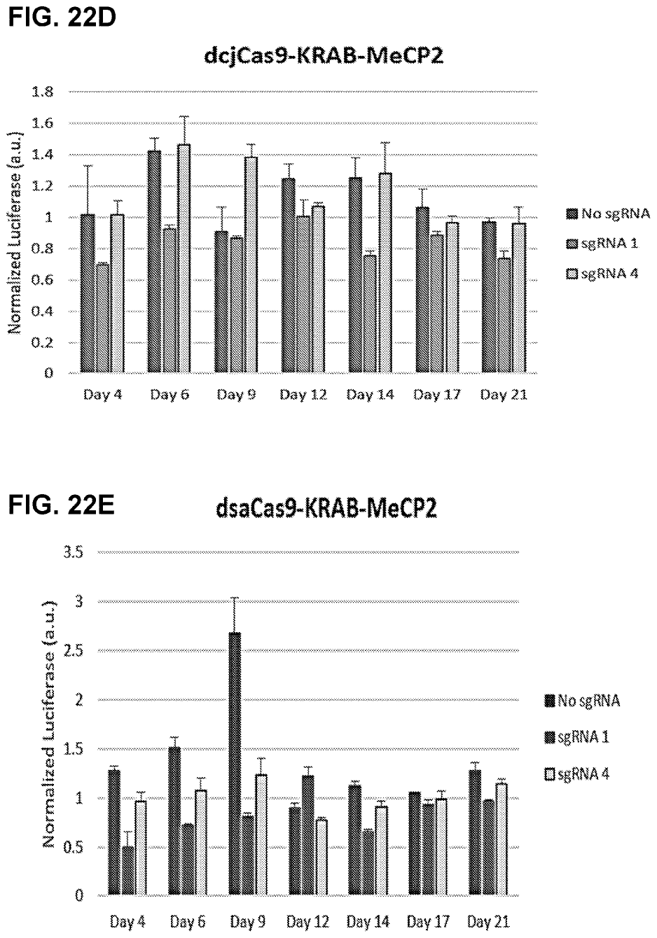

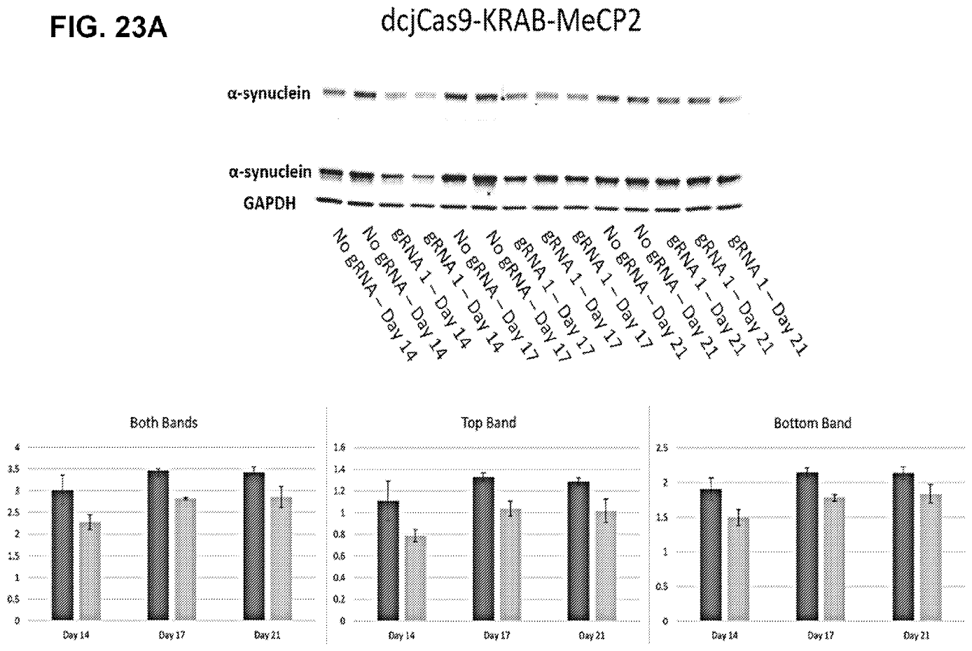

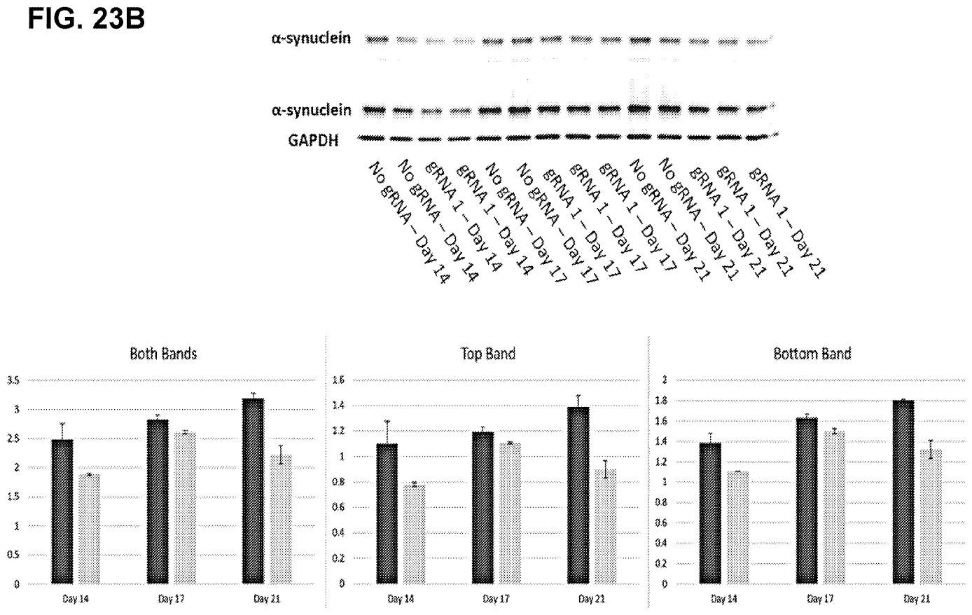

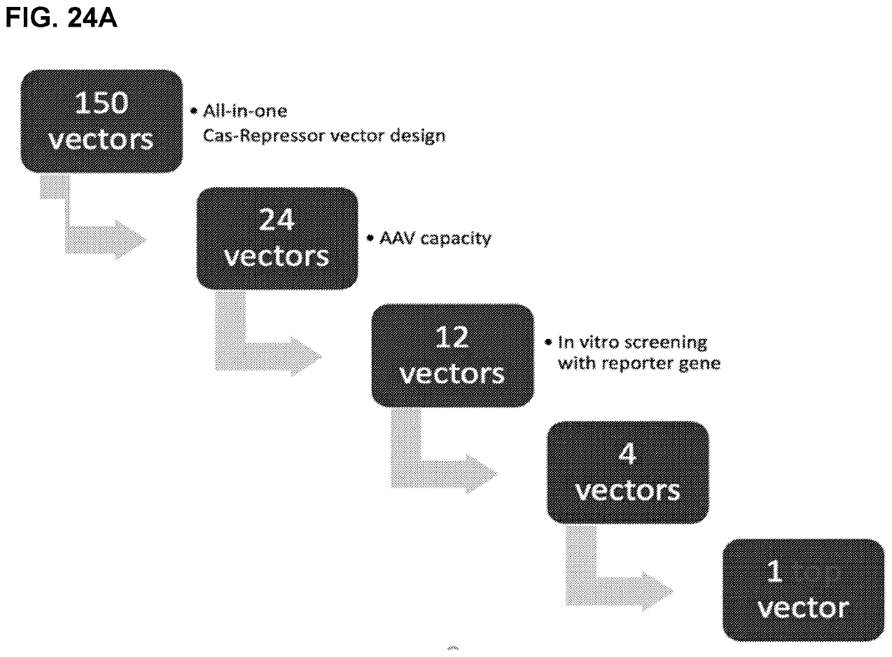

Out of 150 Cas-Repressor pairs, 24 showed efficient packaging in AAV. Those 24AV/dCas9-repressor vectors were screened in vitro using luciferase reporter assay (FIG. 7) to characterize their repression effects on gene expression (FIG. 24A). Four (4) vectors showed greater than 40% repression (FIG. 24B—maroon and grey bars) with the lead selected vector (CasA-repressor 6), demonstrating the most robust reduction (~60%) in a luciferase expression assay (FIG. 24B).

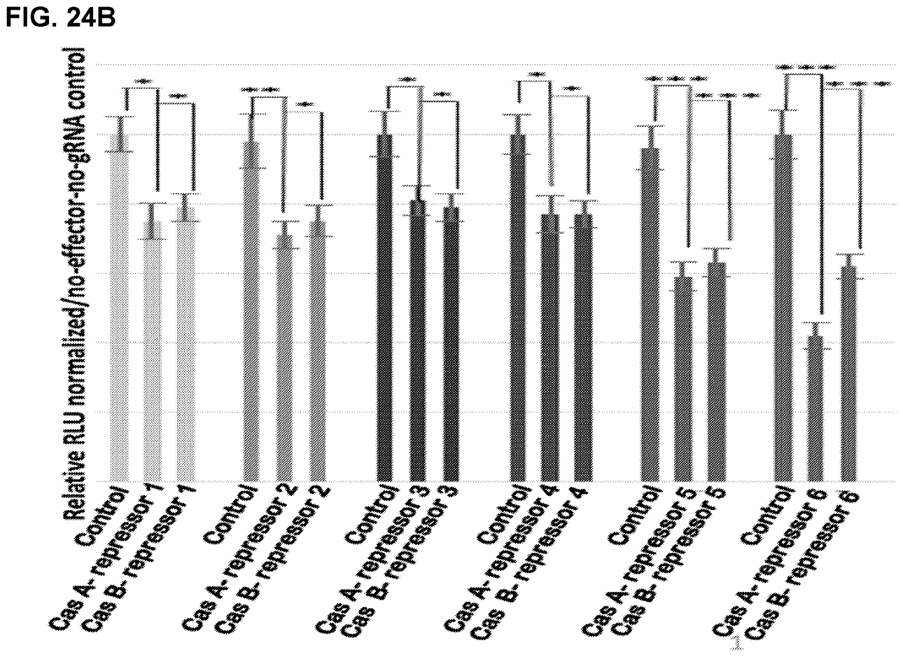

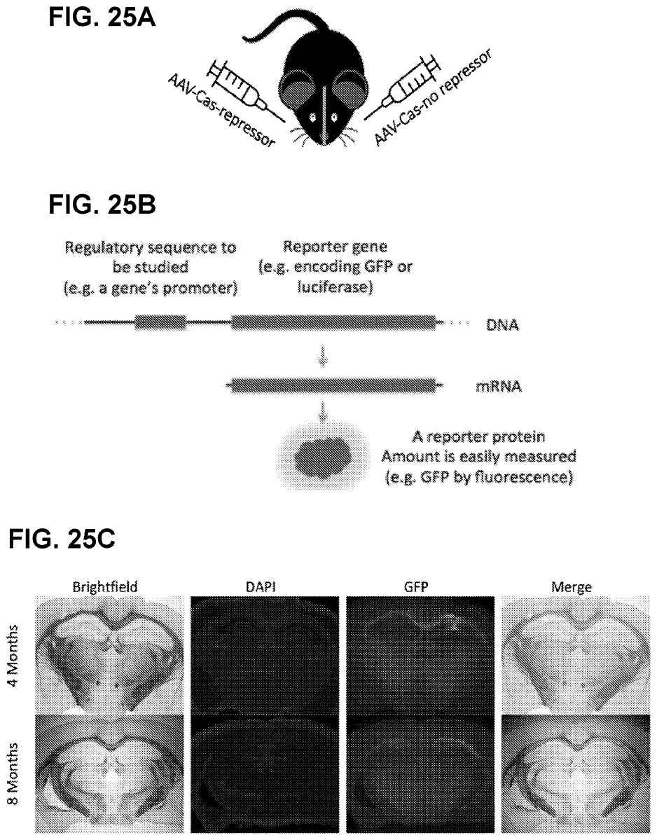

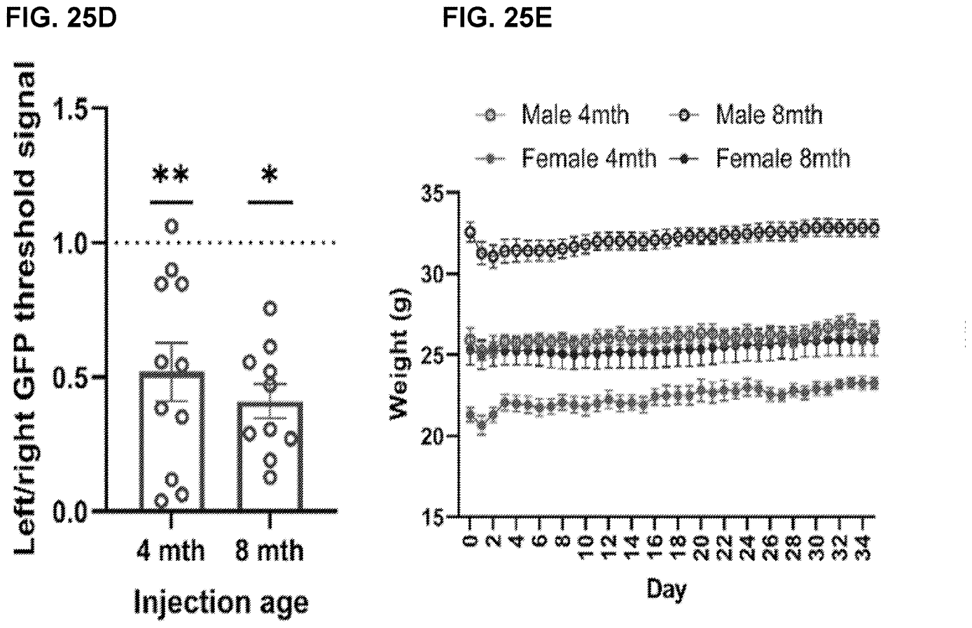

The efficacy and efficiency of the lead candidate AAV/CasA-repressor 6 vector was examined in vivo in mouse brain. A reporter-gene experiment (FIG. 25A) in C57BL/6 mouse hippocampus was performed by bilateral stereotaxic co-injection of the selected AAV/dCas9-repressor platform with a lentivirus GFP-reporter gene vector directly into the left hemisphere of the mouse dorsal hippocampus and the right dorsal hippocampus hemisphere was co-injected with a control AAV/dCas9 vector (no repressor) and the GFP-reporter gene (FIG. 25B). The gRNA was designed to target the promoter of the GFP-reporter gene.

After six weeks mice were sacrificed, and brains were harvested and sectioned for GFP expression analysis. The experiments were performed in two age-groups of C57BL/6 mice: mature adult mice (4 months, n=11) and middle-aged mice (8 months, n=10). A robust decrease in GFP-signal was observed in the left dorsal hippocampus when compared to the right dorsal hippocampus. These data demonstrated strong repression by AAV/CRISPR-dCas9 platform, consistent in the two age groups (FIG. 25C).

The GFP signals were measured, and the left/right ratios were calculated to determine the effect of AAV/dCas9-repressor vector. The results showed lower GFP expression in the left DH relative to the right DH for each mouse. As no significant sex differences in GFP expression were seen, males and females were combined for further statistical analysis. Statistical analysis demonstrated a consistent and significant repression effect of the lead AAV/dCas9-repressor platform on GFP expression in both mice cohorts—the mature mice (n=11, 52% reduction, p=0.004) and the middle-aged mice (n=10, 41% reduction, p=0.028). (FIG. 25D). The results validated (i) the efficient delivery of the all-in-one AAV vector in vivo into the dorsal hippocampus, and (ii) the efficacy of the lead candidate platform to repress gene expression in vivo in mouse-brain.

Additionally, initial safety evaluations were conducted by measuring the daily weights of the mice and monitoring several well-being criterions. While both age and sex (4 months; male=26.09±0.06, female=22.37±0.1, p=0.0003 and 8 months; male=32.17±0.09, female=25.43±0.05, p=0.0001) had significant influence on mouse weights, there was no significant impact of viral vector injection. The mice also displayed normal grooming and eating/drinking behaviors (FIG. 25E). These data provide initial safety evidence.

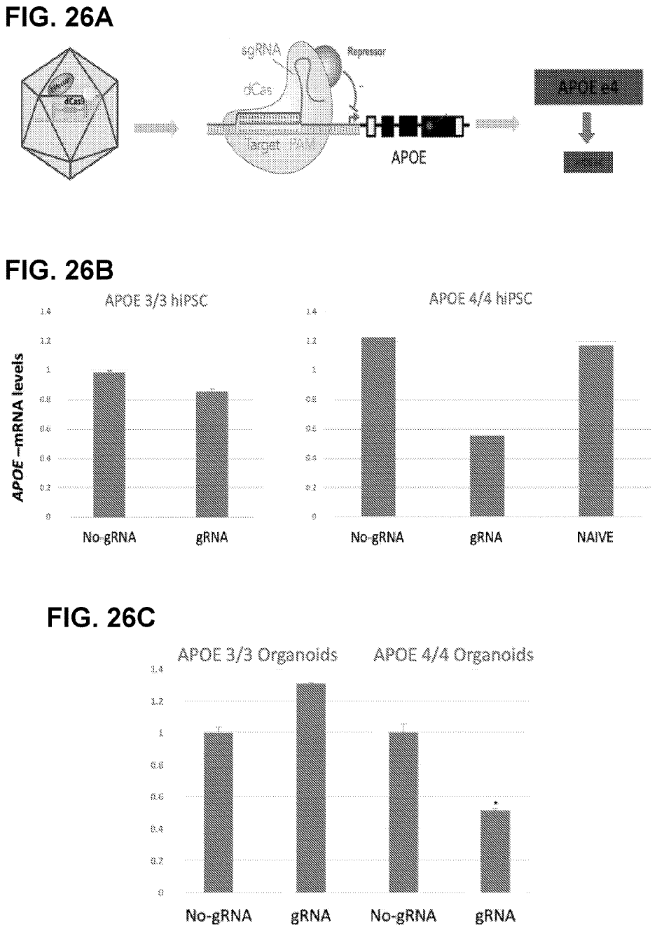

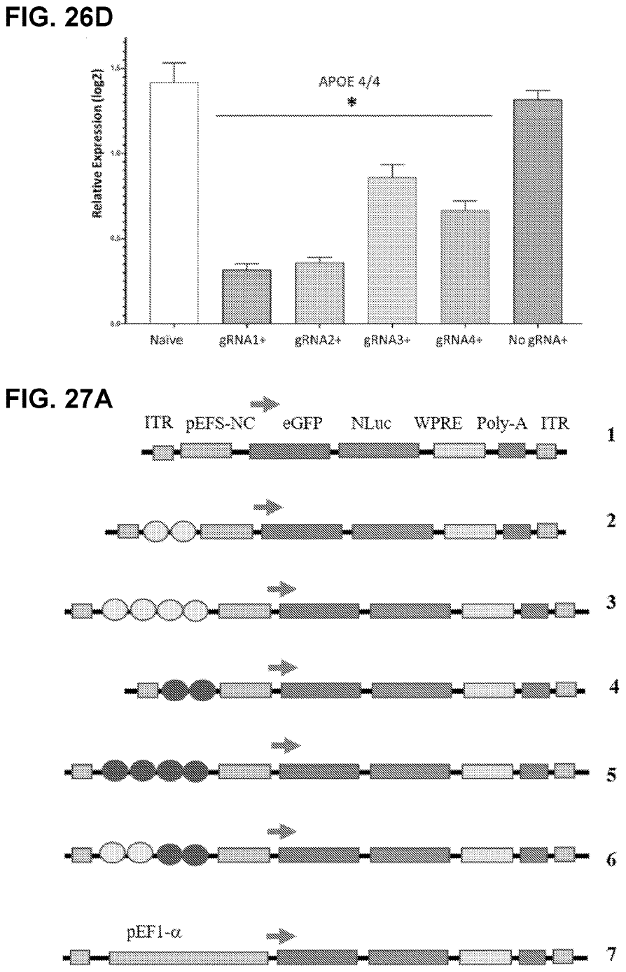

APOE e4-targeted repression in vitro was validated in a human-based induced Pluripotent Stem Cells (hiPSC)-derived model. Target engagement and specificity of the gRNAs fused with the dCas9-variant was tested by transducing the all-in-one gRNA/dCas9-repressor platform (FIG. 26A) into two isogenic hiPSC-lines that differ only by their APOE genotype, homozygous for the e4 allele vs e3. The gRNA/dCas9-repressor vector targeting specifically the APOE e4 allele showed a reduction in APOE-mRNA levels in the APOE e4/4-hiPSC compared to control vector with no-gRNA while there was no effect in the isogeneic APOE e3/3-hiPSC (FIG. 26B). Next, the two isogenic hiPSC-lines, e4/4 and e3/3, were differentiated into cerebellar organoids. The transduction experiment with the e4 allele-specific gRNA/dCas9-repressor vector was repeated. Similarly, a robust reduction in APOE-mRNA levels was observed in the APOE e4/4-derived organoids when compared to the control. There was no difference in APOE-mRNA expression in the isogenic APOE e3/3-derived organoids (FIG. 26C). The consistent results in both 2D and 3D (organoids) models demonstrated the ability of the platform to precisely target APOE and lower its expression as well as the specificity of the effect to the e4 allele. To optimize the lead vector candidate, hiPSC-derived neurons from an AD patient e4-homozygous were transduced with the dCas9-repressor platform harboring four gRNAs targeting different regulatory sequences within the APOE locus (FIG. 26A). All four gRNAs (Table 2) lowered APOE expression with gRNA1 and gRNA2 showing the most robust reduction of APOE-mRNA (FIG. 26D).

TABLE 2
Table of gRNAs.

| Identifier | Sequence |
|---|---|
| SEQ ID NO: 85 | gacagggggagccctataat |
| SEQ ID NO: 92 | tcaggagagctactcggggt |
| SEQ ID NO: 86 | actgggatgtaagccatagc |
| SEQ ID NO: 87 | gttggagcttagaatgtgaa |

### Example 2

Description of an all-In-One CRISPR/dCas9 AAV Vector

Clustered Regularly Interspaced Palindromic Repeats (CRISPR)/dCas9-based epigenetic editing is an emerging field in developing safe and effective gene engineering tools. The system consists mainly of a single guide RNA (sgRNA) and a deactivated-Cas9 nuclease (dCas9).

Importantly, epigenome-editing applications supported via AAV delivery require an efficient pairing of dCas9 with small effector molecules or a fused effector molecule. This is not straightforward to achieve considering that CRISPR epigenetic engineering tools developed so far generally induce lower fold changes than standard CRISPRa/CRISPRi systems delivered via the plasmid or lentiviral vectors (Schmidt F, et al. (2015). Biotechnol J. 10(2):258-272; Mingozzi F, et al. (2011) Nat Rev Genet. 12:341-355; Dropulić B. (2011) Hum Gene Ther. 22(6):649-657).

Due to the small packaging capacity of adeno-associated viral (AAV) vectors, the most common technique to deliver the CRISPR/dCas9-based transcriptional repression system is through a dual-AAV-vector delivery platform. In this system, the sgRNA and dCas9 are packaged in separate AAVs, which increases the viral production need and decreases efficacy in gene repression. As such, the development of all-in-one AAV systems harbored multiple effectors paired with dCas9 is somewhat more challenging process. Furthermore, the physical and functional titers of AAVs are more sensitive to the transgenic size and composition. As such, optimization of the viral backbone in cis is required for efficient manufacturing of the AAV vector systems, especially those delivering bulky and complex transgenes.

The design and implementation of an Sp1-NF-kB harbored vector backbone carrying a compact CRISPR-dCas9-based transcriptional repression tool that can fit all-in-one within a single AAV vector to achieve efficient epigenetic editing is described herein. This novel system has shown efficient and long-term repression in the neurological disease—relevant settings in vitro and in vivo. The development of this robust epigenetic editing tool enables novel architectures for synthetic gene circuits and potential safe gene therapy for several diseases.

### Example 3

Identification of dCas9 Variant and Repressor Domain

The addition of Sp1 and NF-kB binding sites was found to correspond with higher packaging efficiency and expression of IDLV vectors used for in vitro and in vivo CRISPR/Cas-9 delivery. These modified vectors remained episomal and showed lower levels of undesirable off-target effects when compared with integrase-competent LVs (ICLVs) (Yang Y, et al. (2016) Nat Biotechnol. 34:334-338). IDLV shows a great deal of the similarity to AAV and other non-integrating vectors in terms of the genetic organization and inherent prone-silencing propensity.

Like LVs, recombinant, rAAVs carry no Sp1 and NF-kB sites in the expression cassettes comparing to the wild-type counterparts (wtAAV) (Lau C H, et al. (2017) F1000Res. 6:2153; Epstein B E, et al. (2017) Adv Exp Med Biol. 1016:29-42; Mendell J R, et al. (2017) N. Engl. J. Med. 377:1713-1722.

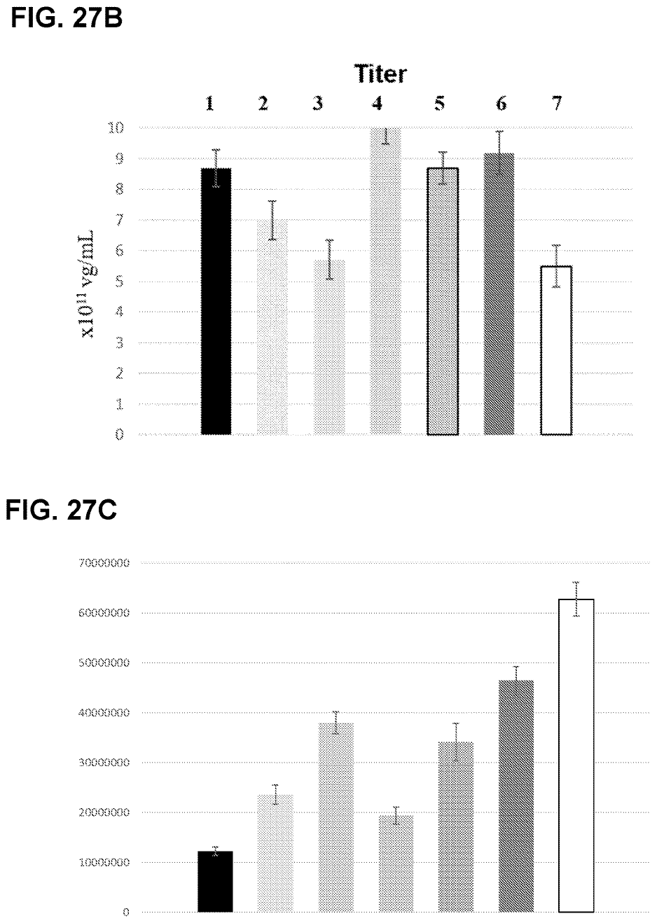

To improve packaging efficiency of AAV vectors, binding sites for the transcription factor Sp1 and NF-kB were reinstated into the expression cassette of the vector. To that end, two copies of the Sp1 and NF-kB (AAV-2×Sp1 or AAV-2×NF-kB) were cloned into the AAV expression cassette carrying a dual reporting system (eGFP and NLuc). Similarly, four copies of the Sp1 (AAV-4×Sp1) and NF-kB (AAV-4×NF-kB) were cloned into the AAV expression cassette carrying the same dual reporting system. Finally, 2 copies of Sp1 and NF-kB (AAV-2×Sp1-2×NF-kB) were cloned into the AAV expression cassette carrying the dual reporter system. (FIG. 27A).

The transcription binding sites were inserted upstream of the core version of EF-1a promoter (EFS-NC), which had neither Sp1 nor NF-kB sites. The complete EF-1a promoter having multiple Sp1 and NF-kB sites was used as a control. The strong, but bulky (~1500 bps) EF-1a promoter is restrained from the use with AAV vectors. Conversely, a miniature EFS-NC promoter (212 bps long) was highly suitable for the delivery with the AAV systems. The designed vectors were produced in the non-concentrated format and tittered by real-time PCR.

As shown in FIG. 27B, no significant difference in the physical titers of the vectors was detected. Next, the produced vectors were used for the transduction into HEK293T cells at the MOI=10,000. As shown in FIG. 27C, the vectors carrying Sp1 and NF-kB binding sites demonstrated a higher level of NLuc expression when compared to the naïve counterpart. Furthermore, the vectors carrying four repeats showed higher level of expression when compared to those vectors carrying two repeats of the transcription factor binding sites (FIG. 27C).

Furthermore, the vector carrying a repeat of both Sp1 and NF-kB cloned upstream of EFS-NC promoter in tandem showed slightly higher level of the reporter expression, nearly reaching the capacity of the vector expressed NLuc from EF-1a promoter (FIG. 27C). DNMT3A was too bulky to be efficiently packaged into an all-in-one AAV.

Here, the development of an all-in-one system capable of efficient AAV-packaging and expression was undertaken.

To that end, the following repressors were selected for screening: Krüppel-associated box (KRAB); methyl CpG binding protein 2-transcription repression domain (MeCP2-TRD), heterochromatin proteins HP1a and HP1b, methyl-CpG binding proteins 1 and 2 (MBD1 and MBD2), nuclear inhibitor of PP1′ (NIPP1), and the fused version of KRAB-MeCP2-TRD. These effector molecules were paired with two small dead or deactivated Cas9 (dCas9)—Staphylococcus aureus (SaCas9) and Campylobacter jejuni (CjCas9).

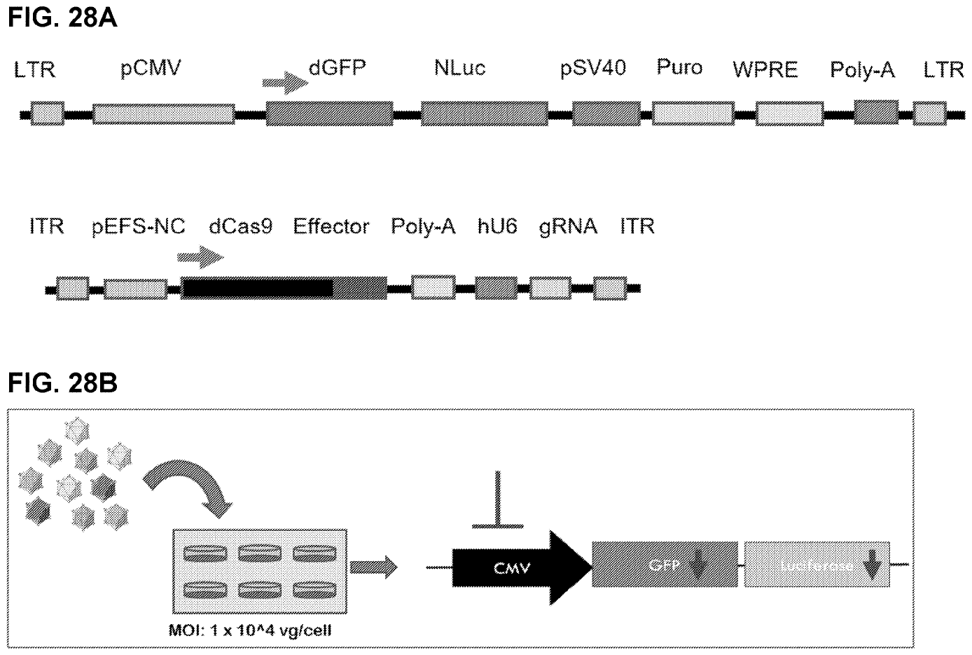

The vectors were transduced into HEK293T cells stably introduced with lentiviral vector expressing CMV-destabilized GFP (dGFP)-NLuc. Two gRNA targeting different regions of CMV promoter were used to test the repression capacity of the vectors. The initial assessment of the repression capacity of the constructs was done by the visualization of the dGFP expressions. the data were then quantified using the NLuc testing (FIG. 28A-FIG. 28B).

To first validate the constructs, all eight plasmids and no sgRNA equivalents, and the control groups were introduced to HEK293T/pBK1340 cells by lipofectamine 3000 transfection. After 4 days of transfection, the cells were harvested and NanoLuc and BCA assays were performed. The measured luciferase levels were compared against the control group of no-sgRNA-dCjCas9 and no-sgRNA-dSaCas9.

The results showed that constructs containing KRAB-MeCP2 constructs had the most significant downregulation of gene expression. Specifically for construct dCjCas9-KRAB-MeCP2, the luciferase levels in both sgRNA constructs were statistically lower compared to the no sgRNA control group (amounting to ~40% reduction (p<0.001, Student's t test)).

To further test the efficiency of the constructs, each construct was packaged and concentrated in AAV9 and transduced HEK293T/pBK1340 cells. The cells were harvested over a course of 2 weeks and NanoLuc and BCA assays were performed.

Constructs containing HP1A did not have any significant transcriptional repression, while constructs containing KRAB-MeCP2 generated statistically lower the luciferase production.

AAV/sgRNA1-dSaCas9-KRAB-MeCP2 demonstrated a long-lasting effect of up to 6 days, with the most significant effect on day 6 (~50% reduction (p<0.0001, Student's t test)).

It was evident that the reduction in luciferase levels for groups with sgRNA decreased over time, which could be a result of the rapid dividing nature of HEK293T cells and/or the transient expression of AAVs. Overall, these sequential experiments indicated that out of the two transcriptional repressors, bipartite repressor KRAB-MeCP2 was the most efficient, while out of the two selected dCas9 proteins, dSaCas9 has proven to be the effective.

These experiments describe the development of a compact epigenetic editing tool, which includes a sgRNA, a CRISPR/dCas9 nuclease, and the catalytic domain of the bipartite repressor domain KRAB-MeCP2. This system achieved effective transcriptional repression when entirely packaged within a single AAV system.

Through qPCR, it was found that even by day 21, there was minimal integration of the AAV viral genome in the transduced cells, which reflects the prolonged transgene expression properties of AAV. This characteristic, along with its immunogenicity and wide spectrum of serotypes or high tissue tropism, makes AAV a desirable vehicle for gene editing therapies.

For neurodegenerative diseases, AAV is an ideal platform for viral gene delivery into the central nervous system. AAV pseudotypes, such as AAV2/5 and AAV2/1, demonstrate high affinity for neuronal and glial cells, while AAV2/9 has shown to cross the blood brain barrier of mice and cats when administered intravenously (Rittiner J E, et al. (2020) Front Mol Neurosci. 13:148).

In conclusion, CRISPR/Cas9 is a powerful gene editing tool in modifying genes or regulating gene expression. With the increasing understanding in the genetic risk factors of neurodegenerative diseases, such as SNCA gene for Parkinson's Disease and APOE for Alzheimer's Disease, gene therapy is a potential therapeutic strategy for preventing diseases or inhibiting disease progression.

### Example 4

### Long Lasting and Targeted Repression of SNCA Intron 1

The hypomethylation of the SNCA intron 1 lead to reduction of the endogenous expression level of SNCA mRNA and α-synuclein protein. Similarly, PD-related cellular phenotype characteristics in SNCA triplication hiPSC-derived dopaminergic neurons were reduced, which lead to a decrease in mitochondrial ROS production and an increase in cellular viability (Kantor B, et al. (2018) Mol Ther. 26(11):2638-2649).

Like the CMV experiment, we designed cell line HEK293T/pCL35 to express CAG-α-synuclein-dGFP-luciferase and analyzed the same four dCas9 variant and repressor domain combinations (dCjCas9-HP1A, dCjCas9-KRAB-MeCP2, dSaCas9-HP1A, and dSaCas9-KRAB-MeCP2). Two sgRNAs were designed to target SNCA intron 1, the same target site in previous studies. By targeting SNCA intron 1, we hope to test whether the compact CRISPR/dCas9 based transcriptional repressor in an all-in-one AAV could achieve a significant level of downregulation in dGFP, luciferase, and α-synuclein production.

First, the plasmid constructs were introduced to HEK293T/pCL35 cells by lipofectamine 3000 transfection. After 4 days and 6 days of transfection, the cells were harvested and NanoLuc and BCA assays were performed. Like the CMV experiments, the results showed that construct sgRNA1-dSaCas9-KRAB-MeCP2 had the most significant reduction of luciferase levels by ~40% on both days (p<0.0001 on day 4, p<0.001 on day 6, Student's t test). However, there was no statistically evident downregulation of luciferase levels in the dCjCas9-HP1A group, the dCjCas9-KRAB-MeCP2 group, or the dSaCas9-HP1A group.

Then, the effect of the CRISPR/dCas9 system delivered through AAV was evaluated. The construct was packaged into AAV9 and HEK293T/pCL35 cells were transduced. The cells were harvested over a course of 3 weeks and NanoLuc and BCA assays were performed. Constructs containing HP1A did not have any significant transcriptional repression, while those constructs containing KRAB-MeCP2 had an evident reduction in luciferase levels. In particular, AAV/sgRNA1-dSaCas9-KRAB-MeCP2 was shown to have an impressive long-lasting effect of up to 21 days. At day 21, the luciferase levels in cell group treated with AAV/sgRNA1-dSaCas9-KRAB-MeCP2 were ~25% lower than the control group (p=0.01, Student's t test).

Downregulation in SNCA Protein Levels and Minimal AAV Vector Integration. To further verify the long-lasting effect of the compact CRISPR/dCas9-based system with KRAB-MeCP2 in an all-in-one AAV system, the downstream α-synuclein protein expression levels in HEK293T/pCL35 cells that were transduced with AAV/sgRNA1-dCjCas9-KRAB-MeCP2 and AAV/sgRNA1-dSaCas9-KRAB-MeCP2 were examined on days 14, 17, and 21. These levels were then compared with the control no gRNA group through Western Blot.

The results showed that there was a significant reduction in α-synuclein protein levels on days 14, 17, and 21, further confirming the stable long-lasting effect of this system. Specifically, HEK293T/pcL35 cells treated with AAV/sgRNA1-dCjCas9-KRAB-MeCP2 had a reduction of ~30% in α-synuclein protein levels on day 21 (p=0.0066, Student's t test).

To verify that this long-lasting expression of α-synuclein protein reduction is not due to AAV vector integration, qPCR was performed for dCjCas9 and dSaCas9 for the same samples examined through Western Blot.

If the AAV were to integrate into the cells, then there would be a significant replication in dCjCas9 or dSaCas9 from the integrated viral genome. It was found that past cycle 30 on the qPCR, there were no replication of dCjCas9 and dSaCas9 sequences, which indicated minimal AAV vector integration. This indicates that in consideration of the transient properties of AAV, KRAB-MeCP2 was able to potentially create an epigenetic medication that no longer required the presence of CRISPR/dCas9, leading to a stable and long effect.

### Example 5

### In Vivo Validation of the Efficacy and Specificity of Lead AAV-Vector Candidates to Reduce the APOE-e4 Expression

In this experiment, knock-in mouse models in which the endogenous murine Apoe gene is replaced with the human APOE gene either the e4 allele (B6.129P2-Apoe^{tm3(APOE*4)Mae} N8 (hereafter E4KI)) or e3 allele (B6.129P2-Apoe^{tm3(APOE*4)Mae} N8 (hereafter, E3KI)). (Sullivan P M, et al. (1997). J Biol Chem 272:17972-17980). The E4KI mice express only the human APOE e4 while the E3KI mice only express the human APOEe3 gene. The genetic background of the mice is C57BL/6.

### Example 6

Determination of the Efficient gRNA and Optimal Dosage of the AAV Vector to Obtain Robust Overexpression

As detailed below in Table 3, adult E4KI mice (4.5 months old) are divided randomly into 7 groups with each group having an equal amount of males and females).

TABLE 3
Experiment Design

| | Treatment | Left DH | Right DH |
|---|---|---|---|
| (i) | Control | Saline | Saline |
| (ii) | Low Dose | AAV-gRNA1/ | AAV-gRNA1/dCas9-NO |
| | (2 × 10⁹ vg) | dCas9-repressor | repressor |
| (iii) | Medium Dose | AAV-gRNA1/ | AAV-gRNA1/dCas9-NO |
| | (1 × 10¹⁰ vg) | dCas9-repressor | repressor |
| (iv) | High Dose | AAV-gRNA1/ | AAV-gRNA1/dCas9-NO |
| | (5 × 10¹⁰ vg) | dCas9-repressor | repressor |
| (v) | Low Dose | AAV-gRNA2/ | AAV-gRNA2/dCas9-NO |
| | (2 × 10⁹ vg) | dCas9-repressor | repressor |
| (vi) | Medium Dose | AAV-gRNA2/ | AAV-gRNA2/dCas9-NO |
| | (1 × 10¹⁰ vg) | dCas9-repressor | repressor |
| (vii) | High Dose | AAV-gRNA2/ | AAV-gRNA2/dCas9-NO |
| | (5 × 10¹⁰ vg) | dCas9-repressor | repressor |

AAV Vectors: The AAV-gRNA/dCas9-repressor and the control vectors are manufactured in in vivo grade as previously described (Grieger J C, et al. (2006). Nat Protoc 1:1412-1428). The AAV vectors for the injections are quantified by real-time PCR (SYBR Green qPCR) method and by flow cytometry (to determine the functional titers) (Ortinski P I, et al. (2017). Molecular Therapy—Methods & Clinical Development 5:153-164).

Mice Stereotaxic Surgery: Adult mice, age 4.5 months, receive bilateral stereotaxic microinjections of the virus into the dorsal hippocampus (DH; 1.75 mm anterior, ±1.5 mm lateral, and 1.55 mm ventral). AAV/dCas-repressor (1 μL) is injected into the left DH, and AAV/dCas-no repressor (1 μL) is injected into the right DH, providing a within animal control (FIG. 25A). Post-operative monitoring occurs every day for 10 days after viral infusion. Each mouse is evaluated on the at least the following well-being criterions: (i) incision site, (ii) behavior and appearance, (iii) eating, (iv) drinking, (v) feces and urine, and (vi) whether additional analgesia or intervention is needed.

Molecular Analyses: Six weeks post-surgery, the experiment is terminated, and the brain tissues are harvested and used to perform molecular analyses. Analyses focus on the DH while the cerebellum serves as a control brain region. Measures of molecular phenotypes include: (i) human APOE-mRNA level as quantified by qRT-PCR using TaqMan assay, and (ii) human APOE protein expression as measured by Western Blot (WB) and ELISA.

Statistical Analyses: For each group of mice, the effect of the AAV-gRNA/dCas9-repressor vector on the APOE e4 expression is determined by calculating for each mouse the normalized expression levels (mRNA or protein) in the left DH when compared to the right DH. The average of the left DH/right DH ratios of the mice within a group (mean±SEM) is calculated and the statistical significance of the expression change. The average for the left DH compared to that of the right DH within each group is analyzed by Mann-Whitney U Test using GraphPad Prism 9.0. the difference in the efficacy between the various groups of gRNA by dose combinations (i.e., group (ii)-group (vii)). The control group (i) serves as the baseline for the APOE e4 levels. The significance of the differences in APOE e4-mRNA and protein repression between the groups is analyzed using two-way ANOVA via GraphPad Prism 9.0. The Bonferroni and Tukey's post hoc tests are utilized for correction for multiple comparisons.

Expected Outcomes: This experiment determines the efficacy of the epigenome-editing approach using AAV-gRNA/dCas9-repressor to repress APOE expression in vivo. The generated data will inform the selection of the lead candidate AAV vector with the most efficient gRNA and the optimal viral dose that achieves the best efficacy.

### Example 7

### Evaluation of the Safety of the AAV/dCas9-Repressor Vector

Again, as set forth in Table 4, adult E4KI mice (4.5 months old) are divided randomly into 7 groups with each group having an equal number of males and females.

TABLE 4
Experiment Design

| | Treatment | Left DH | Right DH |
|---|---|---|---|
| (i) | Control | Saline | Saline |
| (ii) | Low Dose | AAV-gRNA1/ | AAV-gRNA1/dCas9-NO |
| | (2 × 10⁹ vg) | dCas9-repressor | repressor |
| (iii) | Medium Dose | AAV-gRNA1/ | AAV-gRNA1/dCas9-NO |
| | (1 × 10¹⁰ vg) | dCas9-repressor | repressor |
| (iv) | High Dose | AAV-gRNA1/ | AAV-gRNA1/dCas9-NO |
| | (5 × 10¹⁰ vg) | dCas9-repressor | repressor |
| (v) | Low Dose | AAV-gRNA2/ | AAV-gRNA2/dCas9-NO |
| | (2 × 10⁹ vg) | dCas9-repressor | repressor |
| (vi) | Medium Dose | AAV-gRNA2/ | AAV-gRNA2/dCas9-NO |
| | (1 × 10¹⁰ vg) | dCas9-repressor | repressor |
| (vii) | High Dose | AAV-gRNA2/ | AAV-gRNA2/dCas9-NO |
| | (5 × 10¹⁰ vg) | dCas9-repressor | repressor |

Safety of the AAV vectors is assessed in the mice according to the FDA's “Guidance for Industry Preclinical Assessment of Investigational Cellular and GT Products”. Briefly, the following safety assessments are used. First, mice survival is examined using the Kaplan-Meier analysis (Kantor B, et al. (2018). Mol Ther 26:2638-2649). Second, daily weights are obtained. Other signs and general mouse welfare (e.g., clinical signs of toxicity, aberrant food consumption, etc.) are observed and monitored. Third, the integration capacity of AAV vectors is examined. Integration events are detected by real-time PCR using genomic DNA (gDNA) from the transduced tissue as the input. Fourth, blood count. The complete blood count (CBC) test is performed on the samples derived from the seven (7) groups animals. Fifth, serum chemistry. At the end of this experiment, the mice are bled for chemistry/enzyme assay immediately before euthanasia. Liver function tests include that least these markers: (i) albumin, (ii)alpha-1 antitrypsin, (iii) alkaline phosphatase (ALP), (iv) alanine aminotransferase (ALT), (v) aspartate aminotransferase (AST), and (vi) gamma-glutamyl transpeptidase (GGT). Sixth, liver histology. Using various stains (e.g., H&E, Masson's trichome staining, Periodic acid-Shiff, etc.) and antibodies, liver histology of all groups (treatment and control) is examined. Seventh, biodistribution. Here, the presence/absence of the viral genomes and the biodistribution is determined by real-time qPCR. All major tissues and organs are examined including other brain regions (cerebellum and cerebrum cortex), liver, kidneys, spleen, lungs, heart, gut, and gonads. Organs are collected from each mouse immediately after euthanasia.

Statistical Analyses: For each safety measure, the effects of the vector injection are compared to that of the saline (control) group and to and between each viral dose (low, medium, and high) using a two-way ANOVA with a mixed model (e.g., GraphPad Prism 9.0). The Bonferroni and Tukey's post hoc tests are utilized for correction for multiple comparisons.

### Example 8

### Validation of the Specificity of the AAV-gRNA/dCas9-Repressor Vector

This experiment includes employs E3KI mice (4.5 months old). The left dorsal hippocampus (DH) of each mouse is treated with AAV-dCas9-repressor vector that contains the most efficient gRNA at the selected dose (as determined in experiment above). The right DH of each mouse is treated with a control vector AAV-dCas9-no repressor vector at the same dose. Mice stereotaxic surgeries and molecular analyses of APOE expression are performed.

Mice stereotaxic surgery: Adult mice, age 4.5 months, receive bilateral stereotaxic microinjections of the virus into the dorsal hippocampus (DH; 1.75 mm anterior, ±1.5 mm lateral, and 1.55 mm ventral). Post-operative monitoring occurs every day for 10 days after viral infusion. Each mouse is evaluated on the at least the following well-being criterions: (i) incision site, (ii) behavior and appearance, (iii) eating, (iv) drinking, (v) feces and urine, and (vi) whether additional analgesia or intervention is needed.

Molecular Analyses: Six weeks post-surgery, the experiment is terminated, and the brain tissues are harvested and used to perform molecular analyses. Analyses focus on the DH while the cerebellum serves as a control brain region. Measures of molecular phenotypes include: (i) human APOE-mRNA level as quantified by qRT-PCR using TaqMan assay, and (ii) human APOE protein expression as measured by Western Blot (WB) and ELISA.

Statistical Analyses: The effect of the most efficient AAV-gRNA/dCas9-repressor vector on the APOEe3 expression is determined by calculating for each mouse the normalized expression levels (mRNA or protein) in the left DH when compared to the right DH. The average of the left DH/right DH ratios of the mice within a group (mean±SEM) is calculated and the statistical significance of the expression change. The average for the left DH compared to that of the right DH is analyzed by Mann-Whitney U Test using GraphPad Prism 9.0.

Changes is APOE expression levels in the E3KI mice are not expected as this model express the human e3 allele. Thus, this experiment validates the specificity of the effect of the AAV-gRNA/dCas9-repressor vector to the e4 allele.

### Example 9

### In Vivo Validation of the Efficacy of the AAV Vector to Ameliorate AD-Neuropathological Endpoints

The AD-mouse model [3×Tg-AD (APP/PSEN1/MAPT)] (Javonillo D I, et al. (2021). Front Neurosci 15:785276; Oddo S, et al. (2003). Neuron 39:409-421) is crossed with the human-APOE e4 knock-in mouse (E4KI) to generate the E4KI/3×Tg-AD mice.

The 3×Tg-AD model is a popular animal model widely used within the AD research community. The model features three familial AD mutations: the Swedish APP mutation (KM670/671NL), the PSEN1 M146V mutation, and the MAPT P301L mutation. These mice exhibit development of age-related and progressive amyloid and tau pathologies with extracellular plaques first appearing at 6-months of age. Additionally, 3×Tg-AD mice display localized neurodegeneration, synaptic impairment, and cognitive deficits at 6 months of age (Drummond E, et al. (2017). Acta Neuropathol 133:155-175). Due to the attractive combination of both plaque and tangle development, the 3×Tg-AD mouse is considered a complete transgenic mouse model of AD pathology (Myers A, et al. (2019). Curr Protoc Neurosci 89:e81). Recently the 3×Tg-AD model was comprehensively characterized using the standardized phenotyping pipeline developed for MODEL-AD (Javonillo D I, et al. (2021). Front Neurosci 15:785276).

Here, adult E4KI/3×Tg-AD mice (4.5 months old) are treated as follows. The left DH of each mouse receives a stereotaxic injection AAV-gRNA/dCas9-repressor vector at a pre-determined selected dose (as informed by the experiments/data discussed above). The right DH of each mouse receives a control vector AAV-dCas9-no repressor vector (FIG. 25A).

Six weeks after surgery (at age of 6 months when AD pathologies are reported for the AD-model (Oddo S, et al. (2003). Neuron 39:409-421)), the mice are divided randomly into 2 groups. The first group of mice are evaluated for the efficacy of the lead candidate AAV-dCas9-repressor vector and dose in the AD-model using the same methods and analyses described below.

Specifically, the AAV vectors for the injections are quantified by real-time PCR (SYBR Green qPCR) method and by flow cytometry (to determine the functional titers) (Ortinski P I, et al. (2017). Molecular Therapy—Methods & Clinical Development 5:153-164).

Adult mice, age 4.5 months, receive bilateral stereotaxic microinjections of the virus into the dorsal hippocampus (DH; 1.75 mm anterior, ±1.5 mm lateral, and 1.55 mm ventral). AAV/dCas-repressor (1 μL) is injected into the left DH, and AAV/dCas-no repressor (1 μL) is injected into the right DH, providing a within animal control (FIG. 25A). Post-operative monitoring occurs every day for 10 days after viral infusion. Each mouse is evaluated on the at least the following well-being criterions: (i) incision site, (ii) behavior and appearance, (iii) eating, (iv) drinking, (v) feces and urine, and (vi) whether additional analgesia or intervention is needed.

Six weeks post-surgery, the experiment is terminated, and the brain tissues are harvested and used to perform molecular analyses. Analyses focus on the DH while the cerebellum serves as a control brain region. Measures of molecular phenotypes include: (i) human APOE-mRNA level as quantified by qRT-PCR using TaqMan assay, and (ii) human APOE protein expression as measured by Western Blot (WB) and ELISA.

The effect of the AAV-gRNA/dCas9-repressor vector on the APOE e4 expression is determined by calculating for each mouse the normalized expression levels (mRNA or protein) in the left DH when compared to the right DH. The average of the left DH/right DH ratios of the mice (mean±SEM) is calculated and the statistical significance of the expression change is determined. The average for the left DH compared to that of the right DH is analyzed by Mann-Whitney U Test using GraphPad Prism 9.0. The significance of the differences in APOE e4-mRNA and protein repression between the left DH and the right DH is analyzed using two-way ANOVA via GraphPad Prism 9.0. The Bonferroni and Tukey's post hoc tests are utilized for correction for multiple comparisons.

This experiment validates the specific reduction of the human APOE e4 expression in the AD disease model and confirms no changes in the expression of other genes. This experiment also validates the accuracy and specificity of the APOE-targeted system in vivo by evaluating off targets effects using whole genome transcriptomic analysis using RNA-seq to identify differential gene expression [log 2(FC)<10.21, adjusted p<0.05].

The second group of mice are evaluated for neuropathological endpoints according to the established AD-related pathologies for 3×Tg-AD model (Javonillo D I, et al. (2021). Front Neurosci 15:785276). The mice are perfused with 10% formalin and slices are prepared. Images are taken of 3 slices per mouse per marker and analyzed in ImageJ with the threshold function. All statistics are carried out in GraphPad Prism 9.0.

The levels of interleukin-6 (IL-6) and C-reactive protein are also assessed in the brain as a measure of inflammation. Both IL-6 and C-reactive protein are particularly relevant to AD neuropathology as levels are elevated in patients with AD compared to healthy controls (Lai K S P, et al. (2017). J Neurol Neurosurg Psychiatry 88:876-882).

A different Alzheimer's mouse model can also be used. The 5 FAD model (Oakley H, et al. (2006). J Neurosci 26:10129-10140; Forner S, et al. (2021). Sci Data 8:270) is also a popular AD model widely used within the AD research community. This APP/PS1 double transgenic mice co-express five FAD mutations (5×FAD mice), additively increase Aβ42 production, and rapidly accumulate massive cerebral Aβ42 levels.

### Example 10

### Additional Experiments

In these Examples, the therapeutic target is APOE e4. The mechanism of action this approach is the downregulation of APOE expression precisely and in e4 allele-specific manner by epigenome editing. Here, the human APOE e4 allele is the target and the human APOE e3 allele serves as the control.

A knock-in mouse model is used. Specifically, the endogenous murine Apoe gene is replaced with either the human APOE e4 allele (B6.129P2-Apoe^{tm3(APOE*4)Mae} N8 (hereafter E4KI)) or the human APOE e3 allele (B6.129P2-Apoe^{tm3(APOE*4)Mae} N8 (hereafter, E3KI)) (Sullivan P M, et al. (1997) J Biol Chem. 272(29):17972-17980). The E4KI express only the human APOE e4 gene while the E3KI mice express only the APOE e3 gene. These models do not express the mouse Apoe. The genetic background of the mice is C57BL/6. These humanized APOE models, E4KI and E3KI, which were developed in 1997 (Sullivan P M, et al. (1997) J Biol Chem. 272(29):17972-17980), have been extensively characterized and utilized in numerous publications of basic research and pre-clinical AD studies. (Luz I, et al. (2016) Curr Alzheimer Res. 13(8):918-929; Liao F, et al. (2018) J Clin Invest. 128(5):2144-2155).

3×Tg-AD mice contain three mutations associated with familial Alzheimer's disease (APP Swedish, MAPT P301L, and PSEN1 M146V). This model has been extensively characterized and is widely used in AD research. These mice develop age-related, progressive neuropathology including extracellular Aβ deposits, tau pathology, and synaptic dysfunction. (Oddo S, et al. (2003) Neuron. 39(3):409-421). The crossing of 3×Tg-AD mice with humanized APOE e4 mice (E4KI) affords the exploration of the effect that reducing human APOE e4 repression on the neuropathology phenotypes associated with AD. Genotypes are confirmed by TaqMan based assays and sequencing analysis. Neuropathological phenotypes are confirmed by immunofluorescence staining of the mice brain sections using markers followed by microscopy analysis.

In this study, the AAV vector is microinjected directly into the dorsal hippocampus, ensuring the target site is reached. Preliminary data shows the repression of GFP reporter with AAV-dCas-repressor vector, which indicates that the AAV vector successfully reaches the target in sufficient concentration to be effective. With this experiment, the different AAV (e.g., 2 different gRNAs) constructs are examined at 3 vector titers for safety and efficacy.

The primary evaluation criteria for efficacy are APOE mRNA and protein levels. In humans, carrying the APOE e4 variant significantly increases the lifetime risk for LOAD (Corder E H, et al. (1993) Science. 261(5123):921-923), and higher APOE expression is implicated in the etiology of LOAD (reviewed in Gottschalk W K, et al. (2016) J Alzheimers Dis Parkinsonism. 6(1):209; Yang A, et al. (2021) Int J Mol Sci. 22(3):1244). Therefore, the effective repression of APOE e4 expression is a promising target for AD therapy.

For assessment of safety, viral toxicity is evaluated by measuring mice survival, daily weights, and other signs of a general mouse welfare. Upon the endpoint of the experiment, measurements of blood counts, serum chemistry (including, ALP, ALT and AST), liver histology, inflammation markers, and viral biodistribution to other brain regions and other organs are examined and recorded.

For these experiments, the evaluation criteria are APOE RNA and protein levels and AD-neuropathological endpoints including intraneuronal phosphorylated-tau/total-tau, secreted Aβ42/40, neuronal degeneration, and microglia activation. The pathogenic effect APOE e4 is implicated in several cellular pathways associated with AD pathologic phenotypes. (Huang Y A, et al. (2017) Cell. 168(3):427-441 e21; Sen A, et al. (2015) J Neurosci. 35(19):7538-7351; Theendakara V, et al. (2013) Proc Natl Acad Sci USA. 110(45):18303-18308; Min S W, et al. (2010) Neuron. 67(6):953-966; Tambini M D, et al. (2016) EMBO Rep. 17(1):27-36; Hatters D M, et al. (2006) J Mol Biol. 361(5):932-944).

Gene editing studies in hiPSC models have shown that conversion of e4/4 with e3/3 can rescue phenotypes such as higher tau phosphorylation, Aβ production, and GABAergic neuron degeneration (reviewed in Yang A, et al. (2021) Int J Mol Sci. 22(3):1244). This aim will allow us to investigate the impact of APOE e4 repression on AD-neuropathological endpoints that are relevant to the human condition.

The pharmacodynamic readout of target engagement is the level of APOE expression. The experimental design employs bilateral microinjection—meaning that the repressor construct is delivered to one hemisphere. This approach allows for each animal to serve as its own control. APOE mRNA and protein levels are measured and are compared between hemispheres to assess the efficacy of the AAV/dCas-repressor to decrease APOE expression at low, mid, and high titers.

The humanized APOE e4 mouse model expresses the human APOE e4 allele, which allows the targeting of the human gene with the AAV/Cas-repressor construct. As described above, APOE expression is a well-established risk factor for LOAD and a promising clinical target. Elevated levels of APOE expression in LOAD brains, and the beneficial effects of reducing its levels on AD-related pathological and behavioral outcomes have been reviewed (Gottschalk W K, et al. (2016) J Alzheimers Dis Parkinsonism. 6(1):209; Yang A, et al. (2021) Int J Mol Sci. 22(3):1244). This makes this therapy highly translatable to human clinical studies.

The method used to calculate the sample size was previously described (Whitley E, et al. (2002) Crit Care. 6:335-341). Specifically, for data analysis, power calculations of the number of animals needed to achieve statistical significance of p=0.05 at 80% power, revealed that the experiments require a sample size of 12 mice per group (6 males and 6 females) to provide sufficient statistical power. The significance of the differences between the hemispheres injected with the AAV/dCas-repressor construct or the AAV/dCas-no repressor-no gRNA construct as well as other treatment groups are analyzed using the following comparisons tests (GraphPad Prism9): (i) the Student's t-test; (ii) the Mann-Whitney U Test; and (iii) the Two-way ANOVA with multiple comparisons using Dunnett's method or Tukey-Kramer HSD, as applicable.

The experimental design, procedures, and related animal protocols are consistently applied to all animals in the study. Researchers are bling to the experimental grouping of the animals. Each experimental cohort contains age matched animals that are treated with a placebo to control for inter-animal variability. The experimental design for this experiment is set forth below in Table 5. Using 4.5-month-old mice, bilateral injections into the dorsal hippocampus are performed.

TABLE 5
Experimental Treatment Groups

| | Left Hemisphere | Right Hemisphere |
|---|---|---|
| hAPOE e4 Mice | AAV-gRNA1- | AAV-gRNA1- |
| | dCas9-repressor | dCas9-NO repressor |
| hAPOE e4 Mice | AAV-gRNA2- | AAV-gRNA2- |
| | dCas9-repressor | dCas9-NO repressor |
| Control Mice | Saline | Saline |
| hAPOE e3 Mice | AAV-dCas9- | AAV-dCas9-NO |
| | repressor | repressor |

Welfare monitoring of the mice is performed for 6 weeks until animals are sacrificed. Hippocampal levels are APOE mRNA and proteins are measured, and the plasma is examined for inflammatory markers. In this experiment, 4.5-month-old hAPOE e4×3×TG-AD mice receive bilateral injections in the dorsal hippocampus (Table 6).

TABLE 6
Experimental Treatment Groups

| | Left Hemisphere | Right Hemisphere |
|---|---|---|
| hAPOE e4 × 3xTG-AD Mice | AAV-dCas9- | AAV-dCas9-NO |
| | repressor | repressor |
| hAPOE e4 × 3xTG-AD Mice | Saline | Saline |

In the left hemisphere, AAV-dCas9-repressor is injected. In the right hemisphere, AAV-dCas9-NO-repressor is injected. After 6 weeks of welfare monitoring, the hippocampus is retrieved and APOE RNA and proteins levels are measured. Other AD-neuropathological endpoints are examined, and a transcriptomics analysis is performed to identify off-target changes.

Moreover, neuropathological endpoints are examined in the mice according to the established AD-related pathologies for the 3×Tg-AD model. Specifically, hippocampal regions are examined for the following neuropathological phenotypes: (i) Aβ42, Aβ40 levels and Aβ42/40 ratio, (ii) phosphorylated-Tau species (p-tau) and total-Tau (t-tau) levels and the p-tau/t-tau ration, (iii) neuronal degeneration, (iv) gliosis-microglial activation, and (v) gliosis-density of reactive astrocytes. Biochemical analysis and immunofluorescent staining are used.

For the biochemical analysis, mice are euthanized by cardiac puncture blood collection and the hippocampal region micro-dissected and flash-frozen. Once homogenized in Tissue Protein Extraction Reagent, the homogenate is centrifuged to separate into soluble and insoluble factions. The Aβ42/40 ratio is assessed in the soluble fraction using the Human Amyloid 040 and Amyloid 042 Brain ELISA kit. Phosphorylated-tau/total-tau in the soluble fraction is quantified using the Total Tau and Tau (Phospho) [pT231] Human ELISA Kits.

For the immunofluorescent staining, mice are transcardially perfused with 4% formalin, brains are removed, and coronal hippocampal sections (100 μm) are prepared. Brain sections are treated with primary antibodies to evaluate the expression and localization of Aβ, tau, and phosphorylated tau: Aβ1-16, human tau, poly-tau, phospho-tau Ser202, Thr 205, phospho-tau Thr217. To assess microglial activation, sections are stained for ionized calcium-binding adapter molecule 1 (IBA1). To assess gliosis, the densities and distribution of reactive and homeostatic astrocytes are characterized by immunostaining with the markers GFAP and S1000, respectively. Neuronal degeneration is measured with immunohistochemical analysis of the specific neuronal marker NeuN to assess the number of neurons in the hippocampus. Immunostained sections are mounted on slides and visualized with the Keyence All-in-One Fluorescence Microscope BZ-X700 to get high resolution images with 2× and 20× objectives. Images are taken of 3 slices per mouse per marker and analyzed in ImageJ with the threshold function.

Statistical Analyses: The experimental design of bilateral microinjection, with the repressor only present in one hemisphere, allows for within animal controls. All measurements are compared between hemispheres to assess the effect of APOEe4 repression on neuropathological phenotypes. The significance is analyzed using the Mann-Whitney U Test. The Bonferroni and Tukey's post hoc tests are utilized for correction for multiple comparisons. All statistics are carried out in GraphPad Prism 9.0.

### SUMMARY OF EXPERIMENTS

As described herein, an all-in-one CRISPR/dCas9 system for efficient AAV packaging was developed. First, the AAV backbone was optimized for high packaging and delivery efficiencies of the CRISPR/Cas9 components. Second, this all-in-one vector was suitable for a broad range of cellular tropisms. Third, the all-in-one vector demonstrated low cytotoxicity and immunogenicity. Fourth, the all-in-one vector demonstrated long-term and sustainable expression to ensure the durability of the epigenetic changes. The development of this robust epigenetic editing tool enables novel architectures for synthetic gene circuits and potential safe gene therapy for a number of diseases.

What is claimed is:

1. An isolated nucleic acid molecule, comprising: a nucleic acid sequence encoding (i) a deactivated Cas9 (dCas9) endonuclease, (ii) at least one polypeptide having an enzymatic activity, and (iii) at least one guide RNA targeting a gene of interest or portion thereof.

2. The isolated nucleic acid molecule of claim 1, wherein the dCas9 endonuclease comprises a deactivated Staphylococcus aureus Cas9 (dSaCas9), a deactivated Streptococcus pyogenes Cas9 (dSpCas9), a deactivated Campylobacter jejuni Cas9 (dCjCas9), or a variant dCas9 endonuclease.

3. The isolated nucleic acid molecule of claim 2, wherein the dSaCas9 comprises the sequence set forth in SEQ ID NO:24 or a fragment thereof.

4. The isolated nucleic acid molecule of claim 2, wherein the dCjCas9 comprises the sequence set forth in SEQ ID NO:25, SEQ ID NO:26, or a fragment thereof.

5. The isolated nucleic acid molecule of claim 1, wherein the enzymatic activity comprises transcription activation activity, transcription repression activity, transcription release factor activity, histone modification activity, nucleic acid association activity, methyltransferase activity, demethylase activity, acetyltransferase activity, deacetylase activity, or any combination thereof

6. The isolated nucleic acid molecule of claim 1, wherein the at least one encoded polypeptide comprises HP1a, HP1b, MBD1, MBD2, Krüppel-Associated Box (KRAB), NIPP1, Methyl-CpG Binding Protein 2 (MeCP2), DNMT3A, any combination thereof, or any fusion thereof.

7. The isolated nucleic acid molecule of claim 6, wherein the fusion comprises KRAB-MeCP2 (KRAB-MeCP2).

8. The isolated nucleic acid molecule of claim 7, wherein the KRAB-MeCP2 comprises the sequence set forth in SEQ ID NO:58, SEQ ID NO:59, or a fragment thereof

9. The isolated nucleic acid molecule of claim 1, wherein the at least one gRNA targets the APOE gene or the promoter of the APOE gene.

10. The isolated nucleic acid molecule of claim 1, wherein the at least one gRNA targets the SNCA gene or the promoter of the SNCA gene.

11. A recombinant adenovirus-associated virus (rAAV) vector comprising the isolated nucleic acid molecule of any one of claims 1-10.

12. The rAAV vector of claim 11, further comprising one or more promoters operably linked to the isolated nucleic acid molecule.

13. The rAAV vector of claim 12, wherein the one or more promoters is operably linked to the dCas9 endonuclease.

14. The rAAV vector of claim 13, wherein the one or more promoters operably linked to the dCas9 endonuclease comprises an EF-1a promoter.

15. The rAAV vector of claim 13, wherein the one or more promoters operably linked to the dCas9 endonuclease is operably linked to the at least one polypeptide having enzymatic activity.

16. The rAAV vector of claim 13, wherein the one or more promoters is operably linked to the at least one guide RNA targeting a gene of interest or portion thereof.

17. The rAAV vector of claim 16, wherein the one or more promoters operably linked to the at least one guide RNA comprises a U6 promoter.

18. The rAAV vector of claim 12, further comprising one or more regulatory elements.

19. A pharmaceutical formulation comprising the isolated nucleic acid molecule of any one of claims 1-10 or the rAAV vector of any one of claims 11-18, and pharmaceutically acceptable carrier.

20. A method of effecting precision epigenetic modulation, the method comprising:
contacting one or more cells with a therapeutically effective amount of the isolated nucleic acid molecule of any one of claims 1-10 or the rAAV vector of any one of claims 11-18,
wherein the expression and/or activity of one or more genes of interest in the one or more cells is modulated.

21. The method of claim 20, wherein modulating comprises increasing or augmenting the expression and/or activity of the one or more genes of interest.

22. The method of claim 21, wherein the enzymatic activity comprises transcription activation activity.

23. The method of claim 22, wherein the gene of interest or portion thereof demonstrates a loss of function or is characterized by a loss of function.

24. The method of any claim 20, wherein modulating comprises decreasing or reducing the expression and/or activity of the one or more genes of interest.

25. The method of any claim 20, wherein the cells are in a subject.

26. The method of claim 25, wherein the subject is suspected of having or has been diagnosed with having Alzheimer's disease.

27. The method of claim 26, further comprising reducing the pathological phenotype associated with Alzheimer's disease.

28. The method of claim 26, wherein the gene of interest or portion thereof comprises APOE or the promoter region of APOE.

29. The method of claim 28, further comprising measuring the expression and/or activity level of APOE.

30. The method of claim 29, wherein the expression and/or activity level of APOE is decreased or reduced when compared to a pre-contacting step level.

31. The method of claim 25, wherein the subject is suspected of having or has been diagnosed with having Parkinson's disease.

32. The method of claim 31, further comprising reducing the pathological phenotype associated with Parkinson's disease.

33. The method of claim 31, wherein the gene of interest or portion thereof comprises SCNA or the promoter region of SCNA.

34. The method of claim 33, wherein the expression and/or activity level of SCNA is decreased or reduced when compared to a pre-contacting step level.

35. The method of claim 25, wherein contacting the one or more cells in a subject comprises administering the rAAV vector through intravenous administration, intracerebral administration, intra-CSF administration, intracerebroventricular (ICV) administration, intraventricular administration, intra-cisterna magna (ICM) administration, intraparenchymal administration, intrathecal (lumbar, cisternal, or both) administration, or any combination thereof.

36. The method of claim 20, further comprising administering to the subject a therapeutically effective amount of one or more immune modulators.

37. The method of claim 36, wherein the one or more immune modulators comprise methotrexate, rituximab, intravenous gamma globulin, Tacrolimus, SVP-Rapamycin, bortezomib, or a combination thereof.

38. A method of treating and/or preventing Alzheimer's disease progression in a subject, the method comprising:
administering to a subject in need thereof a therapeutically effective amount of the rAAV vector of any one of claims 11-18, thereby reducing the pathological phenotype associated with Alzheimer's disease.

39. The method of claim 38, wherein administering the rAAV vector comprises intravenous administration, intracerebral administration, intra-CSF administration, intracerebroventricular (ICV) administration, intraventricular administration, intra-cisterna magna (ICM) administration, intraparenchymal administration, intrathecal (lumbar, cisternal, or both) administration, or any combination thereof.

40. The method of claim 38, further comprising administering to the subject a therapeutically effective amount of a therapeutic agent.

41. The method of claim 38, further comprising administering to the subject a therapeutically effective amount of the one or more immune modulators.

42. The method of claim 41, wherein the one or more immune modulators comprise methotrexate, rituximab, intravenous gamma globulin, Tacrolimus, SVP-Rapamycin, bortezomib, or a combination thereof.
